(19) United States
(12) Patent Application Publication
(10) Pub. No.: US 2023/0257365 A1
(43) Pub. Date: Aug. 17, 2023

(54) SMALL MOLECULE ANDROGEN RECEPTOR PROTEIN DEGRADERS

(71) Applicant: THE REGENTS OF THE UNIVERSITY OF MICHIGAN, Ann Arbor, MI (US)

(72) Inventors: Shaomeng Wang, Superior Township, MI (US); Xin Han, Ann Arbor, MI (US); Weiguo Xiang, Ypsilanti, MI (US); Chong Qin, Ann Arbor, MI (US); Tianfeng Xu, Ypsilanti, MI (US); Lijie Zhao, Ann Arbor, MI (US)

(21) Appl. No.: 18/012,369

(22) PCT Filed: Jul. 9, 2021
(86) PCT No.: PCT/US21/40988

§ 371 Date: Dec. 22, 2022

Related U.S. Application Data
(60) Provisional application No. 63/166,059, filed on Mar. 25, 2021.
(60) Provisional application No. 63/050,233, filed on Jul. 10, 2020.

(51) Int. Cl. C07D 401/14; C07D 451/06; C07D 451/14; C07D 519/00; C07D 487/04; C07D 487/10; C07D 471/04; C07D 491/08

(52) U.S. Cl. CPC C07D 401/14; C07D 451/06; C07D 451/14; C07D 519/00; C07D 487/04; C07D 487/10; C07D 471/04; C07D 491/08

(57) ABSTRACT

The present disclosure provides compounds represented by Formula I: A-L-B¹ I, and the salts or solvates thereof, wherein A, L, and B1 are as defined in the specification. Compounds having Formula I are androgen receptor degraders useful for the treatment of cancer and other diseases.

### GOVERNMENT SUPPORT

This invention was made with government support under CA186786 awarded by the National Institutes of Health. The government has certain rights in the invention.

### BACKGROUND OF THE INVENTION

### Field of the Invention

The present disclosure provides heterobifunctional small molecules as androgen receptor (AR) protein degraders. AR degraders are useful for the treatment of a variety of diseases including cancer.

### Background

Despite improvements in medical treatments over the past three decades, prostate cancer is significant cause of cancer-related death, and is second only to lung cancer among men in developed countries. Hamdy et al., N Engl J Med, 2016, 375, 1415-1424; Litwin and Tan, H. J. JAMA, 2017, 317, 2532-2542. In addition to surgery and radiotherapy, androgen deprivation therapies (ADT) are front-line treatments for prostate cancer patients with high-risk localized disease, and second-generation anti-androgens such as abiraterone and enzalutamide have been shown to benefit patients with advanced prostate cancer. Karantanos et al., Oncogene. 2013, 32, 5501-511; Harris et al., Nat Clin Pract Urol, 2009, 6, 76-85. Nevertheless, patients who progress to metastatic castration-resistant prostate cancer (mCRPC), a hormone-refractory form of the disease, face a high mortality rate and no cure is currently available. Narayanan et al., Oncoscience. 2017, 4, 175-177; Crowder et al., Endocrinology. 2018, 159, 980-993.

The androgen receptor (AR) and its downstream signaling play a critical role in the development and progression of both localized and metastatic prostate cancer. Previous strategies that successfully target AR signaling have focused on blocking androgen synthesis by drugs such as abiraterone and inhibition of AR function by AR antagonists such as enzalutamide and apalutamide (ARN-509). Watson et al., Nat Rev Cancer. 2015, 15, 701-711. However, such agents become ineffective in advanced prostate cancer with AR gene amplification, mutation, and alternate splicing. Balbas et al., Elife. 2013, 2, e00499; Lottrup et al., J Clin Endocrinol Metab. 2013, 98, 2223-2229. But in most patients with CRPC, the AR protein continues to be expressed and tumors are still dependent upon AR signaling. Consequently, AR is an attractive therapeutic target for mCRPC. Zhu et al., Nat Commun. 2018, 9, 500; Munuganti et al., Chem Biol. 2014, 21, 1476-485.

The Proteolysis Targeting Chimera (PROTAC) strategy has gained momentum with its promise in the discovery and development of completely new types of small molecule therapeutics by inducing targeted protein degradation. Raina et al., Proc Natl Acad Sci USA. 2016, 113, 7124-7129; Zhou et al., J. Med. Chem. 2018, 61, 462-481.

A PROTAC molecule is a heterobifunctional small molecule containing one ligand, which binds to the target protein of interest, and a second ligand for an E3 ligase system, tethered together by a chemical linker. Bondeson, D. P.; Crews, C. M. Targeted Protein Degradation by Small Molecules. Annu Rev Pharmacol Toxicol. 2017, 57, 107-123. Because AR protein plays a key role in CRPC, AR degraders designed based upon the PROTAC concept could be effective for the treatment of CRPC when the disease becomes resistant to AR antagonists or to androgen synthesis inhibitors. Salami et al., Commun Biol. 2018, 1, 100; Pal et al., Cancer. 2018, 124, 1216-1224; Wang et al., Clin Cancer Res. 2018, 24, 708-723; Gustafson et al., Angew. Chem. Int. Ed. 2015, 54, 9659-9662. Naito et al. have recently reported AR degraders designed based upon the PROTAC concept, which were named Specific and Nongenetic IAP-dependent Protein Erasers (SNIPERs). Shibata et al., J. Med. Chem. 2018, 61, 543-575.

While SNIPER AR degraders are effective in inducing partial degradation of the AR protein in cells, they also induce the auto-ubiquitylation and proteasomal degradation of the cIAP1 protein, the E3 ligase needed for induced degradation of AR protein, thus limiting their AR degradation efficiency and therapeutic efficacy.

(4R)-1-((S)-2-(2-(4-((4′-(3-(4-cyano-3-(trifluoromethyl)phenyl)-5,5-dimethyl-4-oxo-2-thioxoimidazolidin-1-yl)-[1,1′-biphenyl]-4-yl)oxy)butoxy)acetamido)-3,3-dimethylbutanoyl)-4-hydroxy-N-(4-(4-methylthiazol-5-yl)benzyl)pyrrolidine-2-carboxamide ((ARCC-4) was recently reported as another PROTAC degrader, which was designed using enzalutamide as the AR antagonist and a von Hippel-Lindau (VHL) ligand. Salami et al., Commun Biol. 2018, 1, 100; US 20170327469. ARCC-4 was shown to be more potent and effective than enzalutamide at inducing apoptosis and inhibiting proliferation of AR-amplified prostate cancer cells. ARD-69 was also recently reported as a PROTAC AR degrader. Han et al., J. Med. Chem. 62:941-964 (2019).

There is a need in the art for additional AR degraders to treat prostate cancer and other diseases.

### BRIEF SUMMARY OF THE INVENTION

In one aspect, the present disclosure provides heterobifunctional small molecules represented by Formula I, below, and the pharmaceutically acceptable salts and solvates, e.g., hydrates, thereof. These compounds, and the salts and solvates thereof are collectively referred to herein as “Compounds of the Disclosure.” Compounds of the Disclosure are androgen receptor (AR) degraders and are thus useful in treating diseases or conditions wherein degradation of the androgen receptor protein provides a therapeutic benefit to a subject.

In another aspect, the present disclosure provides methods of treating a condition or disease by administering a therapeutically effective amount of a Compound of the Disclosure to a subject, e.g., a human cancer patient, in need thereof. The disease or condition treatable by degradation of the androgen receptor is, for example, a cancer, e.g., prostate cancer, e.g., metastatic castration-resistant prostate cancer.

In another aspect, the present disclosure provides a method of degrading, e.g., reducing the level of, of androgen receptor protein in a subject in need thereof, comprising administering to the individual an effective amount of at least one Compound of the Disclosure.

In another aspect, the present disclosure provides a pharmaceutical composition comprising a Compound of the Disclosure and an excipient and/or pharmaceutically acceptable carrier.

In another aspect, the present disclosure provides a composition comprising a Compound of the Disclosure and an excipient and/or pharmaceutically acceptable carrier for use treating diseases or conditions wherein degradation of the androgen receptor provides a benefit, e.g., cancer.

In another aspect, the present disclosure provides a composition comprising: (a) a Compound of the Disclosure; (b) a second therapeutically active agent; and (c) optionally an excipient and/or pharmaceutically acceptable carrier.

In another aspect, the present disclosure provides a Compound of the Disclosure for use in treatment of a disease or condition of interest, e.g., cancer.

In another aspect, the present disclosure provides a use of a Compound of the Disclosure for the manufacture of a medicament for treating a disease or condition of interest, e.g., cancer.

In another aspect, the present disclosure provides a kit comprising a Compound of the Disclosure, and, optionally, a packaged composition comprising a second therapeutic agent useful in the treatment of a disease or condition of interest, and a package insert containing directions for use in the treatment of a disease or condition, e.g., cancer.

In another aspect, the present disclosure provides methods of preparing Compounds of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims. It is to be understood that both the foregoing summary and the following detailed description are exemplary and explanatory only, and are not restrictive of the invention as claimed.

### DETAILED DESCRIPTION OF THE INVENTION

### I. Compounds of the Disclosure

Compounds of the Disclosure are heterobifunctional AR degraders. In one embodiment, Compounds of the Disclosure are compounds of Formula I:

A-L-B¹ I,

or a pharmaceutically acceptable salt or solvate thereof, wherein:

A is selected from the group consisting of:

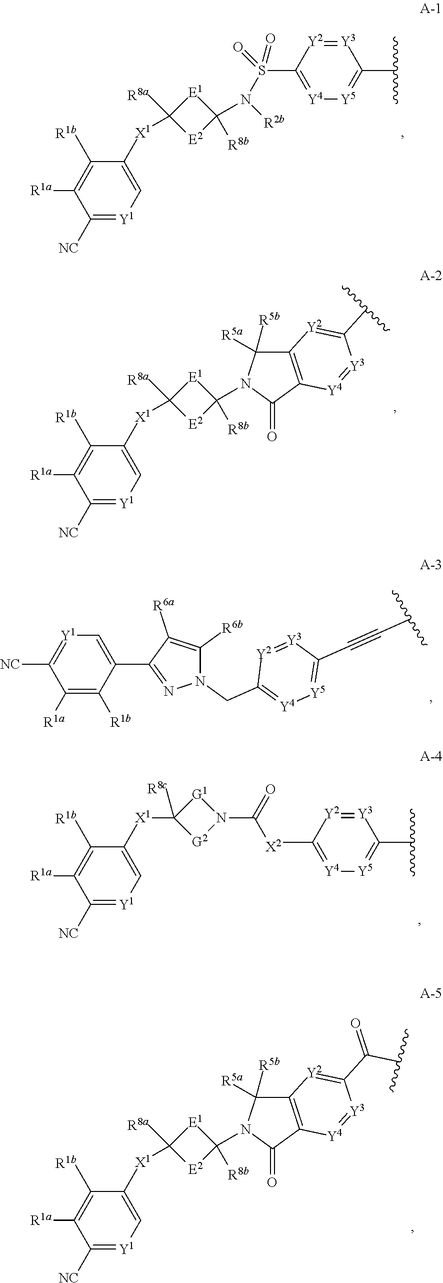

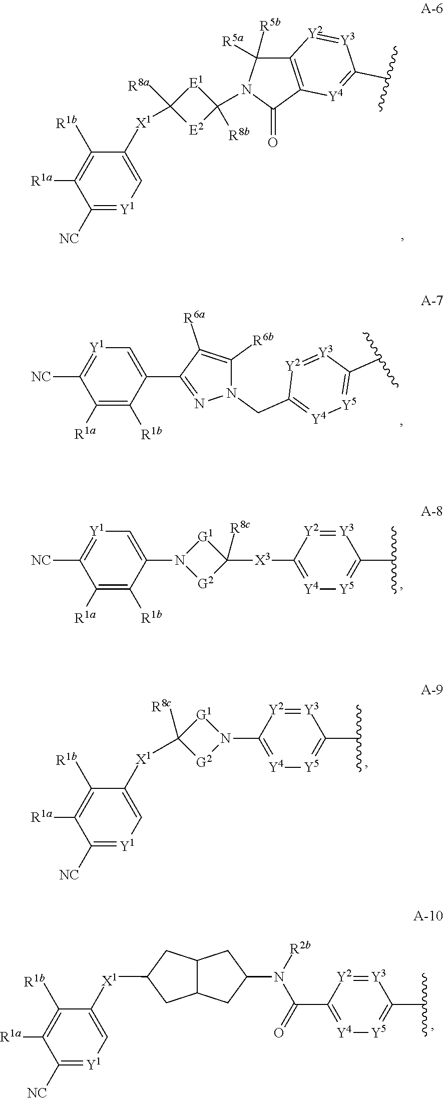

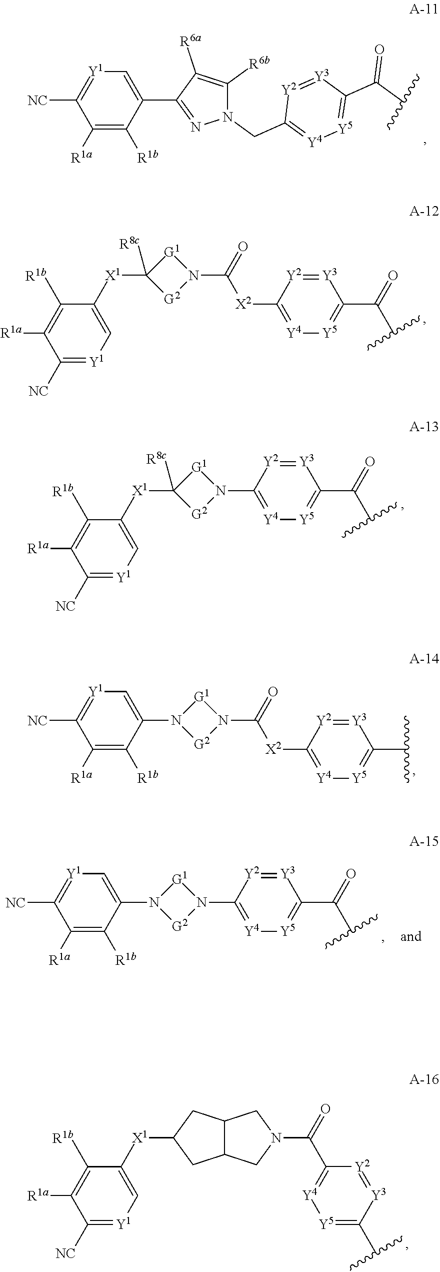

Y¹ is selected from the group consisting of —C(R^{1c})═ and —N═;

R^{1a}, R^{1b}, and R^{1c} are independently selected from the group consisting of hydrogen, halo, C₁-C₃ alkyl, and C₁-C₃ haloalkyl;

X¹ is selected from the group consisting of —O— and —N(R^{2a})—;

R^{2a} and R^{2b} are independently selected from the group consisting of hydrogen, C₁-C₄ alkyl, and C₃-C₆ cycloalkyl;

E¹ is —(CR^{3a}R^{3b})ₐ—;

E² is —(CR^{3c}R^{3d})_{b}—;

a and b are independently 1, 2, or 3;

each R^{3a}, R^{3b}, R^{3c}, and R^{3d} is independently selected from the group consisting of hydrogen and C₁-C₃ alkyl;

Y² is selected from the group consisting of —C(R^{4a})═ and —N═;

Y³ is selected from the group consisting of —C(R^{4b})═ and —N═;

Y⁴ is selected from the group consisting of —C(R^{4c})═ and —N═;

Y⁵ is selected from the group consisting of —C(R^{4d})═ and —N═;

R^{4a}, R^{4b}, R^{4c}, and R^{4d} are independently selected from the group consisting of hydrogen, halo, C₁-C₃ alkyl, C₁-C₃ haloalkyl, and C₁-C₃ alkoxy;

R^{8a} and R^{8b} are independently selected from the group consisting of hydrogen and C₁-C₃ alkyl; or R^{8a} and R^{8b} taken together form a C₁-C₃ alkylenyl;

R^{5a} and R^{5b} are independently selected from the group consisting of hydrogen and C₁-C₃ alkyl;

R^{6a} and R^{6b} are independently selected from the group consisting of hydrogen and C₁-C₃ alkyl; or R^{6a} and R^{6b} taken together with the carbon atoms to which they are attached from a C₅-C₇ cycloalkyl;

G¹ is —(CR^{7a}R^{7b})_{f}—;

G² is —(CR^{7c}R^{7d})_{g}—;

each R^{7a}, R^{7b}, R^{7c}, and R^{7d} is independently selected from the group consisting of hydrogen and C₁-C₃ alkyl; or one of R^{7a} and one of R^{7c} taken together with the carbon atoms to which they are attached form a C₁-C₃ alkylenyl or C₁-C₃ heteroalkylenyl; or one of R^{7a} and one of R^{7b} taken together with the carbon atom to which they are attached form a C₃-C₆ cycloalkyl;

f and g are independently 1, 2, or 3;

X² is selected from the group consisting of —O— and —N(R^{2c})—; or X² is absent, i.e., X² is a bond;

R^{2c} is selected from the group consisting of hydrogen, C₁-C₄ alkyl, and C₃-C₆ cycloalkyl

R^{8c} is selected from the group consisting of hydrogen and C₁-C₃ alkyl;

X³ is selected from the group consisting of —O— and —N(R^{2b})—;

L is -J¹-J²-J³-J⁴-J⁵-,

wherein J¹ is attached to A;

J¹ is selected from the group consisting of alkylenyl, cycloalkylenyl and heterocyclenyl; or

J¹ is absent;

J² is selected from the group consisting of —C(═O)—, —C(═O)NH—, —(CH₂)ₒ—, —CH═CH—, and —C≡C—;

o is 0, 1, 2, or 3;

J³ is selected from the group consisting of alkylenyl, heteroalkylenyl, cycloalkylenyl, heterocyclenyl, phenylenyl, and heteroarylenyl; or

J³ is absent;

J⁴ is selected from the group consisting of alkylenyl, cycloalkylenyl, and heterocyclenyl; or

J⁴ is absent;

J⁵ is selected from the group consisting of —C≡C—, —(CH₂)ₚ—, —O—, —N(R¹⁰)—, and —C(═O)—;

p is 0, 1, 2, or 3;

R¹⁰ is selected from the group consisting of hydrogen and C₁-C₃ alkyl;

B¹ is selected from the group consisting of:

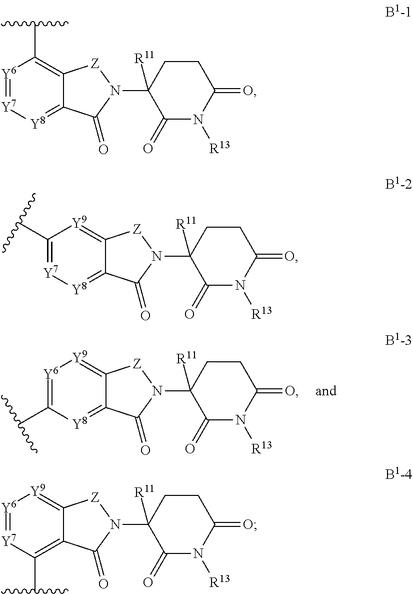

Y⁶ is selected from the group consisting of —C(R^{10a})═ and —N═;

Y⁷ is selected from the group consisting of —C(R^{10b})═ and —N═;

Y⁸ is selected from the group consisting of —C(R^{10c})═ and —N═;

Y⁹ is selected from the group consisting of —C(R^{10d})═ and —N═;

R^{10a}, R^{10b}, R^{10c}, and R^{10d} are independently selected from the group consisting of hydrogen, halo, C₁-C₃ alkyl, C₁-C₃ haloalkyl, and C₁-C₃ alkoxy;

R¹¹ is selected from the group consisting of hydrogen, deuterium, fluoro, and C₁-C₃alkyl;

Z is selected from the group consisting of —CR^{12a}R^{12b}— and —C(═O)—;

Z¹ is —CR^{12a}R^{12b}—;

R^{12a} and R^{12b} are independently selected from the group consisting of hydrogen and C₁-C₃ alkyl; or R^{12a} and R^{12b} taken together with the carbon to which they are attached from a C₃-C₆ cycloalkyl;

R¹³ is selected from the group consisting of hydrogen and C₁-C₃ alkyl.

In another embodiment, Compounds of the Disclosure are compounds of Formula I, or a pharmaceutically acceptable salt or solvate thereof, wherein A is A-1.

In another embodiment, Compounds of the Disclosure are compounds of Formula I, or a pharmaceutically acceptable salt or solvate thereof, wherein A is A-2.

In another embodiment, Compounds of the Disclosure are compounds of Formula I, or a pharmaceutically acceptable salt or solvate thereof, wherein A is A-3.

In another embodiment, Compounds of the Disclosure are compounds of Formula I, or a pharmaceutically acceptable salt or solvate thereof, wherein A is A-4.

In another embodiment, Compounds of the Disclosure are compounds of Formula I, or a pharmaceutically acceptable salt or solvate thereof, wherein A is A-5.

In another embodiment, Compounds of the Disclosure are compounds of Formula I, or a pharmaceutically acceptable salt or solvate thereof, wherein A is A-6.

In another embodiment, Compounds of the Disclosure are compounds of Formula I, or a pharmaceutically acceptable salt or solvate thereof, wherein A is A-7.

In another embodiment, Compounds of the Disclosure are compounds of Formula I, or a pharmaceutically acceptable salt or solvate thereof, wherein A is A-8.

In another embodiment, Compounds of the Disclosure are compounds of Formula I, or a pharmaceutically acceptable salt or solvate thereof, wherein A is A-9.

In another embodiment, Compounds of the Disclosure are compounds of Formula I, or a pharmaceutically acceptable salt or solvate thereof, wherein A is A-10.

In another embodiment, Compounds of the Disclosure are compounds of Formula I, or a pharmaceutically acceptable salt or solvate thereof, wherein A is A-11.

In another embodiment, Compounds of the Disclosure are compounds of Formula I, or a pharmaceutically acceptable salt or solvate thereof, wherein A is A-12.

In another embodiment, Compounds of the Disclosure are compounds of Formula I, or a pharmaceutically acceptable salt or solvate thereof, wherein A is A-13.

In another embodiment, Compounds of the Disclosure are compounds of Formula I, or a pharmaceutically acceptable salt or solvate thereof, wherein A is A-14.

In another embodiment, Compounds of the Disclosure are compounds of Formula I, or a pharmaceutically acceptable salt or solvate thereof, wherein A is A-15.

In another embodiment, Compounds of the Disclosure are compounds of Formula I, or a pharmaceutically acceptable salt or solvate thereof, wherein A is A-16.

In another embodiment, Compounds of the Disclosure are compounds of Formula I, or a pharmaceutically acceptable salt or solvate thereof, wherein A is:

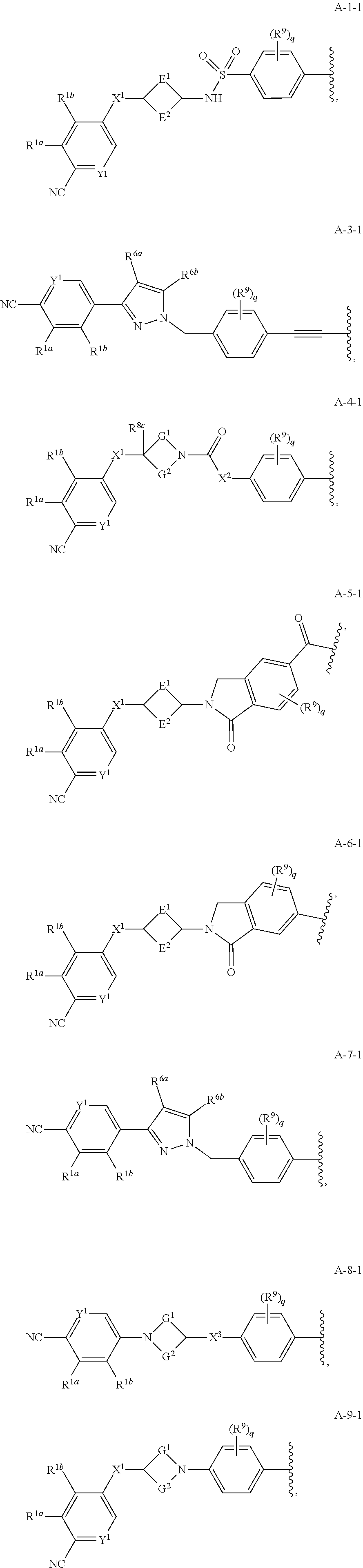

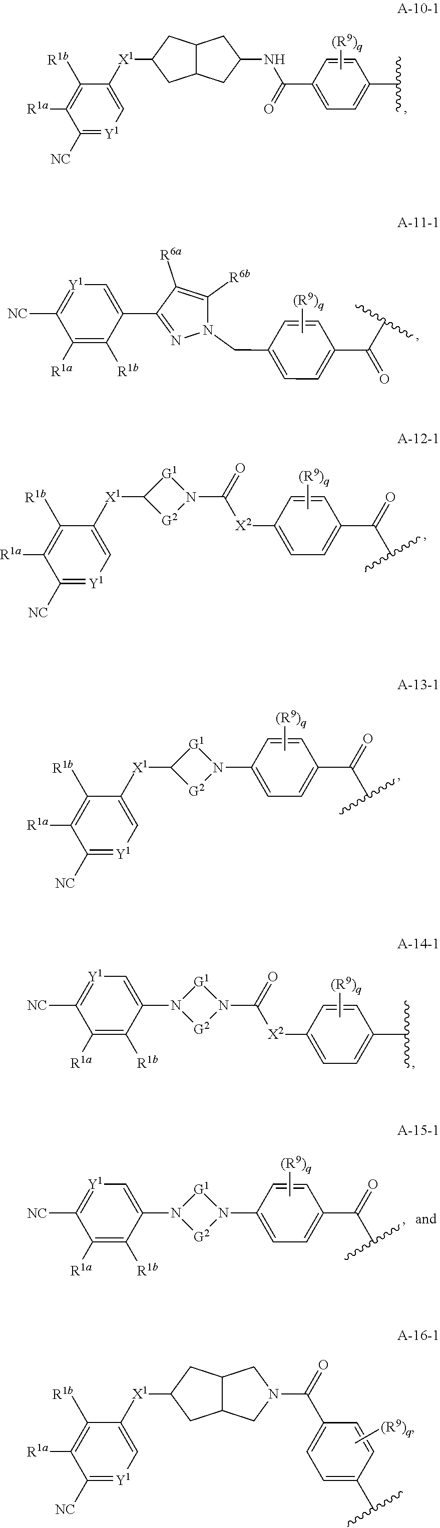

each R⁹ is independently selected from the group consisting of halo, C₁-C₃ alkyl, C₁-C₃ haloalkyl, and C₁-C₃ alkoxy; and

q is 0, 1, or 2.

In another embodiment, Compounds of the Disclosure are compounds of Formula I, or a pharmaceutically acceptable salt or solvate thereof, wherein A is A-1-1. In another embodiment, E¹ and E² are independently selected from the group consisting of —CH₂—, —C(CH₃)H—, —C(CH₃)₂—, —CH₂CH₂—, and —C(CH₃)(H)CH₂—. In another embodiment, X¹ is —O—. In another embodiment, X¹ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A is A-2-1. In another embodiment, E¹ and E² are independently selected from the group consisting of —CH₂—, —C(CH₃)H—, —C(CH₃)₂—, —CH₂CH₂—, and —C(CH₃)(H)CH₂—. In another embodiment, X¹ is —O—. In another embodiment, X¹ is —N(H)—. In another embodiment, Y², Y³, and Y⁴ are —C(H)═.

In another embodiment, Compounds of the Disclosure are compounds of Formula I, or a pharmaceutically acceptable salt or solvate thereof, wherein A is A-3-1. In another embodiment, q is 0 or 1. In another embodiment, q is 0.

In another embodiment, Compounds of the Disclosure are compounds of Formula I, or a pharmaceutically acceptable salt or solvate thereof, wherein A is A-4-1. In another embodiment, G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—. In another embodiment, X¹ is —O—. In another embodiment, X¹ is —N(H)—. In another embodiment, R^{8c} is hydrogen. In another embodiment, X² is —O—. In another embodiment, X² is —N(H)—. In another embodiment, X² is absent, i.e., X² is a bond. In another embodiment, q is 0 or 1. In another embodiment, q is 0.

In another embodiment, Compounds of the Disclosure are compounds of Formula I, or a pharmaceutically acceptable salt or solvate thereof, wherein A is A-5-1. In another embodiment, E¹ and E² are independently selected from the group consisting of —CH₂—, —C(CH₃)H—, —C(CH₃)₂—, —CH₂CH₂—, and —C(CH₃)(H)CH₂—. In another embodiment, X¹ is —O—. In another embodiment, X¹ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A is A-6-1. In another embodiment, E¹ and E² are independently selected from the group consisting of —CH₂—, —C(CH₃)H—, —C(CH₃)₂—, —CH₂CH₂—, and —C(CH₃)(H)CH₂—. In another embodiment, X¹ is —O—. In another embodiment, X¹ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A is A-7-1. In another embodiment, q is 0 or 1. In another embodiment, q is 0.

In another embodiment, Compounds of the Disclosure are compounds of Formula I, or a pharmaceutically acceptable salt or solvate thereof, wherein A is A-8-1. In another embodiment, G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—. In another embodiment, X³ is —O—. In another embodiment, X³ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A is A-9-1. In another embodiment, G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—. In another embodiment, X¹ is —O—. In another embodiment, X¹ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A is A-10-1. In another embodiment, X¹ is —O—. In another embodiment, X¹ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A is A-11-1. In another embodiment, q is 0 or 1. In another embodiment, q is 0.

In another embodiment, Compounds of the Disclosure are compounds of Formula I, or a pharmaceutically acceptable salt or solvate thereof, wherein A is A-12-1. In another embodiment, G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—. In another embodiment, X¹ is —O—. In another embodiment, X¹ is —N(H)—. In another embodiment, X² is —O—. In another embodiment, X² is —N(H)—. In another embodiment, X² is absent, i.e., X² is a bond. In another embodiment, q is 0 or 1. In another embodiment, q is 0.

In another embodiment, Compounds of the Disclosure are compounds of Formula I, or a pharmaceutically acceptable salt or solvate thereof, wherein A is A-13-1. In another embodiment, G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—. In another embodiment, X¹ is —O—. In another embodiment, X¹ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A is A-14-1. In another embodiment, G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—. In another embodiment, X² is —O—. In another embodiment, X² is —N(H)—. In another embodiment, X² is absent, i.e., X² is a bond. In another embodiment, q is 0 or 1. In another embodiment, q is 0.

In another embodiment, Compounds of the Disclosure are compounds of Formula I, or a pharmaceutically acceptable salt or solvate thereof, wherein A is A-15-1. In another embodiment, G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—. In another embodiment, q is 0 or 1. In another embodiment, q is 0.

In another embodiment, Compounds of the Disclosure are compounds of Formula I, or a pharmaceutically acceptable salt or solvate thereof, wherein A is A-16-1. In another embodiment, X¹ is —O—. In another embodiment, X¹ is —N(H)—. In another embodiment, q is 0 or 1. In another embodiment, q is 0.

In another embodiment, Compounds of the Disclosure are compounds of Formula I, or a pharmaceutically acceptable salt or solvate thereof, wherein A-4 is selected from the group consisting of:

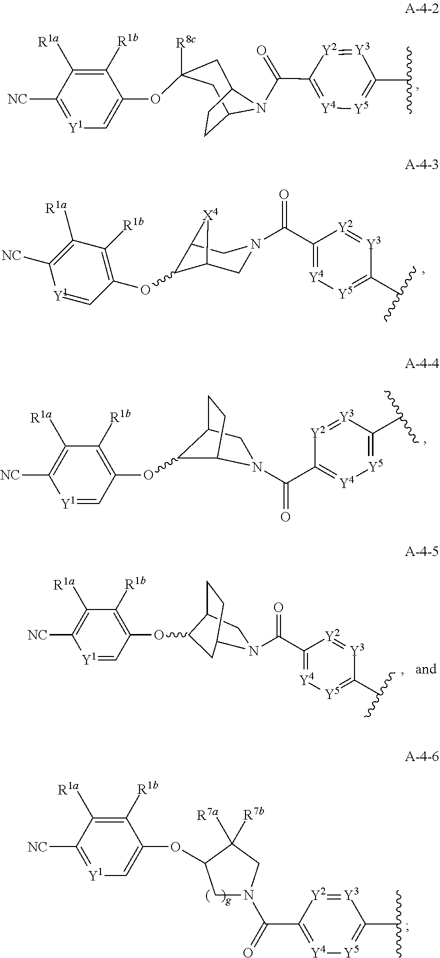

X⁴ is selected from the group consisting of —CH₂CH₂—, —CH₂CH₂CH₂—, and —CH₂OCH₂—.

In another embodiment, Compounds of the Disclosure are compounds of Formula I, or a pharmaceutically acceptable salt or solvate thereof, wherein A is A-4-2. In another embodiment, R^{8c} is hydrogen. In another embodiment, Y², Y³, Y⁴, and Y⁵ are —C(H)═.

In another embodiment, Compounds of the Disclosure are compounds of Formula I, or a pharmaceutically acceptable salt or solvate thereof, wherein A is A-4-3. In another embodiment, X⁴ is —CH₂CH₂—. In another embodiment, X⁴ is —CH₂CH₂CH₂—. In another embodiment, X⁴ is —CH₂OCH₂—. In another embodiment, Y², Y³, Y⁴, and Y⁵ are —C(H)═.

In another embodiment, Compounds of the Disclosure are compounds of Formula I, or a pharmaceutically acceptable salt or solvate thereof, wherein A is A-4-4. In another embodiment, R^{8c} is hydrogen. In another embodiment, Y², Y³, Y⁴, and Y⁵ are —C(H)═.

In another embodiment, Compounds of the Disclosure are compounds of Formula I, or a pharmaceutically acceptable salt or solvate thereof, wherein A is A-4-5. In another embodiment, R^{8c} is hydrogen. In another embodiment, Y², Y³, Y⁴, and Y⁵ are —C(H)═.

In another embodiment, Compounds of the Disclosure are compounds of Formula I, or a pharmaceutically acceptable salt or solvate thereof, wherein A is A-4-6. In another embodiment, g is 1. In another embodiment, g is 2. In another embodiment, R^{7a} and R^{7b} are hydrogen. In another embodiment, R^{7a} and R^{7b} are C₁-C₃ alkyl. In another embodiment, R^{7a} and R^{7b} are methyl. In another embodiment, R^{7a} and R^{7b} taken together with the carbon atom to which they are attached form a C₃-C₆ cycloalkyl. In another embodiment, Y², Y³, Y⁴, and Y⁵ are —C(H)═.

In another embodiment, Compounds of the Disclosure are compounds of Formula I, or a pharmaceutically acceptable salt or solvate thereof, wherein Y¹ is —CH═.

In another embodiment, Compounds of the Disclosure are compounds of Formula I, or a pharmaceutically acceptable salt or solvate thereof, wherein R^{1b} is hydrogen.

In another embodiment, Compounds of the Disclosure are compounds of Formula I, or a pharmaceutically acceptable salt or solvate thereof, wherein R^{1a} is chloro.

In another embodiment, Compounds of the Disclosure are compounds of Formula I, or a pharmaceutically acceptable salt or solvate thereof, wherein A is selected from the group consisting of:

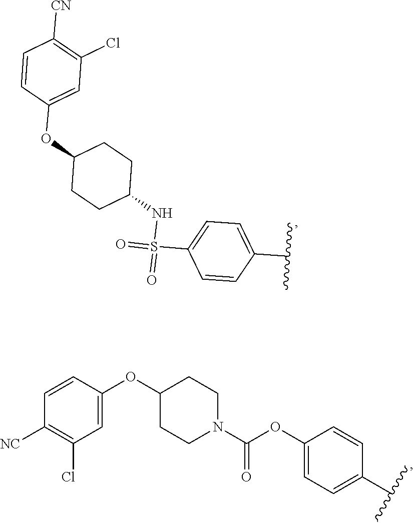 [SMILES: *c1ccc(S(=O)(=O)N[C@H]2CC[C@H](Oc3ccc(C#N)c(Cl)c3)CC2)cc1 ; *c1ccc(OC(=O)N2CCC(Oc3ccc(C#N)c(Cl)c3)CC2)cc1]

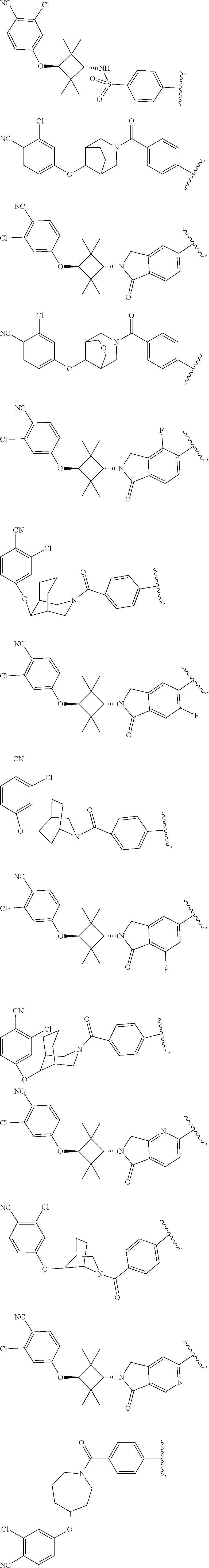

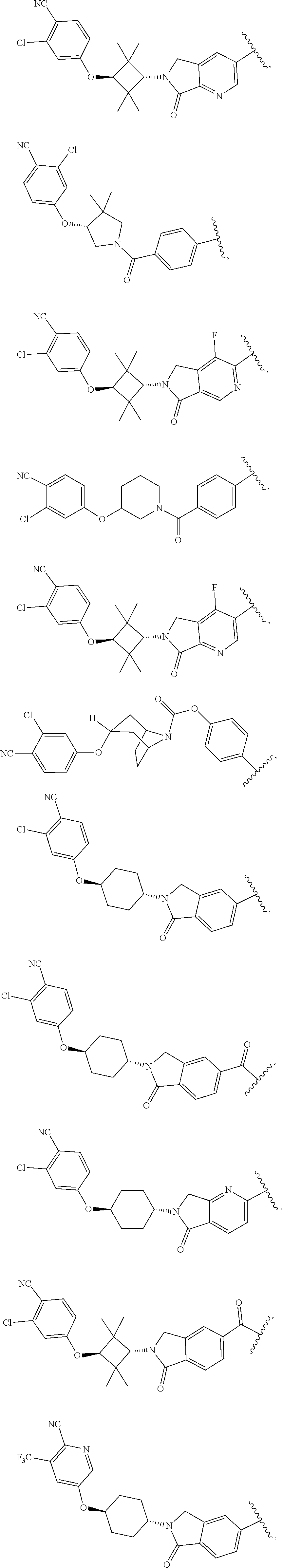

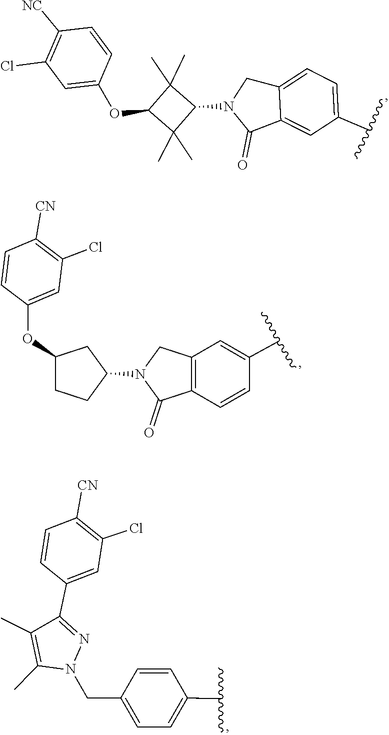 [SMILES: *c1ccc2c(c1)C(=O)N([C@H]1C(C)(C)[C@H](Oc3ccc(C#N)c(Cl)c3)C1(C)C)C2 ; *c1ccc2c(c1)CN([C@@H]1CC[C@@H](Oc3ccc(C#N)c(Cl)c3)C1)C2=O ; *c1ccc(Cn2nc(-c3ccc(C#N)c(Cl)c3)c(C)c2C)cc1]

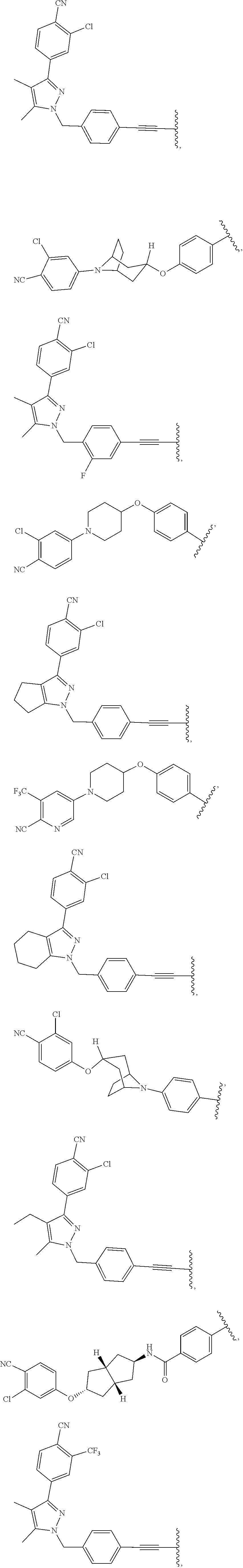

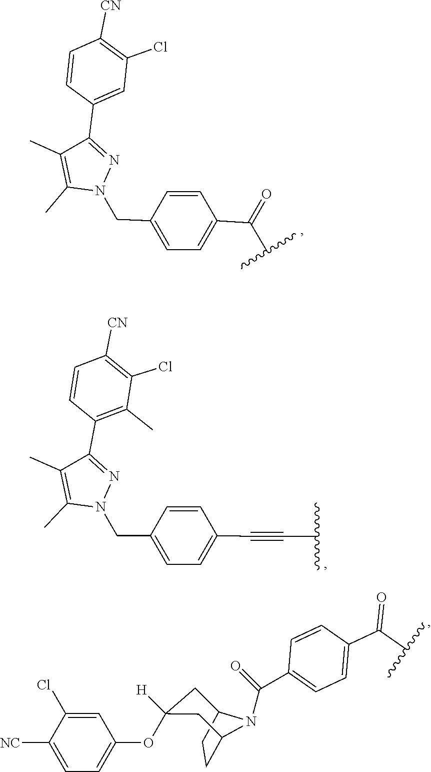 [SMILES: *C(=O)c1ccc(Cn2nc(-c3ccc(C#N)c(Cl)c3)c(C)c2C)cc1 ; *C#Cc1ccc(Cn2nc(-c3ccc(C#N)c(Cl)c3C)c(C)c2C)cc1 ; *C(=O)c1ccc(C(=O)N2C3CCC2CC(Oc2ccc(C#N)c(Cl)c2)C3)cc1]

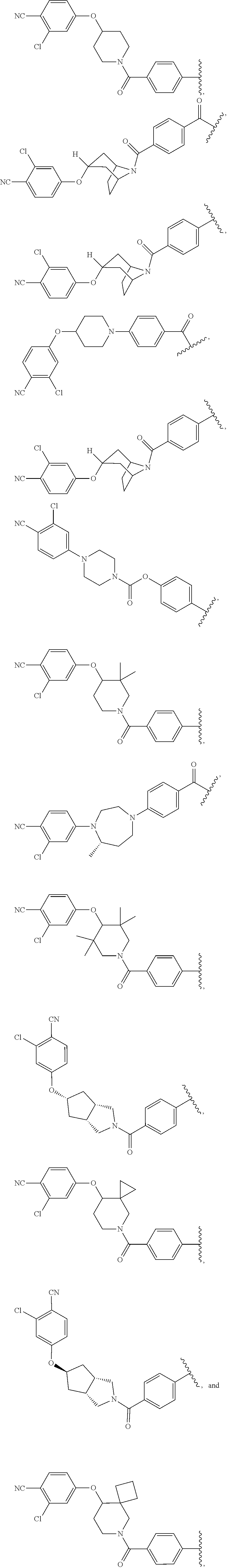

In another embodiment, Compounds of the Disclosure are compounds of Formula I, or a pharmaceutically acceptable salt or solvate thereof, wherein J¹ is heterocyclenyl. In another embodiment, J¹ is a 4- to 10-membered heterocyclenyl.

In another embodiment, Compounds of the Disclosure are compounds of Formula I, or a pharmaceutically acceptable salt or solvate thereof, wherein:

J¹ is selected from the group consisting of:

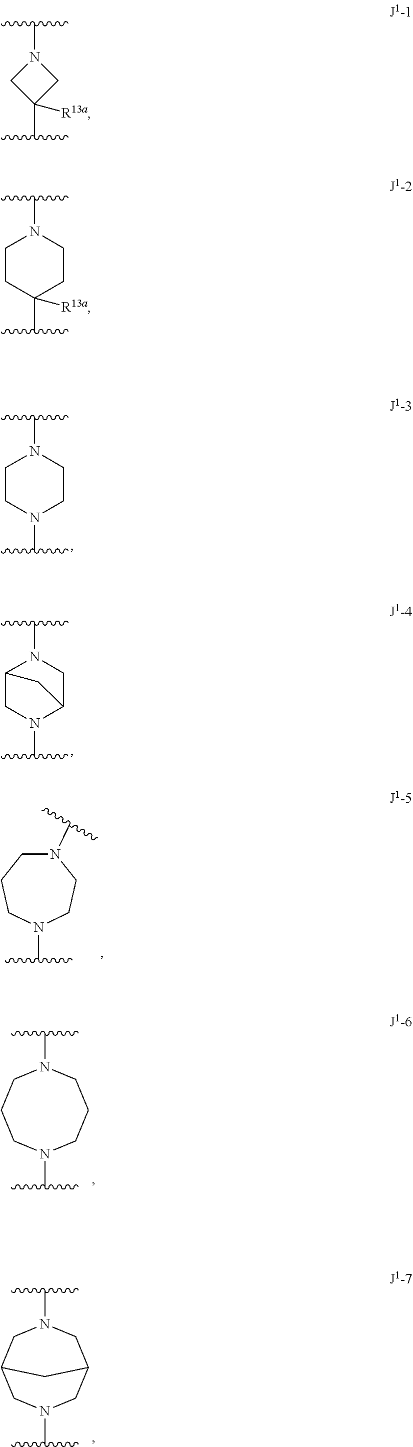

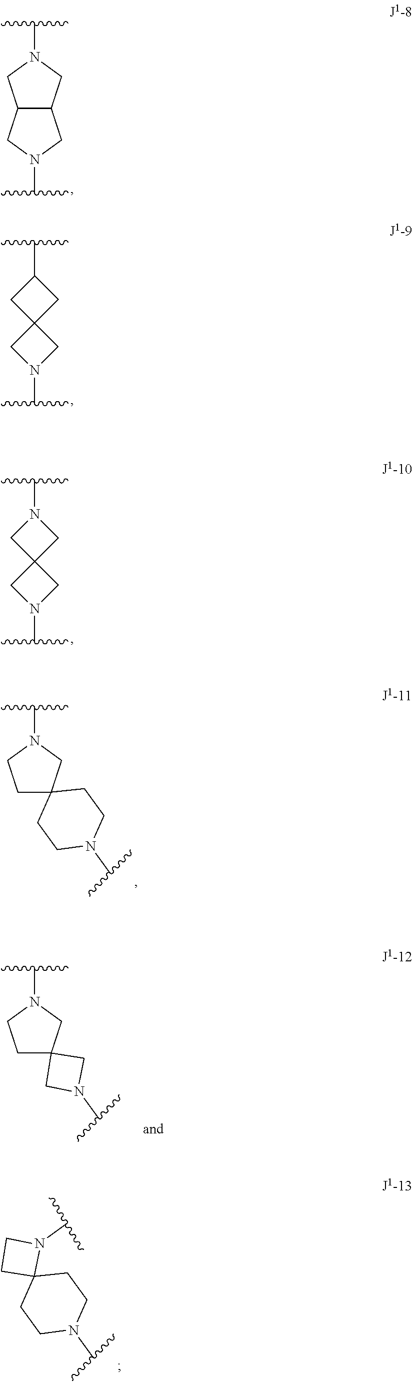 [SMILES: *N1CC2CN(*)CC2C1 ; *C1CC2(C1)CN(*)C2 ; *N1CC2(C1)CN(*)C2 ; *N1CCC2(CC1)CCN(*)C2 ; *N1CCC2(C1)CN(*)C2 ; *N1CCC2(CC1)CCN2*]

and

R^{13a} is selected from the group consisting of hydrogen, halo, hydroxy, cyano, C₁-C₄ alkyl, C₃-C₆ cycloalkyl, and C₁-C₄ alkoxy.

In another embodiment, Compounds of the Disclosure are compounds of Formula I, or a pharmaceutically acceptable salt or solvate thereof, wherein J¹ is J¹-1. In another embodiment, R^{13a} is selected from the group consisting of hydrogen and halo.

In another embodiment, Compounds of the Disclosure are compounds of Formula I, or a pharmaceutically acceptable salt or solvate thereof, wherein J¹ is J¹-2. In another embodiment, R^{13a} is selected from the group consisting of hydrogen and halo.

In another embodiment, Compounds of the Disclosure are compounds of Formula I, or a pharmaceutically acceptable salt or solvate thereof, wherein J¹ is J¹-3.

In another embodiment, Compounds of the Disclosure are compounds of Formula I, or a pharmaceutically acceptable salt or solvate thereof, wherein J¹ is J¹-4.

In another embodiment, Compounds of the Disclosure are compounds of Formula I, or a pharmaceutically acceptable salt or solvate thereof, wherein J¹ is J¹-5.

In another embodiment, Compounds of the Disclosure are compounds of Formula I, or a pharmaceutically acceptable salt or solvate thereof, wherein J¹ is J¹-6.

In another embodiment, Compounds of the Disclosure are compounds of Formula I, or a pharmaceutically acceptable salt or solvate thereof, wherein J¹ is J¹-7.

In another embodiment, Compounds of the Disclosure are compounds of Formula I, or a pharmaceutically acceptable salt or solvate thereof, wherein J¹ is J¹-8.

In another embodiment, Compounds of the Disclosure are compounds of Formula I, or a pharmaceutically acceptable salt or solvate thereof, wherein J¹ is J¹-9.

In another embodiment, Compounds of the Disclosure are compounds of Formula I, or a pharmaceutically acceptable salt or solvate thereof, wherein J¹ is J¹-10.

In another embodiment, Compounds of the Disclosure are compounds of Formula I, or a pharmaceutically acceptable salt or solvate thereof, wherein J¹ is J¹-11.

In another embodiment, Compounds of the Disclosure are compounds of Formula I, or a pharmaceutically acceptable salt or solvate thereof, wherein J¹ is J¹-12.

In another embodiment, Compounds of the Disclosure are compounds of Formula I, or a pharmaceutically acceptable salt or solvate thereof, wherein J¹ is J¹-13.

In another embodiment, Compounds of the Disclosure are compounds of Formula I, or a pharmaceutically acceptable salt or solvate thereof, wherein J¹ is cycloalkylenyl.

In another embodiment, Compounds of the Disclosure are compounds of Formula I, or a pharmaceutically acceptable salt or solvate thereof, wherein J¹ is absent.

In another embodiment, Compounds of the Disclosure are compounds of Formula I, or a pharmaceutically acceptable salt or solvate thereof, wherein J² is selected from the group consisting of —C(═O)—, —C(═O)NH—, —(CH₂)ₒ— and —C≡C—; and o is 0, 1, or 2.

In another embodiment, Compounds of the Disclosure are compounds of Formula I, or a pharmaceutically acceptable salt or solvate thereof, wherein J² is —(CH₂)ₒ—; and o is 0.

In another embodiment, Compounds of the Disclosure are compounds of Formula I, or a pharmaceutically acceptable salt or solvate thereof, wherein J² is —(CH₂)ₒ—; and o is 1.

In another embodiment, Compounds of the Disclosure are compounds of Formula I, or a pharmaceutically acceptable salt or solvate thereof, wherein J² is —C≡C—.

In another embodiment, Compounds of the Disclosure are compounds of Formula I, or a pharmaceutically acceptable salt or solvate thereof, wherein J² is —C(═O)NH—,

In another embodiment, Compounds of the Disclosure are compounds of Formula I, or a pharmaceutically acceptable salt or solvate thereof, wherein J³ is selected from the group consisting of alkylenyl, cycloalkylenyl and heterocyclenyl.

In another embodiment, Compounds of the Disclosure are compounds of Formula I, or a pharmaceutically acceptable salt or solvate thereof, wherein:

J³ is selected from the group consisting of:

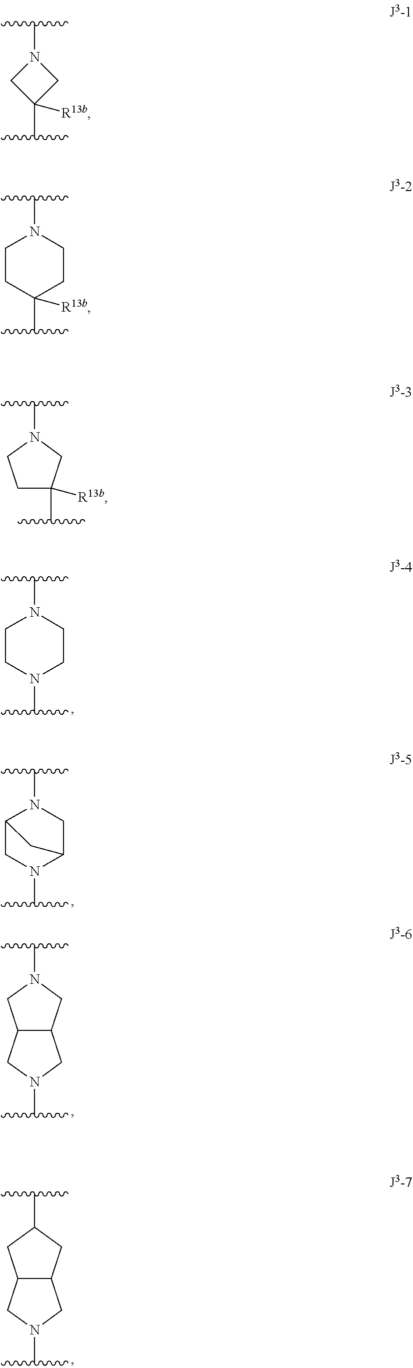

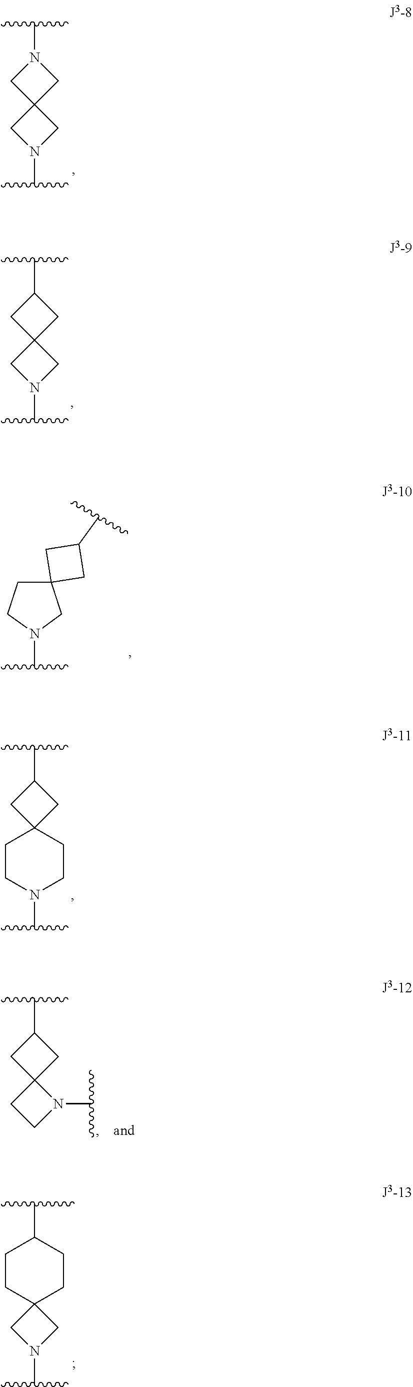 [SMILES: *N1CC2(C1)CN(*)C2 ; *C1CC2(C1)CN(*)C2 ; *C1CC2(CCN(*)C2)C1 ; *C1CC2(CCN(*)CC2)C1 ; *C1CC2(CCN2*)C1 ; *C1CCC2(CC1)CN(*)C2]

and

R^{13b} is selected from the group consisting of hydrogen, halo, hydroxy, cyano, C₁-C₄ alkyl, C₃-C₆ cycloalkyl, and C₁-C₄ alkoxy.

In another embodiment, Compounds of the Disclosure are compounds of Formula I, or a pharmaceutically acceptable salt or solvate thereof, wherein J³ is J³-1. In another embodiment, R^{13b} is selected from the group consisting of hydrogen and halo.

In another embodiment, Compounds of the Disclosure are compounds of Formula I, or a pharmaceutically acceptable salt or solvate thereof, wherein J³ is J³-2. In another embodiment, R^{13a} is selected from the group consisting of hydrogen and halo.

In another embodiment, Compounds of the Disclosure are compounds of Formula I, or a pharmaceutically acceptable salt or solvate thereof, wherein J³ is J³-3.

In another embodiment, Compounds of the Disclosure are compounds of Formula I, or a pharmaceutically acceptable salt or solvate thereof, wherein J³ is J³-4.

In another embodiment, Compounds of the Disclosure are compounds of Formula I, or a pharmaceutically acceptable salt or solvate thereof, wherein J³ is J³-5.

In another embodiment, Compounds of the Disclosure are compounds of Formula I, or a pharmaceutically acceptable salt or solvate thereof, wherein J³ is J³-6.

In another embodiment, Compounds of the Disclosure are compounds of Formula I, or a pharmaceutically acceptable salt or solvate thereof, wherein J³ is J³-7.

In another embodiment, Compounds of the Disclosure are compounds of Formula I, or a pharmaceutically acceptable salt or solvate thereof, wherein J³ is J³-8.

In another embodiment, Compounds of the Disclosure are compounds of Formula I, or a pharmaceutically acceptable salt or solvate thereof, wherein J³ is J³-9.

In another embodiment, Compounds of the Disclosure are compounds of Formula I, or a pharmaceutically acceptable salt or solvate thereof, wherein J³ is J³-10.

In another embodiment, Compounds of the Disclosure are compounds of Formula I, or a pharmaceutically acceptable salt or solvate thereof, wherein J³ is J³-11.

In another embodiment, Compounds of the Disclosure are compounds of Formula I, or a pharmaceutically acceptable salt or solvate thereof, wherein J³ is J³-12.

In another embodiment, Compounds of the Disclosure are compounds of Formula I, or a pharmaceutically acceptable salt or solvate thereof, wherein J³ is J³-13.

In another embodiment, Compounds of the Disclosure are compounds of Formula I, or a pharmaceutically acceptable salt or solvate thereof, wherein J³ is absent.

In another embodiment, Compounds of the Disclosure are compounds of Formula I, or a pharmaceutically acceptable salt or solvate thereof, wherein J⁴ is selected from the group consisting of alkylenyl, cycloalkylenyl, and heterocyclenyl.

In another embodiment, Compounds of the Disclosure are compounds of Formula I, or a pharmaceutically acceptable salt or solvate thereof, wherein J⁴ is absent.

In another embodiment, Compounds of the Disclosure are compounds of Formula I, or a pharmaceutically acceptable salt or solvate thereof, wherein J⁵ is selected from the group consisting of —C≡C—, —(CH₂)ₚ—, —N(H)—, and —C(═O)—; and p is 0, 1, or 2

In another embodiment, Compounds of the Disclosure are compounds of Formula I, or a pharmaceutically acceptable salt or solvate thereof, wherein J⁵ is selected from the group consisting of —(CH₂)ₚ— and —C(═O)—; and p is 0, 1, or 2.

In another embodiment, Compounds of the Disclosure are compounds of Formula I, or a pharmaceutically acceptable salt or solvate thereof, wherein J¹ is selected from the group consisting of J¹-1 and J¹-2; J² is absent, J³ is heterocyclenyl; J⁴ is absent; and J⁵ is —(CH₂)ₚ—.

In another embodiment, Compounds of the Disclosure are compounds of Formula I, or a pharmaceutically acceptable salt or solvate thereof, wherein J¹ is selected from the group consisting of J¹-1 and J¹-2; J², J³, and J⁴ are absent, and J⁵ is —(CH₂)ₚ—.

In another embodiment, Compounds of the Disclosure are compounds of Formula I, or a pharmaceutically acceptable salt or solvate thereof, wherein L is any one or more of the -J¹-, -J¹-J²-, -J¹-J²-J³-, J¹-J²-J³-J⁴-, or -J¹-J²-J³-, -J¹-J²-J³-J⁴-J⁵- groups listed in Table 5.

TABLE 5

| No. | J¹ | J² | J³ | J⁴ | J⁵ |
|---|---|---|---|---|---|
| 1 | alkylenyl | — | — | — | — |
| 2 | cycloalkylenyl | — | — | — | — |
| 3 | heterocyclenyl | — | — | — | — |
| 4 | — | —C(═O)— | — | — | — |
| 5 | alkylenyl | —C(═O)— | — | — | — |
| 6 | cycloalkylenyl | —C(═O)— | — | — | — |
| 7 | heterocyclenyl | —C(═O)— | — | — | — |
| 8 | — | —C(═O)NH— | — | — | — |
| 9 | alkylenyl | —C(═O)NH— | — | — | — |
| 10 | cycloalkylenyl | —C(═O)NH— | — | — | — |
| 11 | heterocyclenyl | —C(═O)NH— | — | — | — |
| 12 | — | —C≡C— | — | — | — |
| 13 | alkylenyl | —C≡C— | — | — | — |
| 14 | cycloalkylenyl | —C≡C— | — | — | — |
| 15 | heterocyclenyl | —C≡C— | — | — | — |
| 16 | alkylenyl | — | heterocyclenyl | — | — |
| 17 | cycloalkylenyl | — | heterocyclenyl | — | — |
| 18 | heterocyclenyl | — | heterocyclenyl | — | — |
| 19 | — | —C(═O)— | heterocyclenyl | — | — |
| 20 | alkylenyl | —C(═O)— | heterocyclenyl | — | — |
| 21 | cycloalkylenyl | —C(═O)— | heterocyclenyl | — | — |
| 22 | heterocyclenyl | —C(═O)— | heterocyclenyl | — | — |
| 23 | — | —C(═O)NH— | heterocyclenyl | — | — |
| 24 | alkylenyl | —C(═O)NH— | heterocyclenyl | — | — |
| 25 | cycloalkylenyl | —C(═O)NH— | heterocyclenyl | — | — |
| 26 | heterocyclenyl | —C(═O)NH— | heterocyclenyl | — | — |
| 27 | — | —C≡C— | heterocyclenyl | — | — |
| 28 | alkylenyl | —C≡C— | heterocyclenyl | — | — |
| 29 | cycloalkylenyl | —C≡C— | heterocyclenyl | — | — |
| 30 | heterocyclenyl | —C≡C— | heterocyclenyl | — | — |
| 31 | cycloalkylenyl | — | alkylenyl | heterocyclenyl | — |
| 32 | heterocyclenyl | — | alkylenyl | heterocyclenyl | — |
| 33 | — | —C(═O)— | alkylenyl | heterocyclenyl | — |
| 34 | alkylenyl | —C(═O)— | alkylenyl | heterocyclenyl | — |
| 35 | cycloalkylenyl | —C(═O)— | alkylenyl | heterocyclenyl | — |
| 36 | heterocyclenyl | —C(═O)— | alkylenyl | heterocyclenyl | — |
| 37 | — | —C(═O)NH— | alkylenyl | heterocyclenyl | — |
| 38 | alkylenyl | —C(═O)NH— | alkylenyl | heterocyclenyl | — |
| 39 | cycloalkylenyl | —C(═O)NH— | alkylenyl | heterocyclenyl | — |
| 40 | heterocyclenyl | —C(═O)NH— | alkylenyl | heterocyclenyl | — |
| 41 | — | —C≡C— | alkylenyl | heterocyclenyl | — |
| 42 | alkylenyl | —C≡C— | alkylenyl | heterocyclenyl | — |
| 43 | cycloalkylenyl | —C≡C— | alkylenyl | heterocyclenyl | — |
| 44 | heterocyclenyl | —C≡C— | alkylenyl | heterocyclenyl | — |
| 45 | alkylenyl | — | cycloalkylenyl | heterocyclenyl | — |
| 46 | cycloalkylenyl | — | cycloalkylenyl | heterocyclenyl | — |
| 47 | heterocyclenyl | — | cycloalkylenyl | heterocyclenyl | — |
| 48 | — | —C(═O)— | cycloalkylenyl | heterocyclenyl | — |
| 49 | alkylenyl | —C(═O)— | cycloalkylenyl | heterocyclenyl | — |
| 50 | cycloalkylenyl | —C(═O)— | cycloalkylenyl | heterocyclenyl | — |
| 51 | heterocyclenyl | —C(═O)— | cycloalkylenyl | heterocyclenyl | — |
| 52 | — | —C(═O)NH— | cycloalkylenyl | heterocyclenyl | — |
| 53 | alkylenyl | —C(═O)NH— | cycloalkylenyl | heterocyclenyl | — |
| 54 | cycloalkylenyl | —C(═O)NH— | cycloalkylenyl | heterocyclenyl | — |
| 55 | heterocyclenyl | —C(═O)NH— | cycloalkylenyl | heterocyclenyl | — |
| 56 | — | —C≡C— | cycloalkylenyl | heterocyclenyl | — |
| 57 | alkylenyl | —C≡C— | cycloalkylenyl | heterocyclenyl | — |
| 58 | cycloalkylenyl | —C≡C— | cycloalkylenyl | heterocyclenyl | — |
| 59 | heterocyclenyl | —C≡C— | cycloalkylenyl | heterocyclenyl | — |
| 60 | alkylenyl | — | phenylenyl | heterocyclenyl | — |
| 61 | cycloalkylenyl | — | phenylenyl | heterocyclenyl | — |
| 62 | heterocyclenyl | — | phenylenyl | heterocyclenyl | — |
| 63 | — | —C(═O)— | phenylenyl | heterocyclenyl | — |
| 64 | alkylenyl | —C(═O)— | phenylenyl | heterocyclenyl | — |
| 65 | cycloalkylenyl | —C(═O)— | phenylenyl | heterocyclenyl | — |
| 66 | heterocyclenyl | —C(═O)— | phenylenyl | heterocyclenyl | — |
| 67 | — | —C(═O)NH— | phenylenyl | heterocyclenyl | — |
| 68 | alkylenyl | —C(═O)NH— | phenylenyl | heterocyclenyl | — |
| 69 | cycloalkylenyl | —C(═O)NH— | phenylenyl | heterocyclenyl | — |
| 70 | heterocyclenyl | —C(═O)NH— | phenylenyl | heterocyclenyl | — |
| 71 | — | —C≡C— | phenylenyl | heterocyclenyl | — |
| 72 | alkylenyl | —C≡C— | phenylenyl | heterocyclenyl | — |
| 73 | cycloalkylenyl | —C≡C— | phenylenyl | heterocyclenyl | — |
| 74 | heterocyclenyl | —C≡C— | phenylenyl | heterocyclenyl | — |
| 75 | cycloalkylenyl | — | alkylenyl | — | —C≡C— |
| 76 | heterocyclenyl | — | alkylenyl | — | —C≡C— |
| 77 | — | —C(═O)— | alkylenyl | — | —C≡C— |
| 78 | alkylenyl | —C(═O)— | alkylenyl | — | —C≡C— |
| 79 | cycloalkylenyl | —C(═O)— | alkylenyl | — | —C≡C— |
| 80 | heterocyclenyl | —C(═O)— | alkylenyl | — | —C≡C— |
| 81 | — | —C(═O)NH— | alkylenyl | — | —C≡C— |
| 82 | alkylenyl | —C(═O)NH— | alkylenyl | — | —C≡C— |
| 83 | cycloalkylenyl | —C(═O)NH— | alkylenyl | — | —C≡C— |
| 84 | heterocyclenyl | —C(═O)NH— | alkylenyl | — | —C≡C— |
| 85 | — | —C≡C— | alkylenyl | — | —C≡C— |
| 86 | alkylenyl | —C≡C— | alkylenyl | — | —C≡C— |
| 87 | cycloalkylenyl | —C≡C— | alkylenyl | — | —C≡C— |
| 88 | heterocyclenyl | —C≡C— | alkylenyl | — | —C≡C— |
| 89 | — | — | heteroalkylenyl | — | —C≡C— |
| 90 | alkylenyl | — | heteroalkylenyl | — | —C≡C— |
| 91 | cycloalkylenyl | — | heteroalkylenyl | — | —C≡C— |
| 92 | heterocyclenyl | — | heteroalkylenyl | — | —C≡C— |
| 93 | — | —C(═O)— | heteroalkylenyl | — | —C≡C— |
| 94 | alkylenyl | —C(═O)— | heteroalkylenyl | — | —C≡C— |
| 95 | cycloalkylenyl | —C(═O)— | heteroalkylenyl | — | —C≡C— |
| 96 | heterocyclenyl | —C(═O)— | heteroalkylenyl | — | —C≡C— |
| 97 | — | —C(═O)NH— | heteroalkylenyl | — | —C≡C— |
| 98 | alkylenyl | —C(═O)NH— | heteroalkylenyl | — | —C≡C— |
| 99 | cycloalkylenyl | —C(═O)NH— | heteroalkylenyl | — | —C≡C— |
| 100 | heterocyclenyl | —C(═O)NH— | heteroalkylenyl | — | —C≡C— |
| 101 | — | —C≡C— | heteroalkylenyl | — | —C≡C— |
| 102 | alkylenyl | —C≡C— | heteroalkylenyl | — | —C≡C— |
| 103 | cycloalkylenyl | —C≡C— | heteroalkylenyl | — | —C≡C— |
| 104 | heterocyclenyl | —C≡C— | heteroalkylenyl | — | —C≡C— |
| 105 | alkylenyl | — | heterocyclenyl | — | —C≡C— |
| 106 | cycloalkylenyl | — | heterocyclenyl | — | —C≡C— |
| 107 | heterocyclenyl | — | heterocyclenyl | — | —C≡C— |
| 108 | — | —C(═O)— | heterocyclenyl | — | —C≡C— |
| 109 | alkylenyl | —C(═O)— | heterocyclenyl | — | —C≡C— |
| 110 | cycloalkylenyl | —C(═O)— | heterocyclenyl | — | —C≡C— |
| 111 | heterocyclenyl | —C(═O)— | heterocyclenyl | — | —C≡C— |
| 112 | — | —C(═O)NH— | heterocyclenyl | — | —C≡C— |
| 113 | alkylenyl | —C(═O)NH— | heterocyclenyl | — | —C≡C— |
| 114 | cycloalkylenyl | —C(═O)NH— | heterocyclenyl | — | —C≡C— |
| 115 | heterocyclenyl | —C(═O)NH— | heterocyclenyl | — | —C≡C— |
| 116 | — | —C≡C— | heterocyclenyl | — | —C≡C— |
| 117 | alkylenyl | —C≡C— | heterocyclenyl | — | —C≡C— |
| 118 | cycloalkylenyl | —C≡C— | heterocyclenyl | — | —C≡C— |
| 119 | heterocyclenyl | —C≡C— | heterocyclenyl | — | —C≡C— |
| 120 | alkylenyl | — | alkylenyl | heterocyclenyl | —C≡C— |
| 121 | cycloalkylenyl | — | alkylenyl | heterocyclenyl | —C≡C— |
| 122 | heterocyclenyl | — | alkylenyl | heterocyclenyl | —C≡C— |
| 123 | — | —C(═O)— | alkylenyl | heterocyclenyl | —C≡C— |
| 124 | alkylenyl | —C(═O)— | alkylenyl | heterocyclenyl | —C≡C— |
| 125 | cycloalkylenyl | —C(═O)— | alkylenyl | heterocyclenyl | —C≡C— |
| 126 | heterocyclenyl | —C(═O)— | alkylenyl | heterocyclenyl | —C≡C— |
| 127 | — | —C(═O)NH— | alkylenyl | heterocyclenyl | —C≡C— |
| 128 | alkylenyl | —C(═O)NH— | alkylenyl | heterocyclenyl | —C≡C— |
| 129 | cycloalkylenyl | —C(═O)NH— | alkylenyl | heterocyclenyl | —C≡C— |
| 130 | heterocyclenyl | —C(═O)NH— | alkylenyl | heterocyclenyl | —C≡C— |
| 131 | — | —C≡C— | alkylenyl | heterocyclenyl | —C≡C— |
| 132 | alkylenyl | —C≡C— | alkylenyl | heterocyclenyl | —C≡C— |
| 133 | cycloalkylenyl | —C≡C— | alkylenyl | heterocyclenyl | —C≡C— |
| 134 | heterocyclenyl | —C≡C— | alkylenyl | heterocyclenyl | —C≡C— |
| 135 | alkylenyl | — | heterocyclenyl | alkylenyl | —C≡C— |
| 136 | cycloalkylenyl | — | heterocyclenyl | alkylenyl | —C≡C— |
| 137 | heterocyclenyl | — | heterocyclenyl | alkylenyl | —C≡C— |
| 138 | — | —C(═O)— | heterocyclenyl | alkylenyl | —C≡C— |
| 139 | alkylenyl | —C(═O)— | heterocyclenyl | alkylenyl | —C≡C— |
| 140 | cycloalkylenyl | —C(═O)— | heterocyclenyl | alkylenyl | —C≡C— |
| 141 | heterocyclenyl | —C(═O)— | heterocyclenyl | alkylenyl | —C≡C— |
| 142 | — | —C(═O)NH— | heterocyclenyl | alkylenyl | —C≡C— |
| 143 | alkylenyl | —C(═O)NH— | heterocyclenyl | alkylenyl | —C≡C— |
| 144 | cycloalkylenyl | —C(═O)NH— | heterocyclenyl | alkylenyl | —C≡C— |
| 145 | heterocyclenyl | —C(═O)NH— | heterocyclenyl | alkylenyl | —C≡C— |
| 146 | — | —C≡C— | heterocyclenyl | alkylenyl | —C≡C— |
| 147 | alkylenyl | —C≡C— | heterocyclenyl | alkylenyl | —C≡C— |
| 148 | cycloalkylenyl | —C≡C— | heterocyclenyl | alkylenyl | —C≡C— |
| 149 | heterocyclenyl | —C≡C— | heterocyclenyl | alkylenyl | —C≡C— |
| 150 | alkylenyl | — | — | cycloalkylenyl | —C≡C— |
| 151 | cycloalkylenyl | — | — | cycloalkylenyl | —C≡C— |
| 152 | heterocyclenyl | — | — | cycloalkylenyl | —C≡C— |
| 153 | — | —C(═O)— | — | cycloalkylenyl | —C≡C— |
| 154 | alkylenyl | —C(═O)— | — | cycloalkylenyl | —C≡C— |
| 155 | cycloalkylenyl | —C(═O)— | — | cycloalkylenyl | —C≡C— |
| 156 | heterocyclenyl | —C(═O)— | — | cycloalkylenyl | —C≡C— |
| 157 | — | —C(═O)NH— | — | cycloalkylenyl | —C≡C— |
| 158 | alkylenyl | —C(═O)NH— | — | cycloalkylenyl | —C≡C— |
| 159 | cycloalkylenyl | —C(═O)NH— | — | cycloalkylenyl | —C≡C— |
| 160 | heterocyclenyl | —C(═O)NH— | — | cycloalkylenyl | —C≡C— |
| 161 | — | —C≡C— | — | cycloalkylenyl | —C≡C— |
| 162 | alkylenyl | —C≡C— | — | cycloalkylenyl | —C≡C— |
| 163 | cycloalkylenyl | —C≡C— | — | cycloalkylenyl | —C≡C— |
| 164 | heterocyclenyl | —C≡C— | — | cycloalkylenyl | —C≡C— |
| 165 | alkylenyl | — | alkylenyl | cycloalkylenyl | —C≡C— |
| 166 | cycloalkylenyl | — | alkylenyl | cycloalkylenyl | —C≡C— |
| 167 | heterocyclenyl | — | alkylenyl | cycloalkylenyl | —C≡C— |
| 168 | — | —C(═O)— | alkylenyl | cycloalkylenyl | —C≡C— |
| 169 | alkylenyl | —C(═O)— | alkylenyl | cycloalkylenyl | —C≡C— |
| 170 | cycloalkylenyl | —C(═O)— | alkylenyl | cycloalkylenyl | —C≡C— |
| 171 | heterocyclenyl | —C(═O)— | alkylenyl | cycloalkylenyl | —C≡C— |
| 172 | — | —C(═O)NH— | alkylenyl | cycloalkylenyl | —C≡C— |
| 173 | alkylenyl | —C(═O)NH— | alkylenyl | cycloalkylenyl | —C≡C— |
| 174 | cycloalkylenyl | —C(═O)NH— | alkylenyl | cycloalkylenyl | —C≡C— |
| 175 | heterocyclenyl | —C(═O)NH— | alkylenyl | cycloalkylenyl | —C≡C— |
| 176 | — | —C≡C— | alkylenyl | cycloalkylenyl | —C≡C— |
| 177 | alkylenyl | —C≡C— | alkylenyl | cycloalkylenyl | —C≡C— |
| 178 | cycloalkylenyl | —C≡C— | alkylenyl | cycloalkylenyl | —C≡C— |
| 179 | heterocyclenyl | —C≡C— | alkylenyl | cycloalkylenyl | —C≡C— |
| 180 | alkylenyl | — | heterocyclenyl | cycloalkylenyl | —C≡C— |
| 181 | cycloalkylenyl | — | heterocyclenyl | cycloalkylenyl | —C≡C— |
| 182 | heterocyclenyl | — | heterocyclenyl | cycloalkylenyl | —C≡C— |
| 183 | — | —C(═O)— | heterocyclenyl | cycloalkylenyl | —C≡C— |
| 184 | alkylenyl | —C(═O)— | heterocyclenyl | cycloalkylenyl | —C≡C— |
| 185 | cycloalkylenyl | —C(═O)— | heterocyclenyl | cycloalkylenyl | —C≡C— |
| 186 | heterocyclenyl | —C(═O)— | heterocyclenyl | cycloalkylenyl | —C≡C— |
| 187 | — | —C(═O)NH— | heterocyclenyl | cycloalkylenyl | —C≡C— |
| 188 | alkylenyl | —C(═O)NH— | heterocyclenyl | cycloalkylenyl | —C≡C— |
| 189 | cycloalkylenyl | —C(═O)NH— | heterocyclenyl | cycloalkylenyl | —C≡C— |
| 190 | heterocyclenyl | —C(═O)NH— | heterocyclenyl | cycloalkylenyl | —C≡C— |
| 191 | — | —C≡C— | heterocyclenyl | cycloalkylenyl | —C≡C— |
| 192 | alkylenyl | —C≡C— | heterocyclenyl | cycloalkylenyl | —C≡C— |
| 193 | cycloalkylenyl | —C≡C— | heterocyclenyl | cycloalkylenyl | —C≡C— |
| 194 | heterocyclenyl | —C≡C— | heterocyclenyl | cycloalkylenyl | —C≡C— |
| 195 | alkylenyl | — | — | — | —O— |
| 196 | cycloalkylenyl | — | — | — | —O— |
| 197 | heterocyclenyl | — | — | — | —O— |
| 198 | cycloalkylenyl | — | alkylenyl | — | —O— |
| 199 | heterocyclenyl | — | alkylenyl | — | —O— |
| 200 | — | —C(═O)— | alkylenyl | — | —O— |
| 201 | alkylenyl | —C(═O)— | alkylenyl | — | —O— |
| 202 | cycloalkylenyl | —C(═O)— | alkylenyl | — | —O— |
| 203 | heterocyclenyl | —C(═O)— | alkylenyl | — | —O— |
| 204 | — | —C(═O)NH— | alkylenyl | — | —O— |
| 205 | alkylenyl | —C(═O)NH— | alkylenyl | — | —O— |
| 206 | cycloalkylenyl | —C(═O)NH— | alkylenyl | — | —O— |
| 207 | heterocyclenyl | —C(═O)NH— | alkylenyl | — | —O— |
| 208 | — | —C≡C— | alkylenyl | — | —O— |
| 209 | alkylenyl | —C≡C— | alkylenyl | — | —O— |
| 210 | cycloalkylenyl | —C≡C— | alkylenyl | — | —O— |
| 211 | heterocyclenyl | —C≡C— | alkylenyl | — | —O— |
| 212 | alkylenyl | — | heterocyclenyl | — | —O— |
| 213 | cycloalkylenyl | — | heterocyclenyl | — | —O— |
| 214 | heterocyclenyl | — | heterocyclenyl | — | —O— |
| 215 | — | —C(═O)— | heterocyclenyl | — | —O— |
| 216 | alkylenyl | —C(═O)— | heterocyclenyl | — | —O— |
| 217 | cycloalkylenyl | —C(═O)— | heterocyclenyl | — | —O— |
| 218 | heterocyclenyl | —C(═O)— | heterocyclenyl | — | —O— |
| 219 | — | —C(═O)NH— | heterocyclenyl | — | —O— |
| 220 | alkylenyl | —C(═O)NH— | heterocyclenyl | — | —O— |
| 221 | cycloalkylenyl | —C(═O)NH— | heterocyclenyl | — | —O— |
| 222 | heterocyclenyl | —C(═O)NH— | heterocyclenyl | — | —O— |
| 223 | — | —C≡C— | heterocyclenyl | — | —O— |
| 224 | alkylenyl | —C≡C— | heterocyclenyl | — | —O— |
| 225 | cycloalkylenyl | —C≡C— | heterocyclenyl | — | —O— |
| 226 | heterocyclenyl | —C≡C— | heterocyclenyl | — | —O— |
| 227 | alkylenyl | — | alkylenyl | heterocyclenyl | —O— |
| 228 | cycloalkylenyl | — | alkylenyl | heterocyclenyl | —O— |
| 229 | heterocyclenyl | — | alkylenyl | heterocyclenyl | —O— |
| 230 | — | —C(═O)— | alkylenyl | heterocyclenyl | —O— |
| 231 | alkylenyl | —C(═O)— | alkylenyl | heterocyclenyl | —O— |
| 232 | cycloalkylenyl | —C(═O)— | alkylenyl | heterocyclenyl | —O— |
| 233 | heterocyclenyl | —C(═O)— | alkylenyl | heterocyclenyl | —O— |
| 234 | — | —C(═O)NH— | alkylenyl | heterocyclenyl | —O— |
| 235 | alkylenyl | —C(═O)NH— | alkylenyl | heterocyclenyl | —O— |
| 236 | cycloalkylenyl | —C(═O)NH— | alkylenyl | heterocyclenyl | —O— |
| 237 | heterocyclenyl | —C(═O)NH— | alkylenyl | heterocyclenyl | —O— |
| 238 | — | —C≡C— | alkylenyl | heterocyclenyl | —O— |
| 239 | alkylenyl | —C≡C— | alkylenyl | heterocyclenyl | —O— |
| 240 | cycloalkylenyl | —C≡C— | alkylenyl | heterocyclenyl | —O— |
| 241 | heterocyclenyl | —C≡C— | alkylenyl | heterocyclenyl | —O— |
| 242 | alkylenyl | — | heterocyclenyl | alkylenyl | —O— |
| 243 | cycloalkylenyl | — | heterocyclenyl | alkylenyl | —O— |
| 244 | heterocyclenyl | — | heterocyclenyl | alkylenyl | —O— |
| 245 | — | —C(═O)— | heterocyclenyl | alkylenyl | —O— |
| 246 | alkylenyl | —C(═O)— | heterocyclenyl | alkylenyl | —O— |
| 247 | cycloalkylenyl | —C(═O)— | heterocyclenyl | alkylenyl | —O— |
| 248 | heterocyclenyl | —C(═O)— | heterocyclenyl | alkylenyl | —O— |
| 249 | — | —C(═O)NH— | heterocyclenyl | alkylenyl | —O— |
| 250 | alkylenyl | —C(═O)NH— | heterocyclenyl | alkylenyl | —O— |
| 251 | cycloalkylenyl | —C(═O)NH— | heterocyclenyl | alkylenyl | —O— |
| 252 | heterocyclenyl | —C(═O)NH— | heterocyclenyl | alkylenyl | —O— |
| 253 | — | —C≡C— | heterocyclenyl | alkylenyl | —O— |
| 254 | alkylenyl | —C≡C— | heterocyclenyl | alkylenyl | —O— |
| 255 | cycloalkylenyl | —C≡C— | heterocyclenyl | alkylenyl | —O— |
| 256 | heterocyclenyl | —C≡C— | heterocyclenyl | alkylenyl | —O— |
| 257 | alkylenyl | — | — | cycloalkylenyl | —O— |
| 258 | cycloalkylenyl | — | — | cycloalkylenyl | —O— |
| 259 | heterocyclenyl | — | — | cycloalkylenyl | —O— |
| 260 | — | —C(═O)— | — | cycloalkylenyl | —O— |
| 261 | alkylenyl | —C(═O)— | — | cycloalkylenyl | —O— |
| 262 | cycloalkylenyl | —C(═O)— | — | cycloalkylenyl | —O— |
| 263 | heterocyclenyl | —C(═O)— | — | cycloalkylenyl | —O— |
| 264 | — | —C(═O)NH— | — | cycloalkylenyl | —O— |
| 265 | alkylenyl | —C(═O)NH— | — | cycloalkylenyl | —O— |
| 266 | cycloalkylenyl | —C(═O)NH— | — | cycloalkylenyl | —O— |
| 267 | heterocyclenyl | —C(═O)NH— | — | cycloalkylenyl | —O— |
| 268 | — | —C≡C— | — | cycloalkylenyl | —O— |
| 269 | alkylenyl | —C≡C— | — | cycloalkylenyl | —O— |
| 270 | cycloalkylenyl | —C≡C— | — | cycloalkylenyl | —O— |
| 271 | heterocyclenyl | —C≡C— | — | cycloalkylenyl | —O— |
| 272 | alkylenyl | — | alkylenyl | cycloalkylenyl | —O— |
| 273 | cycloalkylenyl | — | alkylenyl | cycloalkylenyl | —O— |
| 274 | heterocyclenyl | — | alkylenyl | cycloalkylenyl | —O— |
| 275 | — | —C(═O)— | alkylenyl | cycloalkylenyl | —O— |
| 276 | alkylenyl | —C(═O)— | alkylenyl | cycloalkylenyl | —O— |
| 277 | cycloalkylenyl | —C(═O)— | alkylenyl | cycloalkylenyl | —O— |
| 278 | heterocyclenyl | —C(═O)— | alkylenyl | cycloalkylenyl | —O— |
| 279 | — | —C(═O)NH— | alkylenyl | cycloalkylenyl | —O— |
| 280 | alkylenyl | —C(═O)NH— | alkylenyl | cycloalkylenyl | —O— |
| 281 | cycloalkylenyl | —C(═O)NH— | alkylenyl | cycloalkylenyl | —O— |
| 282 | heterocyclenyl | —C(═O)NH— | alkylenyl | cycloalkylenyl | —O— |
| 283 | — | —C≡C— | alkylenyl | cycloalkylenyl | —O— |
| 284 | alkylenyl | —C≡C— | alkylenyl | cycloalkylenyl | —O— |
| 285 | cycloalkylenyl | —C≡C— | alkylenyl | cycloalkylenyl | —O— |
| 286 | heterocyclenyl | —C≡C— | alkylenyl | cycloalkylenyl | —O— |
| 287 | alkylenyl | — | heterocyclenyl | cycloalkylenyl | —O— |
| 288 | cycloalkylenyl | — | heterocyclenyl | cycloalkylenyl | —O— |
| 289 | heterocyclenyl | — | heterocyclenyl | cycloalkylenyl | —O— |
| 290 | — | —C(═O)— | heterocyclenyl | cycloalkylenyl | —O— |
| 291 | alkylenyl | —C(═O)— | heterocyclenyl | cycloalkylenyl | —O— |
| 292 | cycloalkylenyl | —C(═O)— | heterocyclenyl | cycloalkylenyl | —O— |
| 293 | heterocyclenyl | —C(═O)— | heterocyclenyl | cycloalkylenyl | —O— |
| 294 | — | —C(═O)NH— | heterocyclenyl | cycloalkylenyl | —O— |
| 295 | alkylenyl | —C(═O)NH— | heterocyclenyl | cycloalkylenyl | —O— |
| 296 | cycloalkylenyl | —C(═O)NH— | heterocyclenyl | cycloalkylenyl | —O— |
| 297 | heterocyclenyl | —C(═O)NH— | heterocyclenyl | cycloalkylenyl | —O— |
| 298 | — | —C≡C— | heterocyclenyl | cycloalkylenyl | —O— |
| 299 | alkylenyl | —C≡C— | heterocyclenyl | cycloalkylenyl | —O— |
| 300 | cycloalkylenyl | —C≡C— | heterocyclenyl | cycloalkylenyl | —O— |
| 301 | heterocyclenyl | —C≡C— | heterocyclenyl | cycloalkylenyl | —O— |
| 302 | alkylenyl | — | — | — | —NH— |
| 303 | cycloalkylenyl | — | — | — | —NH— |
| 304 | heterocyclenyl | — | — | — | —NH— |
| 305 | cycloalkylenyl | — | alkylenyl | — | —NH— |
| 306 | heterocyclenyl | — | alkylenyl | — | —NH— |
| 307 | — | —C(═O)— | alkylenyl | — | —NH— |
| 308 | alkylenyl | —C(═O)— | alkylenyl | — | —NH— |
| 309 | cycloalkylenyl | —C(═O)— | alkylenyl | — | —NH— |
| 310 | heterocyclenyl | —C(═O)— | alkylenyl | — | —NH— |
| 311 | — | —C(═O)NH— | alkylenyl | — | —NH— |
| 312 | alkylenyl | —C(═O)NH— | alkylenyl | — | —NH— |
| 313 | cycloalkylenyl | —C(═O)NH— | alkylenyl | — | —NH— |
| 314 | heterocyclenyl | —C(═O)NH— | alkylenyl | — | —NH— |
| 315 | — | —C≡C— | alkylenyl | — | —NH— |
| 316 | alkylenyl | —C≡C— | alkylenyl | — | —NH— |
| 317 | cycloalkylenyl | —C≡C— | alkylenyl | — | —NH— |
| 318 | heterocyclenyl | —C≡C— | alkylenyl | — | —NH— |
| 319 | alkylenyl | — | heterocyclenyl | — | —NH— |
| 320 | cycloalkylenyl | — | heterocyclenyl | — | —NH— |
| 321 | heterocyclenyl | — | heterocyclenyl | — | —NH— |
| 322 | — | —C(═O)— | heterocyclenyl | — | —NH— |
| 323 | alkylenyl | —C(═O)— | heterocyclenyl | — | —NH— |
| 324 | cycloalkylenyl | —C(═O)— | heterocyclenyl | — | —NH— |
| 325 | heterocyclenyl | —C(═O)— | heterocyclenyl | — | —NH— |
| 326 | — | —C(═O)NH— | heterocyclenyl | — | —NH— |
| 327 | alkylenyl | —C(═O)NH— | heterocyclenyl | — | —NH— |
| 328 | cycloalkylenyl | —C(═O)NH— | heterocyclenyl | — | —NH— |
| 329 | heterocyclenyl | —C(═O)NH— | heterocyclenyl | — | —NH— |
| 330 | — | —C≡C— | heterocyclenyl | — | —NH— |
| 331 | alkylenyl | —C≡C— | heterocyclenyl | — | —NH— |
| 332 | cycloalkylenyl | —C≡C— | heterocyclenyl | — | —NH— |
| 333 | heterocyclenyl | —C≡C— | heterocyclenyl | — | —NH— |
| 334 | alkylenyl | — | alkylenyl | heterocyclenyl | —NH— |
| 335 | cycloalkylenyl | — | alkylenyl | heterocyclenyl | —NH— |
| 336 | heterocyclenyl | — | alkylenyl | heterocyclenyl | —NH— |
| 337 | — | —C(═O)— | alkylenyl | heterocyclenyl | —NH— |
| 338 | alkylenyl | —C(═O)— | alkylenyl | heterocyclenyl | —NH— |
| 339 | cycloalkylenyl | —C(═O)— | alkylenyl | heterocyclenyl | —NH— |
| 340 | heterocyclenyl | —C(═O)— | alkylenyl | heterocyclenyl | —NH— |
| 341 | — | —C(═O)NH— | alkylenyl | heterocyclenyl | —NH— |
| 342 | alkylenyl | —C(═O)NH— | alkylenyl | heterocyclenyl | —NH— |
| 343 | cycloalkylenyl | —C(═O)NH— | alkylenyl | heterocyclenyl | —NH— |
| 344 | heterocyclenyl | —C(═O)NH— | alkylenyl | heterocyclenyl | —NH— |
| 345 | — | —C≡C— | alkylenyl | heterocyclenyl | —NH— |
| 346 | alkylenyl | —C≡C— | alkylenyl | heterocyclenyl | —NH— |
| 347 | cycloalkylenyl | —C≡C— | alkylenyl | heterocyclenyl | —NH— |
| 348 | heterocyclenyl | —C≡C— | alkylenyl | heterocyclenyl | —NH— |
| 349 | alkylenyl | — | heterocyclenyl | alkylenyl | —NH— |
| 350 | cycloalkylenyl | — | heterocyclenyl | alkylenyl | —NH— |
| 351 | heterocyclenyl | — | heterocyclenyl | alkylenyl | —NH— |
| 352 | — | —C(═O)— | heterocyclenyl | alkylenyl | —NH— |
| 353 | alkylenyl | —C(═O)— | heterocyclenyl | alkylenyl | —NH— |
| 354 | cycloalkylenyl | —C(═O)— | heterocyclenyl | alkylenyl | —NH— |
| 355 | heterocyclenyl | —C(═O)— | heterocyclenyl | alkylenyl | —NH— |
| 356 | — | —C(═O)NH— | heterocyclenyl | alkylenyl | —NH— |
| 357 | alkylenyl | —C(═O)NH— | heterocyclenyl | alkylenyl | —NH— |
| 358 | cycloalkylenyl | —C(═O)NH— | heterocyclenyl | alkylenyl | —NH— |
| 359 | heterocyclenyl | —C(═O)NH— | heterocyclenyl | alkylenyl | —NH— |
| 360 | — | —C≡C— | heterocyclenyl | alkylenyl | —NH— |
| 361 | alkylenyl | —C≡C— | heterocyclenyl | alkylenyl | —NH— |
| 362 | cycloalkylenyl | —C≡C— | heterocyclenyl | alkylenyl | —NH— |
| 363 | heterocyclenyl | —C≡C— | heterocyclenyl | alkylenyl | —NH— |
| 364 | alkylenyl | — | — | cycloalkylenyl | —NH— |
| 365 | cycloalkylenyl | — | — | cycloalkylenyl | —NH— |
| 366 | heterocyclenyl | — | — | cycloalkylenyl | —NH— |
| 367 | — | —C(═O)— | — | cycloalkylenyl | —NH— |
| 368 | alkylenyl | —C(═O)— | — | cycloalkylenyl | —NH— |
| 369 | cycloalkylenyl | —C(═O)— | — | cycloalkylenyl | —NH— |
| 370 | heterocyclenyl | —C(═O)— | — | cycloalkylenyl | —NH— |
| 371 | — | —C(═O)NH— | — | cycloalkylenyl | —NH— |
| 372 | alkylenyl | —C(═O)NH— | — | cycloalkylenyl | —NH— |
| 373 | cycloalkylenyl | —C(═O)NH— | — | cycloalkylenyl | —NH— |
| 374 | heterocyclenyl | —C(═O)NH— | — | cycloalkylenyl | —NH— |
| 375 | — | —C≡C— | — | cycloalkylenyl | —NH— |
| 376 | alkylenyl | —C≡C— | — | cycloalkylenyl | —NH— |
| 377 | cycloalkylenyl | —C≡C— | — | cycloalkylenyl | —NH— |
| 378 | heterocyclenyl | —C≡C— | — | cycloalkylenyl | —NH— |
| 379 | alkylenyl | — | alkylenyl | cycloalkylenyl | —NH— |
| 380 | cycloalkylenyl | — | alkylenyl | cycloalkylenyl | —NH— |
| 381 | heterocyclenyl | — | alkylenyl | cycloalkylenyl | —NH— |
| 382 | — | —C(═O)— | alkylenyl | cycloalkylenyl | —NH— |
| 383 | alkylenyl | —C(═O)— | alkylenyl | cycloalkylenyl | —NH— |
| 384 | cycloalkylenyl | —C(═O)— | alkylenyl | cycloalkylenyl | —NH— |
| 385 | heterocyclenyl | —C(═O)— | alkylenyl | cycloalkylenyl | —NH— |
| 386 | — | —C(═O)NH— | alkylenyl | cycloalkylenyl | —NH— |
| 387 | alkylenyl | —C(═O)NH— | alkylenyl | cycloalkylenyl | —NH— |
| 388 | cycloalkylenyl | —C(═O)NH— | alkylenyl | cycloalkylenyl | —NH— |
| 389 | heterocyclenyl | —C(═O)NH— | alkylenyl | cycloalkylenyl | —NH— |
| 390 | — | —C≡C— | alkylenyl | cycloalkylenyl | —NH— |
| 391 | alkylenyl | —C≡C— | alkylenyl | cycloalkylenyl | —NH— |
| 392 | cycloalkylenyl | —C≡C— | alkylenyl | cycloalkylenyl | —NH— |
| 393 | heterocyclenyl | —C≡C— | alkylenyl | cycloalkylenyl | —NH— |
| 394 | alkylenyl | — | heterocyclenyl | cycloalkylenyl | —NH— |
| 395 | cycloalkylenyl | — | heterocyclenyl | cycloalkylenyl | —NH— |
| 396 | heterocyclenyl | — | heterocyclenyl | cycloalkylenyl | —NH— |
| 397 | — | —C(═O)— | heterocyclenyl | cycloalkylenyl | —NH— |
| 398 | alkylenyl | —C(═O)— | heterocyclenyl | cycloalkylenyl | —NH— |
| 399 | cycloalkylenyl | —C(═O)— | heterocyclenyl | cycloalkylenyl | —NH— |
| 400 | heterocyclenyl | —C(═O)— | heterocyclenyl | cycloalkylenyl | —NH— |
| 401 | — | —C(═O)NH— | heterocyclenyl | cycloalkylenyl | —NH— |
| 402 | alkylenyl | —C(═O)NH— | heterocyclenyl | cycloalkylenyl | —NH— |
| 403 | cycloalkylenyl | —C(═O)NH— | heterocyclenyl | cycloalkylenyl | —NH— |
| 404 | heterocyclenyl | —C(═O)NH— | heterocyclenyl | cycloalkylenyl | —NH— |
| 405 | — | —C≡C— | heterocyclenyl | cycloalkylenyl | —NH— |
| 406 | alkylenyl | —C≡C— | heterocyclenyl | cycloalkylenyl | —NH— |
| 407 | cycloalkylenyl | —C≡C— | heterocyclenyl | cycloalkylenyl | —NH— |
| 408 | heterocyclenyl | —C≡C— | heterocyclenyl | cycloalkylenyl | —NH— |

In another embodiment, the each alkylenyl group listed in Table 5 is independently a C₁-C₆ alkylenyl.

In another embodiment, the each heterocyclenyl group listed in Table 5 is independently a 4- to 8-membered heterocyclenyl.

In another embodiment, Compounds of the Disclosure are compounds of Formula I, or a pharmaceutically acceptable salt or solvate thereof, wherein B¹ is B¹-1. In another embodiment, R¹¹ is hydrogen. In another embodiment, R¹³ is hydrogen. In another embodiment, Z is —CH₂—. In another embodiment, Z is —C(═O)—. In another embodiment, Y⁶ is —C(R^{10a})═, Y⁷ is —C(R^{10b})═, and Y⁸ is —C(R^{10c})═, and R^{10a}, R^{10b}, and R^{10c} are independently selected from the group consisting of hydrogen and halo. In another embodiment, R^{10a}, R^{10b}, and R^{10c} are hydrogen. In another embodiment, Y⁶ is —N═, Y⁷ is —C(R^{10b})═, and Y⁸ is —C(R^{10c})═, and R^{10b} and R^{10c} are independently selected from the group consisting of hydrogen and halo. In another embodiment, R^{10b} and R^{10c} are hydrogen. In another embodiment, Y⁶ is —C(R^{10a})═, Y⁷ is —N═, and Y⁸ is —C(R^{10c})═, and R^{10a} and R^{10c} are independently selected from the group consisting of hydrogen and halo. In another embodiment, R^{10a} and R^{10b} are hydrogen. In another embodiment, Y⁶ is —C(R^{10a})═, Y⁷ is —C(R^{10b})═, and Y⁸ is —N═, and R^{10a} and R^{10b} are independently selected from the group consisting of hydrogen and halo. In another embodiment, R^{10a} and R^{10b} are hydrogen.

In another embodiment, Compounds of the Disclosure are compounds of Formula I, or a pharmaceutically acceptable salt or solvate thereof, wherein B¹ is B¹-2. In another embodiment, R¹¹ is hydrogen. In another embodiment, R¹³ is hydrogen. In another embodiment, Z is —CH₂—. In another embodiment, Z is —C(═O)—. In another embodiment, Y⁹ is —C(R^{10d})═, Y⁷ is —C(R^{10b})═, and Y⁸ is —C(R^{10c})═, and R^{10d}, R^{10b}, and R^{10c} are independently selected from the group consisting of hydrogen and halo. In another embodiment, R^{10d}, R^{10b}, and R^{10c} are hydrogen. In another embodiment, Y⁹ is N═, Y⁷ is —C(R^{10b})═, and Y⁸ is —C(R^{10d})═, and R^{10b} and R^{10c} are independently selected from the group consisting of hydrogen and halo. In another embodiment, R^{10b} and R^{10c} are hydrogen. In another embodiment, Y⁹ is —C(R^{10d})═, Y⁷ is —N═, and Y⁸ is —C(R^{10c})═, and R^{10d} and R^{10c} are independently selected from the group consisting of hydrogen and halo. In another embodiment, R^{10d} and R^{10c} are hydrogen. In another embodiment, Y⁹ is —C(R^{10d})═, Y⁷ is —C(R^{10b})═, and Y⁸ is —N═, and R^{10d} and R^{10b} are independently selected from the group consisting of hydrogen and halo. In another embodiment, R^{10d} and R^{10b} are hydrogen.

In another embodiment, Compounds of the Disclosure are compounds of Formula I, or a pharmaceutically acceptable salt or solvate thereof, wherein B¹ is B¹-3. In another embodiment, R¹¹ is hydrogen. In another embodiment, R¹³ is hydrogen. In another embodiment, Z is —CH₂—. In another embodiment, Z is —C(═O)—. In another embodiment, Y⁶ is —C(R^{10a})═, Y⁹ is —C(R^{10d})═, and Y⁸ is —C(R^{10c})═, and R^{10a}, R^{10d}, and R^{10c} are independently selected from the group consisting of hydrogen and halo. In another embodiment, R^{10a}, R^{10d}, and R^{10c} are hydrogen. In another embodiment, Y⁶ is —N═, Y⁹ is —C(R^{10d})═, and Y⁸ is —C(R^{10c})═, and R^{10d} and R^{10c} are independently selected from the group consisting of hydrogen and halo. In another embodiment, R^{10d} and R^{10c} are hydrogen. In another embodiment, Y⁶ is —C(R^{10a})═, Y⁹ is —N═, and Y⁸ is —C(R¹⁰)═, and R^{10a} and R^{10b} are independently selected from the group consisting of hydrogen and halo. In another embodiment, R^{10a} and R^{10c} are hydrogen. In another embodiment, Y⁶ is —C(R^{10a})═, Y⁹ is —C(R^{10d})═, and Y⁸ is —N═, and R^{10a} and R^{10d} are independently selected from the group consisting of hydrogen and halo. In another embodiment, R^{10a} and R^{10d} are hydrogen.

In another embodiment, Compounds of the Disclosure are compounds of Formula I, or a pharmaceutically acceptable salt or solvate thereof, wherein B¹ is B¹-4. In another embodiment, R¹¹ is hydrogen. In another embodiment, R¹³ is hydrogen. In another embodiment, Z¹ is —CH₂—. In another embodiment, Y⁶ is —C(R^{10a})═, Y⁷ is —C(R^{10b})═, and Y⁹ is —C(R^{10d})═, and R^{10a}, R^{10b}, and R^{10d} are independently selected from the group consisting of hydrogen and halo. In another embodiment, R^{10a}, R^{10b}, and R^{10d} are hydrogen. In another embodiment, Y⁶ is —N═, Y⁷ is —C(R^{10b})═, and Y⁹ is —C(R^{10d})═, and R^{10b} and R^{10d} are independently selected from the group consisting of hydrogen and halo. In another embodiment, R^{10b} and R^{10d} are hydrogen. In another embodiment, Y⁶ is —C(R^{10a})═, Y⁷ is —N═, and Y⁹ is —C(R^{10d})═, and R^{10a} and R^{10d} are independently selected from the group consisting of hydrogen and halo. In another embodiment, R^{10a} and R^{10d} are hydrogen. In another embodiment, Y⁶ is —C(R^{10a})═, Y⁷ is —C(R^{10b})═, and Y⁹ is —N═, and R^{10a} and R^{10b} are independently selected from the group consisting of hydrogen and halo. In another embodiment, R^{10a} and R^{10b} are hydrogen.

In another embodiment, Compounds of the Disclosure are compounds of Formula I, or a pharmaceutically acceptable salt or solvate thereof, wherein B¹ is selected from the group consisting of:

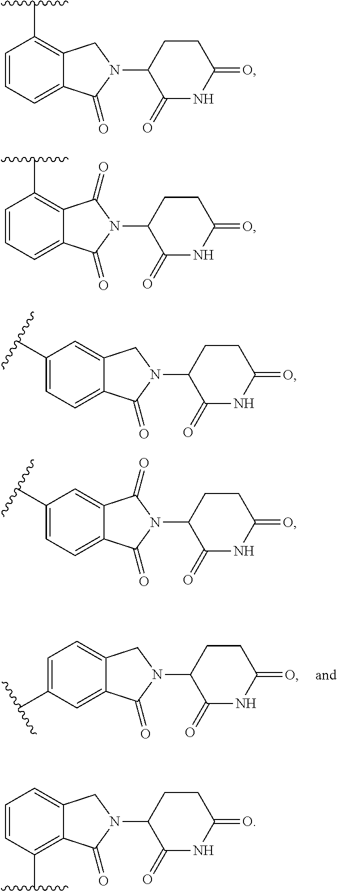

In another embodiment, Compounds of the Disclosure are compounds of Formula I, or a pharmaceutically acceptable salt or solvate thereof, wherein B¹ is:

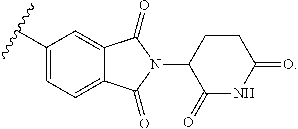 [SMILES: *c1ccc2c(c1)C(=O)N(C1CCC(=O)NC1=O)C2=O]

In another embodiment, Compounds of the Disclosure are any one or more of the compounds of Table 1, or a pharmaceutically acceptable salt or solvate thereof.

TABLE 1

| Cpd. | |
|---|---|
| No. | Structure |
| 1 | 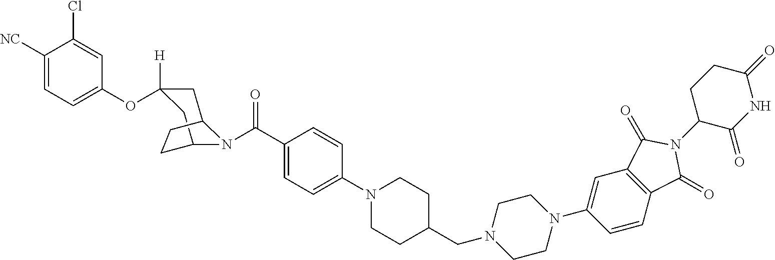 [SMILES: N#Cc1ccc(OC2CC3CCC(C2)N3C(=O)c2ccc(N3CCC(CN4CCN(c5ccc6c(c5)C(=O)N(C5CCC(=O)NC5=O)C6=O)CC4)CC3)cc2)cc1Cl] |
| 2 | 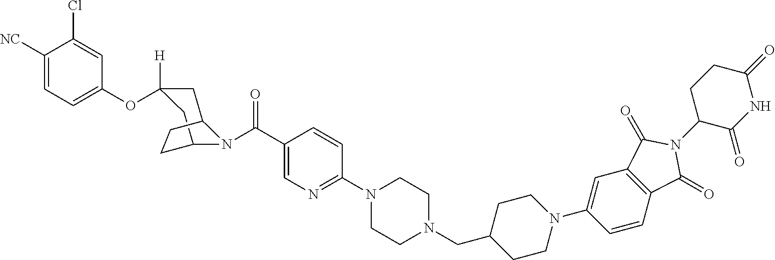 [SMILES: N#Cc1ccc(OC2CC3CCC(C2)N3C(=O)c2ccc(N3CCN(CC4CCN(c5ccc6c(c5)C(=O)N(C5CCC(=O)NC5=O)C6=O)CC4)CC3)nc2)cc1Cl] |
| 3 | 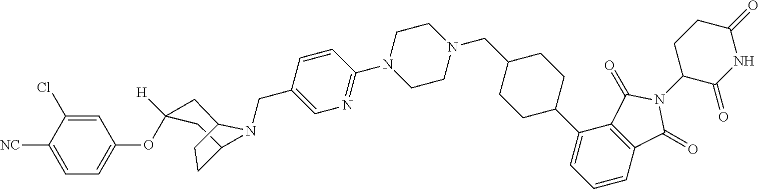 [SMILES: N#Cc1ccc(OC2CC3CCC(C2)N3Cc2ccc(N3CCN(CC4CCC(c5cccc6c5C(=O)N(C5CCC(=O)NC5=O)C6=O)CC4)CC3)nc2)cc1Cl] |
| 4 | 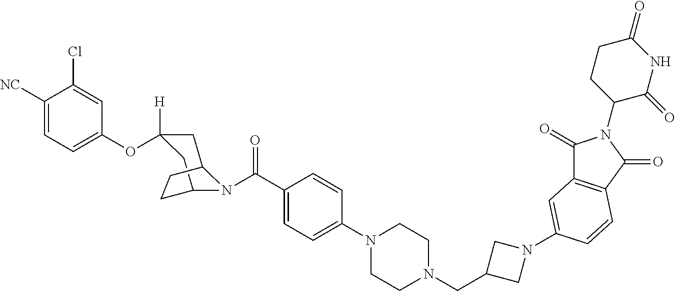 [SMILES: N#Cc1ccc(OC2CC3CCC(C2)N3C(=O)c2ccc(N3CCN(CC4CN(c5ccc6c(c5)C(=O)N(C5CCC(=O)NC5=O)C6=O)C4)CC3)cc2)cc1Cl] |
| 5 | 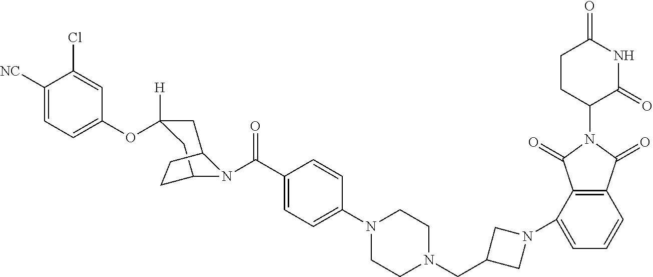 [SMILES: N#Cc1ccc(OC2CC3CCC(C2)N3C(=O)c2ccc(N3CCN(CC4CN(c5cccc6c5C(=O)N(C5CCC(=O)NC5=O)C6=O)C4)CC3)cc2)cc1Cl] |
| 6 | 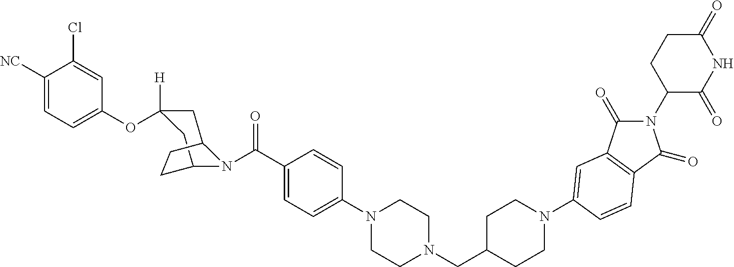 [SMILES: N#Cc1ccc(OC2CC3CCC(C2)N3C(=O)c2ccc(N3CCN(CC4CCN(c5ccc6c(c5)C(=O)N(C5CCC(=O)NC5=O)C6=O)CC4)CC3)cc2)cc1Cl] |
| 7 | 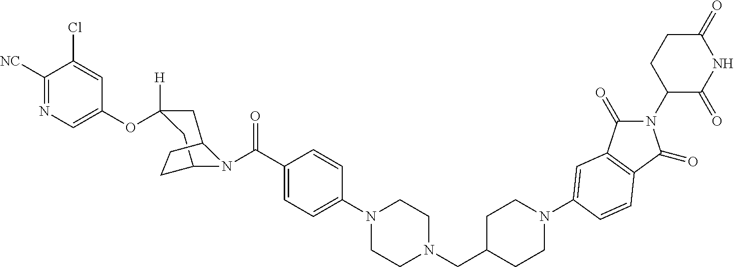 [SMILES: N#Cc1ncc(OC2CC3CCC(C2)N3C(=O)c2ccc(N3CCN(CC4CCN(c5ccc6c(c5)C(=O)N(C5CCC(=O)NC5=O)C6=O)CC4)CC3)cc2)cc1Cl] |
| 8 | 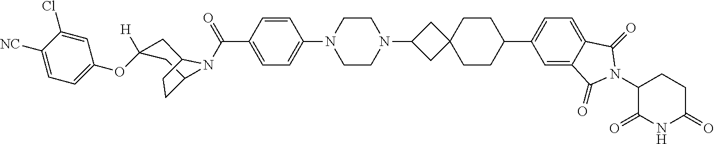 [SMILES: N#Cc1ccc(OC2CC3CCC(C2)N3C(=O)c2ccc(N3CCN(C4CC5(CCC(c6ccc7c(c6)C(=O)N(C6CCC(=O)NC6=O)C7=O)CC5)C4)CC3)cc2)cc1Cl] |
| 9 | 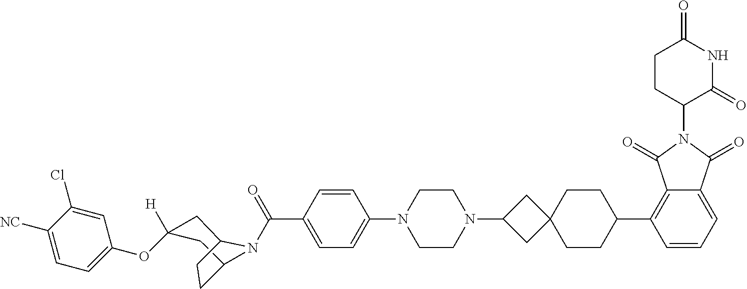 [SMILES: N#Cc1ccc(OC2CC3CCC(C2)N3C(=O)c2ccc(N3CCN(C4CC5(CCC(c6cccc7c6C(=O)N(C6CCC(=O)NC6=O)C7=O)CC5)C4)CC3)cc2)cc1Cl] |
| 10 | 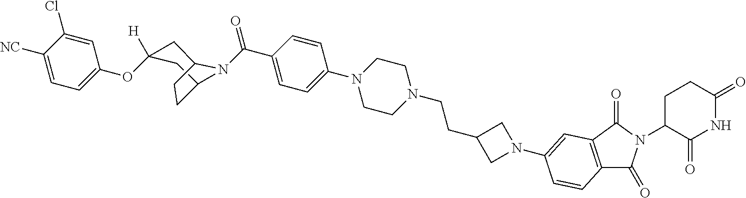 [SMILES: N#Cc1ccc(OC2CC3CCC(C2)N3C(=O)c2ccc(N3CCN(CCC4CN(c5ccc6c(c5)C(=O)N(C5CCC(=O)NC5=O)C6=O)C4)CC3)cc2)cc1Cl] |
| 11 | 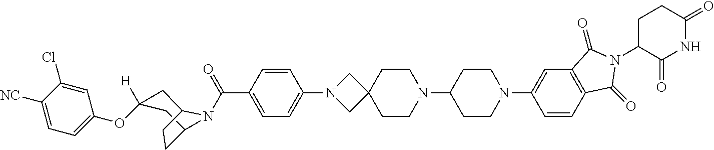 [SMILES: N#Cc1ccc(OC2CC3CCC(C2)N3C(=O)c2ccc(N3CC4(CCN(C5CCN(c6ccc7c(c6)C(=O)N(C6CCC(=O)NC6=O)C7=O)CC5)CC4)C3)cc2)cc1Cl] |
| 12 | 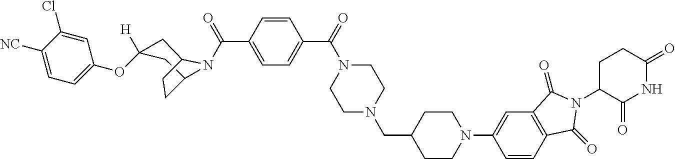 [SMILES: N#Cc1ccc(OC2CC3CCC(C2)N3C(=O)c2ccc(C(=O)N3CCN(CC4CCN(c5ccc6c(c5)C(=O)N(C5CCC(=O)NC5=O)C6=O)CC4)CC3)cc2)cc1Cl] |
| 13 | 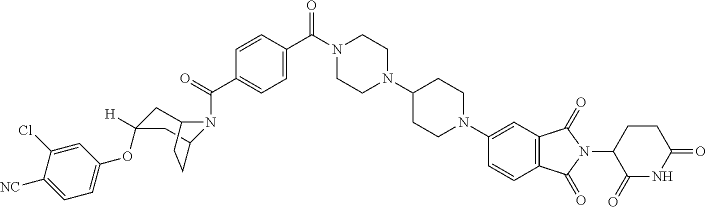 [SMILES: N#Cc1ccc(OC2CC3CCC(C2)N3C(=O)c2ccc(C(=O)N3CCN(C4CCN(c5ccc6c(c5)C(=O)N(C5CCC(=O)NC5=O)C6=O)CC4)CC3)cc2)cc1Cl] |
| 14 | 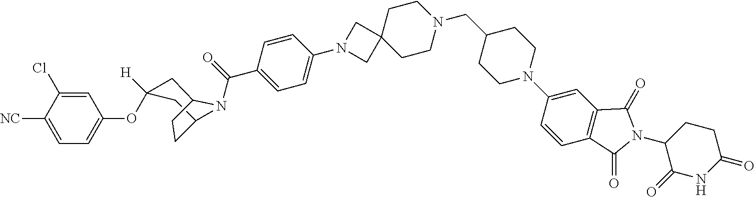 [SMILES: N#Cc1ccc(OC2CC3CCC(C2)N3C(=O)c2ccc(N3CC4(CCN(CC5CCN(c6ccc7c(c6)C(=O)N(C6CCC(=O)NC6=O)C7=O)CC5)CC4)C3)cc2)cc1Cl] |
| 15 | 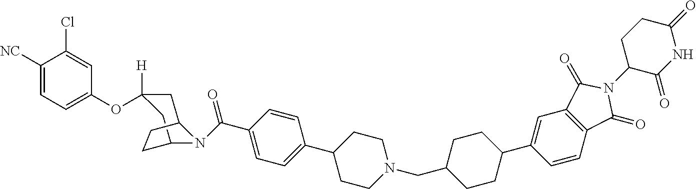 [SMILES: N#Cc1ccc(OC2CC3CCC(C2)N3C(=O)c2ccc(C3CCN(CC4CCC(c5ccc6c(c5)C(=O)N(C5CCC(=O)NC5=O)C6=O)CC4)CC3)cc2)cc1Cl] |
| 16 | 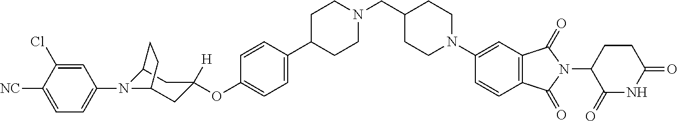 [SMILES: N#Cc1ccc(N2C3CCC2CC(Oc2ccc(C4CCN(CC5CCN(c6ccc7c(c6)C(=O)N(C6CCC(=O)NC6=O)C7=O)CC5)CC4)cc2)C3)cc1Cl] |
| 17 | 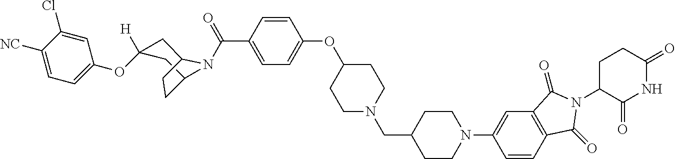 [SMILES: N#Cc1ccc(OC2CC3CCC(C2)N3C(=O)c2ccc(OC3CCN(CC4CCN(c5ccc6c(c5)C(=O)N(C5CCC(=O)NC5=O)C6=O)CC4)CC3)cc2)cc1Cl] |
| 18 | 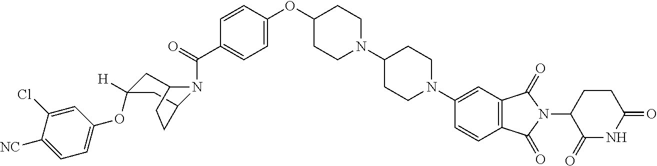 [SMILES: N#Cc1ccc(OC2CC3CCC(C2)N3C(=O)c2ccc(OC3CCN(C4CCN(c5ccc6c(c5)C(=O)N(C5CCC(=O)NC5=O)C6=O)CC4)CC3)cc2)cc1Cl] |
| 19 | 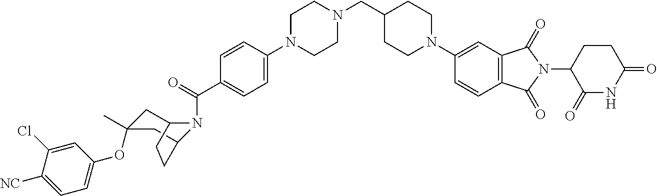 [SMILES: CC1(Oc2ccc(C#N)c(Cl)c2)CC2CCC(C1)N2C(=O)c1ccc(N2CCN(CC3CCN(c4ccc5c(c4)C(=O)N(C4CCC(=O)NC4=O)C5=O)CC3)CC2)cc1] |
| 20 | 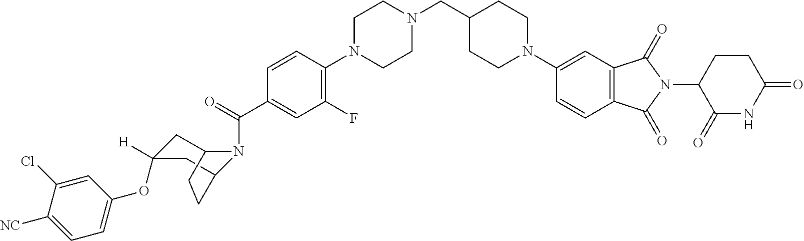 [SMILES: N#Cc1ccc(OC2CC3CCC(C2)N3C(=O)c2ccc(N3CCN(CC4CCN(c5ccc6c(c5)C(=O)N(C5CCC(=O)NC5=O)C6=O)CC4)CC3)c(F)c2)cc1Cl] |
| 21 | 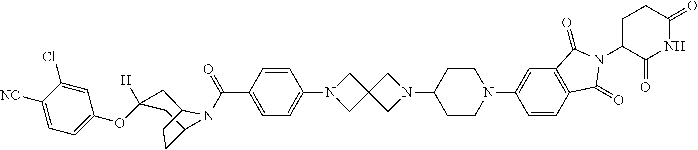 [SMILES: N#Cc1ccc(OC2CC3CCC(C2)N3C(=O)c2ccc(N3CC4(C3)CN(C3CCN(c5ccc6c(c5)C(=O)N(C5CCC(=O)NC5=O)C6=O)CC3)C4)cc2)cc1Cl] |
| 22 | 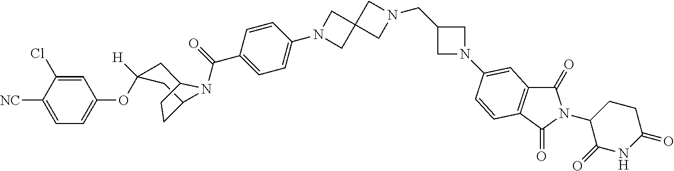 [SMILES: N#Cc1ccc(OC2CC3CCC(C2)N3C(=O)c2ccc(N3CC4(CN(CC5CN(c6ccc7c(c6)C(=O)N(C6CCC(=O)NC6=O)C7=O)C5)C4)C3)cc2)cc1Cl] |
| 23 | 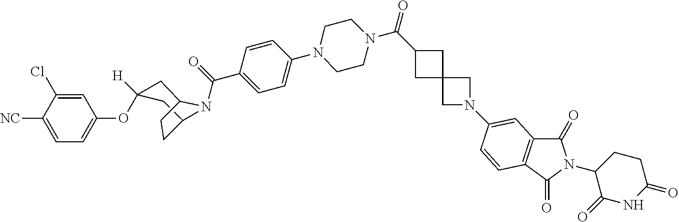 [SMILES: N#Cc1ccc(OC2CC3CCC(C2)N3C(=O)c2ccc(N3CCN(C(=O)C4CC5(C4)CN(c4ccc6c(c4)C(=O)N(C4CCC(=O)NC4=O)C6=O)C5)CC3)cc2)cc1Cl] |
| 24 | 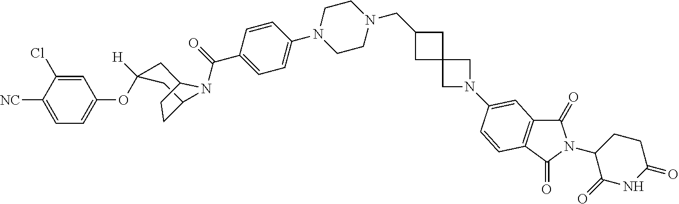 [SMILES: N#Cc1ccc(OC2CC3CCC(C2)N3C(=O)c2ccc(N3CCN(CC4CC5(C4)CN(c4ccc6c(c4)C(=O)N(C4CCC(=O)NC4=O)C6=O)C5)CC3)cc2)cc1Cl] |
| 25 | 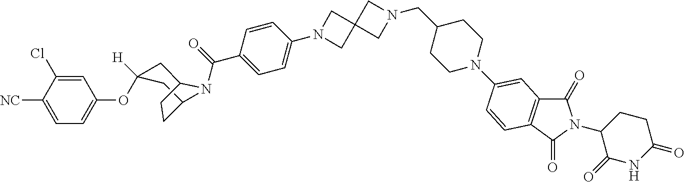 [SMILES: N#Cc1ccc(OC2CC3CCC(C2)N3C(=O)c2ccc(N3CC4(CN(CC5CCN(c6ccc7c(c6)C(=O)N(C6CCC(=O)NC6=O)C7=O)CC5)C4)C3)cc2)cc1Cl] |
| 26 | 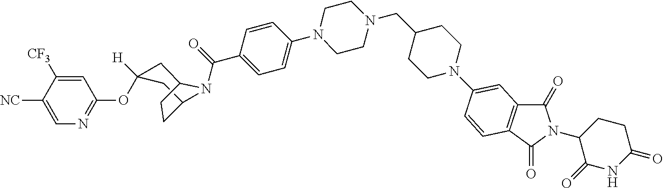 [SMILES: N#Cc1cnc(OC2CC3CCC(C2)N3C(=O)c2ccc(N3CCN(CC4CCN(c5ccc6c(c5)C(=O)N(C5CCC(=O)NC5=O)C6=O)CC4)CC3)cc2)cc1C(F)(F)F] |
| 27 | 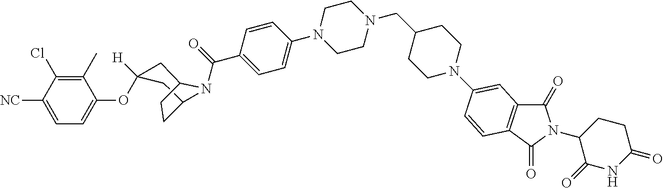 [SMILES: Cc1c(OC2CC3CCC(C2)N3C(=O)c2ccc(N3CCN(CC4CCN(c5ccc6c(c5)C(=O)N(C5CCC(=O)NC5=O)C6=O)CC4)CC3)cc2)ccc(C#N)c1Cl] |
| 28 | 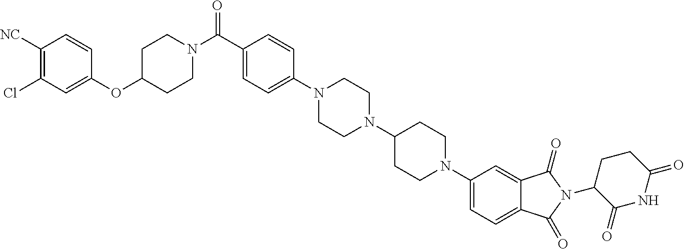 [SMILES: N#Cc1ccc(OC2CCN(C(=O)c3ccc(N4CCN(C5CCN(c6ccc7c(c6)C(=O)N(C6CCC(=O)NC6=O)C7=O)CC5)CC4)cc3)CC2)cc1Cl] |
| 29 | 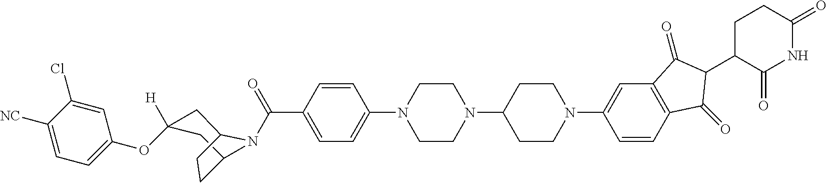 [SMILES: N#Cc1ccc(OC2CC3CCC(C2)N3C(=O)c2ccc(N3CCN(C4CCN(c5ccc6c(c5)C(=O)C(C5CCC(=O)NC5=O)C6=O)CC4)CC3)cc2)cc1Cl] |
| 30 | 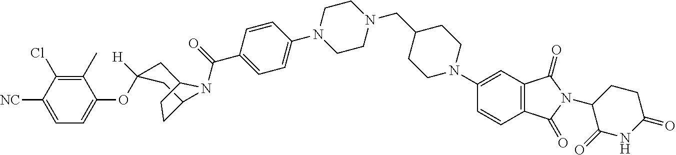 [SMILES: Cc1c(OC2CC3CCC(C2)N3C(=O)c2ccc(N3CCN(CC4CCN(c5ccc6c(c5)C(=O)N(C5CCC(=O)NC5=O)C6=O)CC4)CC3)cc2)ccc(C#N)c1Cl] |
| 31 | 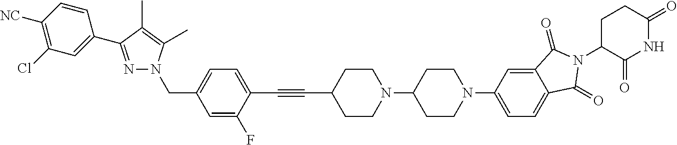 [SMILES: Cc1c(-c2ccc(C#N)c(Cl)c2)nn(Cc2ccc(C#CC3CCN(C4CCN(c5ccc6c(c5)C(=O)N(C5CCC(=O)NC5=O)C6=O)CC4)CC3)c(F)c2)c1C] |
| 32 | 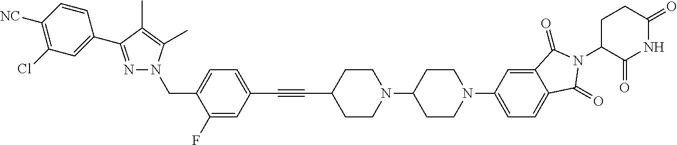 [SMILES: Cc1c(-c2ccc(C#N)c(Cl)c2)nn(Cc2ccc(C#CC3CCN(C4CCN(c5ccc6c(c5)C(=O)N(C5CCC(=O)NC5=O)C6=O)CC4)CC3)cc2F)c1C] |
| 33 | 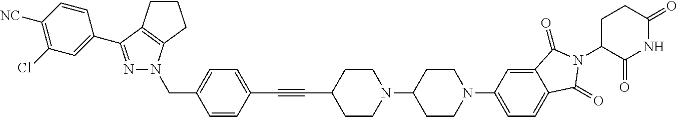 [SMILES: N#Cc1ccc(-c2nn(Cc3ccc(C#CC4CCN(C5CCN(c6ccc7c(c6)C(=O)N(C6CCC(=O)NC6=O)C7=O)CC5)CC4)cc3)c3c2CCC3)cc1Cl] |
| 34 | 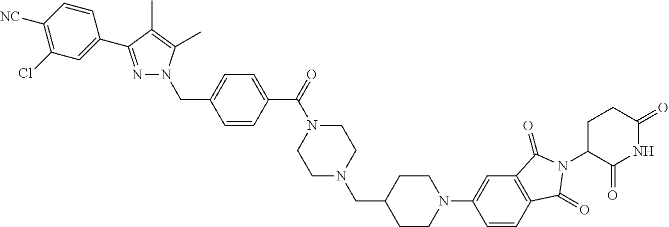 [SMILES: Cc1c(-c2ccc(C#N)c(Cl)c2)nn(Cc2ccc(C(=O)N3CCN(CC4CCN(c5ccc6c(c5)C(=O)N(C5CCC(=O)NC5=O)C6=O)CC4)CC3)cc2)c1C] |
| 35 | 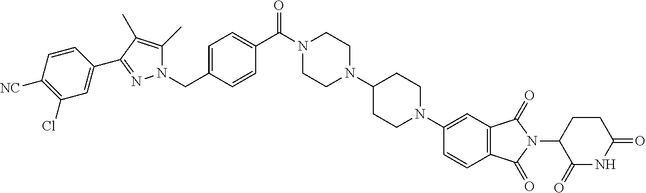 [SMILES: Cc1c(-c2ccc(C#N)c(Cl)c2)nn(Cc2ccc(C(=O)N3CCN(C4CCN(c5ccc6c(c5)C(=O)N(C5CCC(=O)NC5=O)C6=O)CC4)CC3)cc2)c1C] |
| 36 | 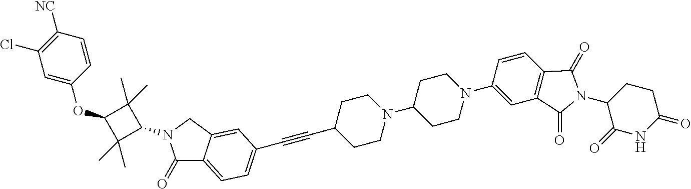 [SMILES: CC1(C)[C@H](Oc2ccc(C#N)c(Cl)c2)C(C)(C)[C@H]1N1Cc2cc(C#CC3CCN(C4CCN(c5ccc6c(c5)C(=O)N(C5CCC(=O)NC5=O)C6=O)CC4)CC3)ccc2C1=O] |
| 37 | 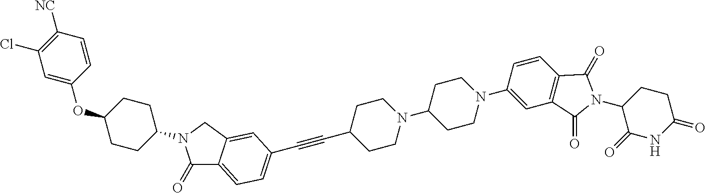 [SMILES: N#Cc1ccc(O[C@H]2CC[C@H](N3Cc4cc(C#CC5CCN(C6CCN(c7ccc8c(c7)C(=O)N(C7CCC(=O)NC7=O)C8=O)CC6)CC5)ccc4C3=O)CC2)cc1Cl] |
| 38 | 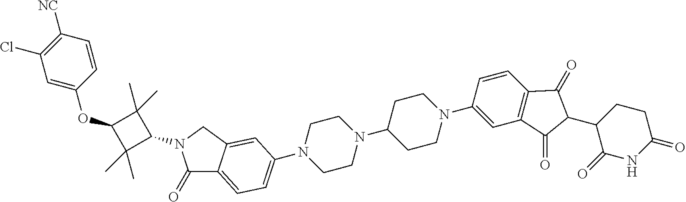 [SMILES: CC1(C)[C@H](Oc2ccc(C#N)c(Cl)c2)C(C)(C)[C@H]1N1Cc2cc(N3CCN(C4CCN(c5ccc6c(c5)C(=O)C(C5CCC(=O)NC5=O)C6=O)CC4)CC3)ccc2C1=O] |
| 39 | 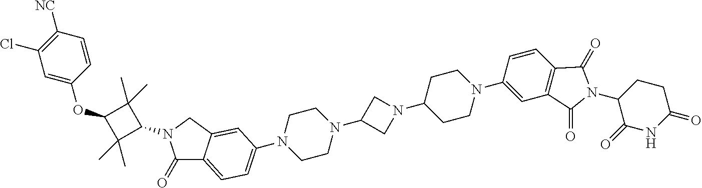 [SMILES: CC1(C)[C@H](Oc2ccc(C#N)c(Cl)c2)C(C)(C)[C@H]1N1Cc2cc(N3CCN(C4CN(C5CCN(c6ccc7c(c6)C(=O)N(C6CCC(=O)NC6=O)C7=O)CC5)C4)CC3)ccc2C1=O] |
| 40 | 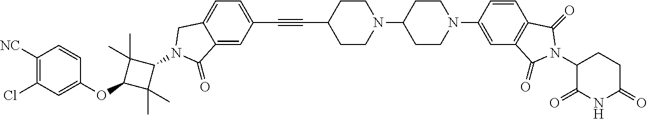 [SMILES: CC1(C)[C@H](Oc2ccc(C#N)c(Cl)c2)C(C)(C)[C@H]1N1Cc2ccc(C#CC3CCN(C4CCN(c5ccc6c(c5)C(=O)N(C5CCC(=O)NC5=O)C6=O)CC4)CC3)cc2C1=O] |
| 41 | 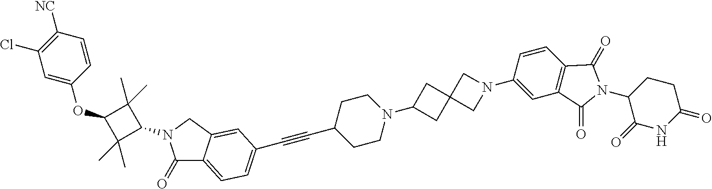 [SMILES: CC1(C)[C@H](Oc2ccc(C#N)c(Cl)c2)C(C)(C)[C@H]1N1Cc2cc(C#CC3CCN(C4CC5(C4)CN(c4ccc6c(c4)C(=O)N(C4CCC(=O)NC4=O)C6=O)C5)CC3)ccc2C1=O] |
| 42 | 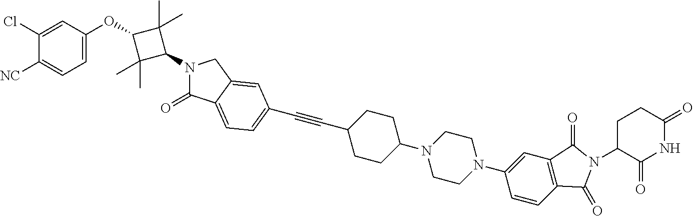 [SMILES: CC1(C)[C@H](Oc2ccc(C#N)c(Cl)c2)C(C)(C)[C@H]1N1Cc2cc(C#CC3CCC(N4CCN(c5ccc6c(c5)C(=O)N(C5CCC(=O)NC5=O)C6=O)CC4)CC3)ccc2C1=O] |
| 43 | 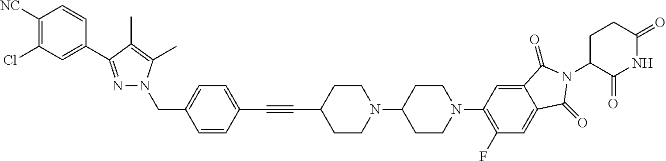 [SMILES: Cc1c(-c2ccc(C#N)c(Cl)c2)nn(Cc2ccc(C#CC3CCN(C4CCN(c5cc6c(cc5F)C(=O)N(C5CCC(=O)NC5=O)C6=O)CC4)CC3)cc2)c1C] |
| 44 | 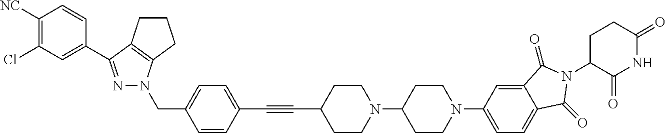 [SMILES: N#Cc1ccc(-c2nn(Cc3ccc(C#CC4CCN(C5CCN(c6ccc7c(c6)C(=O)N(C6CCC(=O)NC6=O)C7=O)CC5)CC4)cc3)c3c2CCC3)cc1Cl] |
| 45 | 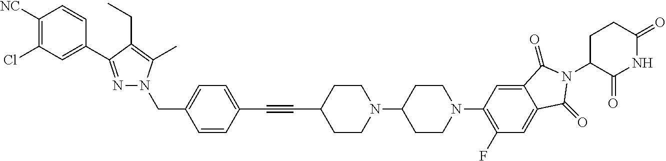 [SMILES: CCc1c(-c2ccc(C#N)c(Cl)c2)nn(Cc2ccc(C#CC3CCN(C4CCN(c5cc6c(cc5F)C(=O)N(C5CCC(=O)NC5=O)C6=O)CC4)CC3)cc2)c1C] |
| 46 | 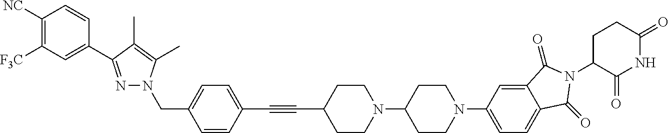 [SMILES: Cc1c(-c2ccc(C#N)c(C(F)(F)F)c2)nn(Cc2ccc(C#CC3CCN(C4CCN(c5ccc6c(c5)C(=O)N(C5CCC(=O)NC5=O)C6=O)CC4)CC3)cc2)c1C] |
| 47 | 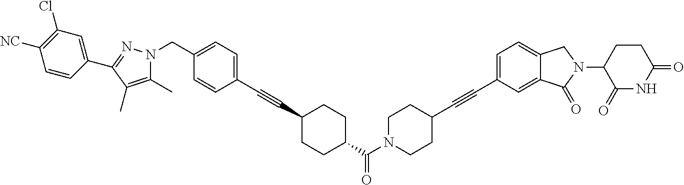 [SMILES: Cc1c(-c2ccc(C#N)c(Cl)c2)nn(Cc2ccc(C#C[C@H]3CC[C@H](C(=O)N4CCC(C#Cc5ccc6c(c5)C(=O)N(C5CCC(=O)NC5=O)C6)CC4)CC3)cc2)c1C] |
| 48 | 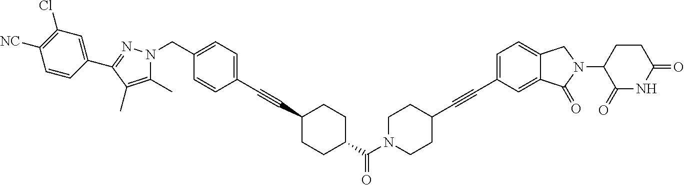 [SMILES: Cc1c(-c2ccc(C#N)c(Cl)c2)nn(Cc2ccc(C#C[C@H]3CC[C@H](C(=O)N4CCC(C#Cc5ccc6c(c5)C(=O)N(C5CCC(=O)NC5=O)C6)CC4)CC3)cc2)c1C] |
| 49 | 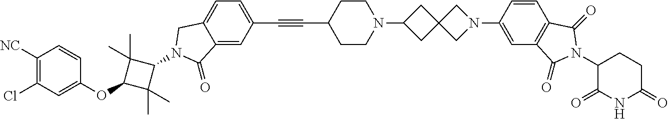 [SMILES: CC1(C)[C@H](Oc2ccc(C#N)c(Cl)c2)C(C)(C)[C@H]1N1Cc2ccc(C#CC3CCN(C4CC5(C4)CN(c4ccc6c(c4)C(=O)N(C4CCC(=O)NC4=O)C6=O)C5)CC3)cc2C1=O] |
| 50 | 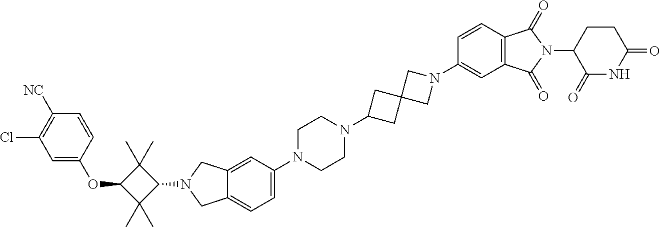 [SMILES: CC1(C)[C@H](Oc2ccc(C#N)c(Cl)c2)C(C)(C)[C@H]1N1Cc2ccc(N3CCN(C4CC5(C4)CN(c4ccc6c(c4)C(=O)N(C4CCC(=O)NC4=O)C6=O)C5)CC3)cc2C1] |
| 51 | 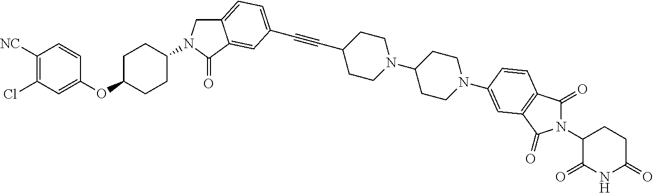 [SMILES: N#Cc1ccc(O[C@H]2CC[C@H](N3Cc4ccc(C#CC5CCN(C6CCN(c7ccc8c(c7)C(=O)N(C7CCC(=O)NC7=O)C8=O)CC6)CC5)cc4C3=O)CC2)cc1Cl] |
| 52 | 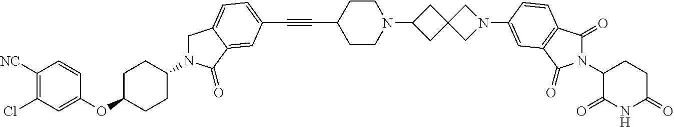 [SMILES: N#Cc1ccc(O[C@H]2CC[C@H](N3Cc4ccc(C#CC5CCN(C6CC7(C6)CN(c6ccc8c(c6)C(=O)N(C6CCC(=O)NC6=O)C8=O)C7)CC5)cc4C3=O)CC2)cc1Cl] |
| 53 | 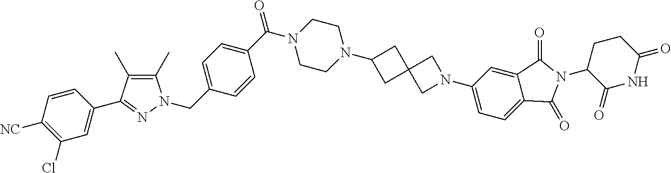 [SMILES: Cc1c(-c2ccc(C#N)c(Cl)c2)nn(Cc2ccc(C(=O)N3CCN(C4CC5(C4)CN(c4ccc6c(c4)C(=O)N(C4CCC(=O)NC4=O)C6=O)C5)CC3)cc2)c1C] |
| 54 | 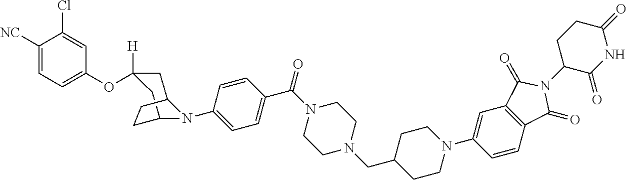 [SMILES: N#Cc1ccc(OC2CC3CCC(C2)N3c2ccc(C(=O)N3CCN(CC4CCN(c5ccc6c(c5)C(=O)N(C5CCC(=O)NC5=O)C6=O)CC4)CC3)cc2)cc1Cl] |
| 55 | 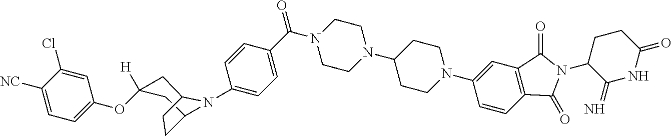 [SMILES: N#Cc1ccc(OC2CC3CCC(C2)N3c2ccc(C(=O)N3CCN(C4CCN(c5ccc6c(c5)C(=O)N(C5CCC(=O)NC5=N)C6=O)CC4)CC3)cc2)cc1Cl] |
| 56 | 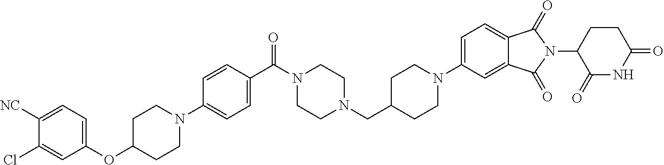 [SMILES: N#Cc1ccc(OC2CCN(c3ccc(C(=O)N4CCN(CC5CCN(c6ccc7c(c6)C(=O)N(C6CCC(=O)NC6=O)C7=O)CC5)CC4)cc3)CC2)cc1Cl] |
| 57 | 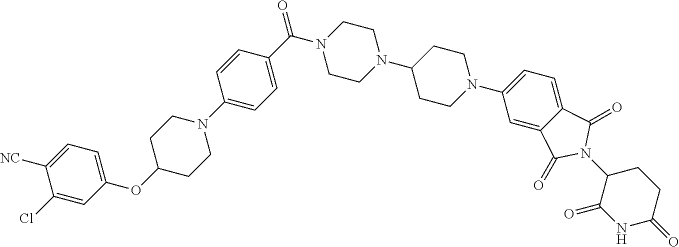 [SMILES: N#Cc1ccc(OC2CCN(c3ccc(C(=O)N4CCN(C5CCN(c6ccc7c(c6)C(=O)N(C6CCC(=O)NC6=O)C7=O)CC5)CC4)cc3)CC2)cc1Cl] |
| 58 | 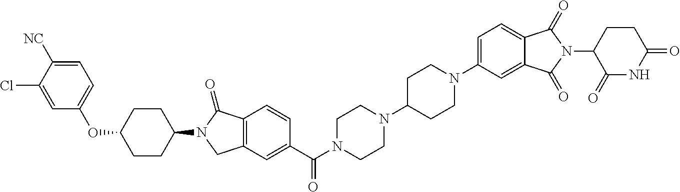 [SMILES: N#Cc1ccc(O[C@H]2CC[C@H](N3Cc4cc(C(=O)N5CCN(C6CCN(c7ccc8c(c7)C(=O)N(C7CCC(=O)NC7=O)C8=O)CC6)CC5)ccc4C3=O)CC2)cc1Cl] |
| 59 | 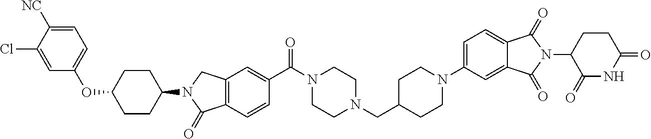 [SMILES: N#Cc1ccc(O[C@H]2CC[C@H](N3Cc4cc(C(=O)N5CCN(CC6CCN(c7ccc8c(c7)C(=O)N(C7CCC(=O)NC7=O)C8=O)CC6)CC5)ccc4C3=O)CC2)cc1Cl] |
| 60 | 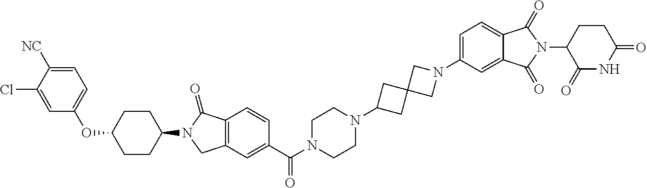 [SMILES: N#Cc1ccc(O[C@H]2CC[C@H](N3Cc4cc(C(=O)N5CCN(C6CC7(C6)CN(c6ccc8c(c6)C(=O)N(C6CCC(=O)NC6=O)C8=O)C7)CC5)ccc4C3=O)CC2)cc1Cl] |
| 61 | 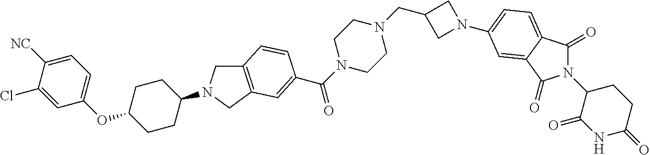 [SMILES: N#Cc1ccc(O[C@H]2CC[C@H](N3Cc4ccc(C(=O)N5CCN(CC6CN(c7ccc8c(c7)C(=O)N(C7CCC(=O)NC7=O)C8=O)C6)CC5)cc4C3)CC2)cc1Cl] |
| 62 | 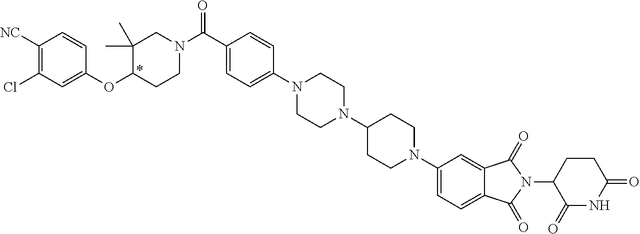 [SMILES: CC1(C)CN(C(=O)c2ccc(N3CCN(C4CCN(c5ccc6c(c5)C(=O)N(C5CCC(=O)NC5=O)C6=O)CC4)CC3)cc2)CCC1Oc1ccc(C#N)c(Cl)c1] |
| 63 | 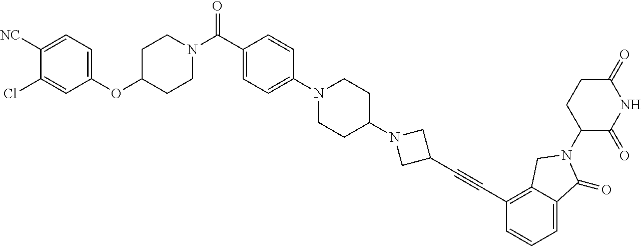 [SMILES: N#Cc1ccc(OC2CCN(C(=O)c3ccc(N4CCC(N5CC(C#Cc6cccc7c6CN(C6CCC(=O)NC6=O)C7=O)C5)CC4)cc3)CC2)cc1Cl] |
| 64 | 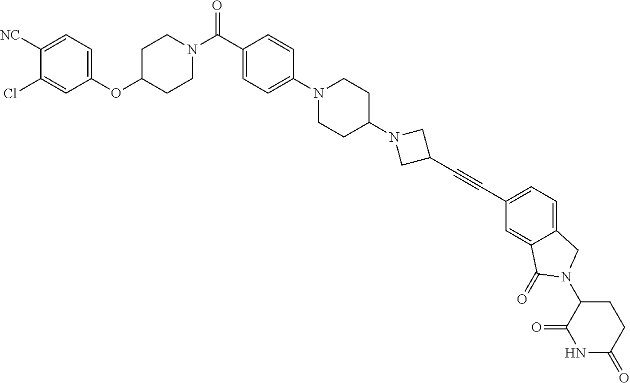 [SMILES: N#Cc1ccc(OC2CCN(C(=O)c3ccc(N4CCC(N5CC(C#Cc6ccc7c(c6)C(=O)N(C6CCC(=O)NC6=O)C7)C5)CC4)cc3)CC2)cc1Cl] |
| 65 | 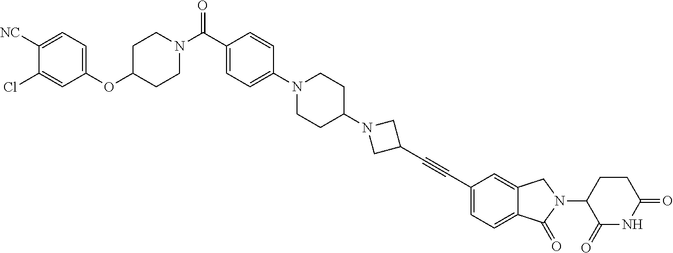 [SMILES: N#Cc1ccc(OC2CCN(C(=O)c3ccc(N4CCC(N5CC(C#Cc6ccc7c(c6)CN(C6CCC(=O)NC6=O)C7=O)C5)CC4)cc3)CC2)cc1Cl] |
| 66 | 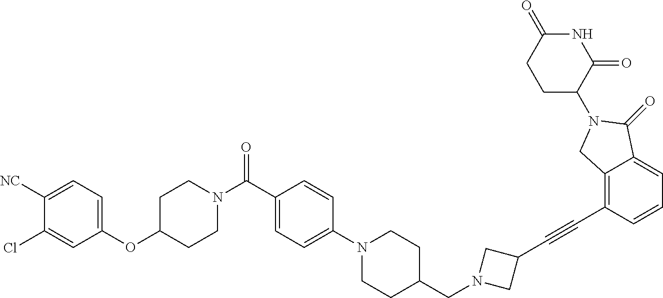 [SMILES: N#Cc1ccc(OC2CCN(C(=O)c3ccc(N4CCC(CN5CC(C#Cc6cccc7c6CN(C6CCC(=O)NC6=O)C7=O)C5)CC4)cc3)CC2)cc1Cl] |
| 67 | 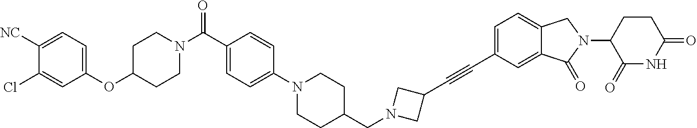 [SMILES: N#Cc1ccc(OC2CCN(C(=O)c3ccc(N4CCC(CN5CC(C#Cc6ccc7c(c6)C(=O)N(C6CCC(=O)NC6=O)C7)C5)CC4)cc3)CC2)cc1Cl] |
| 68 | 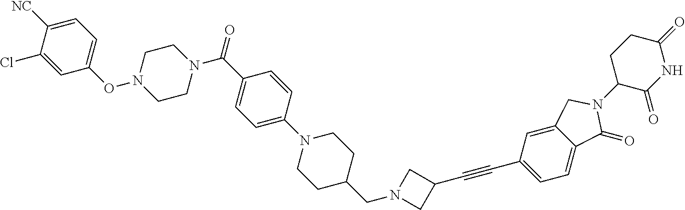 [SMILES: N#Cc1ccc(ON2CCN(C(=O)c3ccc(N4CCC(CN5CC(C#Cc6ccc7c(c6)CN(C6CCC(=O)NC6=O)C7=O)C5)CC4)cc3)CC2)cc1Cl] |
| 69 | 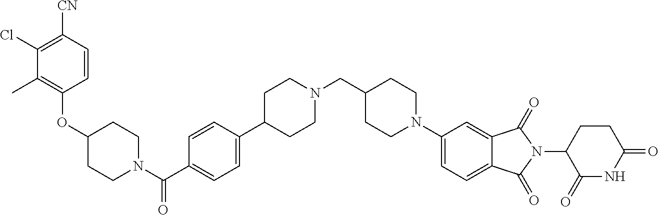 [SMILES: Cc1c(OC2CCN(C(=O)c3ccc(C4CCN(CC5CCN(c6ccc7c(c6)C(=O)N(C6CCC(=O)NC6=O)C7=O)CC5)CC4)cc3)CC2)ccc(C#N)c1Cl] |
| 70 | 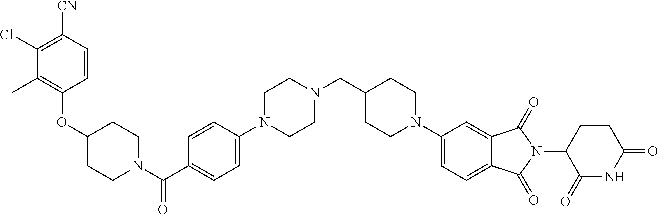 [SMILES: Cc1c(OC2CCN(C(=O)c3ccc(N4CCN(CC5CCN(c6ccc7c(c6)C(=O)N(C6CCC(=O)NC6=O)C7=O)CC5)CC4)cc3)CC2)ccc(C#N)c1Cl] |
| 71 | 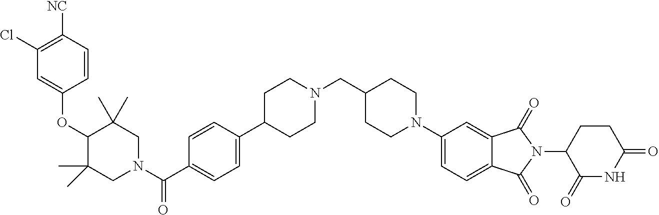 [SMILES: CC1(C)CN(C(=O)c2ccc(C3CCN(CC4CCN(c5ccc6c(c5)C(=O)N(C5CCC(=O)NC5=O)C6=O)CC4)CC3)cc2)CC(C)(C)C1Oc1ccc(C#N)c(Cl)c1] |
| 72 | 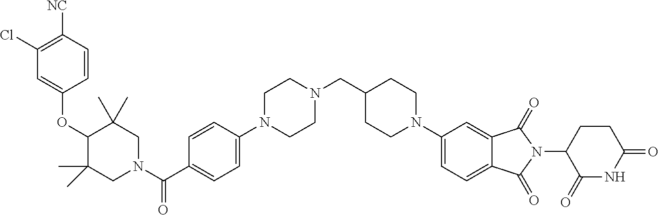 [SMILES: CC1(C)CN(C(=O)c2ccc(N3CCN(CC4CCN(c5ccc6c(c5)C(=O)N(C5CCC(=O)NC5=O)C6=O)CC4)CC3)cc2)CC(C)(C)C1Oc1ccc(C#N)c(Cl)c1] |
| 73 | 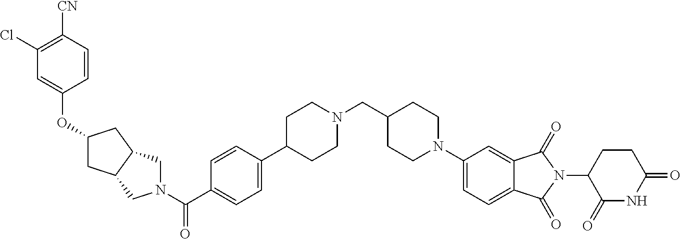 [SMILES: N#Cc1ccc(O[C@@H]2C[C@@H]3CN(C(=O)c4ccc(C5CCN(CC6CCN(c7ccc8c(c7)C(=O)N(C7CCC(=O)NC7=O)C8=O)CC6)CC5)cc4)C[C@@H]3C2)cc1Cl] |
| 74 | 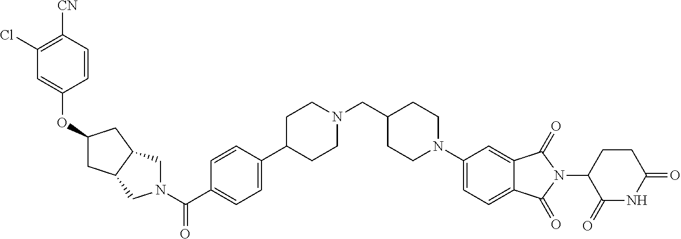 [SMILES: N#Cc1ccc(O[C@H]2C[C@@H]3CN(C(=O)c4ccc(C5CCN(CC6CCN(c7ccc8c(c7)C(=O)N(C7CCC(=O)NC7=O)C8=O)CC6)CC5)cc4)C[C@@H]3C2)cc1Cl] |
| 75 | 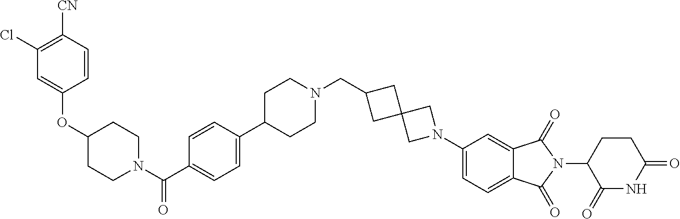 [SMILES: N#Cc1ccc(OC2CCN(C(=O)c3ccc(C4CCN(CC5CC6(C5)CN(c5ccc7c(c5)C(=O)N(C5CCC(=O)NC5=O)C7=O)C6)CC4)cc3)CC2)cc1Cl] |
| 76 | 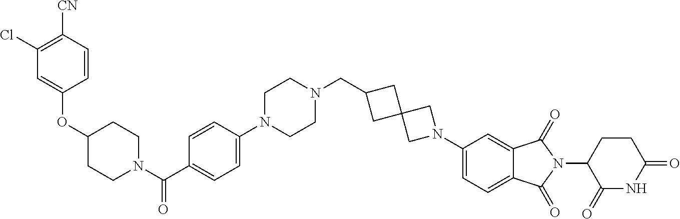 [SMILES: N#Cc1ccc(OC2CCN(C(=O)c3ccc(N4CCN(CC5CC6(C5)CN(c5ccc7c(c5)C(=O)N(C5CCC(=O)NC5=O)C7=O)C6)CC4)cc3)CC2)cc1Cl] |
| 77 | 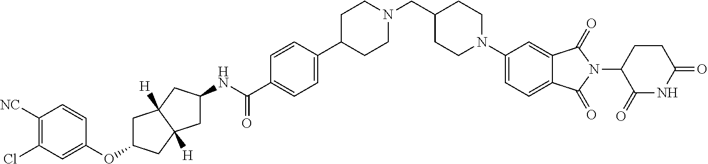 [SMILES: N#Cc1ccc(O[C@H]2C[C@H]3C[C@H](NC(=O)c4ccc(C5CCN(CC6CCN(c7ccc8c(c7)C(=O)N(C7CCC(=O)NC7=O)C8=O)CC6)CC5)cc4)C[C@H]3C2)cc1Cl] |
| 78 | 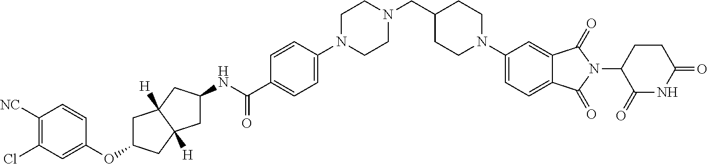 [SMILES: N#Cc1ccc(O[C@H]2C[C@H]3C[C@H](NC(=O)c4ccc(N5CCN(CC6CCN(c7ccc8c(c7)C(=O)N(C7CCC(=O)NC7=O)C8=O)CC6)CC5)cc4)C[C@H]3C2)cc1Cl] |
| 79 | 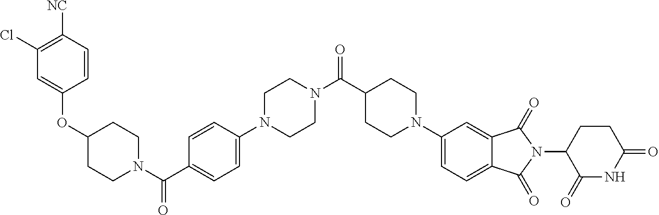 [SMILES: N#Cc1ccc(OC2CCN(C(=O)c3ccc(N4CCN(C(=O)C5CCN(c6ccc7c(c6)C(=O)N(C6CCC(=O)NC6=O)C7=O)CC5)CC4)cc3)CC2)cc1Cl] |
| 80 | 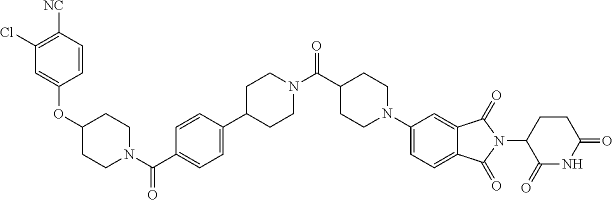 [SMILES: N#Cc1ccc(OC2CCN(C(=O)c3ccc(C4CCN(C(=O)C5CCN(c6ccc7c(c6)C(=O)N(C6CCC(=O)NC6=O)C7=O)CC5)CC4)cc3)CC2)cc1Cl] |
| 81 | 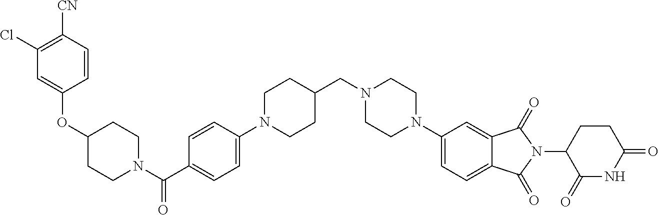 [SMILES: N#Cc1ccc(OC2CCN(C(=O)c3ccc(N4CCC(CN5CCN(c6ccc7c(c6)C(=O)N(C6CCC(=O)NC6=O)C7=O)CC5)CC4)cc3)CC2)cc1Cl] |
| 82 | 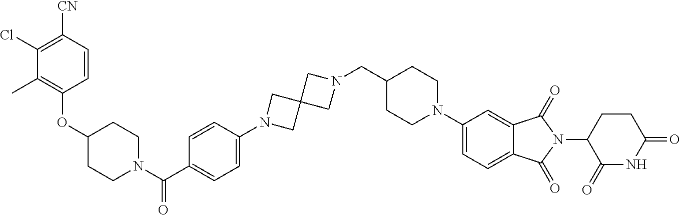 [SMILES: Cc1c(OC2CCN(C(=O)c3ccc(N4CC5(CN(CC6CCN(c7ccc8c(c7)C(=O)N(C7CCC(=O)NC7=O)C8=O)CC6)C5)C4)cc3)CC2)ccc(C#N)c1Cl] |
| 83 | 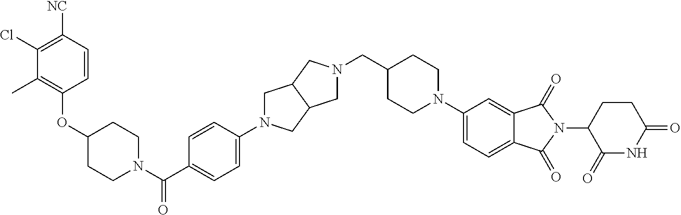 [SMILES: Cc1c(OC2CCN(C(=O)c3ccc(N4CC5CN(CC6CCN(c7ccc8c(c7)C(=O)N(C7CCC(=O)NC7=O)C8=O)CC6)CC5C4)cc3)CC2)ccc(C#N)c1Cl] |
| 84 | 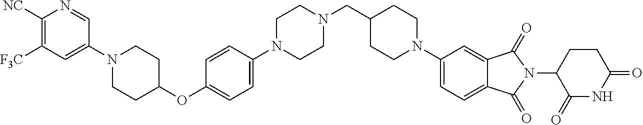 [SMILES: N#Cc1ncc(N2CCC(Oc3ccc(N4CCN(CC5CCN(c6ccc7c(c6)C(=O)N(C6CCC(=O)NC6=O)C7=O)CC5)CC4)cc3)CC2)cc1C(F)(F)F] |
| 85 | 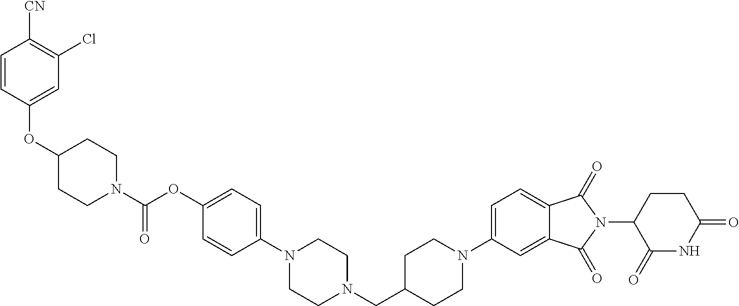 [SMILES: N#Cc1ccc(OC2CCN(C(=O)Oc3ccc(N4CCN(CC5CCN(c6ccc7c(c6)C(=O)N(C6CCC(=O)NC6=O)C7=O)CC5)CC4)cc3)CC2)cc1Cl] |
| 86 | 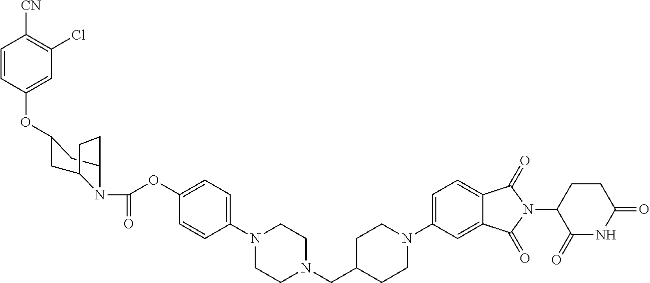 [SMILES: N#Cc1ccc(OC2CC3CCC(C2)N3C(=O)Oc2ccc(N3CCN(CC4CCN(c5ccc6c(c5)C(=O)N(C5CCC(=O)NC5=O)C6=O)CC4)CC3)cc2)cc1Cl] |
| 87 | 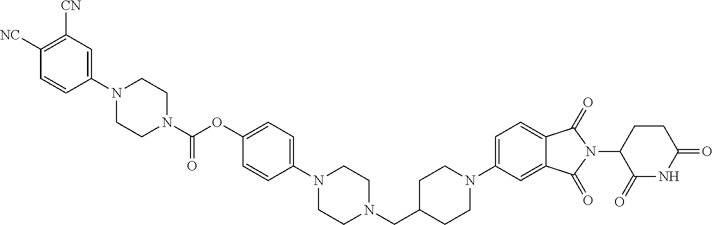 [SMILES: N#Cc1ccc(N2CCN(C(=O)Oc3ccc(N4CCN(CC5CCN(c6ccc7c(c6)C(=O)N(C6CCC(=O)NC6=O)C7=O)CC5)CC4)cc3)CC2)cc1C#N] |
| 88 | 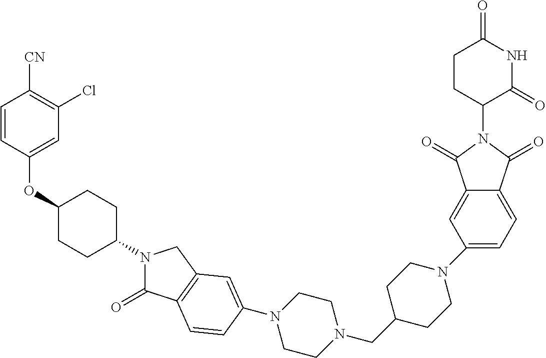 [SMILES: N#Cc1ccc(O[C@H]2CC[C@H](N3Cc4cc(N5CCN(CC6CCN(c7ccc8c(c7)C(=O)N(C7CCC(=O)NC7=O)C8=O)CC6)CC5)ccc4C3=O)CC2)cc1Cl] |
| 89 | 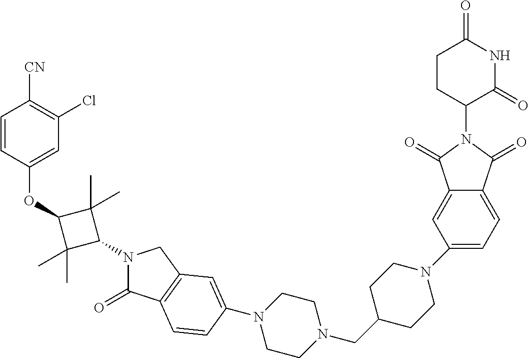 [SMILES: CC1(C)[C@H](Oc2ccc(C#N)c(Cl)c2)C(C)(C)[C@H]1N1Cc2cc(N3CCN(CC4CCN(c5ccc6c(c5)C(=O)N(C5CCC(=O)NC5=O)C6=O)CC4)CC3)ccc2C1=O] |
| 90 | 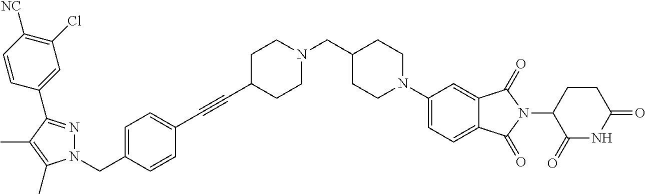 [SMILES: Cc1c(-c2ccc(C#N)c(Cl)c2)nn(Cc2ccc(C#CC3CCN(CC4CCN(c5ccc6c(c5)C(=O)N(C5CCC(=O)NC5=O)C6=O)CC4)CC3)cc2)c1C] |
| 91 | 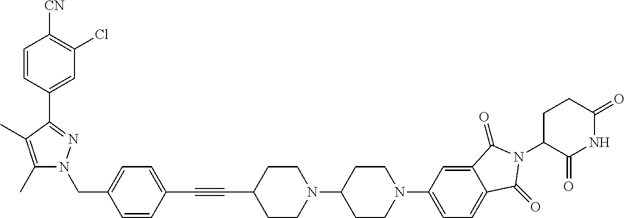 [SMILES: Cc1c(-c2ccc(C#N)c(Cl)c2)nn(Cc2ccc(C#CC3CCN(C4CCN(c5ccc6c(c5)C(=O)N(C5CCC(=O)NC5=O)C6=O)CC4)CC3)cc2)c1C] |
| 92 | 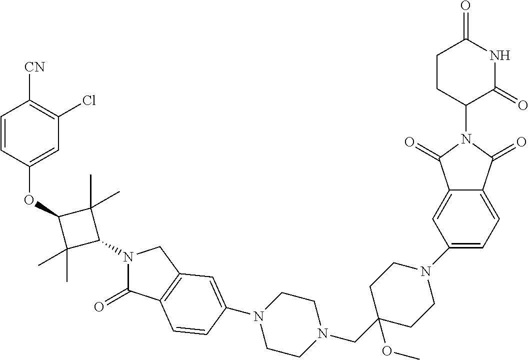 [SMILES: COC1(CN2CCN(c3ccc4c(c3)CN([C@H]3C(C)(C)[C@H](Oc5ccc(C#N)c(Cl)c5)C3(C)C)C4=O)CC2)CCN(c2ccc3c(c2)C(=O)N(C2CCC(=O)NC2=O)C3=O)CC1] |
| 93 | 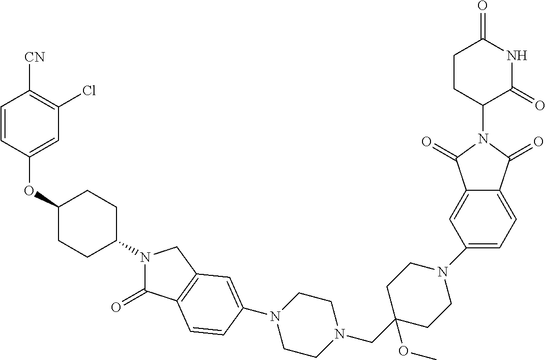 [SMILES: COC1(CN2CCN(c3ccc4c(c3)CN([C@H]3CC[C@H](Oc5ccc(C#N)c(Cl)c5)CC3)C4=O)CC2)CCN(c2ccc3c(c2)C(=O)N(C2CCC(=O)NC2=O)C3=O)CC1] |
| 94 | 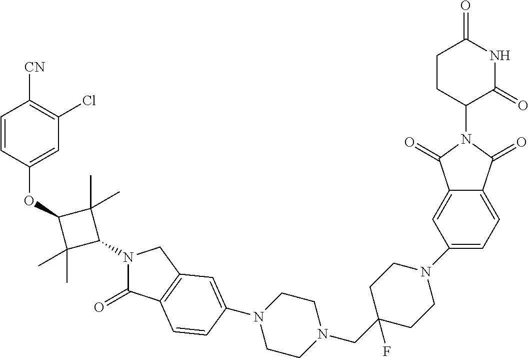 [SMILES: CC1(C)[C@H](Oc2ccc(C#N)c(Cl)c2)C(C)(C)[C@H]1N1Cc2cc(N3CCN(CC4(F)CCN(c5ccc6c(c5)C(=O)N(C5CCC(=O)NC5=O)C6=O)CC4)CC3)ccc2C1=O] |
| 95 | 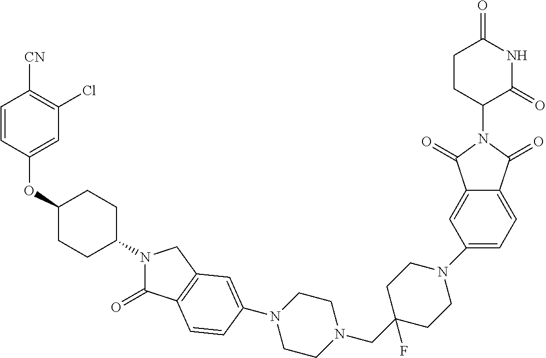 [SMILES: N#Cc1ccc(O[C@H]2CC[C@H](N3Cc4cc(N5CCN(CC6(F)CCN(c7ccc8c(c7)C(=O)N(C7CCC(=O)NC7=O)C8=O)CC6)CC5)ccc4C3=O)CC2)cc1Cl] |
| 96 | 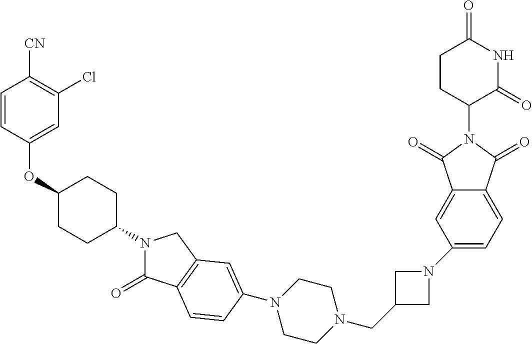 [SMILES: N#Cc1ccc(O[C@H]2CC[C@H](N3Cc4cc(N5CCN(CC6CN(c7ccc8c(c7)C(=O)N(C7CCC(=O)NC7=O)C8=O)C6)CC5)ccc4C3=O)CC2)cc1Cl] |
| 97 | 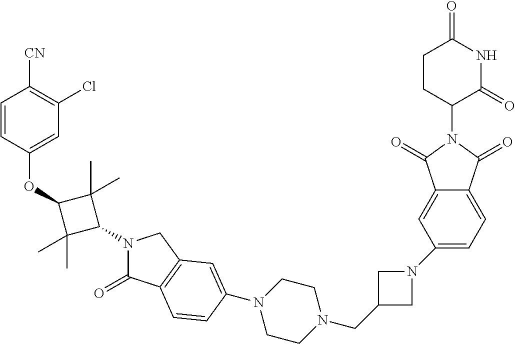 [SMILES: CC1(C)[C@H](Oc2ccc(C#N)c(Cl)c2)C(C)(C)[C@H]1N1Cc2cc(N3CCN(CC4CN(c5ccc6c(c5)C(=O)N(C5CCC(=O)NC5=O)C6=O)C4)CC3)ccc2C1=O] |
| 98 | 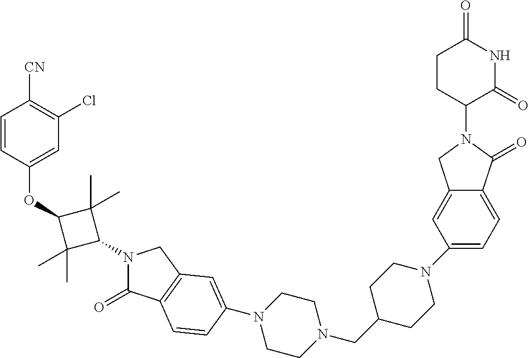 [SMILES: CC1(C)[C@H](Oc2ccc(C#N)c(Cl)c2)C(C)(C)[C@H]1N1Cc2cc(N3CCN(CC4CCN(c5ccc6c(c5)CN(C5CCC(=O)NC5=O)C6=O)CC4)CC3)ccc2C1=O] |
| 99 | 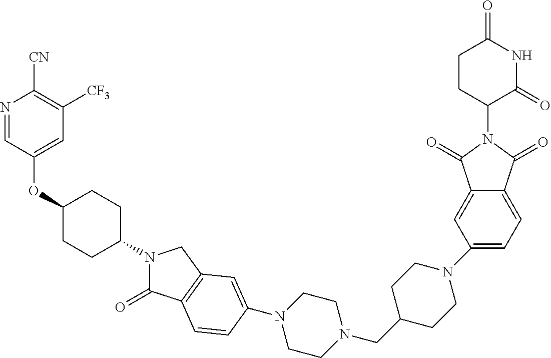 [SMILES: N#Cc1ncc(O[C@H]2CC[C@H](N3Cc4cc(N5CCN(CC6CCN(c7ccc8c(c7)C(=O)N(C7CCC(=O)NC7=O)C8=O)CC6)CC5)ccc4C3=O)CC2)cc1C(F)(F)F] |
| 100 | 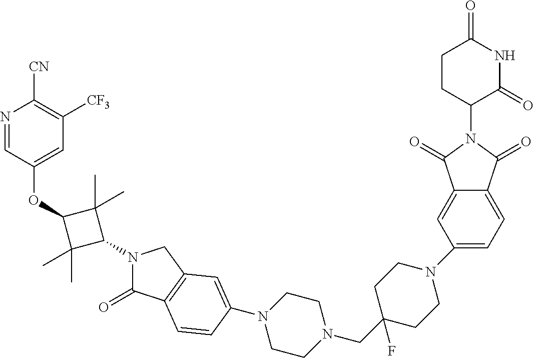 [SMILES: CC1(C)[C@H](Oc2cnc(C#N)c(C(F)(F)F)c2)C(C)(C)[C@H]1N1Cc2cc(N3CCN(CC4(F)CCN(c5ccc6c(c5)C(=O)N(C5CCC(=O)NC5=O)C6=O)CC4)CC3)ccc2C1=O] |
| 101 | 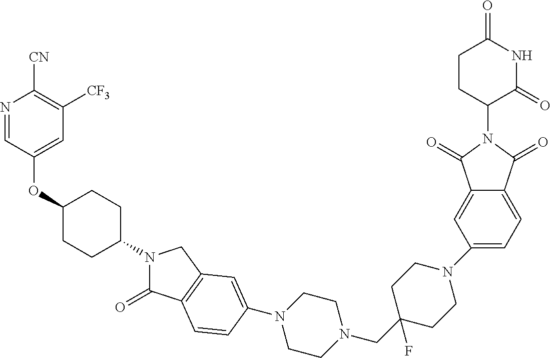 [SMILES: N#Cc1ncc(O[C@H]2CC[C@H](N3Cc4cc(N5CCN(CC6(F)CCN(c7ccc8c(c7)C(=O)N(C7CCC(=O)NC7=O)C8=O)CC6)CC5)ccc4C3=O)CC2)cc1C(F)(F)F] |
| 102 | 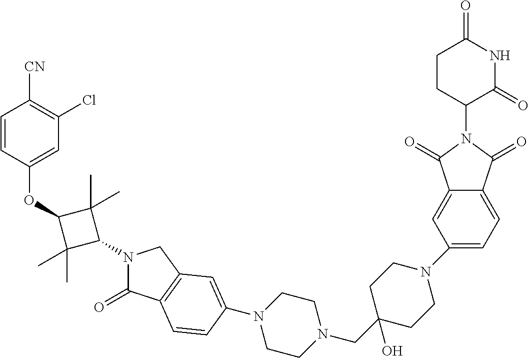 [SMILES: CC1(C)[C@H](Oc2ccc(C#N)c(Cl)c2)C(C)(C)[C@H]1N1Cc2cc(N3CCN(CC4(O)CCN(c5ccc6c(c5)C(=O)N(C5CCC(=O)NC5=O)C6=O)CC4)CC3)ccc2C1=O] |
| 103 | 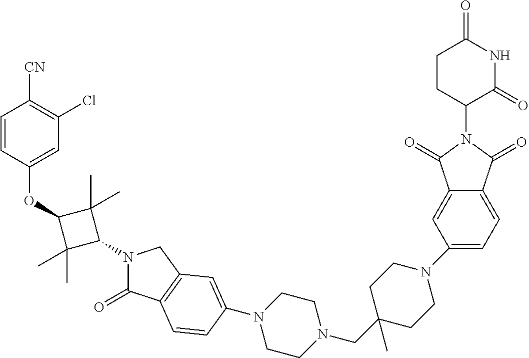 [SMILES: CC1(CN2CCN(c3ccc4c(c3)CN([C@H]3C(C)(C)[C@H](Oc5ccc(C#N)c(Cl)c5)C3(C)C)C4=O)CC2)CCN(c2ccc3c(c2)C(=O)N(C2CCC(=O)NC2=O)C3=O)CC1] |
| 104 | 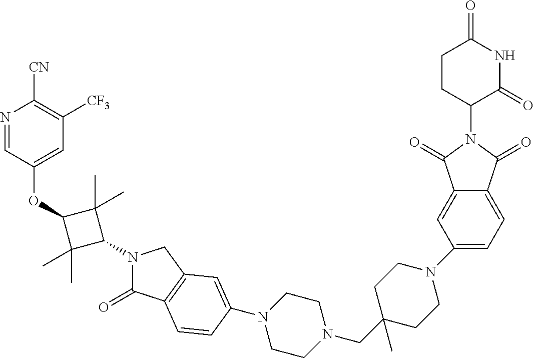 [SMILES: CC1(CN2CCN(c3ccc4c(c3)CN([C@H]3C(C)(C)[C@H](Oc5cnc(C#N)c(C(F)(F)F)c5)C3(C)C)C4=O)CC2)CCN(c2ccc3c(c2)C(=O)N(C2CCC(=O)NC2=O)C3=O)CC1] |
| 105 | 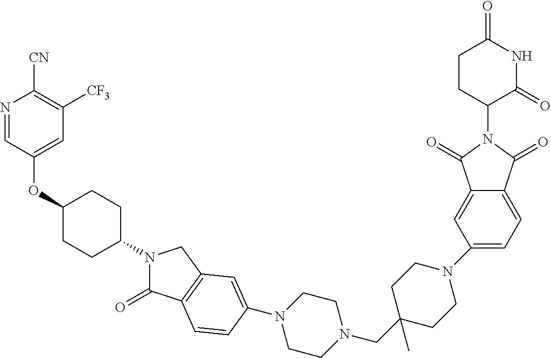 [SMILES: CC1(CN2CCN(c3ccc4c(c3)CN([C@H]3CC[C@H](Oc5cnc(C#N)c(C(F)(F)F)c5)CC3)C4=O)CC2)CCN(c2ccc3c(c2)C(=O)N(C2CCC(=O)NC2=O)C3=O)CC1] |
| 106 | 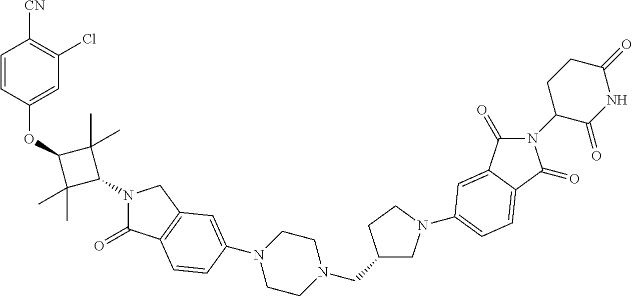 [SMILES: CC1(C)[C@H](Oc2ccc(C#N)c(Cl)c2)C(C)(C)[C@H]1N1Cc2cc(N3CCN(C[C@@H]4CCN(c5ccc6c(c5)C(=O)N(C5CCC(=O)NC5=O)C6=O)C4)CC3)ccc2C1=O] |
| 107 | 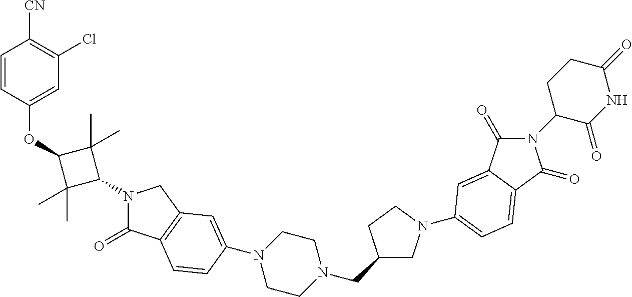 [SMILES: CC1(C)[C@H](Oc2ccc(C#N)c(Cl)c2)C(C)(C)[C@H]1N1Cc2cc(N3CCN(C[C@H]4CCN(c5ccc6c(c5)C(=O)N(C5CCC(=O)NC5=O)C6=O)C4)CC3)ccc2C1=O] |
| 108 | 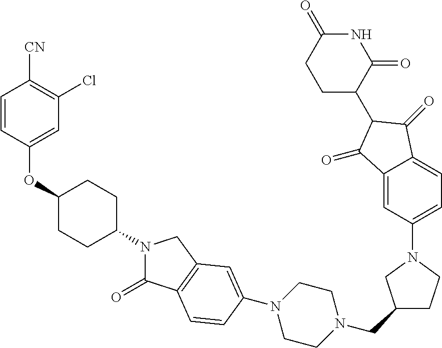 [SMILES: N#Cc1ccc(O[C@H]2CC[C@H](N3Cc4cc(N5CCN(C[C@@H]6CCN(c7ccc8c(c7)C(=O)C(C7CCC(=O)NC7=O)C8=O)C6)CC5)ccc4C3=O)CC2)cc1Cl] |
| 109 | 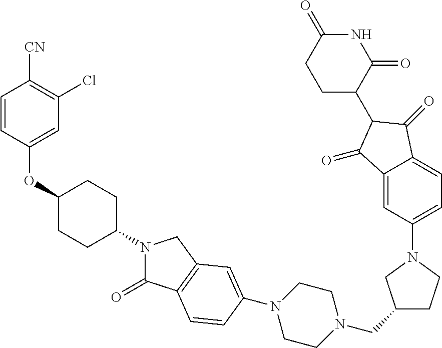 [SMILES: N#Cc1ccc(O[C@H]2CC[C@H](N3Cc4cc(N5CCN(C[C@H]6CCN(c7ccc8c(c7)C(=O)C(C7CCC(=O)NC7=O)C8=O)C6)CC5)ccc4C3=O)CC2)cc1Cl] |
| 110 | 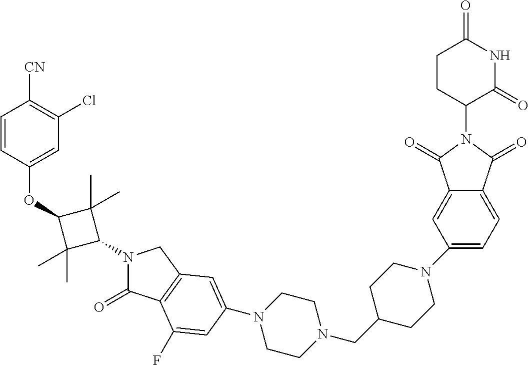 [SMILES: CC1(C)[C@H](Oc2ccc(C#N)c(Cl)c2)C(C)(C)[C@H]1N1Cc2cc(N3CCN(CC4CCN(c5ccc6c(c5)C(=O)N(C5CCC(=O)NC5=O)C6=O)CC4)CC3)cc(F)c2C1=O] |
| 111 | 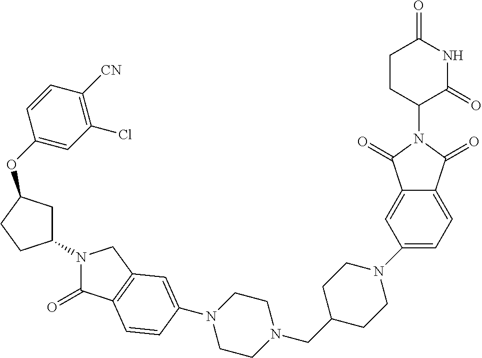 [SMILES: N#Cc1ccc(O[C@@H]2CC[C@@H](N3Cc4cc(N5CCN(CC6CCN(c7ccc8c(c7)C(=O)N(C7CCC(=O)NC7=O)C8=O)CC6)CC5)ccc4C3=O)C2)cc1Cl] |
| 112 | 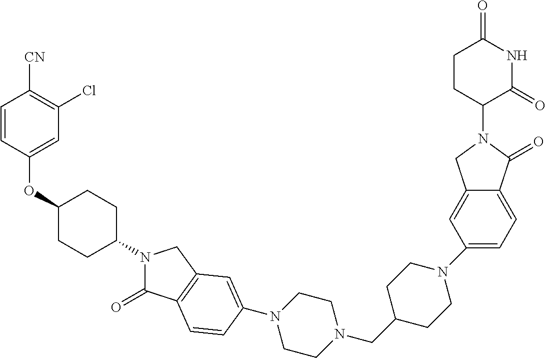 [SMILES: N#Cc1ccc(O[C@H]2CC[C@H](N3Cc4cc(N5CCN(CC6CCN(c7ccc8c(c7)CN(C7CCC(=O)NC7=O)C8=O)CC6)CC5)ccc4C3=O)CC2)cc1Cl] |
| 113 | 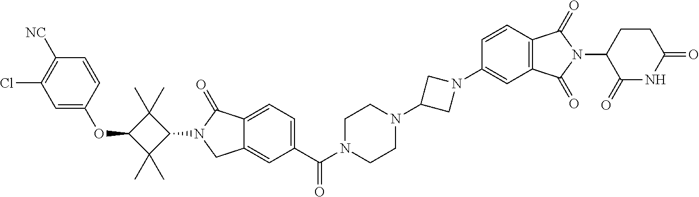 [SMILES: CC1(C)[C@H](Oc2ccc(C#N)c(Cl)c2)C(C)(C)[C@H]1N1Cc2cc(C(=O)N3CCN(C4CN(c5ccc6c(c5)C(=O)N(C5CCC(=O)NC5=O)C6=O)C4)CC3)ccc2C1=O] |
| 114 | 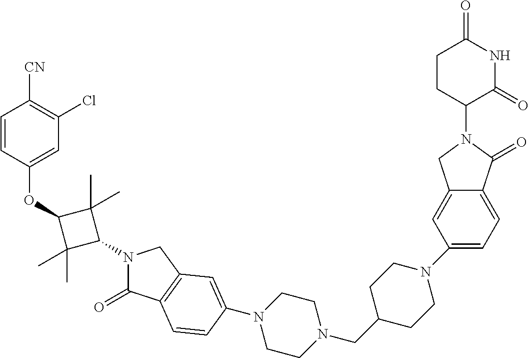 [SMILES: CC1(C)[C@H](Oc2ccc(C#N)c(Cl)c2)C(C)(C)[C@H]1N1Cc2cc(N3CCN(CC4CCN(c5ccc6c(c5)CN(C5CCC(=O)NC5=O)C6=O)CC4)CC3)ccc2C1=O] |
| 115 | 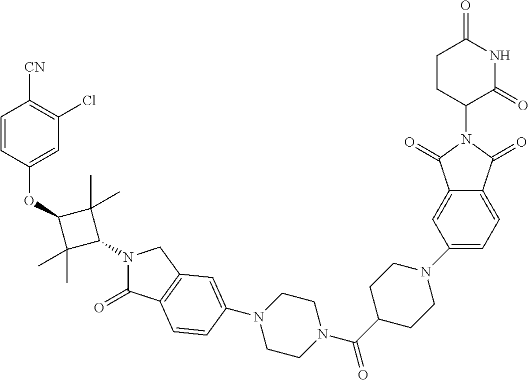 [SMILES: CC1(C)[C@H](Oc2ccc(C#N)c(Cl)c2)C(C)(C)[C@H]1N1Cc2cc(N3CCN(C(=O)C4CCN(c5ccc6c(c5)C(=O)N(C5CCC(=O)NC5=O)C6=O)CC4)CC3)ccc2C1=O] |
| 116 | 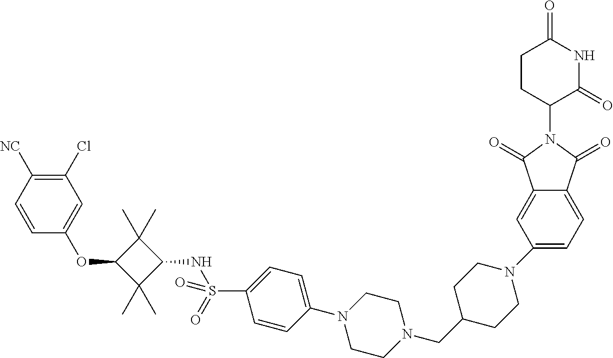 [SMILES: CC1(C)[C@H](NS(=O)(=O)c2ccc(N3CCN(CC4CCN(c5ccc6c(c5)C(=O)N(C5CCC(=O)NC5=O)C6=O)CC4)CC3)cc2)C(C)(C)[C@H]1Oc1ccc(C#N)c(Cl)c1] |
| 117 | 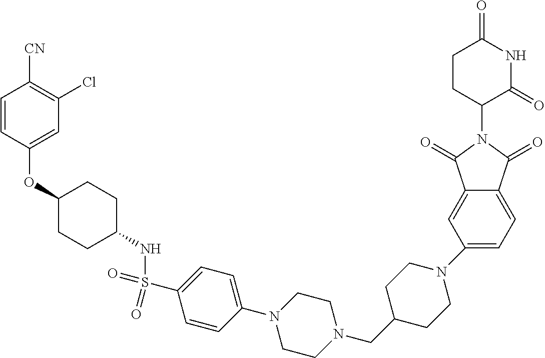 [SMILES: N#Cc1ccc(O[C@H]2CC[C@H](NS(=O)(=O)c3ccc(N4CCN(CC5CCN(c6ccc7c(c6)C(=O)N(C6CCC(=O)NC6=O)C7=O)CC5)CC4)cc3)CC2)cc1Cl] |
| 118 | 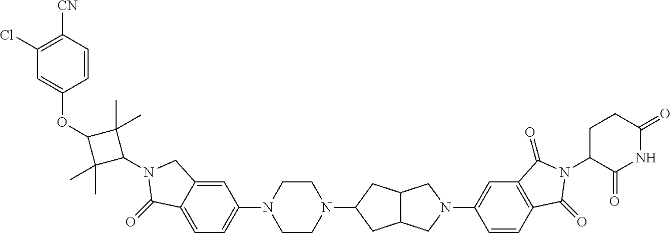 [SMILES: CC1(C)C(Oc2ccc(C#N)c(Cl)c2)C(C)(C)C1N1Cc2cc(N3CCN(C4CC5CN(c6ccc7c(c6)C(=O)N(C6CCC(=O)NC6=O)C7=O)CC5C4)CC3)ccc2C1=O] |
| 119 | 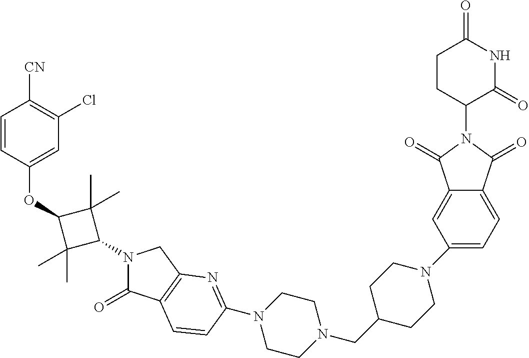 [SMILES: CC1(C)[C@H](Oc2ccc(C#N)c(Cl)c2)C(C)(C)[C@H]1N1Cc2nc(N3CCN(CC4CCN(c5ccc6c(c5)C(=O)N(C5CCC(=O)NC5=O)C6=O)CC4)CC3)ccc2C1=O] |
| 120 | 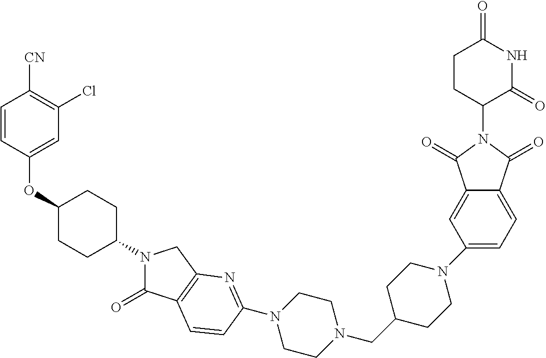 [SMILES: N#Cc1ccc(O[C@H]2CC[C@H](N3Cc4nc(N5CCN(CC6CCN(c7ccc8c(c7)C(=O)N(C7CCC(=O)NC7=O)C8=O)CC6)CC5)ccc4C3=O)CC2)cc1Cl] |
| 121 | 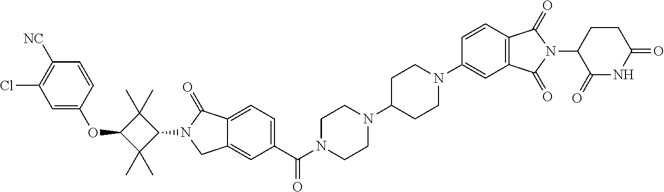 [SMILES: CC1(C)[C@H](Oc2ccc(C#N)c(Cl)c2)C(C)(C)[C@H]1N1Cc2cc(C(=O)N3CCN(C4CCN(c5ccc6c(c5)C(=O)N(C5CCC(=O)NC5=O)C6=O)CC4)CC3)ccc2C1=O] |
| 122 | 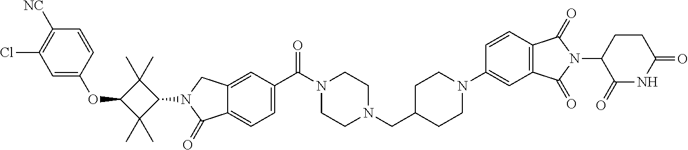 [SMILES: CC1(C)[C@H](Oc2ccc(C#N)c(Cl)c2)C(C)(C)[C@H]1N1Cc2cc(C(=O)N3CCN(CC4CCN(c5ccc6c(c5)C(=O)N(C5CCC(=O)NC5=O)C6=O)CC4)CC3)ccc2C1=O] |
| 123 | 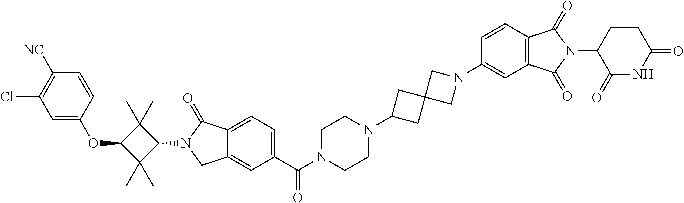 [SMILES: CC1(C)[C@H](Oc2ccc(C#N)c(Cl)c2)C(C)(C)[C@H]1N1Cc2cc(C(=O)N3CCN(C4CC5(C4)CN(c4ccc6c(c4)C(=O)N(C4CCC(=O)NC4=O)C6=O)C5)CC3)ccc2C1=O] |
| 124 | 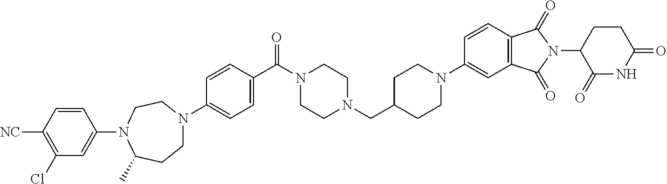 [SMILES: C[C@H]1CCN(c2ccc(C(=O)N3CCN(CC4CCN(c5ccc6c(c5)C(=O)N(C5CCC(=O)NC5=O)C6=O)CC4)CC3)cc2)CCN1c1ccc(C#N)c(Cl)c1] |
| 125 | 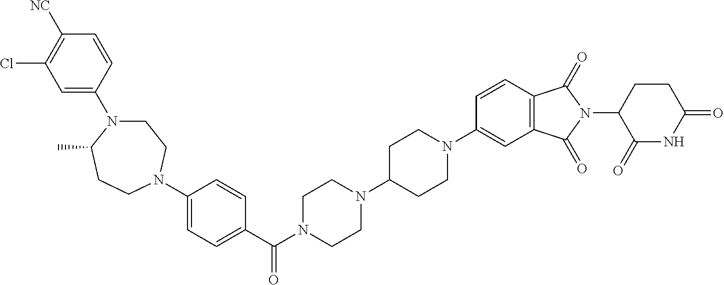 [SMILES: C[C@H]1CCN(c2ccc(C(=O)N3CCN(C4CCN(c5ccc6c(c5)C(=O)N(C5CCC(=O)NC5=O)C6=O)CC4)CC3)cc2)CCN1c1ccc(C#N)c(Cl)c1] |
| 126 | 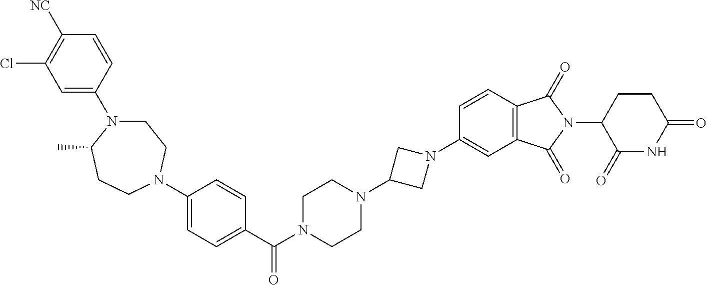 [SMILES: C[C@H]1CCN(c2ccc(C(=O)N3CCN(C4CN(c5ccc6c(c5)C(=O)N(C5CCC(=O)NC5=O)C6=O)C4)CC3)cc2)CCN1c1ccc(C#N)c(Cl)c1] |
| 127 | 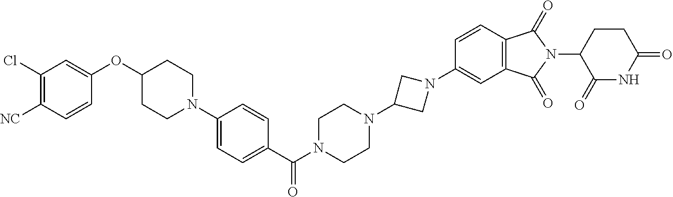 [SMILES: N#Cc1ccc(OC2CCN(c3ccc(C(=O)N4CCN(C5CN(c6ccc7c(c6)C(=O)N(C6CCC(=O)NC6=O)C7=O)C5)CC4)cc3)CC2)cc1Cl] |
| 128 | 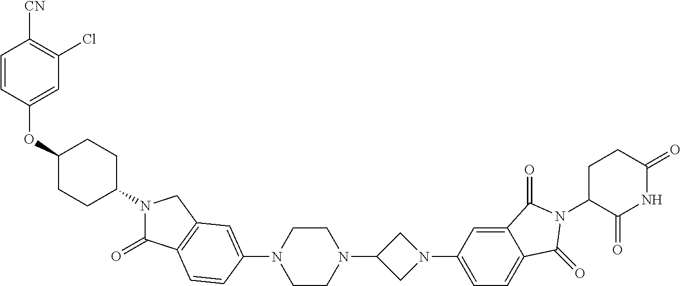 [SMILES: N#Cc1ccc(O[C@H]2CC[C@H](N3Cc4cc(N5CCN(C6CN(c7ccc8c(c7)C(=O)N(C7CCC(=O)NC7=O)C8=O)C6)CC5)ccc4C3=O)CC2)cc1Cl] |
| 129 | 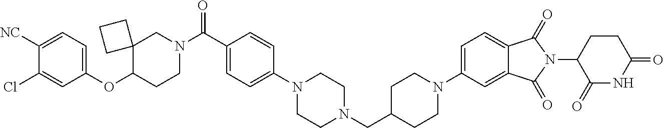 [SMILES: N#Cc1ccc(OC2CCN(C(=O)c3ccc(N4CCN(CC5CCN(c6ccc7c(c6)C(=O)N(C6CCC(=O)NC6=O)C7=O)CC5)CC4)cc3)CC23CCC3)cc1Cl] |
| 130 | 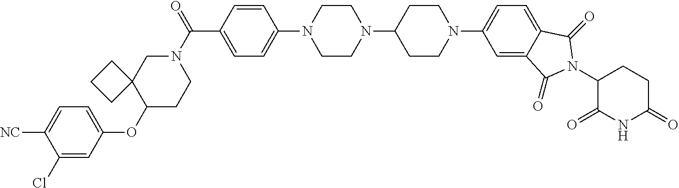 [SMILES: N#Cc1ccc(OC2CCN(C(=O)c3ccc(N4CCN(C5CCN(c6ccc7c(c6)C(=O)N(C6CCC(=O)NC6=O)C7=O)CC5)CC4)cc3)CC23CCC3)cc1Cl] |
| 131 | 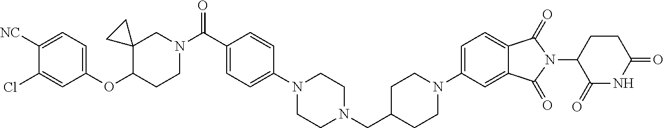 [SMILES: N#Cc1ccc(OC2CCN(C(=O)c3ccc(N4CCN(CC5CCN(c6ccc7c(c6)C(=O)N(C6CCC(=O)NC6=O)C7=O)CC5)CC4)cc3)CC23CC3)cc1Cl] |
| 132 | 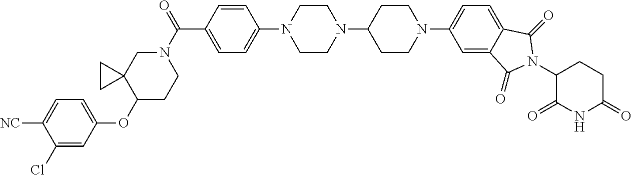 [SMILES: N#Cc1ccc(OC2CCN(C(=O)c3ccc(N4CCN(C5CCN(c6ccc7c(c6)C(=O)N(C6CCC(=O)NC6=O)C7=O)CC5)CC4)cc3)CC23CC3)cc1Cl] |
| 133 | 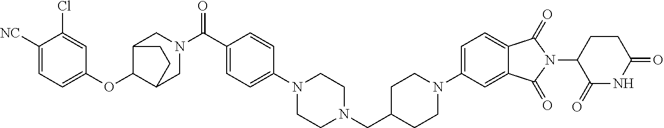 [SMILES: N#Cc1ccc(OC2C3CCC2CN(C(=O)c2ccc(N4CCN(CC5CCN(c6ccc7c(c6)C(=O)N(C6CCC(=O)NC6=O)C7=O)CC5)CC4)cc2)C3)cc1Cl] |
| 134 | 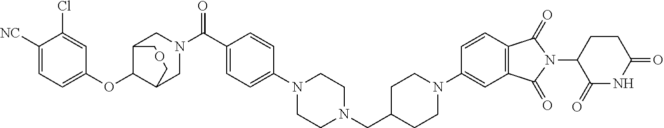 [SMILES: N#Cc1ccc(OC2C3COCC2CN(C(=O)c2ccc(N4CCN(CC5CCN(c6ccc7c(c6)C(=O)N(C6CCC(=O)NC6=O)C7=O)CC5)CC4)cc2)C3)cc1Cl] |
| 135 | 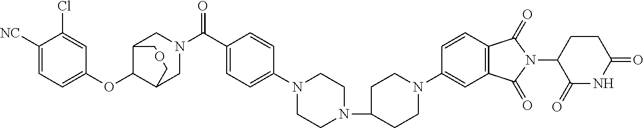 [SMILES: N#Cc1ccc(OC2C3COCC2CN(C(=O)c2ccc(N4CCN(C5CCN(c6ccc7c(c6)C(=O)N(C6CCC(=O)NC6=O)C7=O)CC5)CC4)cc2)C3)cc1Cl] |
| 136 | 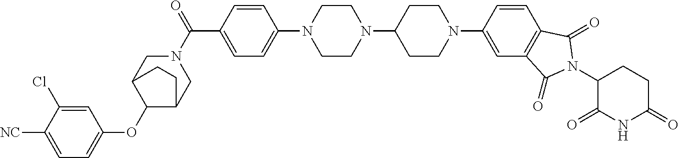 [SMILES: N#Cc1ccc(OC2C3CCC2CN(C(=O)c2ccc(N4CCN(C5CCN(c6ccc7c(c6)C(=O)N(C6CCC(=O)NC6=O)C7=O)CC5)CC4)cc2)C3)cc1Cl] |
| 137 | 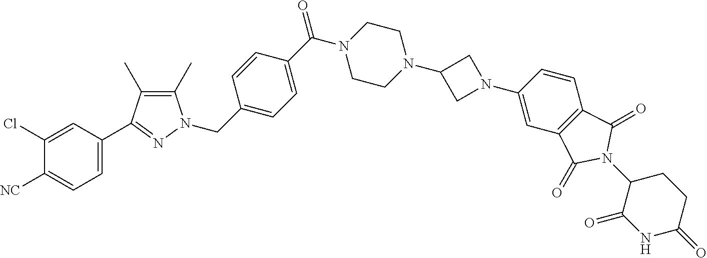 [SMILES: Cc1c(-c2ccc(C#N)c(Cl)c2)nn(Cc2ccc(C(=O)N3CCN(C4CN(c5ccc6c(c5)C(=O)N(C5CCC(=O)NC5=O)C6=O)C4)CC3)cc2)c1C] |
| 138 | 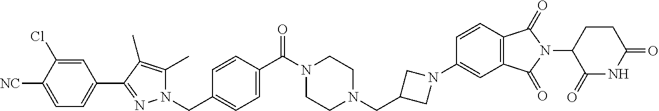 [SMILES: Cc1c(-c2ccc(C#N)c(Cl)c2)nn(Cc2ccc(C(=O)N3CCN(CC4CN(c5ccc6c(c5)C(=O)N(C5CCC(=O)NC5=O)C6=O)C4)CC3)cc2)c1C] |
| 139 | 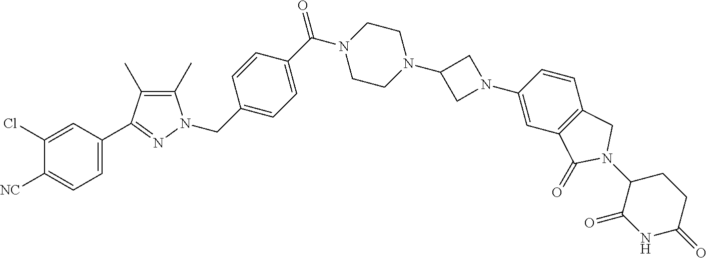 [SMILES: Cc1c(-c2ccc(C#N)c(Cl)c2)nn(Cc2ccc(C(=O)N3CCN(C4CN(c5ccc6c(c5)C(=O)N(C5CCC(=O)NC5=O)C6)C4)CC3)cc2)c1C] |
| 140 | 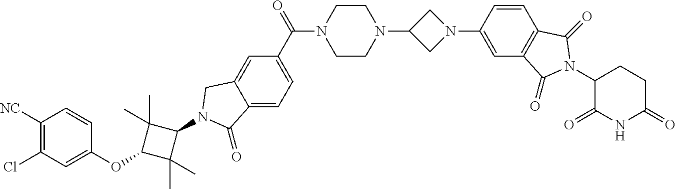 [SMILES: CC1(C)[C@H](Oc2ccc(C#N)c(Cl)c2)C(C)(C)[C@H]1N1Cc2cc(C(=O)N3CCN(C4CN(c5ccc6c(c5)C(=O)N(C5CCC(=O)NC5=O)C6=O)C4)CC3)ccc2C1=O] |
| 141 | 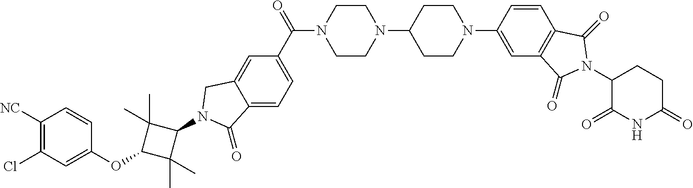 [SMILES: CC1(C)[C@H](Oc2ccc(C#N)c(Cl)c2)C(C)(C)[C@H]1N1Cc2cc(C(=O)N3CCN(C4CCN(c5ccc6c(c5)C(=O)N(C5CCC(=O)NC5=O)C6=O)CC4)CC3)ccc2C1=O] |
| 142 | 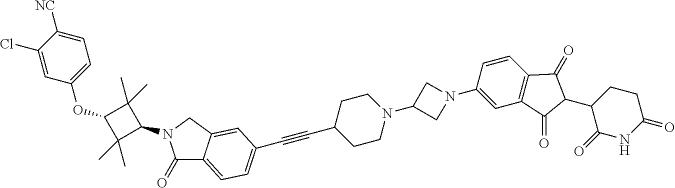 [SMILES: CC1(C)[C@H](Oc2ccc(C#N)c(Cl)c2)C(C)(C)[C@H]1N1Cc2cc(C#CC3CCN(C4CN(c5ccc6c(c5)C(=O)C(C5CCC(=O)NC5=O)C6=O)C4)CC3)ccc2C1=O] |
| 143 | 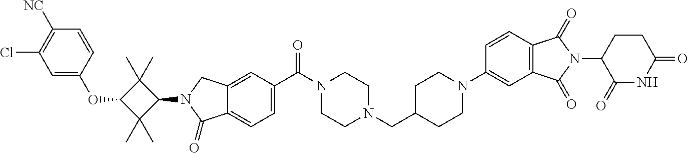 [SMILES: CC1(C)[C@H](Oc2ccc(C#N)c(Cl)c2)C(C)(C)[C@H]1N1Cc2cc(C(=O)N3CCN(CC4CCN(c5ccc6c(c5)C(=O)N(C5CCC(=O)NC5=O)C6=O)CC4)CC3)ccc2C1=O] |
| 144 | 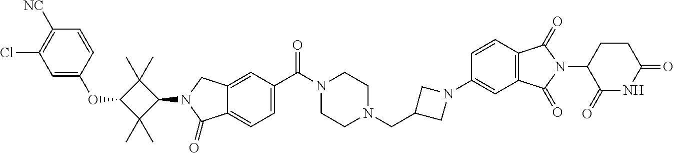 [SMILES: CC1(C)[C@H](Oc2ccc(C#N)c(Cl)c2)C(C)(C)[C@H]1N1Cc2cc(C(=O)N3CCN(CC4CN(c5ccc6c(c5)C(=O)N(C5CCC(=O)NC5=O)C6=O)C4)CC3)ccc2C1=O] |
| 145 | 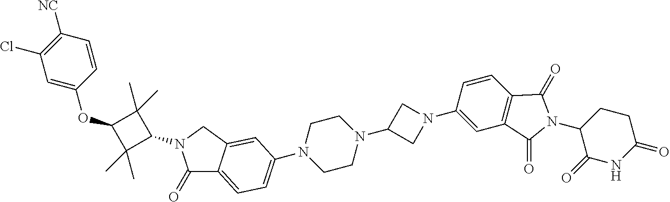 [SMILES: CC1(C)[C@H](Oc2ccc(C#N)c(Cl)c2)C(C)(C)[C@H]1N1Cc2cc(N3CCN(C4CN(c5ccc6c(c5)C(=O)N(C5CCC(=O)NC5=O)C6=O)C4)CC3)ccc2C1=O] |
| 146 | 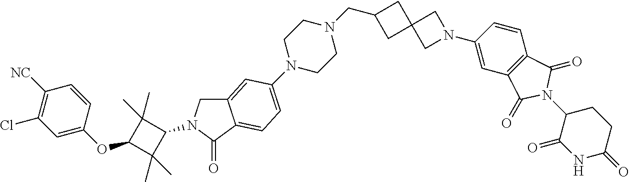 [SMILES: CC1(C)[C@H](Oc2ccc(C#N)c(Cl)c2)C(C)(C)[C@H]1N1Cc2cc(N3CCN(CC4CC5(C4)CN(c4ccc6c(c4)C(=O)N(C4CCC(=O)NC4=O)C6=O)C5)CC3)ccc2C1=O] |
| 147 | 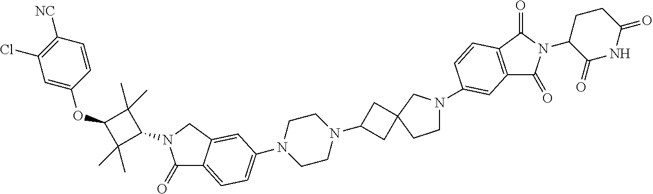 [SMILES: CC1(C)[C@H](Oc2ccc(C#N)c(Cl)c2)C(C)(C)[C@H]1N1Cc2cc(N3CCN(C4CC5(CCN(c6ccc7c(c6)C(=O)N(C6CCC(=O)NC6=O)C7=O)C5)C4)CC3)ccc2C1=O] |
| 148 | 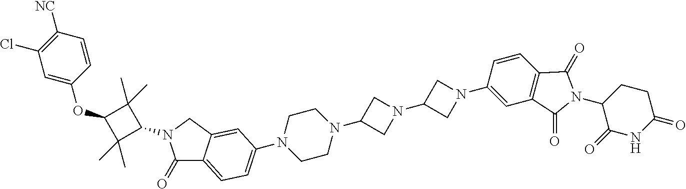 [SMILES: CC1(C)[C@H](Oc2ccc(C#N)c(Cl)c2)C(C)(C)[C@H]1N1Cc2cc(N3CCN(C4CN(C5CN(c6ccc7c(c6)C(=O)N(C6CCC(=O)NC6=O)C7=O)C5)C4)CC3)ccc2C1=O] |
| 149 | 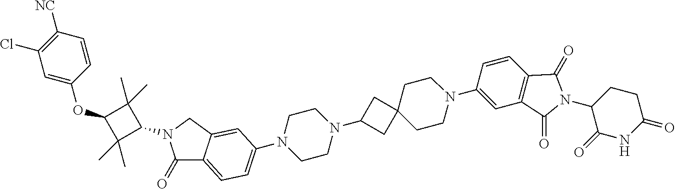 [SMILES: CC1(C)[C@H](Oc2ccc(C#N)c(Cl)c2)C(C)(C)[C@H]1N1Cc2cc(N3CCN(C4CC5(CCN(c6ccc7c(c6)C(=O)N(C6CCC(=O)NC6=O)C7=O)CC5)C4)CC3)ccc2C1=O] |
| 150 | 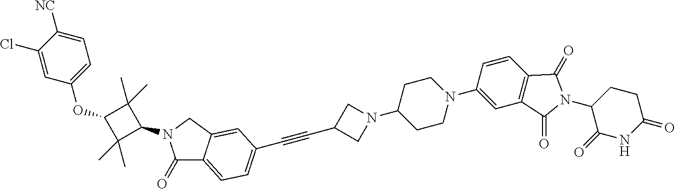 [SMILES: CC1(C)[C@H](Oc2ccc(C#N)c(Cl)c2)C(C)(C)[C@H]1N1Cc2cc(C#CC3CN(C4CCN(c5ccc6c(c5)C(=O)N(C5CCC(=O)NC5=O)C6=O)CC4)C3)ccc2C1=O] |
| 151 | 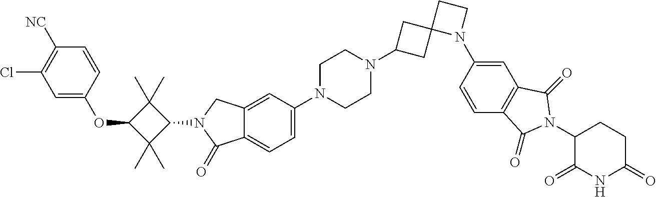 [SMILES: CC1(C)[C@H](Oc2ccc(C#N)c(Cl)c2)C(C)(C)[C@H]1N1Cc2cc(N3CCN(C4CC5(CCN5c5ccc6c(c5)C(=O)N(C5CCC(=O)NC5=O)C6=O)C4)CC3)ccc2C1=O] |
| 152 | 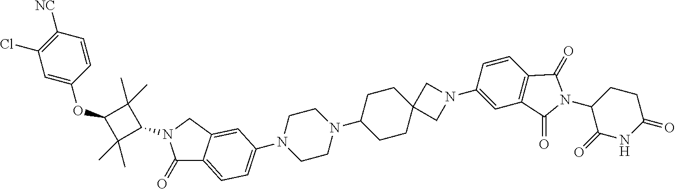 [SMILES: CC1(C)[C@H](Oc2ccc(C#N)c(Cl)c2)C(C)(C)[C@H]1N1Cc2cc(N3CCN(C4CCC5(CC4)CN(c4ccc6c(c4)C(=O)N(C4CCC(=O)NC4=O)C6=O)C5)CC3)ccc2C1=O] |
| 153 | 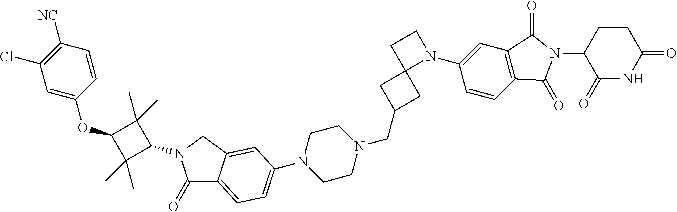 [SMILES: CC1(C)[C@H](Oc2ccc(C#N)c(Cl)c2)C(C)(C)[C@H]1N1Cc2cc(N3CCN(CC4CC5(CCN5c5ccc6c(c5)C(=O)N(C5CCC(=O)NC5=O)C6=O)C4)CC3)ccc2C1=O] |
| 154 | 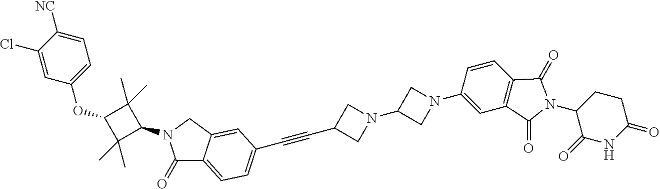 [SMILES: CC1(C)[C@H](Oc2ccc(C#N)c(Cl)c2)C(C)(C)[C@H]1N1Cc2cc(C#CC3CN(C4CN(c5ccc6c(c5)C(=O)N(C5CCC(=O)NC5=O)C6=O)C4)C3)ccc2C1=O] |
| 155 | 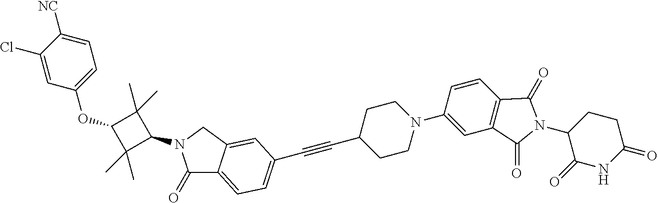 [SMILES: CC1(C)[C@H](Oc2ccc(C#N)c(Cl)c2)C(C)(C)[C@H]1N1Cc2cc(C#CC3CCN(c4ccc5c(c4)C(=O)N(C4CCC(=O)NC4=O)C5=O)CC3)ccc2C1=O] |
| 156 | 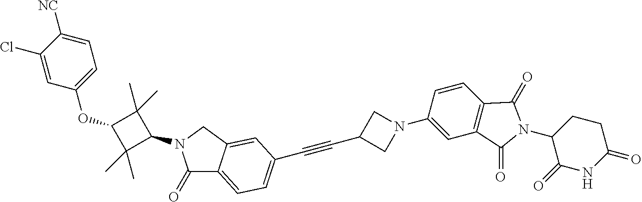 [SMILES: CC1(C)[C@H](Oc2ccc(C#N)c(Cl)c2)C(C)(C)[C@H]1N1Cc2cc(C#CC3CN(c4ccc5c(c4)C(=O)N(C4CCC(=O)NC4=O)C5=O)C3)ccc2C1=O] |
| 157 | 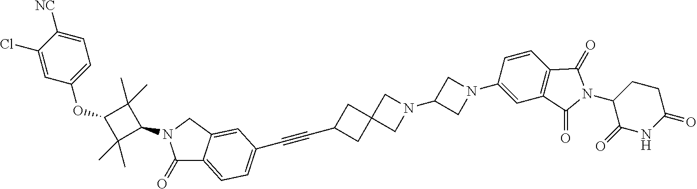 [SMILES: CC1(C)[C@H](Oc2ccc(C#N)c(Cl)c2)C(C)(C)[C@H]1N1Cc2cc(C#CC3CC4(C3)CN(C3CN(c5ccc6c(c5)C(=O)N(C5CCC(=O)NC5=O)C6=O)C3)C4)ccc2C1=O] |
| 158 | 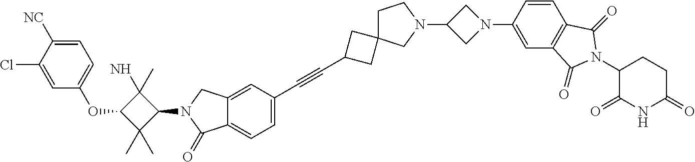 [SMILES: CC1(C)[C@H](Oc2ccc(C#N)c(Cl)c2)C(C)(N)[C@H]1N1Cc2cc(C#CC3CC4(CCN(C5CN(c6ccc7c(c6)C(=O)N(C6CCC(=O)NC6=O)C7=O)C5)C4)C3)ccc2C1=O] |
| 159 | 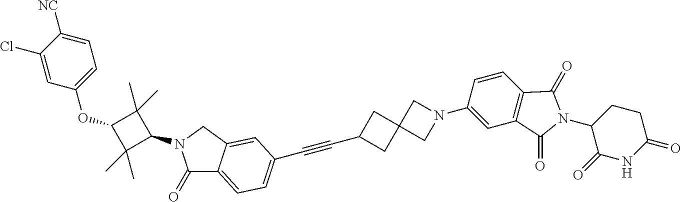 [SMILES: CC1(C)[C@H](Oc2ccc(C#N)c(Cl)c2)C(C)(C)[C@H]1N1Cc2cc(C#CC3CC4(C3)CN(c3ccc5c(c3)C(=O)N(C3CCC(=O)NC3=O)C5=O)C4)ccc2C1=O] |
| 160 | 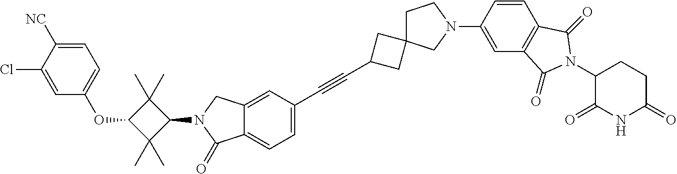 [SMILES: CC1(C)[C@H](Oc2ccc(C#N)c(Cl)c2)C(C)(C)[C@H]1N1Cc2cc(C#CC3CC4(CCN(c5ccc6c(c5)C(=O)N(C5CCC(=O)NC5=O)C6=O)C4)C3)ccc2C1=O] |
| 161 | 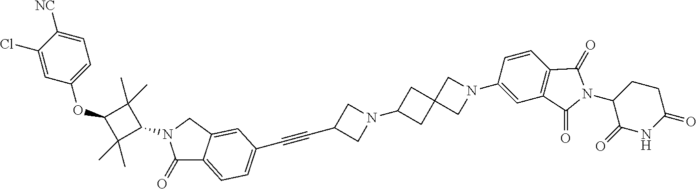 [SMILES: CC1(C)[C@H](Oc2ccc(C#N)c(Cl)c2)C(C)(C)[C@H]1N1Cc2cc(C#CC3CN(C4CC5(C4)CN(c4ccc6c(c4)C(=O)N(C4CCC(=O)NC4=O)C6=O)C5)C3)ccc2C1=O] |
| 162 | 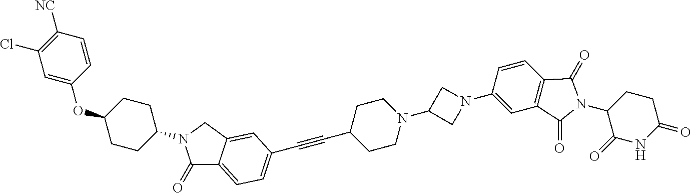 [SMILES: N#Cc1ccc(O[C@H]2CC[C@H](N3Cc4cc(C#CC5CCN(C6CN(c7ccc8c(c7)C(=O)N(C7CCC(=O)NC7=O)C8=O)C6)CC5)ccc4C3=O)CC2)cc1Cl] |
| 163 | 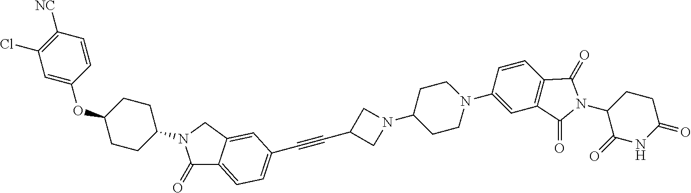 [SMILES: N#Cc1ccc(O[C@H]2CC[C@H](N3Cc4cc(C#CC5CN(C6CCN(c7ccc8c(c7)C(=O)N(C7CCC(=O)NC7=O)C8=O)CC6)C5)ccc4C3=O)CC2)cc1Cl] |
| 164 | 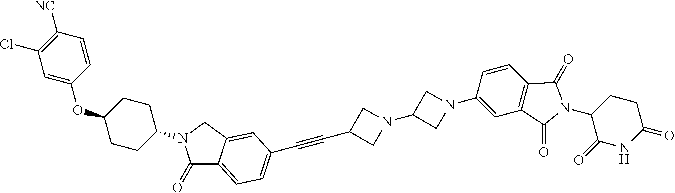 [SMILES: N#Cc1ccc(O[C@H]2CC[C@H](N3Cc4cc(C#CC5CN(C6CN(c7ccc8c(c7)C(=O)N(C7CCC(=O)NC7=O)C8=O)C6)C5)ccc4C3=O)CC2)cc1Cl] |
| 165 | 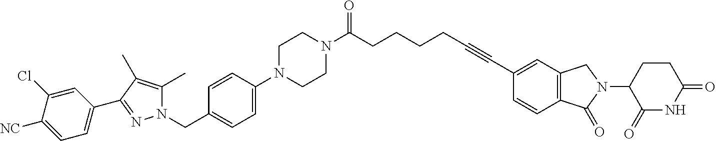 [SMILES: Cc1c(-c2ccc(C#N)c(Cl)c2)nn(Cc2ccc(N3CCN(C(=O)CCCCC#Cc4ccc5c(c4)CN(C4CCC(=O)NC4=O)C5=O)CC3)cc2)c1C] |
| 166 | 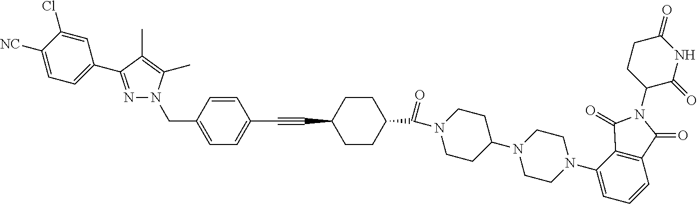 [SMILES: Cc1c(-c2ccc(C#N)c(Cl)c2)nn(Cc2ccc(C#C[C@H]3CC[C@H](C(=O)N4CCC(N5CCN(c6cccc7c6C(=O)N(C6CCC(=O)NC6=O)C7=O)CC5)CC4)CC3)cc2)c1C] |
| 167 | 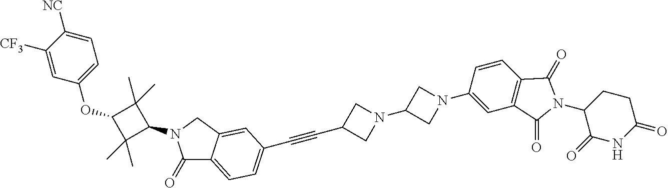 [SMILES: CC1(C)[C@H](Oc2ccc(C#N)c(C(F)(F)F)c2)C(C)(C)[C@H]1N1Cc2cc(C#CC3CN(C4CN(c5ccc6c(c5)C(=O)N(C5CCC(=O)NC5=O)C6=O)C4)C3)ccc2C1=O] |
| 168 | 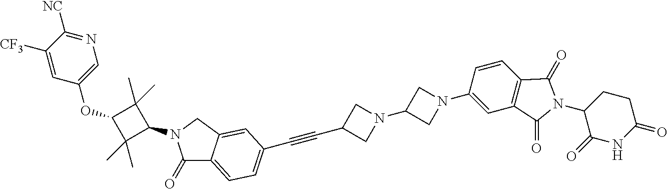 [SMILES: CC1(C)[C@H](Oc2cnc(C#N)c(C(F)(F)F)c2)C(C)(C)[C@H]1N1Cc2cc(C#CC3CN(C4CN(c5ccc6c(c5)C(=O)N(C5CCC(=O)NC5=O)C6=O)C4)C3)ccc2C1=O] |
| 169 | 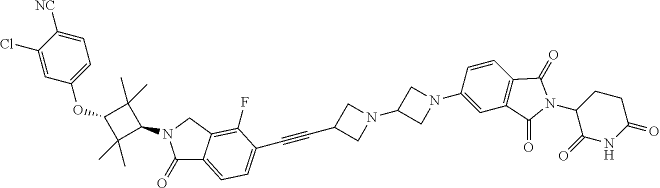 [SMILES: CC1(C)[C@H](Oc2ccc(C#N)c(Cl)c2)C(C)(C)[C@H]1N1Cc2c(ccc(C#CC3CN(C4CN(c5ccc6c(c5)C(=O)N(C5CCC(=O)NC5=O)C6=O)C4)C3)c2F)C1=O] |
| 170 | 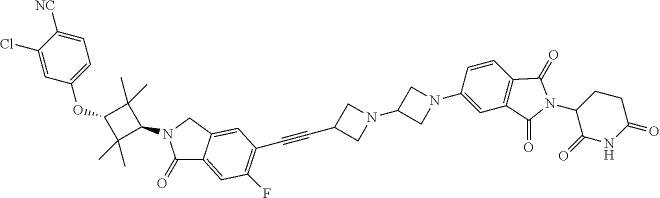 [SMILES: CC1(C)[C@H](Oc2ccc(C#N)c(Cl)c2)C(C)(C)[C@H]1N1Cc2cc(C#CC3CN(C4CN(c5ccc6c(c5)C(=O)N(C5CCC(=O)NC5=O)C6=O)C4)C3)c(F)cc2C1=O] |
| 171 | 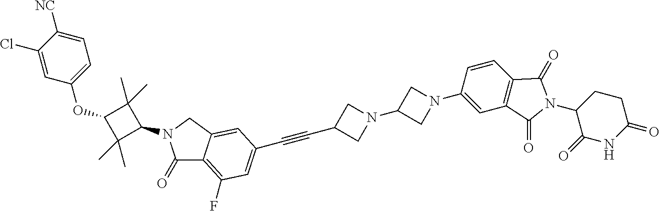 [SMILES: CC1(C)[C@H](Oc2ccc(C#N)c(Cl)c2)C(C)(C)[C@H]1N1Cc2cc(C#CC3CN(C4CN(c5ccc6c(c5)C(=O)N(C5CCC(=O)NC5=O)C6=O)C4)C3)cc(F)c2C1=O] |
| 172 | 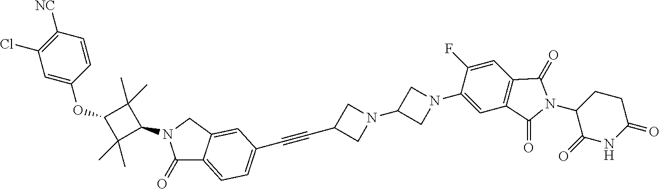 [SMILES: CC1(C)[C@H](Oc2ccc(C#N)c(Cl)c2)C(C)(C)[C@H]1N1Cc2cc(C#CC3CN(C4CN(c5cc6c(cc5F)C(=O)N(C5CCC(=O)NC5=O)C6=O)C4)C3)ccc2C1=O] |
| 173 | 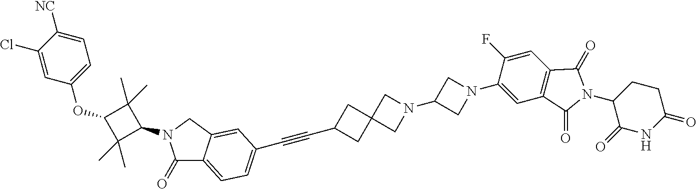 [SMILES: CC1(C)[C@H](Oc2ccc(C#N)c(Cl)c2)C(C)(C)[C@H]1N1Cc2cc(C#CC3CC4(C3)CN(C3CN(c5cc6c(cc5F)C(=O)N(C5CCC(=O)NC5=O)C6=O)C3)C4)ccc2C1=O] |
| 174 | 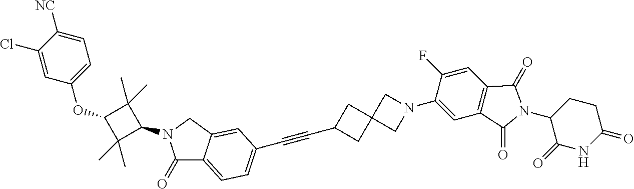 [SMILES: CC1(C)[C@H](Oc2ccc(C#N)c(Cl)c2)C(C)(C)[C@H]1N1Cc2cc(C#CC3CC4(C3)CN(c3cc5c(cc3F)C(=O)N(C3CCC(=O)NC3=O)C5=O)C4)ccc2C1=O] |
| 175 | 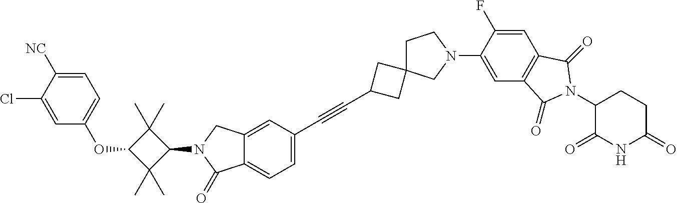 [SMILES: CC1(C)[C@H](Oc2ccc(C#N)c(Cl)c2)C(C)(C)[C@H]1N1Cc2cc(C#CC3CC4(CCN(c5cc6c(cc5F)C(=O)N(C5CCC(=O)NC5=O)C6=O)C4)C3)ccc2C1=O] |
| 176 | 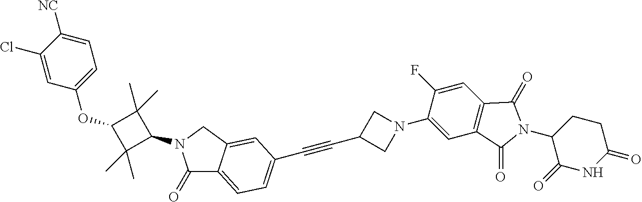 [SMILES: CC1(C)[C@H](Oc2ccc(C#N)c(Cl)c2)C(C)(C)[C@H]1N1Cc2cc(C#CC3CN(c4cc5c(cc4F)C(=O)N(C4CCC(=O)NC4=O)C5=O)C3)ccc2C1=O] |
| 177 | 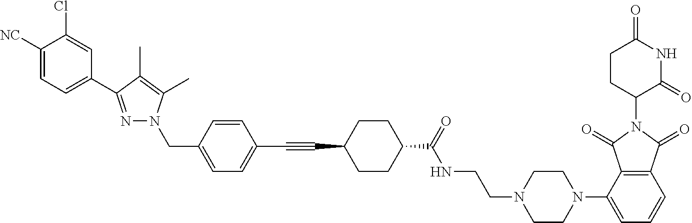 [SMILES: Cc1c(-c2ccc(C#N)c(Cl)c2)nn(Cc2ccc(C#C[C@H]3CC[C@H](C(=O)NCCN4CCN(c5cccc6c5C(=O)N(C5CCC(=O)NC5=O)C6=O)CC4)CC3)cc2)c1C] |
| 178 | 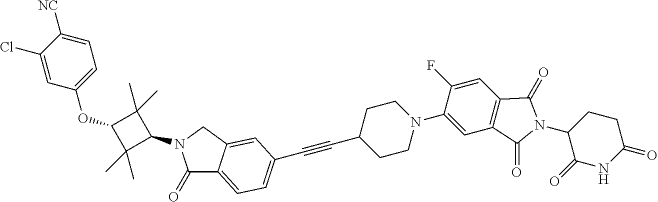 [SMILES: CC1(C)[C@H](Oc2ccc(C#N)c(Cl)c2)C(C)(C)[C@H]1N1Cc2cc(C#CC3CCN(c4cc5c(cc4F)C(=O)N(C4CCC(=O)NC4=O)C5=O)CC3)ccc2C1=O] |
| 179 | 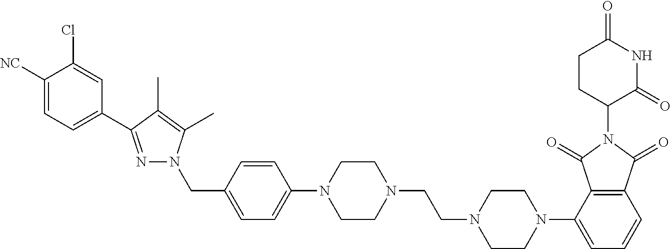 [SMILES: Cc1c(-c2ccc(C#N)c(Cl)c2)nn(Cc2ccc(N3CCN(CCN4CCN(c5cccc6c5C(=O)N(C5CCC(=O)NC5=O)C6=O)CC4)CC3)cc2)c1C] |
| 180 | 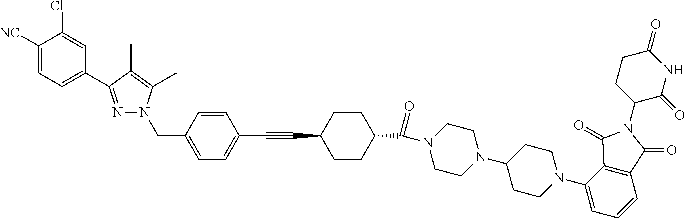 [SMILES: Cc1c(-c2ccc(C#N)c(Cl)c2)nn(Cc2ccc(C#C[C@H]3CC[C@H](C(=O)N4CCN(C5CCN(c6cccc7c6C(=O)N(C6CCC(=O)NC6=O)C7=O)CC5)CC4)CC3)cc2)c1C] |
| 181 | 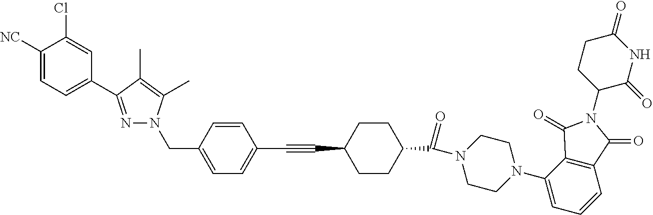 [SMILES: Cc1c(-c2ccc(C#N)c(Cl)c2)nn(Cc2ccc(C#C[C@H]3CC[C@H](C(=O)N4CCN(c5cccc6c5C(=O)N(C5CCC(=O)NC5=O)C6=O)CC4)CC3)cc2)c1C] |
| 182 | 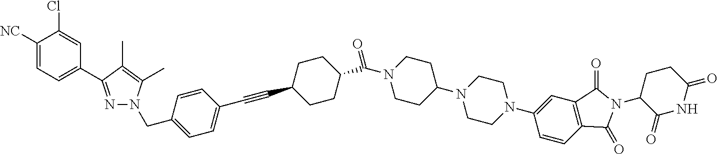 [SMILES: Cc1c(-c2ccc(C#N)c(Cl)c2)nn(Cc2ccc(C#C[C@H]3CC[C@H](C(=O)N4CCC(N5CCN(c6ccc7c(c6)C(=O)N(C6CCC(=O)NC6=O)C7=O)CC5)CC4)CC3)cc2)c1C] |
| 183 | 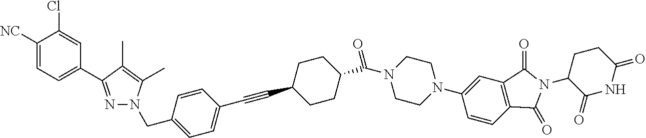 [SMILES: Cc1c(-c2ccc(C#N)c(Cl)c2)nn(Cc2ccc(C#C[C@H]3CC[C@H](C(=O)N4CCN(c5ccc6c(c5)C(=O)N(C5CCC(=O)NC5=O)C6=O)CC4)CC3)cc2)c1C] |
| 184 | 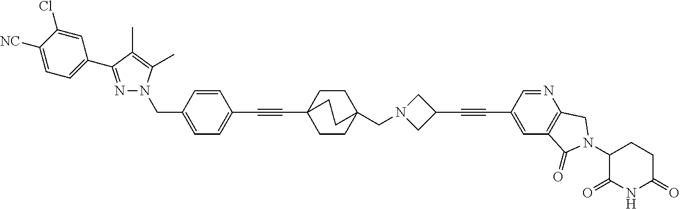 [SMILES: Cc1c(-c2ccc(C#N)c(Cl)c2)nn(Cc2ccc(C#CC34CCC(CN5CC(C#Cc6cnc7c(c6)C(=O)N(C6CCC(=O)NC6=O)C7)C5)(CC3)CC4)cc2)c1C] |
| 185 | 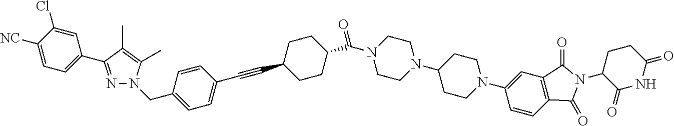 [SMILES: Cc1c(-c2ccc(C#N)c(Cl)c2)nn(Cc2ccc(C#C[C@H]3CC[C@H](C(=O)N4CCN(C5CCN(c6ccc7c(c6)C(=O)N(C6CCC(=O)NC6=O)C7=O)CC5)CC4)CC3)cc2)c1C] |
| 186 | 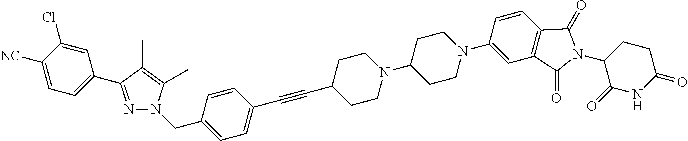 [SMILES: Cc1c(-c2ccc(C#N)c(Cl)c2)nn(Cc2ccc(C#CC3CCN(C4CCN(c5ccc6c(c5)C(=O)N(C5CCC(=O)NC5=O)C6=O)CC4)CC3)cc2)c1C] |
| 187 | 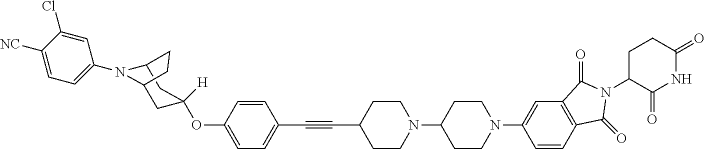 [SMILES: N#Cc1ccc(N2C3CCC2CC(Oc2ccc(C#CC4CCN(C5CCN(c6ccc7c(c6)C(=O)N(C6CCC(=O)NC6=O)C7=O)CC5)CC4)cc2)C3)cc1Cl] |
| 188 | 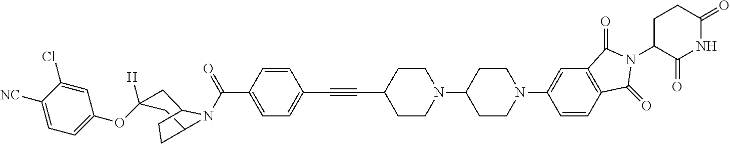 [SMILES: N#Cc1ccc(OC2CC3CCC(C2)N3C(=O)c2ccc(C#CC3CCN(C4CCN(c5ccc6c(c5)C(=O)N(C5CCC(=O)NC5=O)C6=O)CC4)CC3)cc2)cc1Cl] |
| 189 | 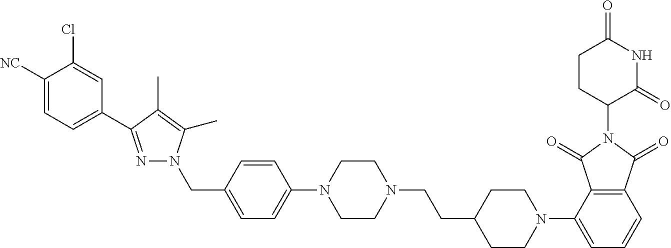 [SMILES: Cc1c(-c2ccc(C#N)c(Cl)c2)nn(Cc2ccc(N3CCN(CCC4CCN(c5cccc6c5C(=O)N(C5CCC(=O)NC5=O)C6=O)CC4)CC3)cc2)c1C] |
| 190 | 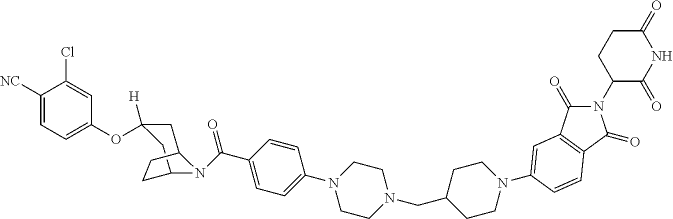 [SMILES: N#Cc1ccc(OC2CC3CCC(C2)N3C(=O)c2ccc(N3CCN(CC4CCN(c5ccc6c(c5)C(=O)N(C5CCC(=O)NC5=O)C6=O)CC4)CC3)cc2)cc1Cl] |
| 191 | 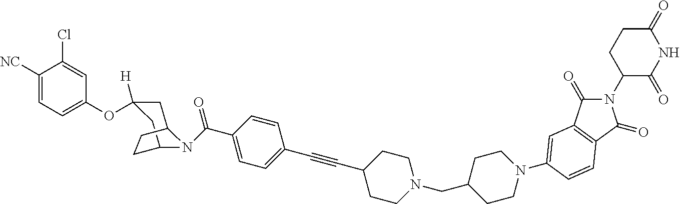 [SMILES: N#Cc1ccc(OC2CC3CCC(C2)N3C(=O)c2ccc(C#CC3CCN(CC4CCN(c5ccc6c(c5)C(=O)N(C5CCC(=O)NC5=O)C6=O)CC4)CC3)cc2)cc1Cl] |
| 192 | 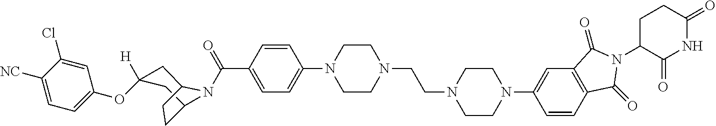 [SMILES: N#Cc1ccc(OC2CC3CCC(C2)N3C(=O)c2ccc(N3CCN(CCN4CCN(c5ccc6c(c5)C(=O)N(C5CCC(=O)NC5=O)C6=O)CC4)CC3)cc2)cc1Cl] |
| 193 | 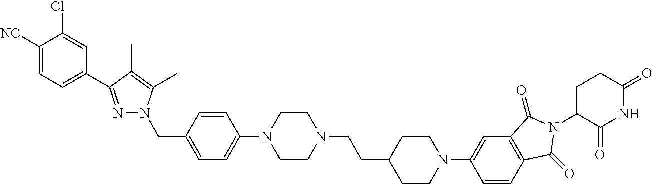 [SMILES: Cc1c(-c2ccc(C#N)c(Cl)c2)nn(Cc2ccc(N3CCN(CCC4CCN(c5ccc6c(c5)C(=O)N(C5CCC(=O)NC5=O)C6=O)CC4)CC3)cc2)c1C] |
| 194 | 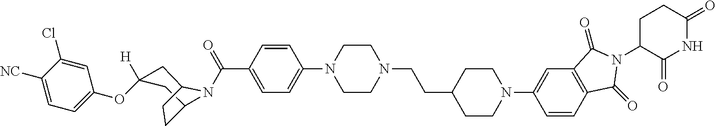 [SMILES: N#Cc1ccc(OC2CC3CCC(C2)N3C(=O)c2ccc(N3CCN(CCC4CCN(c5ccc6c(c5)C(=O)N(C5CCC(=O)NC5=O)C6=O)CC4)CC3)cc2)cc1Cl] |
| 195 | 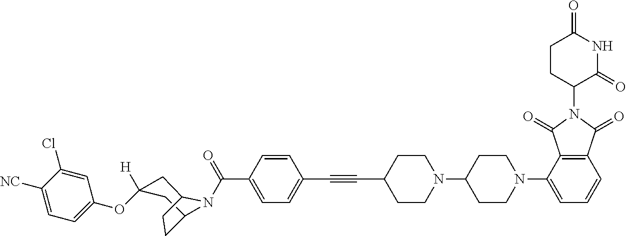 [SMILES: N#Cc1ccc(OC2CC3CCC(C2)N3C(=O)c2ccc(C#CC3CCN(C4CCN(c5cccc6c5C(=O)N(C5CCC(=O)NC5=O)C6=O)CC4)CC3)cc2)cc1Cl] |
| 196 | 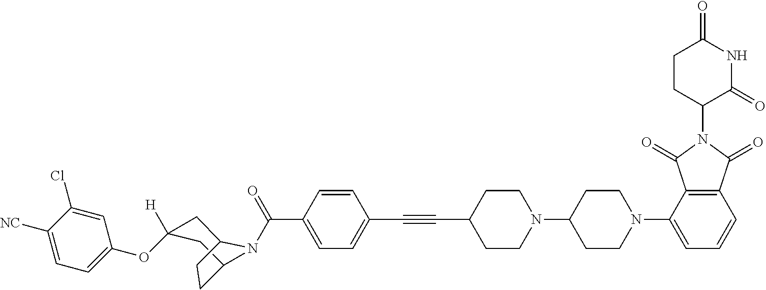 [SMILES: N#Cc1ccc(OC2CC3CCC(C2)N3C(=O)c2ccc(C#CC3CCN(C4CCN(c5cccc6c5C(=O)N(C5CCC(=O)NC5=O)C6=O)CC4)CC3)cc2)cc1Cl] |
| 197 | 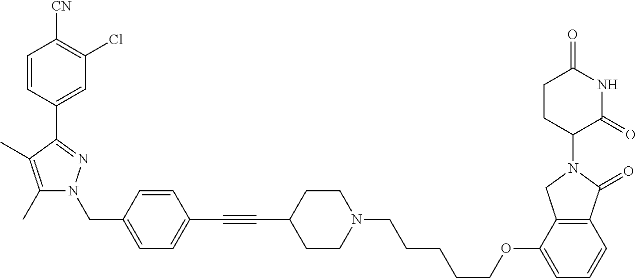 [SMILES: Cc1c(-c2ccc(C#N)c(Cl)c2)nn(Cc2ccc(C#CC3CCN(CCCCCOc4cccc5c4CN(C4CCC(=O)NC4=O)C5=O)CC3)cc2)c1C] |
| 198 | 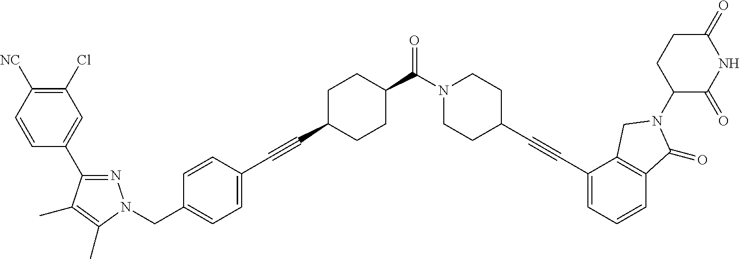 [SMILES: Cc1c(-c2ccc(C#N)c(Cl)c2)nn(Cc2ccc(C#C[C@H]3CC[C@@H](C(=O)N4CCC(C#Cc5cccc6c5CN(C5CCC(=O)NC5=O)C6=O)CC4)CC3)cc2)c1C] |
| 199 | 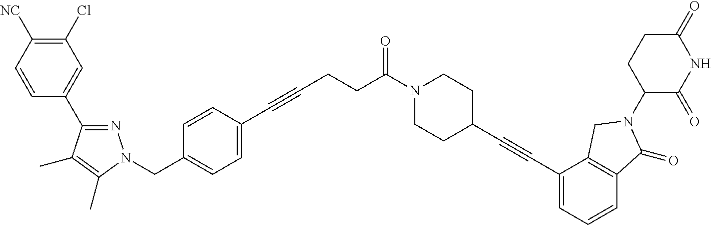 [SMILES: Cc1c(-c2ccc(C#N)c(Cl)c2)nn(Cc2ccc(C#CCCC(=O)N3CCC(C#Cc4cccc5c4CN(C4CCC(=O)NC4=O)C5=O)CC3)cc2)c1C] |
| 200 | 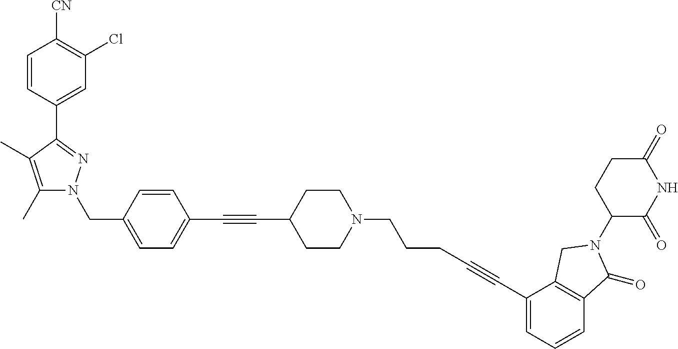 [SMILES: Cc1c(-c2ccc(C#N)c(Cl)c2)nn(Cc2ccc(C#CC3CCN(CCCC#Cc4cccc5c4CN(C4CCC(=O)NC4=O)C5=O)CC3)cc2)c1C] |
| 201 | 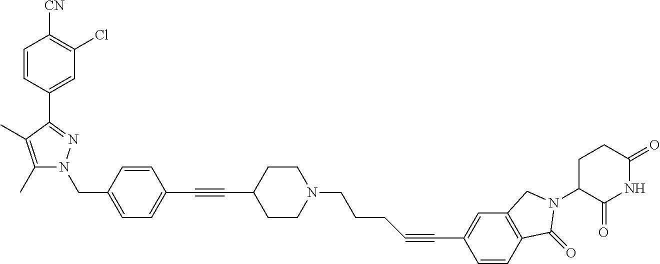 [SMILES: Cc1c(-c2ccc(C#N)c(Cl)c2)nn(Cc2ccc(C#CC3CCN(CCCC#Cc4ccc5c(c4)CN(C4CCC(=O)NC4=O)C5=O)CC3)cc2)c1C] |
| 202 | 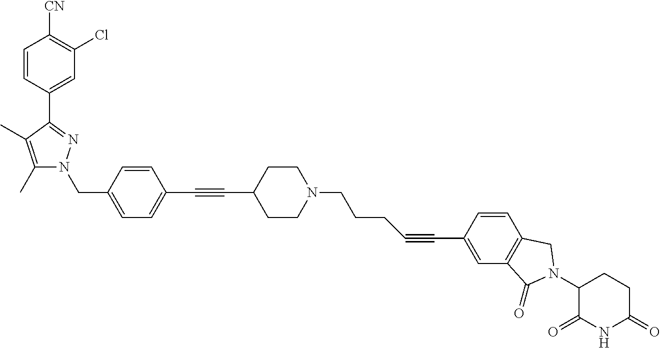 [SMILES: Cc1c(-c2ccc(C#N)c(Cl)c2)nn(Cc2ccc(C#CC3CCN(CCCC#Cc4ccc5c(c4)C(=O)N(C4CCC(=O)NC4=O)C5)CC3)cc2)c1C] |
| 203 | 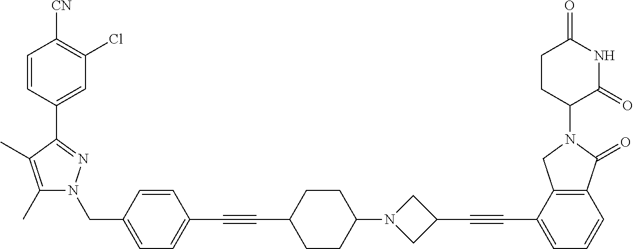 [SMILES: Cc1c(-c2ccc(C#N)c(Cl)c2)nn(Cc2ccc(C#CC3CCC(N4CC(C#Cc5cccc6c5CN(C5CCC(=O)NC5=O)C6=O)C4)CC3)cc2)c1C] |
| 204 | 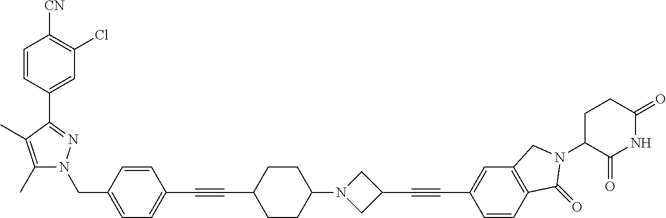 [SMILES: Cc1c(-c2ccc(C#N)c(Cl)c2)nn(Cc2ccc(C#CC3CCC(N4CC(C#Cc5ccc6c(c5)CN(C5CCC(=O)NC5=O)C6=O)C4)CC3)cc2)c1C] |
| 205 | 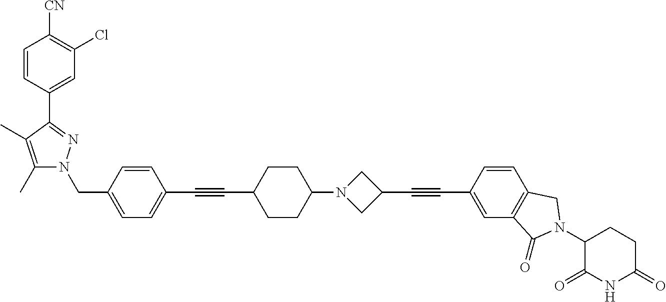 [SMILES: Cc1c(-c2ccc(C#N)c(Cl)c2)nn(Cc2ccc(C#CC3CCC(N4CC(C#Cc5ccc6c(c5)C(=O)N(C5CCC(=O)NC5=O)C6)C4)CC3)cc2)c1C] |
| 206 | 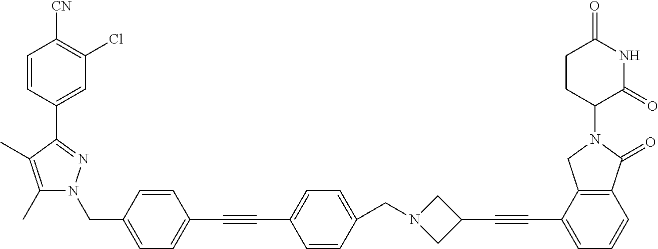 [SMILES: Cc1c(-c2ccc(C#N)c(Cl)c2)nn(Cc2ccc(C#Cc3ccc(CN4CC(C#Cc5cccc6c5CN(C5CCC(=O)NC5=O)C6=O)C4)cc3)cc2)c1C] |
| 207 | 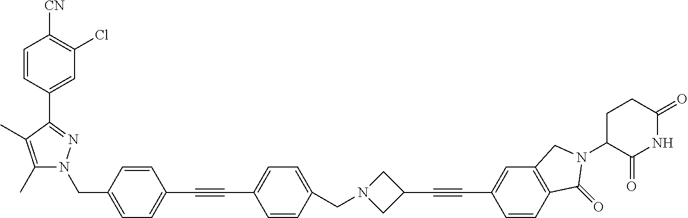 [SMILES: Cc1c(-c2ccc(C#N)c(Cl)c2)nn(Cc2ccc(C#Cc3ccc(CN4CC(C#Cc5ccc6c(c5)CN(C5CCC(=O)NC5=O)C6=O)C4)cc3)cc2)c1C] |
| 208 | 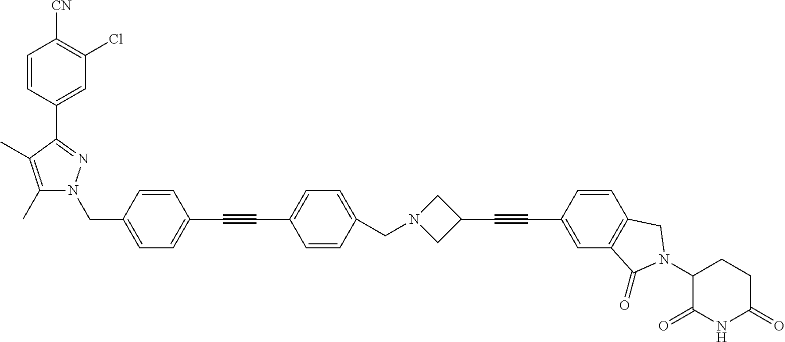 [SMILES: Cc1c(-c2ccc(C#N)c(Cl)c2)nn(Cc2ccc(C#Cc3ccc(CN4CC(C#Cc5ccc6c(c5)C(=O)N(C5CCC(=O)NC5=O)C6)C4)cc3)cc2)c1C] |
| 209 | 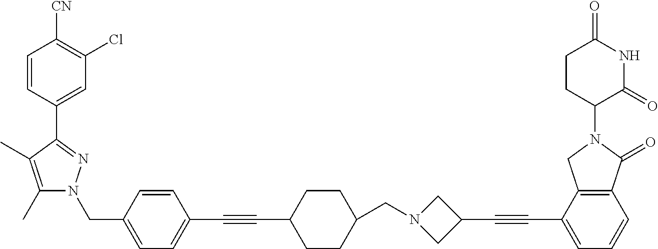 [SMILES: Cc1c(-c2ccc(C#N)c(Cl)c2)nn(Cc2ccc(C#CC3CCC(CN4CC(C#Cc5cccc6c5CN(C5CCC(=O)NC5=O)C6=O)C4)CC3)cc2)c1C] |
| 210 | 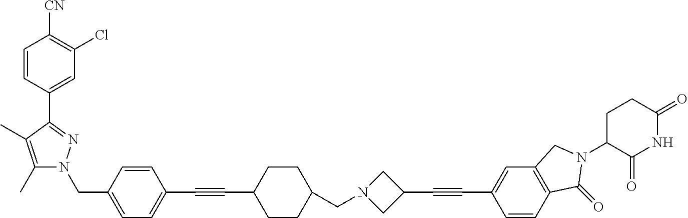 [SMILES: Cc1c(-c2ccc(C#N)c(Cl)c2)nn(Cc2ccc(C#CC3CCC(CN4CC(C#Cc5ccc6c(c5)CN(C5CCC(=O)NC5=O)C6=O)C4)CC3)cc2)c1C] |
| 211 | 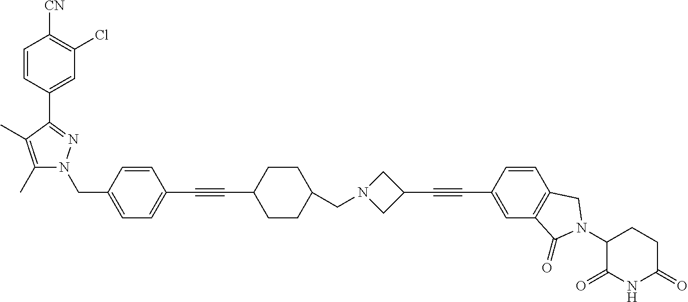 [SMILES: Cc1c(-c2ccc(C#N)c(Cl)c2)nn(Cc2ccc(C#CC3CCC(CN4CC(C#Cc5ccc6c(c5)C(=O)N(C5CCC(=O)NC5=O)C6)C4)CC3)cc2)c1C] |
| 212 | 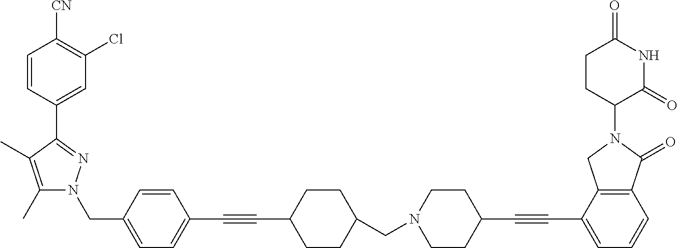 [SMILES: Cc1c(-c2ccc(C#N)c(Cl)c2)nn(Cc2ccc(C#CC3CCC(CN4CCC(C#Cc5cccc6c5CN(C5CCC(=O)NC5=O)C6=O)CC4)CC3)cc2)c1C] |
| 213 | 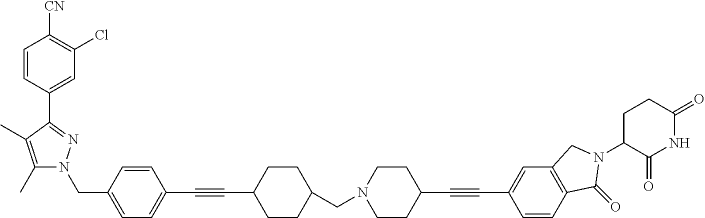 [SMILES: Cc1c(-c2ccc(C#N)c(Cl)c2)nn(Cc2ccc(C#CC3CCC(CN4CCC(C#Cc5ccc6c(c5)CN(C5CCC(=O)NC5=O)C6=O)CC4)CC3)cc2)c1C] |
| 214 | 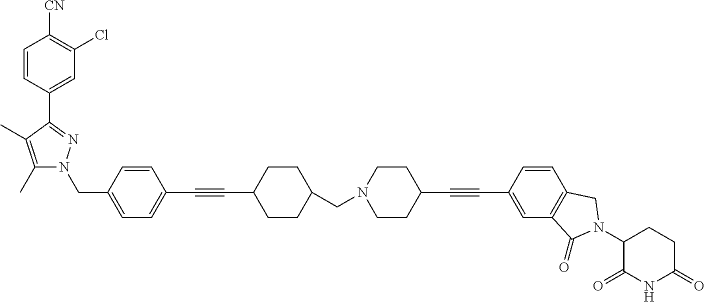 [SMILES: Cc1c(-c2ccc(C#N)c(Cl)c2)nn(Cc2ccc(C#CC3CCC(CN4CCC(C#Cc5ccc6c(c5)C(=O)N(C5CCC(=O)NC5=O)C6)CC4)CC3)cc2)c1C] |
| 215 | 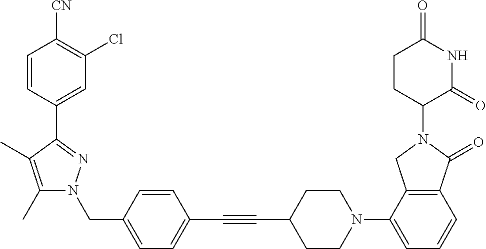 [SMILES: Cc1c(-c2ccc(C#N)c(Cl)c2)nn(Cc2ccc(C#CC3CCN(c4cccc5c4CN(C4CCC(=O)NC4=O)C5=O)CC3)cc2)c1C] |
| 216 | 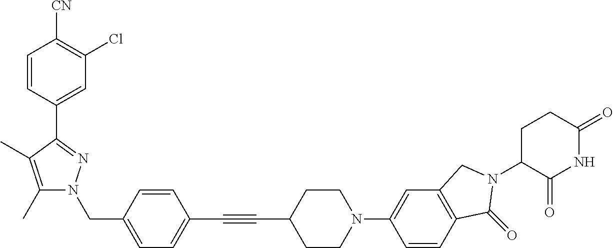 [SMILES: Cc1c(-c2ccc(C#N)c(Cl)c2)nn(Cc2ccc(C#CC3CCN(c4ccc5c(c4)CN(C4CCC(=O)NC4=O)C5=O)CC3)cc2)c1C] |
| 217 | 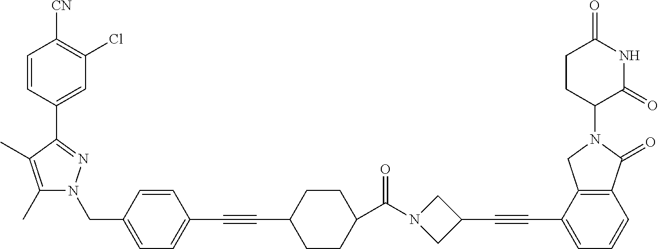 [SMILES: Cc1c(-c2ccc(C#N)c(Cl)c2)nn(Cc2ccc(C#CC3CCC(C(=O)N4CC(C#Cc5cccc6c5CN(C5CCC(=O)NC5=O)C6=O)C4)CC3)cc2)c1C] |
| 218 | 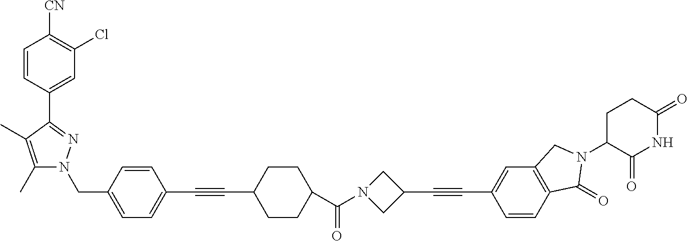 [SMILES: Cc1c(-c2ccc(C#N)c(Cl)c2)nn(Cc2ccc(C#CC3CCC(C(=O)N4CC(C#Cc5ccc6c(c5)CN(C5CCC(=O)NC5=O)C6=O)C4)CC3)cc2)c1C] |
| 219 | 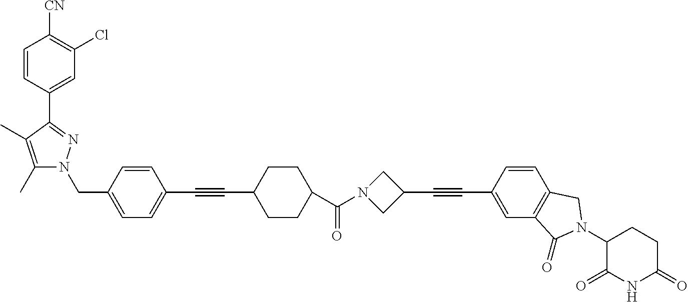 [SMILES: Cc1c(-c2ccc(C#N)c(Cl)c2)nn(Cc2ccc(C#CC3CCC(C(=O)N4CC(C#Cc5ccc6c(c5)C(=O)N(C5CCC(=O)NC5=O)C6)C4)CC3)cc2)c1C] |
| 220 | 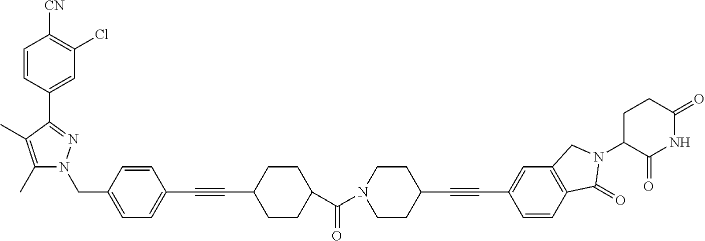 [SMILES: Cc1c(-c2ccc(C#N)c(Cl)c2)nn(Cc2ccc(C#CC3CCC(C(=O)N4CCC(C#Cc5ccc6c(c5)CN(C5CCC(=O)NC5=O)C6=O)CC4)CC3)cc2)c1C] |
| 221 | 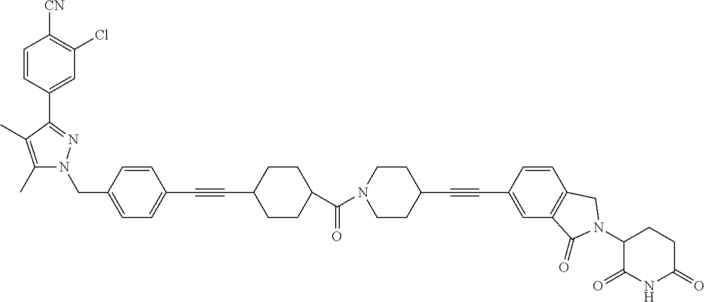 [SMILES: Cc1c(-c2ccc(C#N)c(Cl)c2)nn(Cc2ccc(C#CC3CCC(C(=O)N4CCC(C#Cc5ccc6c(c5)C(=O)N(C5CCC(=O)NC5=O)C6)CC4)CC3)cc2)c1C] |
| 222 | 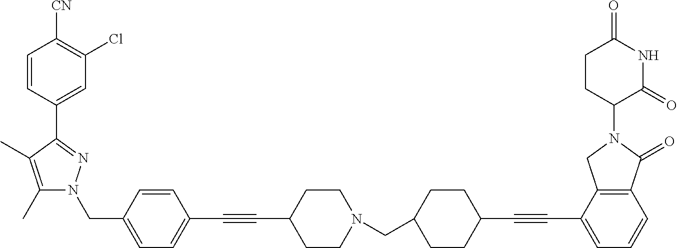 [SMILES: Cc1c(-c2ccc(C#N)c(Cl)c2)nn(Cc2ccc(C#CC3CCN(CC4CCC(C#Cc5cccc6c5CN(C5CCC(=O)NC5=O)C6=O)CC4)CC3)cc2)c1C] |
| 223 | 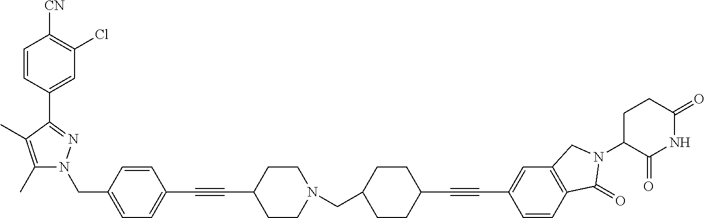 [SMILES: Cc1c(-c2ccc(C#N)c(Cl)c2)nn(Cc2ccc(C#CC3CCN(CC4CCC(C#Cc5ccc6c(c5)CN(C5CCC(=O)NC5=O)C6=O)CC4)CC3)cc2)c1C] |
| 224 | 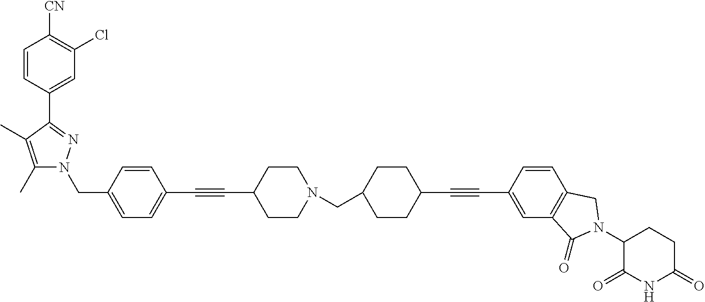 [SMILES: Cc1c(-c2ccc(C#N)c(Cl)c2)nn(Cc2ccc(C#CC3CCN(CC4CCC(C#Cc5ccc6c(c5)C(=O)N(C5CCC(=O)NC5=O)C6)CC4)CC3)cc2)c1C] |
| 225 | 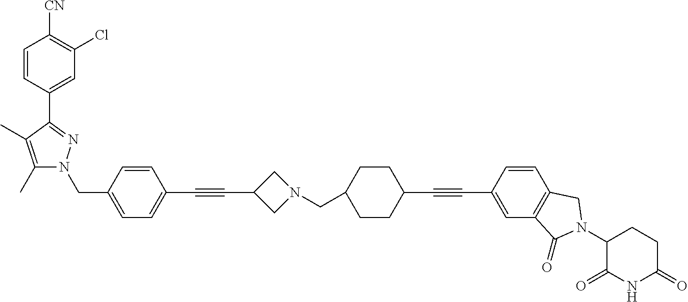 [SMILES: Cc1c(-c2ccc(C#N)c(Cl)c2)nn(Cc2ccc(C#CC3CN(CC4CCC(C#Cc5ccc6c(c5)C(=O)N(C5CCC(=O)NC5=O)C6)CC4)C3)cc2)c1C] |
| 226 | 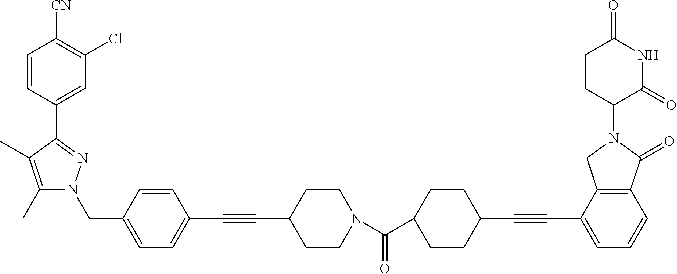 [SMILES: Cc1c(-c2ccc(C#N)c(Cl)c2)nn(Cc2ccc(C#CC3CCN(C(=O)C4CCC(C#Cc5cccc6c5CN(C5CCC(=O)NC5=O)C6=O)CC4)CC3)cc2)c1C] |
| 227 | 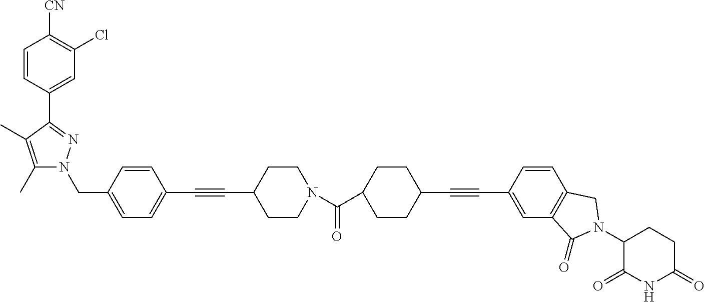 [SMILES: Cc1c(-c2ccc(C#N)c(Cl)c2)nn(Cc2ccc(C#CC3CCN(C(=O)C4CCC(C#Cc5ccc6c(c5)C(=O)N(C5CCC(=O)NC5=O)C6)CC4)CC3)cc2)c1C] |
| 228 | 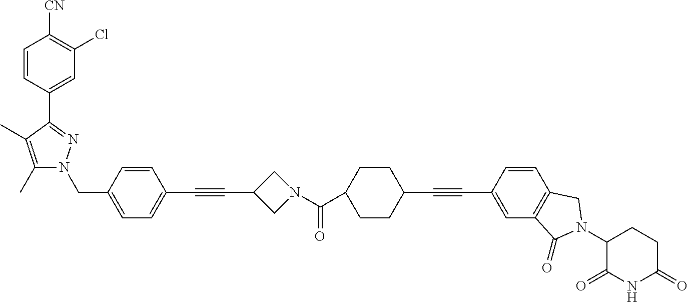 [SMILES: Cc1c(-c2ccc(C#N)c(Cl)c2)nn(Cc2ccc(C#CC3CN(C(=O)C4CCC(C#Cc5ccc6c(c5)C(=O)N(C5CCC(=O)NC5=O)C6)CC4)C3)cc2)c1C] |
| 229 | 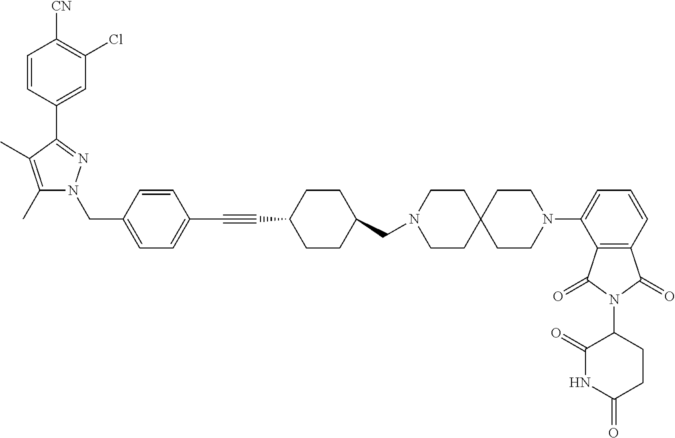 [SMILES: Cc1c(-c2ccc(C#N)c(Cl)c2)nn(Cc2ccc(C#C[C@H]3CC[C@H](CN4CCC5(CC4)CCN(c4cccc6c4C(=O)N(C4CCC(=O)NC4=O)C6=O)CC5)CC3)cc2)c1C] |
| 230 | 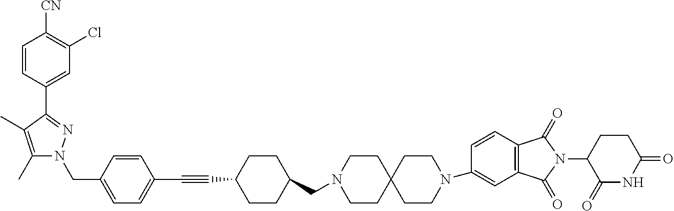 [SMILES: Cc1c(-c2ccc(C#N)c(Cl)c2)nn(Cc2ccc(C#C[C@H]3CC[C@H](CN4CCC5(CC4)CCN(c4ccc6c(c4)C(=O)N(C4CCC(=O)NC4=O)C6=O)CC5)CC3)cc2)c1C] |
| 231 | 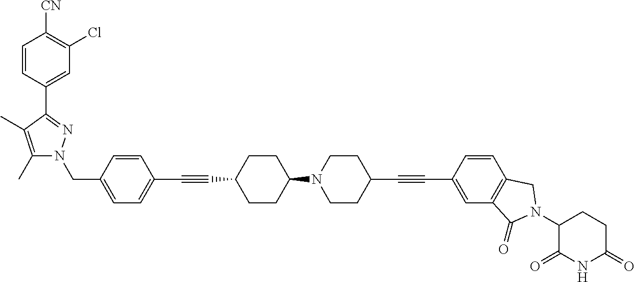 [SMILES: Cc1c(-c2ccc(C#N)c(Cl)c2)nn(Cc2ccc(C#C[C@H]3CC[C@H](N4CCC(C#Cc5ccc6c(c5)C(=O)N(C5CCC(=O)NC5=O)C6)CC4)CC3)cc2)c1C] |
| 232 | 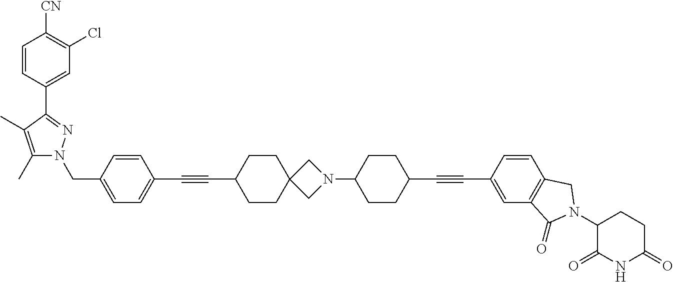 [SMILES: Cc1c(-c2ccc(C#N)c(Cl)c2)nn(Cc2ccc(C#CC3CCC4(CC3)CN(C3CCC(C#Cc5ccc6c(c5)C(=O)N(C5CCC(=O)NC5=O)C6)CC3)C4)cc2)c1C] |
| 233 | 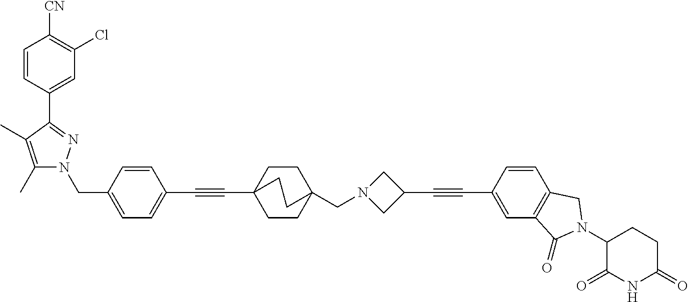 [SMILES: Cc1c(-c2ccc(C#N)c(Cl)c2)nn(Cc2ccc(C#CC34CCC(CN5CC(C#Cc6ccc7c(c6)C(=O)N(C6CCC(=O)NC6=O)C7)C5)(CC3)CC4)cc2)c1C] |
| 234 | 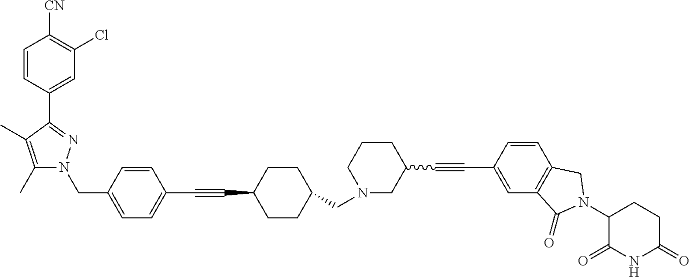 [SMILES: Cc1c(-c2ccc(C#N)c(Cl)c2)nn(Cc2ccc(C#C[C@H]3CC[C@H](CN4CCCC(C#Cc5ccc6c(c5)C(=O)N(C5CCC(=O)NC5=O)C6)C4)CC3)cc2)c1C] |
| 235 | 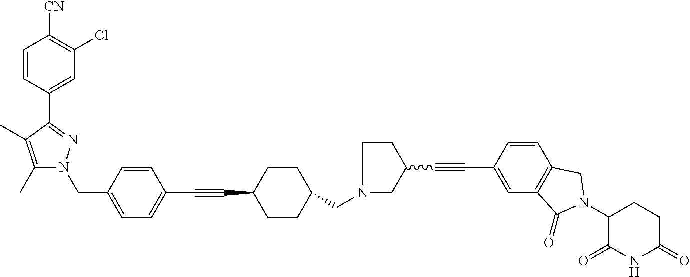 [SMILES: Cc1c(-c2ccc(C#N)c(Cl)c2)nn(Cc2ccc(C#C[C@H]3CC[C@H](CN4CCC(C#Cc5ccc6c(c5)C(=O)N(C5CCC(=O)NC5=O)C6)C4)CC3)cc2)c1C] |
| 236 | 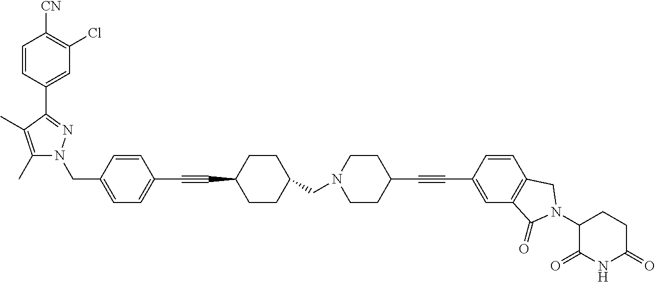 [SMILES: Cc1c(-c2ccc(C#N)c(Cl)c2)nn(Cc2ccc(C#C[C@H]3CC[C@H](CN4CCC(C#Cc5ccc6c(c5)C(=O)N(C5CCC(=O)NC5=O)C6)CC4)CC3)cc2)c1C] |
| 237 | 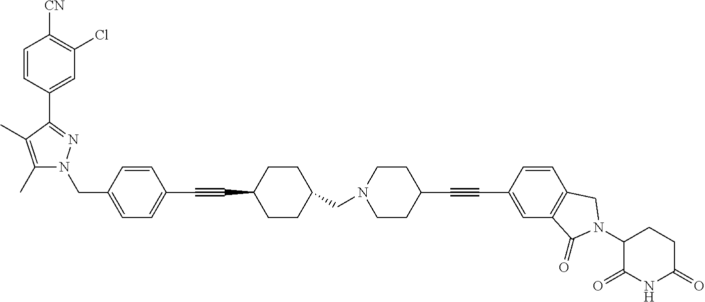 [SMILES: Cc1c(-c2ccc(C#N)c(Cl)c2)nn(Cc2ccc(C#C[C@H]3CC[C@H](CN4CCC(C#Cc5ccc6c(c5)C(=O)N(C5CCC(=O)NC5=O)C6)CC4)CC3)cc2)c1C] |
| 238 | 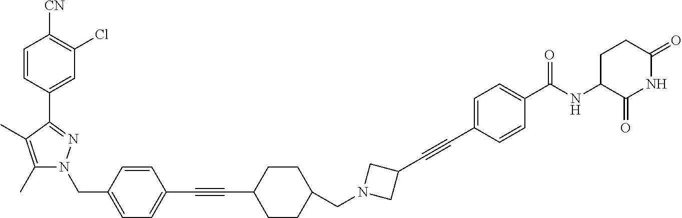 [SMILES: Cc1c(-c2ccc(C#N)c(Cl)c2)nn(Cc2ccc(C#CC3CCC(CN4CC(C#Cc5ccc(C(=O)NC6CCC(=O)NC6=O)cc5)C4)CC3)cc2)c1C] |
| 239 | 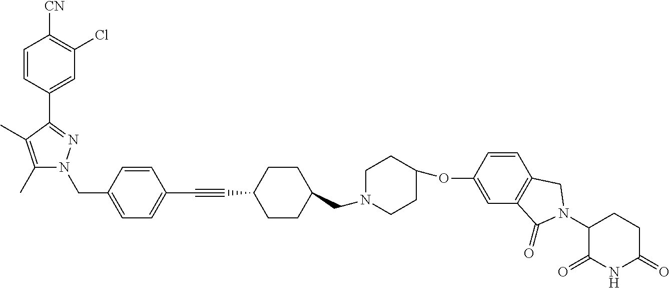 [SMILES: Cc1c(-c2ccc(C#N)c(Cl)c2)nn(Cc2ccc(C#C[C@H]3CC[C@H](CN4CCC(Oc5ccc6c(c5)C(=O)N(C5CCC(=O)NC5=O)C6)CC4)CC3)cc2)c1C] |
| 240 | 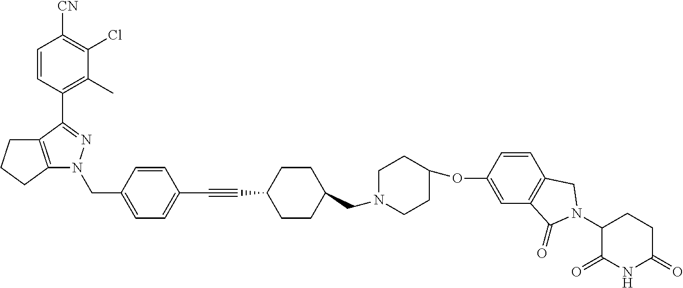 [SMILES: Cc1c(-c2nn(Cc3ccc(C#C[C@H]4CC[C@H](CN5CCC(Oc6ccc7c(c6)C(=O)N(C6CCC(=O)NC6=O)C7)CC5)CC4)cc3)c3c2CCC3)ccc(C#N)c1Cl] |
| 241 | 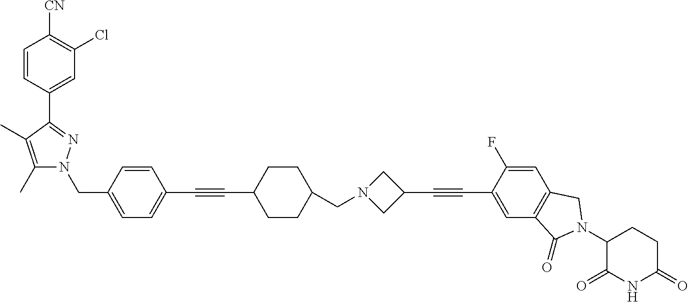 [SMILES: Cc1c(-c2ccc(C#N)c(Cl)c2)nn(Cc2ccc(C#CC3CCC(CN4CC(C#Cc5cc6c(cc5F)CN(C5CCC(=O)NC5=O)C6=O)C4)CC3)cc2)c1C] |
| 242 | 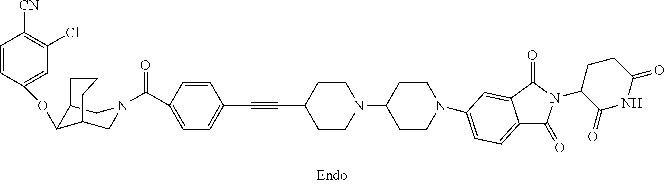 [SMILES: N#Cc1ccc(OC2C3CCCC2CN(C(=O)c2ccc(C#CC4CCN(C5CCN(c6ccc7c(c6)C(=O)N(C6CCC(=O)NC6=O)C7=O)CC5)CC4)cc2)C3)cc1Cl] |
| 243 | 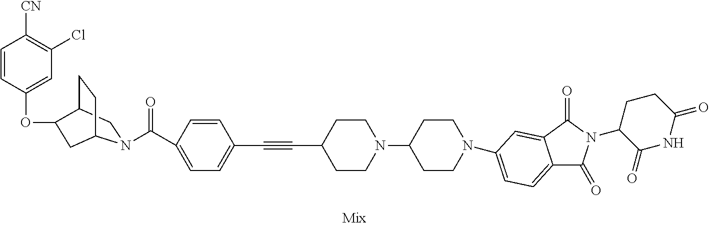 [SMILES: N#Cc1ccc(OC2CC3CCC2CN3C(=O)c2ccc(C#CC3CCN(C4CCN(c5ccc6c(c5)C(=O)N(C5CCC(=O)NC5=O)C6=O)CC4)CC3)cc2)cc1Cl] |
| 244 | 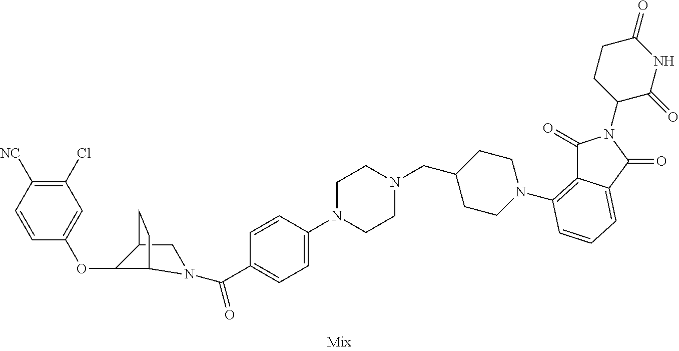 [SMILES: N#Cc1ccc(OC2C3CCC2N(C(=O)c2ccc(N4CCN(CC5CCN(c6cccc7c6C(=O)N(C6CCC(=O)NC6=O)C7=O)CC5)CC4)cc2)C3)cc1Cl] |
| 245 | 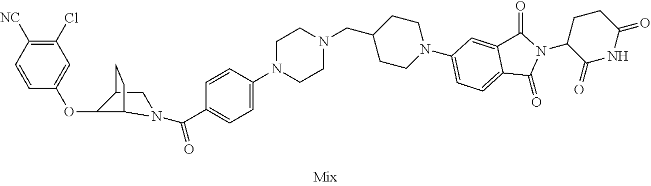 [SMILES: N#Cc1ccc(OC2C3CCC2N(C(=O)c2ccc(N4CCN(CC5CCN(c6ccc7c(c6)C(=O)N(C6CCC(=O)NC6=O)C7=O)CC5)CC4)cc2)C3)cc1Cl] |
| 246 | 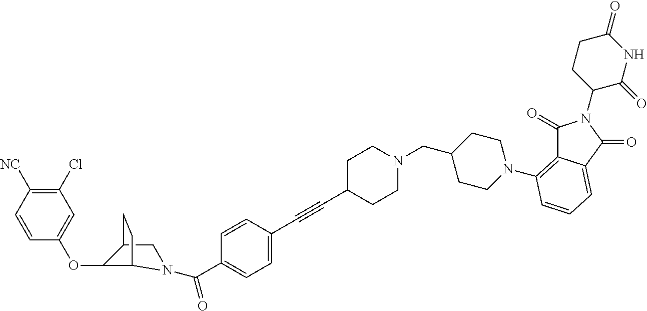 [SMILES: N#Cc1ccc(OC2C3CCC2N(C(=O)c2ccc(C#CC4CCN(CC5CCN(c6cccc7c6C(=O)N(C6CCC(=O)NC6=O)C7=O)CC5)CC4)cc2)C3)cc1Cl] |
| 247 | 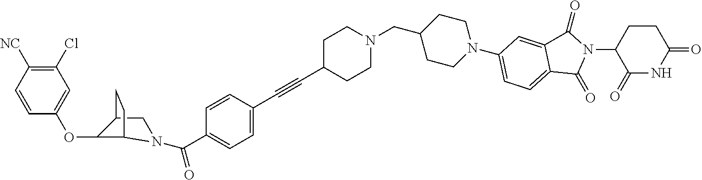 [SMILES: N#Cc1ccc(OC2C3CCC2N(C(=O)c2ccc(C#CC4CCN(CC5CCN(c6ccc7c(c6)C(=O)N(C6CCC(=O)NC6=O)C7=O)CC5)CC4)cc2)C3)cc1Cl] |
| 248 | 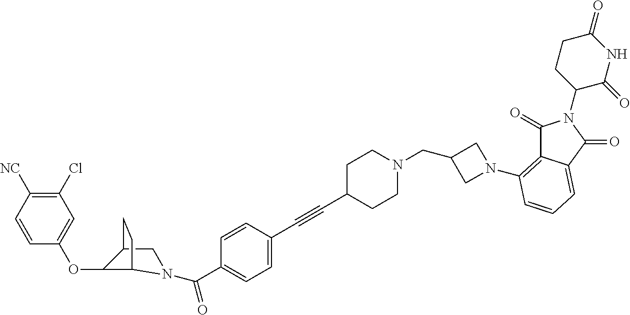 [SMILES: N#Cc1ccc(OC2C3CCC2N(C(=O)c2ccc(C#CC4CCN(CC5CN(c6cccc7c6C(=O)N(C6CCC(=O)NC6=O)C7=O)C5)CC4)cc2)C3)cc1Cl] |
| 249 | 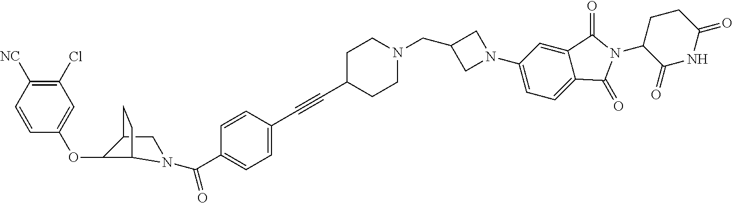 [SMILES: N#Cc1ccc(OC2C3CCC2N(C(=O)c2ccc(C#CC4CCN(CC5CN(c6ccc7c(c6)C(=O)N(C6CCC(=O)NC6=O)C7=O)C5)CC4)cc2)C3)cc1Cl] |
| 250 | 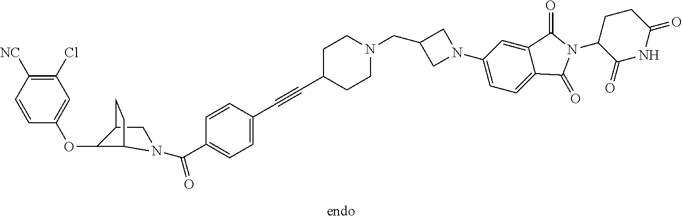 [SMILES: N#Cc1ccc(OC2C3CCC2N(C(=O)c2ccc(C#CC4CCN(CC5CN(c6ccc7c(c6)C(=O)N(C6CCC(=O)NC6=O)C7=O)C5)CC4)cc2)C3)cc1Cl] |
| 251 | 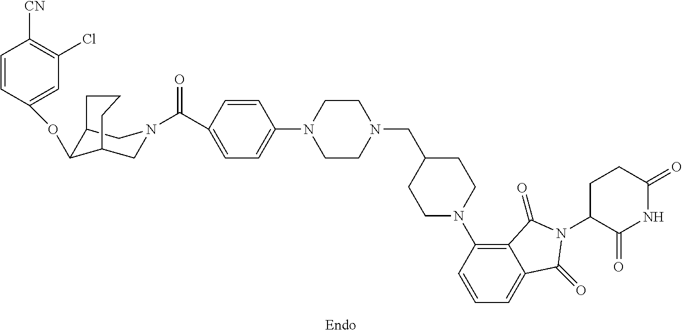 [SMILES: N#Cc1ccc(OC2C3CCCC2CN(C(=O)c2ccc(N4CCN(CC5CCN(c6cccc7c6C(=O)N(C6CCC(=O)NC6=O)C7=O)CC5)CC4)cc2)C3)cc1Cl] |
| 252 | 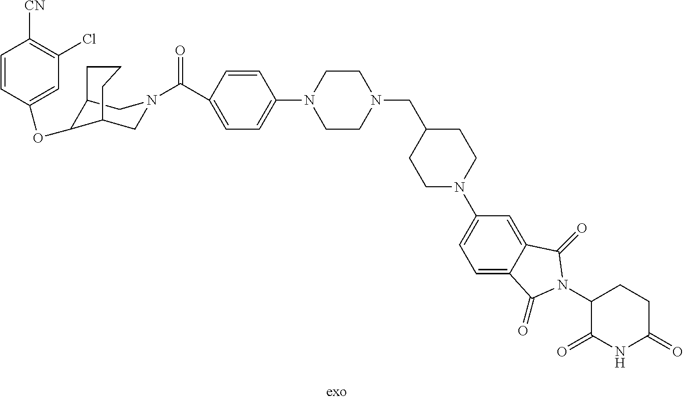 [SMILES: N#Cc1ccc(OC2C3CCCC2CN(C(=O)c2ccc(N4CCN(CC5CCN(c6ccc7c(c6)C(=O)N(C6CCC(=O)NC6=O)C7=O)CC5)CC4)cc2)C3)cc1Cl] |
| 253 | 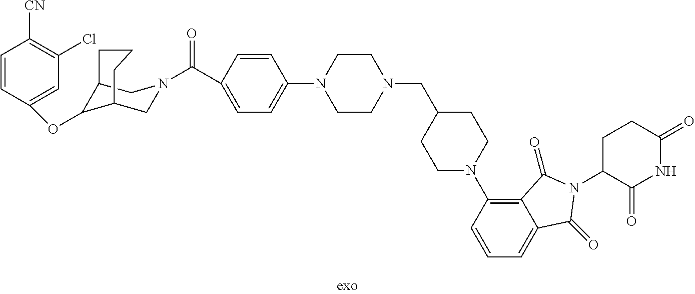 [SMILES: N#Cc1ccc(OC2C3CCCC2CN(C(=O)c2ccc(N4CCN(CC5CCN(c6cccc7c6C(=O)N(C6CCC(=O)NC6=O)C7=O)CC5)CC4)cc2)C3)cc1Cl] |
| 254 | 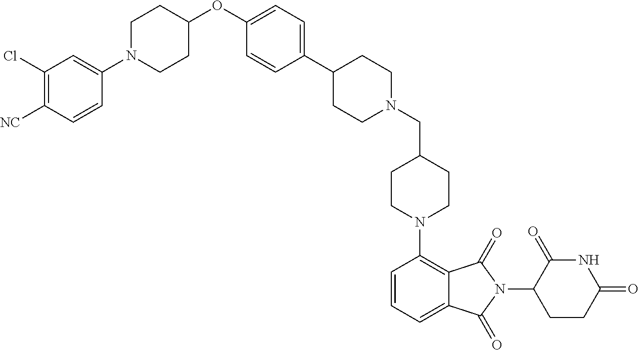 [SMILES: N#Cc1ccc(N2CCC(Oc3ccc(C4CCN(CC5CCN(c6cccc7c6C(=O)N(C6CCC(=O)NC6=O)C7=O)CC5)CC4)cc3)CC2)cc1Cl] |
| 255 | 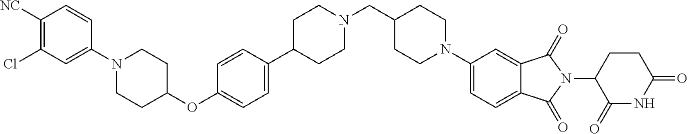 [SMILES: N#Cc1ccc(N2CCC(Oc3ccc(C4CCN(CC5CCN(c6ccc7c(c6)C(=O)N(C6CCC(=O)NC6=O)C7=O)CC5)CC4)cc3)CC2)cc1Cl] |
| 256 | 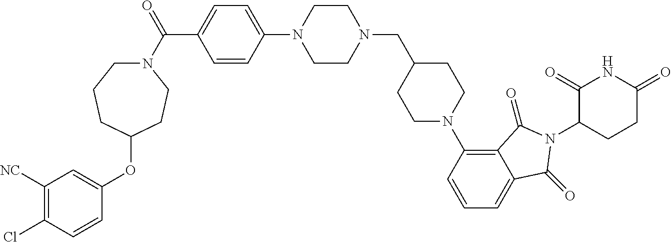 [SMILES: N#Cc1cc(OC2CCCN(C(=O)c3ccc(N4CCN(CC5CCN(c6cccc7c6C(=O)N(C6CCC(=O)NC6=O)C7=O)CC5)CC4)cc3)CC2)ccc1Cl] |
| 257 | 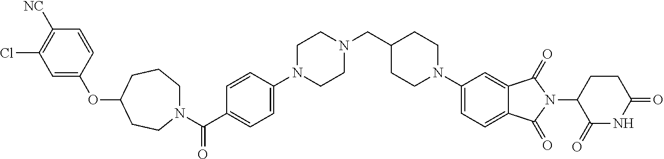 [SMILES: N#Cc1ccc(OC2CCCN(C(=O)c3ccc(N4CCN(CC5CCN(c6ccc7c(c6)C(=O)N(C6CCC(=O)NC6=O)C7=O)CC5)CC4)cc3)CC2)cc1Cl] |
| 258 | 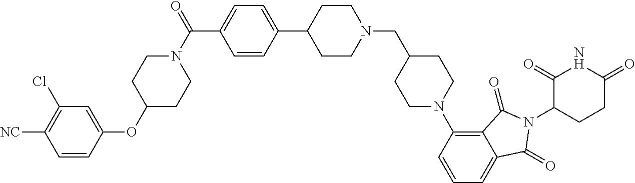 [SMILES: N#Cc1ccc(OC2CCN(C(=O)c3ccc(C4CCN(CC5CCN(c6cccc7c6C(=O)N(C6CCC(=O)NC6=O)C7=O)CC5)CC4)cc3)CC2)cc1Cl] |
| 259 | 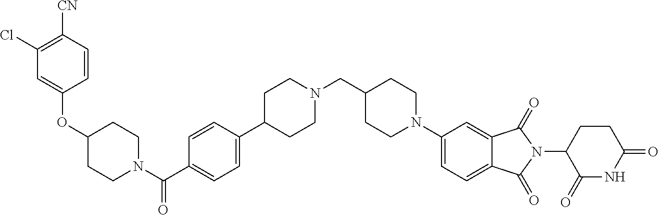 [SMILES: N#Cc1ccc(OC2CCN(C(=O)c3ccc(C4CCN(CC5CCN(c6ccc7c(c6)C(=O)N(C6CCC(=O)NC6=O)C7=O)CC5)CC4)cc3)CC2)cc1Cl] |
| 260 | 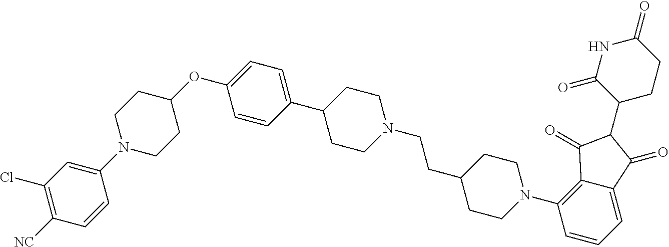 [SMILES: N#Cc1ccc(N2CCC(Oc3ccc(C4CCN(CCC5CCN(c6cccc7c6C(=O)C(C6CCC(=O)NC6=O)C7=O)CC5)CC4)cc3)CC2)cc1Cl] |
| 261 | 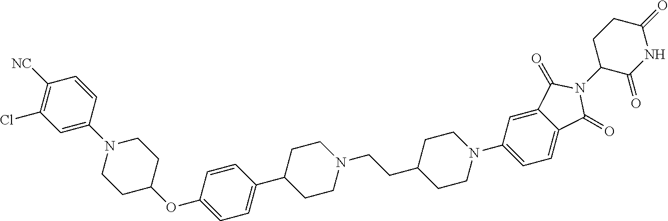 [SMILES: N#Cc1ccc(N2CCC(Oc3ccc(C4CCN(CCC5CCN(c6ccc7c(c6)C(=O)N(C6CCC(=O)NC6=O)C7=O)CC5)CC4)cc3)CC2)cc1Cl] |
| 262 | 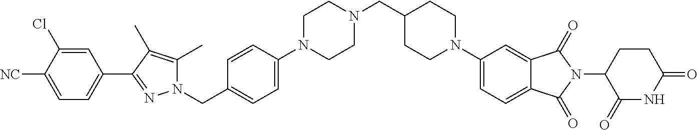 [SMILES: Cc1c(-c2ccc(C#N)c(Cl)c2)nn(Cc2ccc(N3CCN(CC4CCN(c5ccc6c(c5)C(=O)N(C5CCC(=O)NC5=O)C6=O)CC4)CC3)cc2)c1C] |
| 263 | 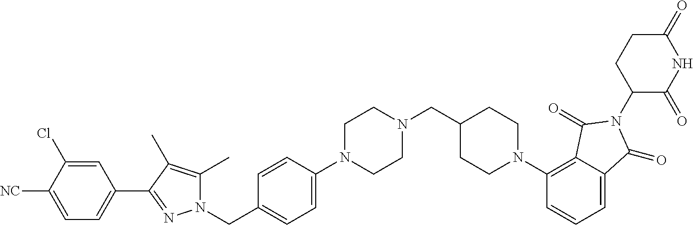 [SMILES: Cc1c(-c2ccc(C#N)c(Cl)c2)nn(Cc2ccc(N3CCN(CC4CCN(c5cccc6c5C(=O)N(C5CCC(=O)NC5=O)C6=O)CC4)CC3)cc2)c1C] |
| 264 | 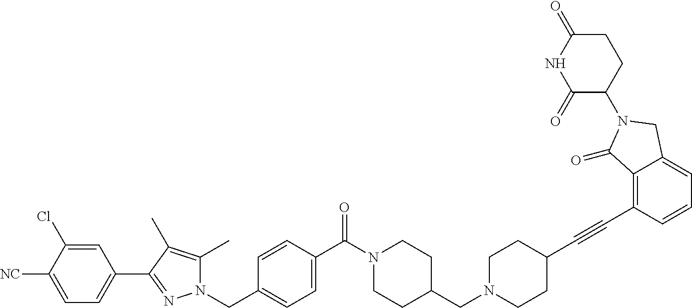 [SMILES: Cc1c(-c2ccc(C#N)c(Cl)c2)nn(Cc2ccc(C(=O)N3CCC(CN4CCC(C#Cc5cccc6c5C(=O)N(C5CCC(=O)NC5=O)C6)CC4)CC3)cc2)c1C] |
| 265 | 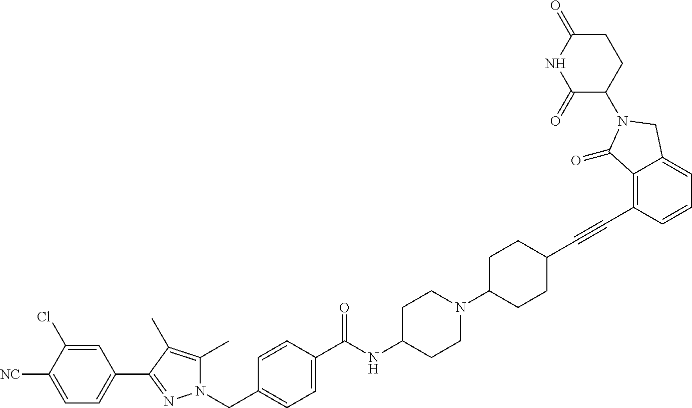 [SMILES: Cc1c(-c2ccc(C#N)c(Cl)c2)nn(Cc2ccc(C(=O)NC3CCN(C4CCC(C#Cc5cccc6c5C(=O)N(C5CCC(=O)NC5=O)C6)CC4)CC3)cc2)c1C] |
| 266 | 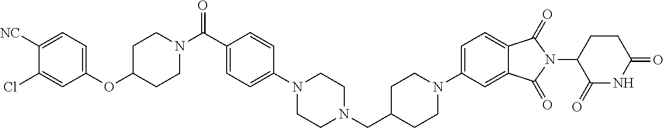 [SMILES: N#Cc1ccc(OC2CCN(C(=O)c3ccc(N4CCN(CC5CCN(c6ccc7c(c6)C(=O)N(C6CCC(=O)NC6=O)C7=O)CC5)CC4)cc3)CC2)cc1Cl] |
| 267 | 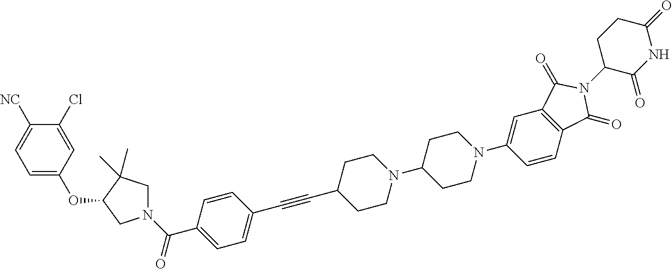 [SMILES: CC1(C)CN(C(=O)c2ccc(C#CC3CCN(C4CCN(c5ccc6c(c5)C(=O)N(C5CCC(=O)NC5=O)C6=O)CC4)CC3)cc2)C[C@@H]1Oc1ccc(C#N)c(Cl)c1] |
| 268 | 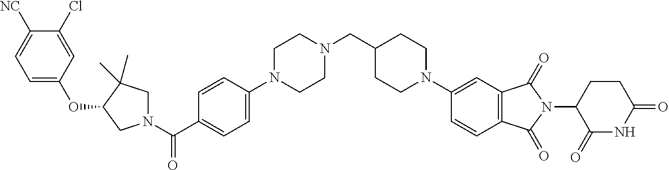 [SMILES: CC1(C)CN(C(=O)c2ccc(N3CCN(CC4CCN(c5ccc6c(c5)C(=O)N(C5CCC(=O)NC5=O)C6=O)CC4)CC3)cc2)C[C@@H]1Oc1ccc(C#N)c(Cl)c1] |
| 269 | 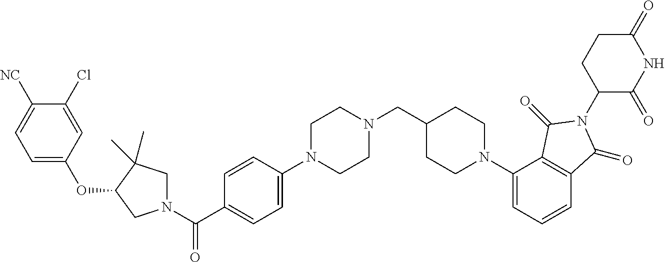 [SMILES: CC1(C)CN(C(=O)c2ccc(N3CCN(CC4CCN(c5cccc6c5C(=O)N(C5CCC(=O)NC5=O)C6=O)CC4)CC3)cc2)C[C@@H]1Oc1ccc(C#N)c(Cl)c1] |
| 270 | 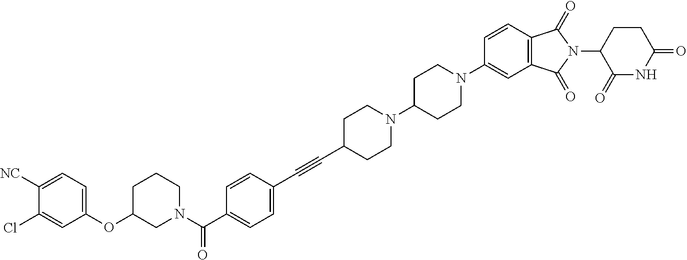 [SMILES: N#Cc1ccc(OC2CCCN(C(=O)c3ccc(C#CC4CCN(C5CCN(c6ccc7c(c6)C(=O)N(C6CCC(=O)NC6=O)C7=O)CC5)CC4)cc3)C2)cc1Cl] |
| 271 | 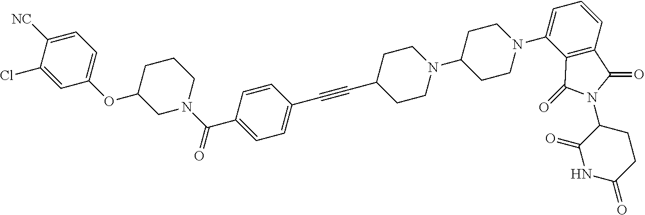 [SMILES: N#Cc1ccc(OC2CCCN(C(=O)c3ccc(C#CC4CCN(C5CCN(c6cccc7c6C(=O)N(C6CCC(=O)NC6=O)C7=O)CC5)CC4)cc3)C2)cc1Cl] |
| 272 | 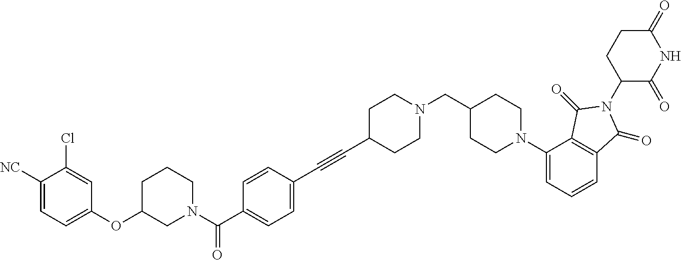 [SMILES: N#Cc1ccc(OC2CCCN(C(=O)c3ccc(C#CC4CCN(CC5CCN(c6cccc7c6C(=O)N(C6CCC(=O)NC6=O)C7=O)CC5)CC4)cc3)C2)cc1Cl] |
| 273 | 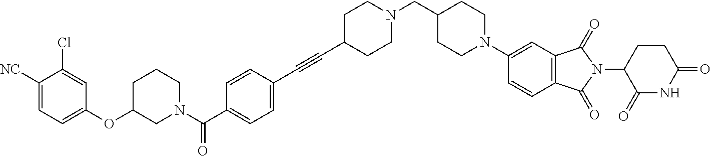 [SMILES: N#Cc1ccc(OC2CCCN(C(=O)c3ccc(C#CC4CCN(CC5CCN(c6ccc7c(c6)C(=O)N(C6CCC(=O)NC6=O)C7=O)CC5)CC4)cc3)C2)cc1Cl] |
| 274 | 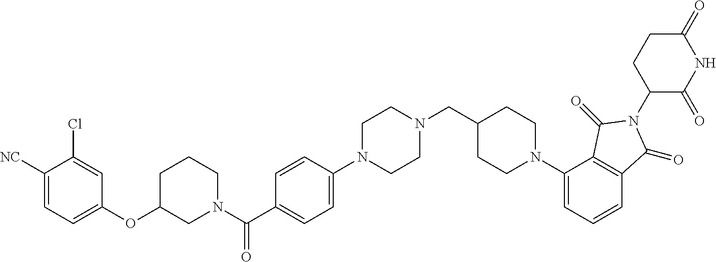 [SMILES: N#Cc1ccc(OC2CCCN(C(=O)c3ccc(N4CCN(CC5CCN(c6cccc7c6C(=O)N(C6CCC(=O)NC6=O)C7=O)CC5)CC4)cc3)C2)cc1Cl] |
| 275 | 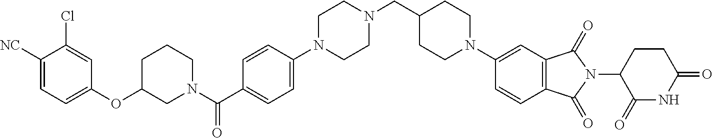 [SMILES: N#Cc1ccc(OC2CCCN(C(=O)c3ccc(N4CCN(CC5CCN(c6ccc7c(c6)C(=O)N(C6CCC(=O)NC6=O)C7=O)CC5)CC4)cc3)C2)cc1Cl] |
| 276 | 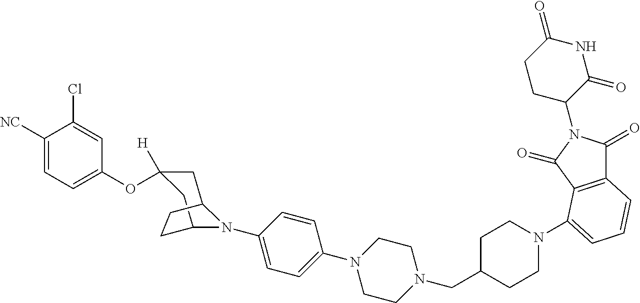 [SMILES: N#Cc1ccc(OC2CC3CCC(C2)N3c2ccc(N3CCN(CC4CCN(c5cccc6c5C(=O)N(C5CCC(=O)NC5=O)C6=O)CC4)CC3)cc2)cc1Cl] |
| 277 | 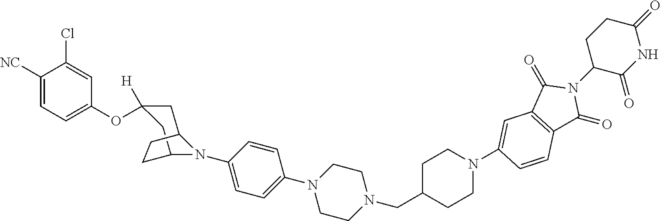 [SMILES: N#Cc1ccc(OC2CC3CCC(C2)N3c2ccc(N3CCN(CC4CCN(c5ccc6c(c5)C(=O)N(C5CCC(=O)NC5=O)C6=O)CC4)CC3)cc2)cc1Cl] |
| 278 | 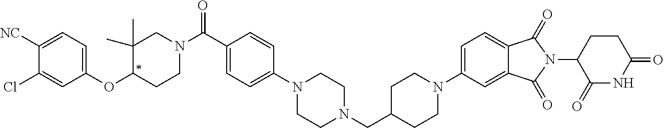 [SMILES: CC1(C)CN(C(=O)c2ccc(N3CCN(CC4CCN(c5ccc6c(c5)C(=O)N(C5CCC(=O)NC5=O)C6=O)CC4)CC3)cc2)CCC1Oc1ccc(C#N)c(Cl)c1] |
| 279 | 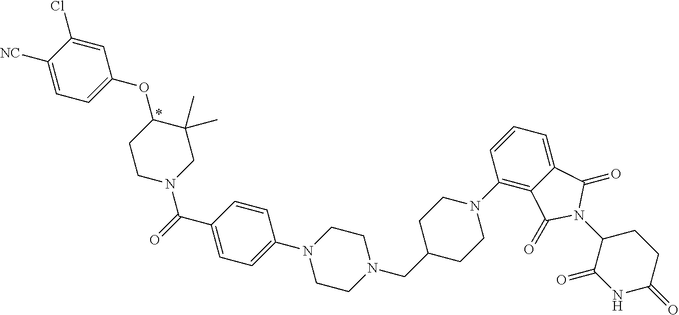 [SMILES: CC1(C)CN(C(=O)c2ccc(N3CCN(CC4CCN(c5cccc6c5C(=O)N(C5CCC(=O)NC5=O)C6=O)CC4)CC3)cc2)CCC1Oc1ccc(C#N)c(Cl)c1] |
| 280 | 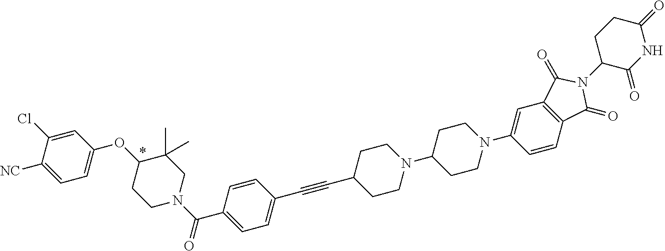 [SMILES: CC1(C)CN(C(=O)c2ccc(C#CC3CCN(C4CCN(c5ccc6c(c5)C(=O)N(C5CCC(=O)NC5=O)C6=O)CC4)CC3)cc2)CCC1Oc1ccc(C#N)c(Cl)c1] |
| 281 | 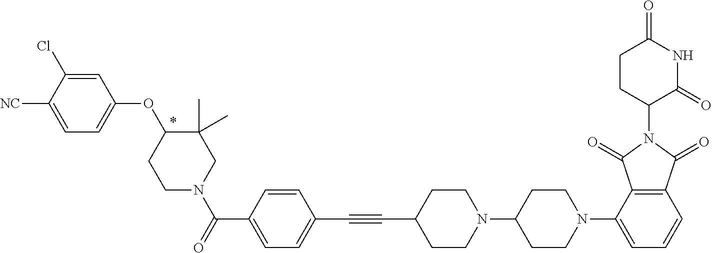 [SMILES: CC1(C)CN(C(=O)c2ccc(C#CC3CCN(C4CCN(c5cccc6c5C(=O)N(C5CCC(=O)NC5=O)C6=O)CC4)CC3)cc2)CCC1Oc1ccc(C#N)c(Cl)c1] |
| 282 | 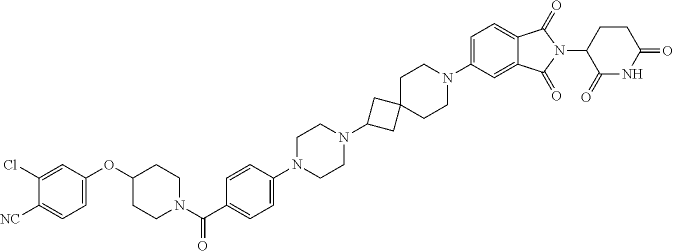 [SMILES: N#Cc1ccc(OC2CCN(C(=O)c3ccc(N4CCN(C5CC6(CCN(c7ccc8c(c7)C(=O)N(C7CCC(=O)NC7=O)C8=O)CC6)C5)CC4)cc3)CC2)cc1Cl] |
| 283 | 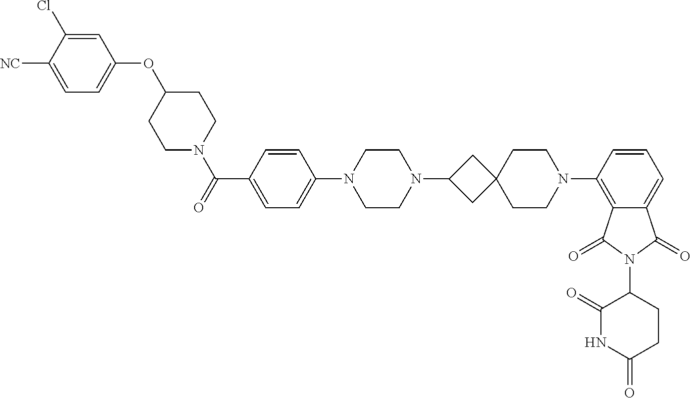 [SMILES: N#Cc1ccc(OC2CCN(C(=O)c3ccc(N4CCN(C5CC6(CCN(c7cccc8c7C(=O)N(C7CCC(=O)NC7=O)C8=O)CC6)C5)CC4)cc3)CC2)cc1Cl] |
| 284 | 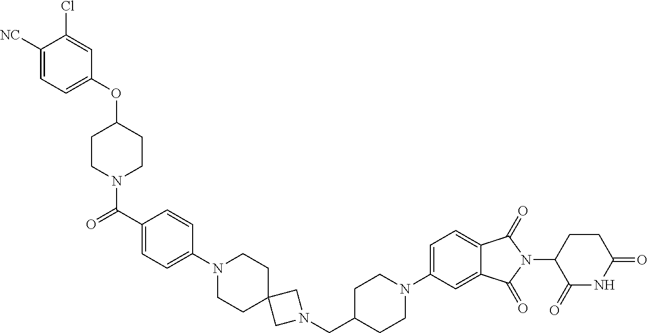 [SMILES: N#Cc1ccc(OC2CCN(C(=O)c3ccc(N4CCC5(CC4)CN(CC4CCN(c6ccc7c(c6)C(=O)N(C6CCC(=O)NC6=O)C7=O)CC4)C5)cc3)CC2)cc1Cl] |
| 285 | 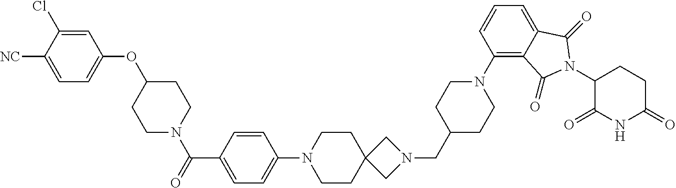 [SMILES: N#Cc1ccc(OC2CCN(C(=O)c3ccc(N4CCC5(CC4)CN(CC4CCN(c6cccc7c6C(=O)N(C6CCC(=O)NC6=O)C7=O)CC4)C5)cc3)CC2)cc1Cl] |
| 286 | 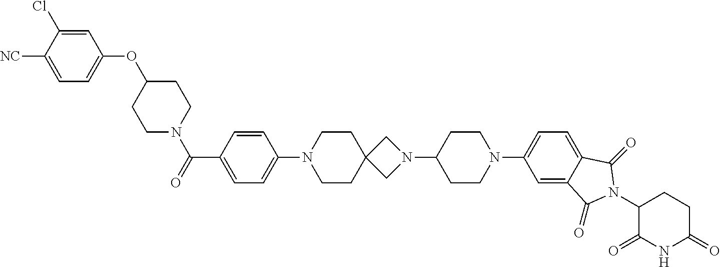 [SMILES: N#Cc1ccc(OC2CCN(C(=O)c3ccc(N4CCC5(CC4)CN(C4CCN(c6ccc7c(c6)C(=O)N(C6CCC(=O)NC6=O)C7=O)CC4)C5)cc3)CC2)cc1Cl] |
| 287 | 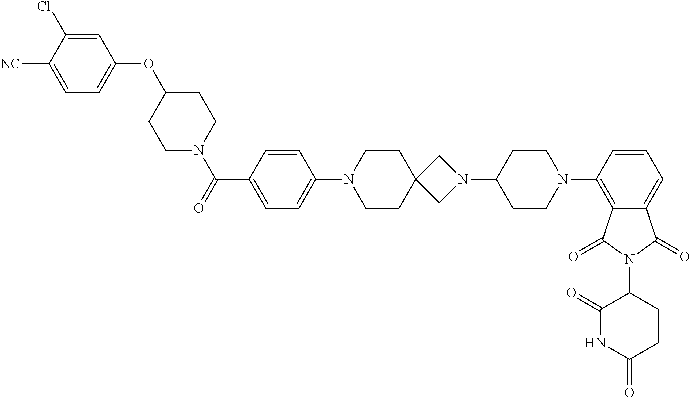 [SMILES: N#Cc1ccc(OC2CCN(C(=O)c3ccc(N4CCC5(CC4)CN(C4CCN(c6cccc7c6C(=O)N(C6CCC(=O)NC6=O)C7=O)CC4)C5)cc3)CC2)cc1Cl] |
| 288 | 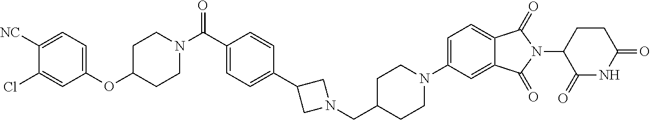 [SMILES: N#Cc1ccc(OC2CCN(C(=O)c3ccc(C4CN(CC5CCN(c6ccc7c(c6)C(=O)N(C6CCC(=O)NC6=O)C7=O)CC5)C4)cc3)CC2)cc1Cl] |
| 289 | 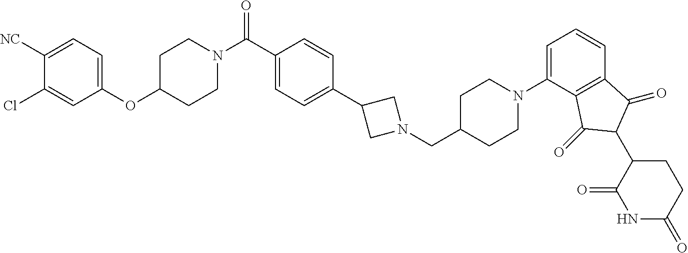 [SMILES: N#Cc1ccc(OC2CCN(C(=O)c3ccc(C4CN(CC5CCN(c6cccc7c6C(=O)C(C6CCC(=O)NC6=O)C7=O)CC5)C4)cc3)CC2)cc1Cl] |
| 290 | 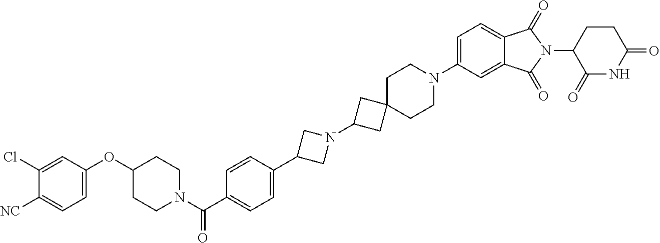 [SMILES: N#Cc1ccc(OC2CCN(C(=O)c3ccc(C4CN(C5CC6(CCN(c7ccc8c(c7)C(=O)N(C7CCC(=O)NC7=O)C8=O)CC6)C5)C4)cc3)CC2)cc1Cl] |
| 291 | 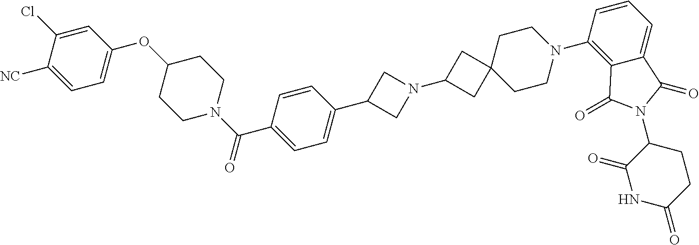 [SMILES: N#Cc1ccc(OC2CCN(C(=O)c3ccc(C4CN(C5CC6(CCN(c7cccc8c7C(=O)N(C7CCC(=O)NC7=O)C8=O)CC6)C5)C4)cc3)CC2)cc1Cl] |
| 292 | 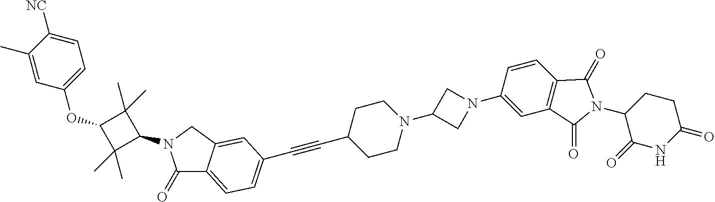 [SMILES: Cc1cc(O[C@H]2C(C)(C)[C@H](N3Cc4cc(C#CC5CCN(C6CN(c7ccc8c(c7)C(=O)N(C7CCC(=O)NC7=O)C8=O)C6)CC5)ccc4C3=O)C2(C)C)ccc1C#N] |
| 293 | 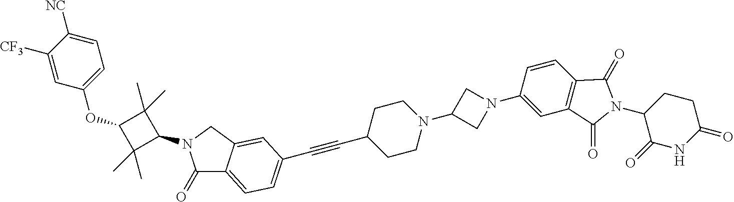 [SMILES: CC1(C)[C@H](Oc2ccc(C#N)c(C(F)(F)F)c2)C(C)(C)[C@H]1N1Cc2cc(C#CC3CCN(C4CN(c5ccc6c(c5)C(=O)N(C5CCC(=O)NC5=O)C6=O)C4)CC3)ccc2C1=O] |
| 294 | 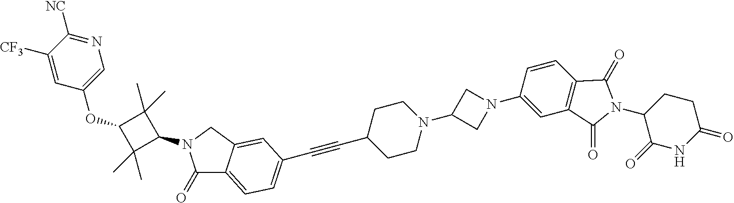 [SMILES: CC1(C)[C@H](Oc2cnc(C#N)c(C(F)(F)F)c2)C(C)(C)[C@H]1N1Cc2cc(C#CC3CCN(C4CN(c5ccc6c(c5)C(=O)N(C5CCC(=O)NC5=O)C6=O)C4)CC3)ccc2C1=O] |
| 295 | 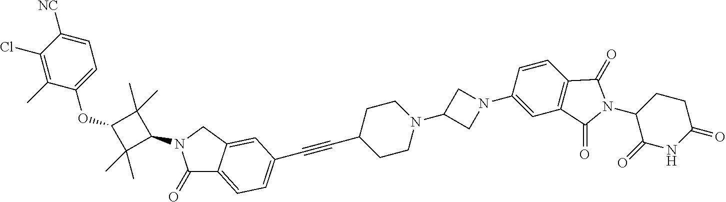 [SMILES: Cc1c(O[C@H]2C(C)(C)[C@H](N3Cc4cc(C#CC5CCN(C6CN(c7ccc8c(c7)C(=O)N(C7CCC(=O)NC7=O)C8=O)C6)CC5)ccc4C3=O)C2(C)C)ccc(C#N)c1Cl] |
| 296 | 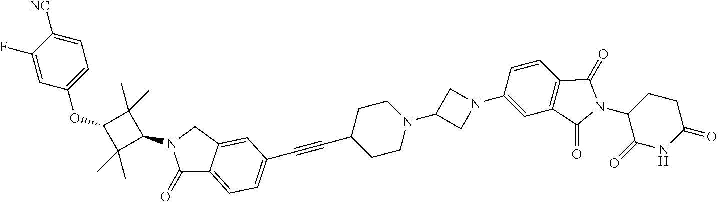 [SMILES: CC1(C)[C@H](Oc2ccc(C#N)c(F)c2)C(C)(C)[C@H]1N1Cc2cc(C#CC3CCN(C4CN(c5ccc6c(c5)C(=O)N(C5CCC(=O)NC5=O)C6=O)C4)CC3)ccc2C1=O] |
| 297 | 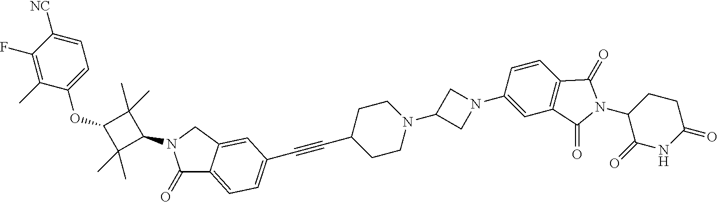 [SMILES: Cc1c(O[C@H]2C(C)(C)[C@H](N3Cc4cc(C#CC5CCN(C6CN(c7ccc8c(c7)C(=O)N(C7CCC(=O)NC7=O)C8=O)C6)CC5)ccc4C3=O)C2(C)C)ccc(C#N)c1F] |
| 298 | 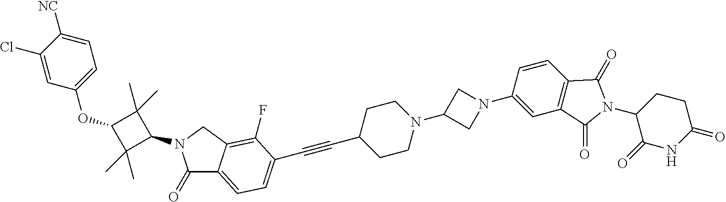 [SMILES: CC1(C)[C@H](Oc2ccc(C#N)c(Cl)c2)C(C)(C)[C@H]1N1Cc2c(ccc(C#CC3CCN(C4CN(c5ccc6c(c5)C(=O)N(C5CCC(=O)NC5=O)C6=O)C4)CC3)c2F)C1=O] |
| 299 | 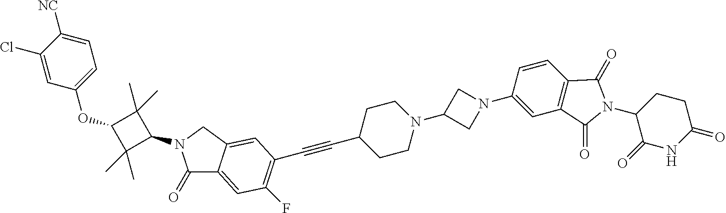 [SMILES: CC1(C)[C@H](Oc2ccc(C#N)c(Cl)c2)C(C)(C)[C@H]1N1Cc2cc(C#CC3CCN(C4CN(c5ccc6c(c5)C(=O)N(C5CCC(=O)NC5=O)C6=O)C4)CC3)c(F)cc2C1=O] |
| 300 | 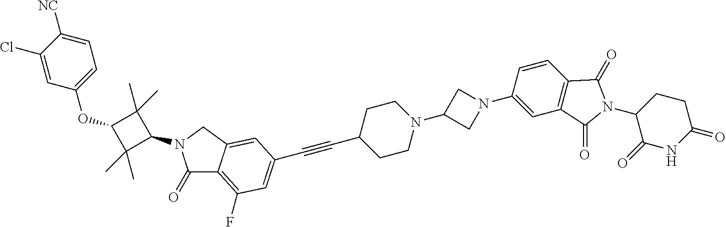 [SMILES: CC1(C)[C@H](Oc2ccc(C#N)c(Cl)c2)C(C)(C)[C@H]1N1Cc2cc(C#CC3CCN(C4CN(c5ccc6c(c5)C(=O)N(C5CCC(=O)NC5=O)C6=O)C4)CC3)cc(F)c2C1=O] |
| 301 | 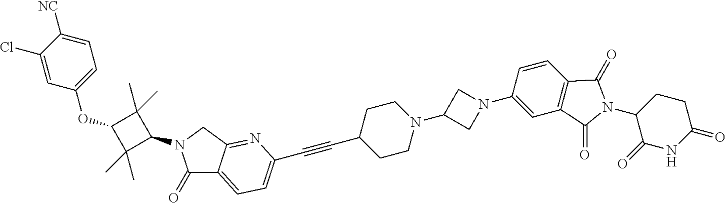 [SMILES: CC1(C)[C@H](Oc2ccc(C#N)c(Cl)c2)C(C)(C)[C@H]1N1Cc2nc(C#CC3CCN(C4CN(c5ccc6c(c5)C(=O)N(C5CCC(=O)NC5=O)C6=O)C4)CC3)ccc2C1=O] |
| 302 | 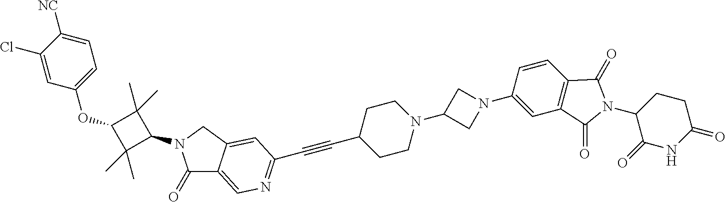 [SMILES: CC1(C)[C@H](Oc2ccc(C#N)c(Cl)c2)C(C)(C)[C@H]1N1Cc2cc(C#CC3CCN(C4CN(c5ccc6c(c5)C(=O)N(C5CCC(=O)NC5=O)C6=O)C4)CC3)ncc2C1=O] |
| 303 | 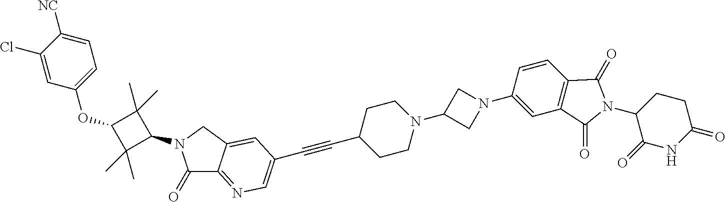 [SMILES: CC1(C)[C@H](Oc2ccc(C#N)c(Cl)c2)C(C)(C)[C@H]1N1Cc2cc(C#CC3CCN(C4CN(c5ccc6c(c5)C(=O)N(C5CCC(=O)NC5=O)C6=O)C4)CC3)cnc2C1=O] |
| 304 | 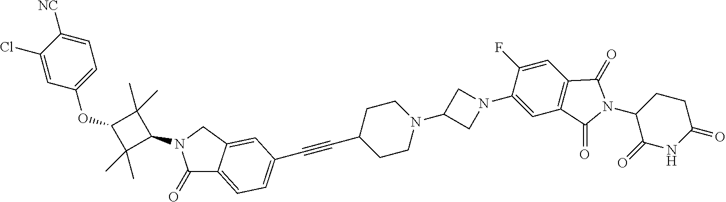 [SMILES: CC1(C)[C@H](Oc2ccc(C#N)c(Cl)c2)C(C)(C)[C@H]1N1Cc2cc(C#CC3CCN(C4CN(c5cc6c(cc5F)C(=O)N(C5CCC(=O)NC5=O)C6=O)C4)CC3)ccc2C1=O] |
| 305 | 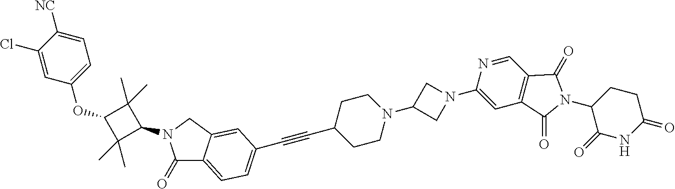 [SMILES: CC1(C)[C@H](Oc2ccc(C#N)c(Cl)c2)C(C)(C)[C@H]1N1Cc2cc(C#CC3CCN(C4CN(c5cc6c(cn5)C(=O)N(C5CCC(=O)NC5=O)C6=O)C4)CC3)ccc2C1=O] |
| 306 | 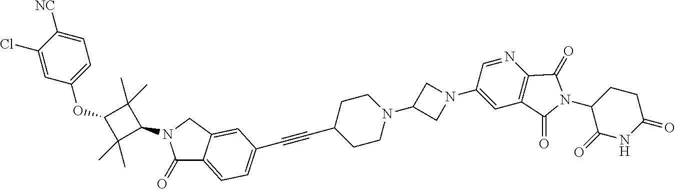 [SMILES: CC1(C)[C@H](Oc2ccc(C#N)c(Cl)c2)C(C)(C)[C@H]1N1Cc2cc(C#CC3CCN(C4CN(c5cnc6c(c5)C(=O)N(C5CCC(=O)NC5=O)C6=O)C4)CC3)ccc2C1=O] |
| 307 | 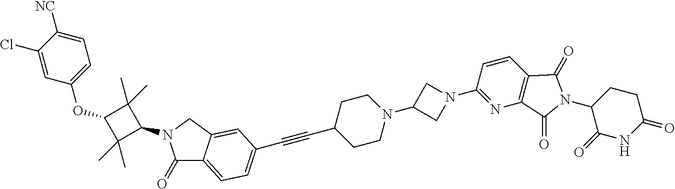 [SMILES: CC1(C)[C@H](Oc2ccc(C#N)c(Cl)c2)C(C)(C)[C@H]1N1Cc2cc(C#CC3CCN(C4CN(c5ccc6c(n5)C(=O)N(C5CCC(=O)NC5=O)C6=O)C4)CC3)ccc2C1=O] |
| 308 | 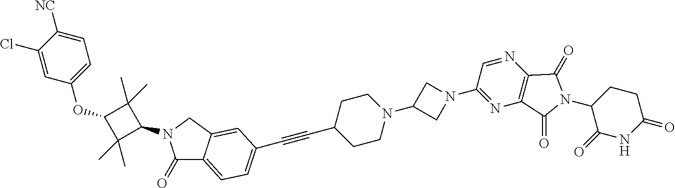 [SMILES: CC1(C)[C@H](Oc2ccc(C#N)c(Cl)c2)C(C)(C)[C@H]1N1Cc2cc(C#CC3CCN(C4CN(c5cnc6c(n5)C(=O)N(C5CCC(=O)NC5=O)C6=O)C4)CC3)ccc2C1=O] |
| 309 | 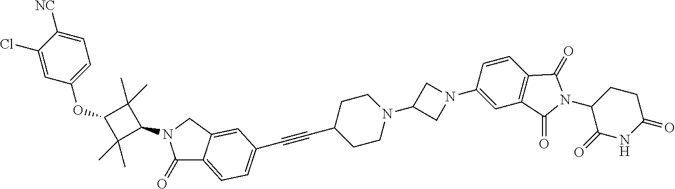 [SMILES: CC1(C)[C@H](Oc2ccc(C#N)c(Cl)c2)C(C)(C)[C@H]1N1Cc2cc(C#CC3CCN(C4CN(c5ccc6c(c5)C(=O)N(C5CCC(=O)NC5=O)C6=O)C4)CC3)ccc2C1=O] |
| 310 | 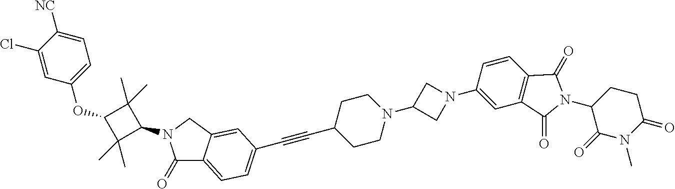 [SMILES: CN1C(=O)CCC(N2C(=O)c3ccc(N4CC(N5CCC(C#Cc6ccc7c(c6)CN([C@H]6C(C)(C)[C@H](Oc8ccc(C#N)c(Cl)c8)C6(C)C)C7=O)CC5)C4)cc3C2=O)C1=O] |
| 311 | 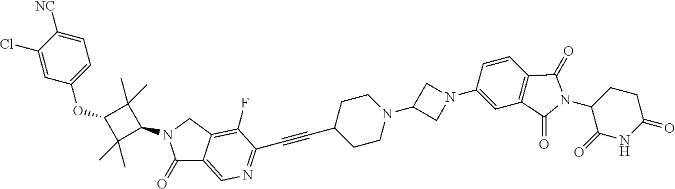 [SMILES: CC1(C)[C@H](Oc2ccc(C#N)c(Cl)c2)C(C)(C)[C@H]1N1Cc2c(cnc(C#CC3CCN(C4CN(c5ccc6c(c5)C(=O)N(C5CCC(=O)NC5=O)C6=O)C4)CC3)c2F)C1=O] |
| 312 | 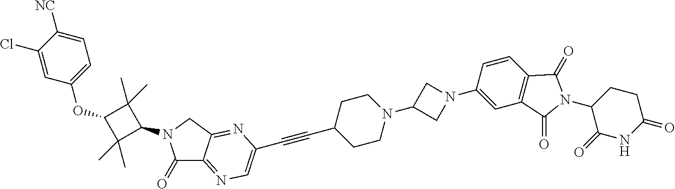 [SMILES: CC1(C)[C@H](Oc2ccc(C#N)c(Cl)c2)C(C)(C)[C@H]1N1Cc2nc(C#CC3CCN(C4CN(c5ccc6c(c5)C(=O)N(C5CCC(=O)NC5=O)C6=O)C4)CC3)cnc2C1=O] |
| 313 | 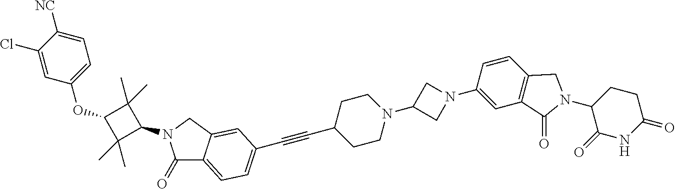 [SMILES: CC1(C)[C@H](Oc2ccc(C#N)c(Cl)c2)C(C)(C)[C@H]1N1Cc2cc(C#CC3CCN(C4CN(c5ccc6c(c5)C(=O)N(C5CCC(=O)NC5=O)C6)C4)CC3)ccc2C1=O] |
| 314 | 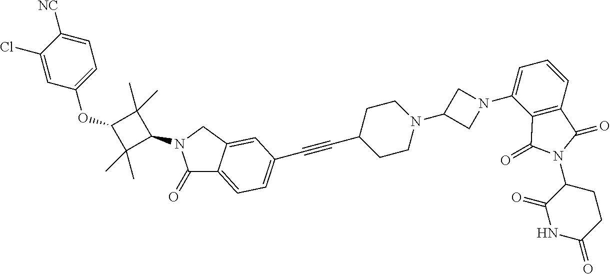 [SMILES: CC1(C)[C@H](Oc2ccc(C#N)c(Cl)c2)C(C)(C)[C@H]1N1Cc2cc(C#CC3CCN(C4CN(c5cccc6c5C(=O)N(C5CCC(=O)NC5=O)C6=O)C4)CC3)ccc2C1=O] |
| 315 | 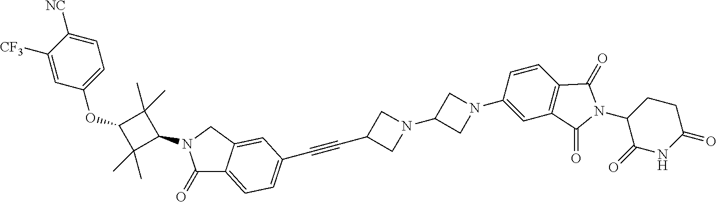 [SMILES: CC1(C)[C@H](Oc2ccc(C#N)c(C(F)(F)F)c2)C(C)(C)[C@H]1N1Cc2cc(C#CC3CN(C4CN(c5ccc6c(c5)C(=O)N(C5CCC(=O)NC5=O)C6=O)C4)C3)ccc2C1=O] |
| 316 | 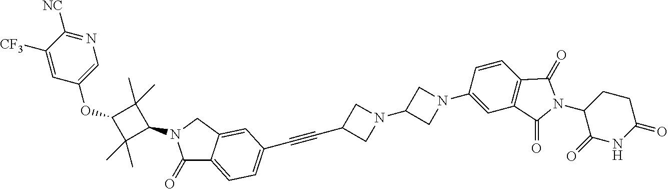 [SMILES: CC1(C)[C@H](Oc2cnc(C#N)c(C(F)(F)F)c2)C(C)(C)[C@H]1N1Cc2cc(C#CC3CN(C4CN(c5ccc6c(c5)C(=O)N(C5CCC(=O)NC5=O)C6=O)C4)C3)ccc2C1=O] |
| 317 | 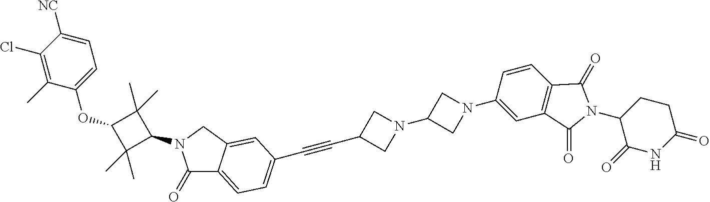 [SMILES: Cc1c(O[C@H]2C(C)(C)[C@H](N3Cc4cc(C#CC5CN(C6CN(c7ccc8c(c7)C(=O)N(C7CCC(=O)NC7=O)C8=O)C6)C5)ccc4C3=O)C2(C)C)ccc(C#N)c1Cl] |
| 318 | 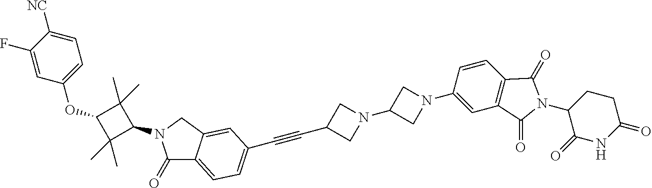 [SMILES: CC1(C)[C@H](Oc2ccc(C#N)c(F)c2)C(C)(C)[C@H]1N1Cc2cc(C#CC3CN(C4CN(c5ccc6c(c5)C(=O)N(C5CCC(=O)NC5=O)C6=O)C4)C3)ccc2C1=O] |
| 319 | 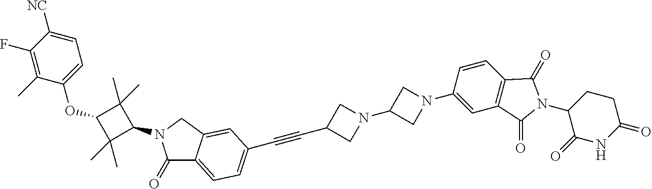 [SMILES: Cc1c(O[C@H]2C(C)(C)[C@H](N3Cc4cc(C#CC5CN(C6CN(c7ccc8c(c7)C(=O)N(C7CCC(=O)NC7=O)C8=O)C6)C5)ccc4C3=O)C2(C)C)ccc(C#N)c1F] |
| 320 | 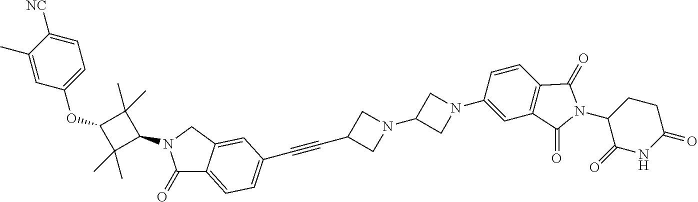 [SMILES: Cc1cc(O[C@H]2C(C)(C)[C@H](N3Cc4cc(C#CC5CN(C6CN(c7ccc8c(c7)C(=O)N(C7CCC(=O)NC7=O)C8=O)C6)C5)ccc4C3=O)C2(C)C)ccc1C#N] |
| 321 | 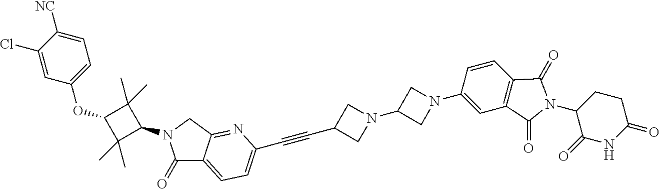 [SMILES: CC1(C)[C@H](Oc2ccc(C#N)c(Cl)c2)C(C)(C)[C@H]1N1Cc2nc(C#CC3CN(C4CN(c5ccc6c(c5)C(=O)N(C5CCC(=O)NC5=O)C6=O)C4)C3)ccc2C1=O] |
| 322 | 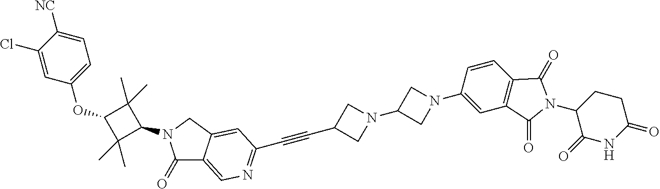 [SMILES: CC1(C)[C@H](Oc2ccc(C#N)c(Cl)c2)C(C)(C)[C@H]1N1Cc2cc(C#CC3CN(C4CN(c5ccc6c(c5)C(=O)N(C5CCC(=O)NC5=O)C6=O)C4)C3)ncc2C1=O] |
| 323 | 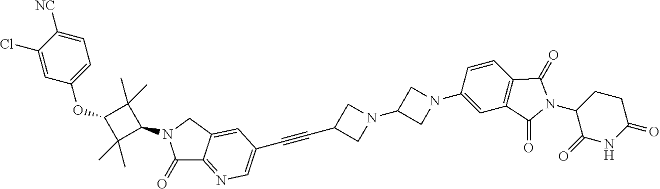 [SMILES: CC1(C)[C@H](Oc2ccc(C#N)c(Cl)c2)C(C)(C)[C@H]1N1Cc2cc(C#CC3CN(C4CN(c5ccc6c(c5)C(=O)N(C5CCC(=O)NC5=O)C6=O)C4)C3)cnc2C1=O] |
| 324 | 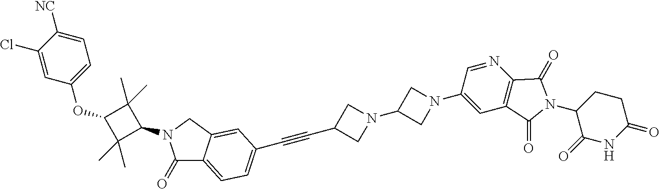 [SMILES: CC1(C)[C@H](Oc2ccc(C#N)c(Cl)c2)C(C)(C)[C@H]1N1Cc2cc(C#CC3CN(C4CN(c5cnc6c(c5)C(=O)N(C5CCC(=O)NC5=O)C6=O)C4)C3)ccc2C1=O] |
| 325 | 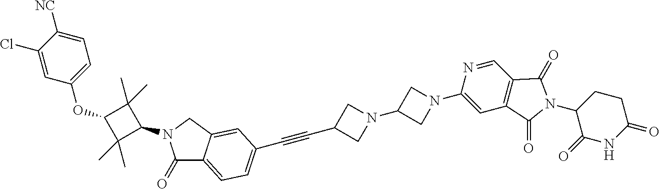 [SMILES: CC1(C)[C@H](Oc2ccc(C#N)c(Cl)c2)C(C)(C)[C@H]1N1Cc2cc(C#CC3CN(C4CN(c5cc6c(cn5)C(=O)N(C5CCC(=O)NC5=O)C6=O)C4)C3)ccc2C1=O] |
| 326 | 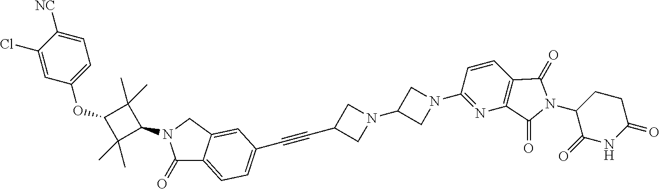 [SMILES: CC1(C)[C@H](Oc2ccc(C#N)c(Cl)c2)C(C)(C)[C@H]1N1Cc2cc(C#CC3CN(C4CN(c5ccc6c(n5)C(=O)N(C5CCC(=O)NC5=O)C6=O)C4)C3)ccc2C1=O] |
| 327 | 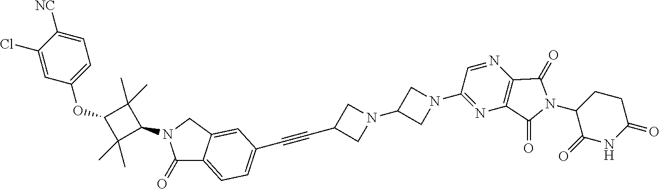 [SMILES: CC1(C)[C@H](Oc2ccc(C#N)c(Cl)c2)C(C)(C)[C@H]1N1Cc2cc(C#CC3CN(C4CN(c5cnc6c(n5)C(=O)N(C5CCC(=O)NC5=O)C6=O)C4)C3)ccc2C1=O] |
| 328 | 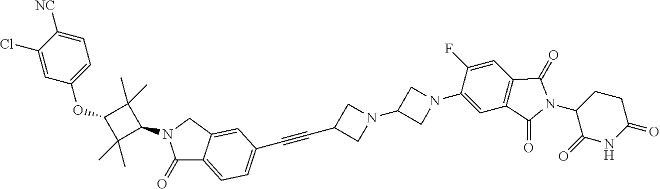 [SMILES: CC1(C)[C@H](Oc2ccc(C#N)c(Cl)c2)C(C)(C)[C@H]1N1Cc2cc(C#CC3CN(C4CN(c5cc6c(cc5F)C(=O)N(C5CCC(=O)NC5=O)C6=O)C4)C3)ccc2C1=O] |
| 329 | 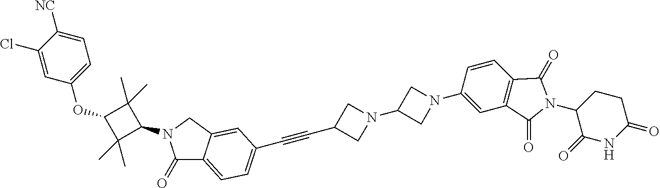 [SMILES: CC1(C)[C@H](Oc2ccc(C#N)c(Cl)c2)C(C)(C)[C@H]1N1Cc2cc(C#CC3CN(C4CN(c5ccc6c(c5)C(=O)N(C5CCC(=O)NC5=O)C6=O)C4)C3)ccc2C1=O] |
| 330 | 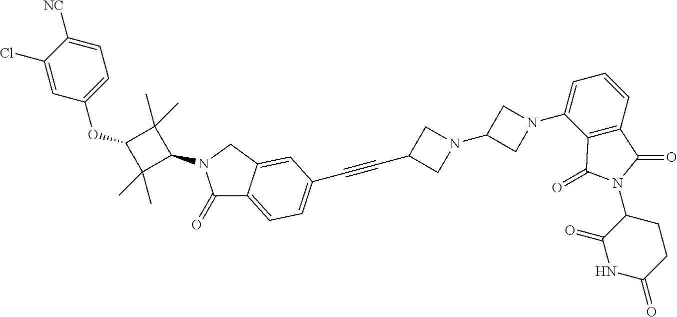 [SMILES: CC1(C)[C@H](Oc2ccc(C#N)c(Cl)c2)C(C)(C)[C@H]1N1Cc2cc(C#CC3CN(C4CN(c5cccc6c5C(=O)N(C5CCC(=O)NC5=O)C6=O)C4)C3)ccc2C1=O] |
| 331 | 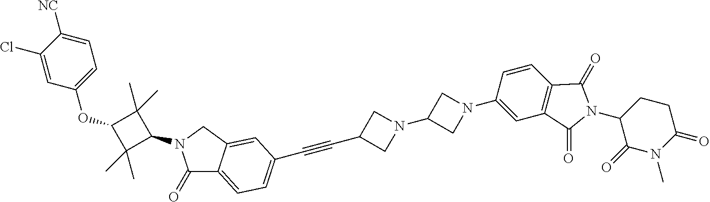 [SMILES: CN1C(=O)CCC(N2C(=O)c3ccc(N4CC(N5CC(C#Cc6ccc7c(c6)CN([C@H]6C(C)(C)[C@H](Oc8ccc(C#N)c(Cl)c8)C6(C)C)C7=O)C5)C4)cc3C2=O)C1=O] |
| 332 | 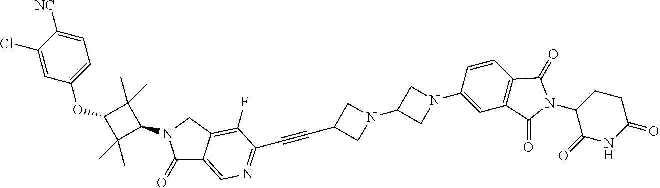 [SMILES: CC1(C)[C@H](Oc2ccc(C#N)c(Cl)c2)C(C)(C)[C@H]1N1Cc2c(cnc(C#CC3CN(C4CN(c5ccc6c(c5)C(=O)N(C5CCC(=O)NC5=O)C6=O)C4)C3)c2F)C1=O] |
| 333 | 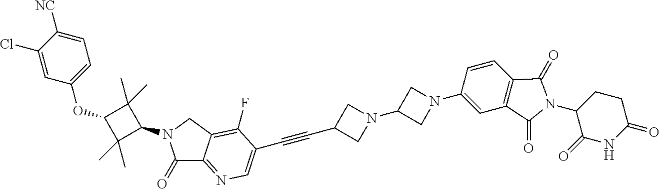 [SMILES: CC1(C)[C@H](Oc2ccc(C#N)c(Cl)c2)C(C)(C)[C@H]1N1Cc2c(ncc(C#CC3CN(C4CN(c5ccc6c(c5)C(=O)N(C5CCC(=O)NC5=O)C6=O)C4)C3)c2F)C1=O] |
| 334 | 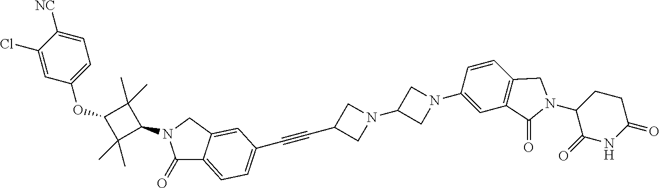 [SMILES: CC1(C)[C@H](Oc2ccc(C#N)c(Cl)c2)C(C)(C)[C@H]1N1Cc2cc(C#CC3CN(C4CN(c5ccc6c(c5)C(=O)N(C5CCC(=O)NC5=O)C6)C4)C3)ccc2C1=O] |
| 335 | 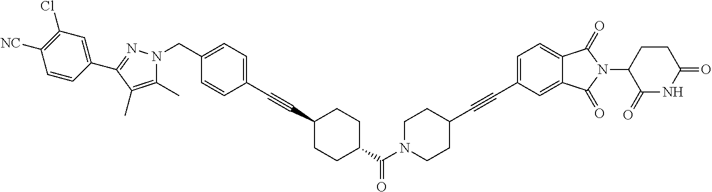 [SMILES: Cc1c(-c2ccc(C#N)c(Cl)c2)nn(Cc2ccc(C#C[C@H]3CC[C@H](C(=O)N4CCC(C#Cc5ccc6c(c5)C(=O)N(C5CCC(=O)NC5=O)C6=O)CC4)CC3)cc2)c1C] |
| 336 | 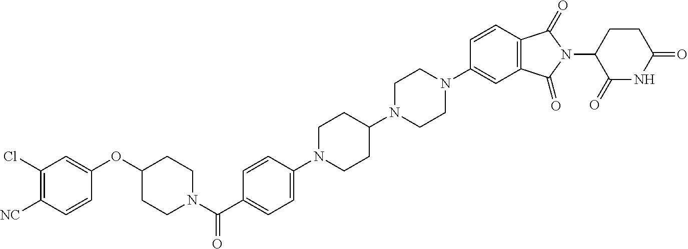 [SMILES: N#Cc1ccc(OC2CCN(C(=O)c3ccc(N4CCC(N5CCN(c6ccc7c(c6)C(=O)N(C6CCC(=O)NC6=O)C7=O)CC5)CC4)cc3)CC2)cc1Cl] |
| 337 | 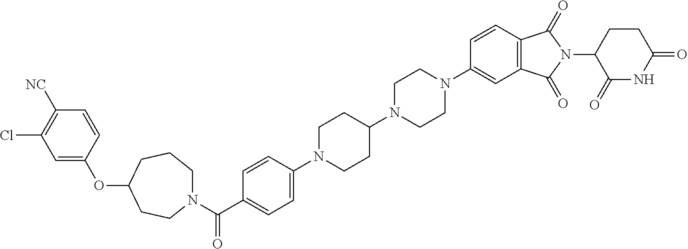 [SMILES: N#Cc1ccc(OC2CCCN(C(=O)c3ccc(N4CCC(N5CCN(c6ccc7c(c6)C(=O)N(C6CCC(=O)NC6=O)C7=O)CC5)CC4)cc3)CC2)cc1Cl] |
| 338 | 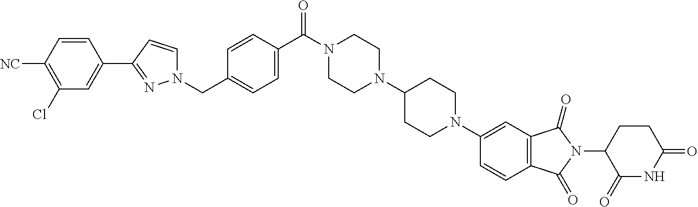 [SMILES: N#Cc1ccc(-c2ccn(Cc3ccc(C(=O)N4CCN(C5CCN(c6ccc7c(c6)C(=O)N(C6CCC(=O)NC6=O)C7=O)CC5)CC4)cc3)n2)cc1Cl] |

In another embodiment, the disclosure provides a pharmaceutical composition comprising a Compound of the Disclosure and a pharmaceutically acceptable carrier or excipient.

Compounds of the Disclosure may contain an asymmetric carbon atom. In some embodiments, Compounds of the Disclosure are racemic compounds. In other embodiments, Compounds of the Disclosure are enantiomerically enriched, e.g., the enantiomeric excess or “ee” of the compound is about 5% or more as measured by chiral HPLC. In another embodiment, the ee is about 10%. In another embodiment, the ee is about 20%. In another embodiment, the ee is about 30%. In another embodiment, the ee is about 40%. In another embodiment, the ee is about 50%. In another embodiment, the ee is about 60%. In another embodiment, the ee is about 70%. In another embodiment, the ee is about 80%. In another embodiment, the ee is about 85%. In another embodiment, the ee is about 90%. In another embodiment, the ee is about 91%. In another embodiment, the ee is about 92%. In another embodiment, the ee is about 93%. In another embodiment, the ee is about 94%. In another embodiment, the ee is about 95%. In another embodiment, the ee is about 96%. In another embodiment, the ee is about 97%. In another embodiment, the ee is about 98%. In another embodiment, the ee is about 99%.

In another embodiment, the cereblon binding portion of a Compound of the Disclosure, i.e., B¹, is enantiomerically enriched. In another embodiment, the cereblon binding portion of the molecule is racemic. The present disclosure encompasses all possible stereoisomeric, e.g., diastereomeric, forms of Compounds of the Disclosure. For example, all possible stereoisomers of Compounds of the Disclosure are encompassed when A or L portion of Formula I is entantiomerically enriched and the cereblon binding portion of the molecule is racemic. When a Compound of the Disclosure is desired as a single enantiomer, it can be obtained either by resolution of the final product or by stereospecific synthesis from either isomerically pure starting material or use of a chiral auxiliary reagent, for example, see Z. Ma et al., Tetrahedron: Asymmetry, 8(6), pages 883-888 (1997). Resolution of the final product, an intermediate, or a starting material can be achieved by any suitable method known in the art. Additionally, in situations where tautomers of the Compounds of the Disclosure are possible, the present disclosure is intended to include all tautomeric forms of the compounds.

The present disclosure encompasses the preparation and use of salts of Compounds of the Disclosure, including pharmaceutically acceptable salts. As used herein, the “pharmaceutically acceptable salt” refers to non-toxic salt forms of Compounds of the Disclosure. See e.g., Gupta et al., Molecules 23:1719 (2018). Salts of Compounds of the Disclosure can be prepared during the final isolation and purification of the compounds or separately by reacting the compound with an acid having a suitable cation. The pharmaceutically acceptable salts of Compounds of the Disclosure can be acid addition salts formed with pharmaceutically acceptable acids. Examples of acids which can be employed to form pharmaceutically acceptable salts include inorganic acids such as nitric, boric, hydrochloric, hydrobromic, sulfuric, and phosphoric, and organic acids such as oxalic, maleic, succinic, and citric. Nonlimiting examples of salts of compounds of the disclosure include, but are not limited to, the hydrochloride, hydrobromide, hydroiodide, sulfate, bisulfate, 2-hydroxyethansulfonate, phosphate, hydrogen phosphate, acetate, adipate, alginate, aspartate, benzoate, bisulfate, butyrate, camphorate, camphorsulfonate, digluconate, glycerolphsphate, hemisulfate, heptanoate, hexanoate, formate, succinate, fumarate, maleate, ascorbate, isethionate, salicylate, methanesulfonate, mesitylenesulfonate, naphthylenesulfonate, nicotinate, 2-naphthalenesulfonate, oxalate, pamoate, pectinate, persulfate, 3-phenylproprionate, picrate, pivalate, propionate, trichloroacetate, trifluoroacetate, phosphate, glutamate, bicarbonate, paratoluenesulfonate, undecanoate, lactate, citrate, tartrate, gluconate, methanesulfonate, ethanedisulfonate, benzene sulfonate, and p-toluenesulfonate salts. In addition, available amino groups present in the compounds of the disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. In light of the foregoing, any reference Compounds of the Disclosure appearing herein is intended to include the actual compound as well as pharmaceutically acceptable salts, hydrates, or solvates thereof.

The present disclosure also encompasses the preparation and use of solvates of Compounds of the Disclosure. Solvates typically do not significantly alter the physiological activity or toxicity of the compounds, and as such may function as pharmacological equivalents. The term “solvate” as used herein is a combination, physical association and/or solvation of a compound of the present disclosure with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds of the Disclosure can be present as solvated forms with a pharmaceutically acceptable solvent, such as water, methanol, and ethanol, and it is intended that the disclosure includes both solvated and unsolvated forms of Compounds of the Disclosure. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a Compound of the Disclosure in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

### II. Therapeutic Methods of the Disclosure

Compounds of the Disclosure degrade AR protein and are thus useful in the treatment of a variety of diseases and conditions. In particular, Compounds of the Disclosure are useful in methods of treating a disease or condition wherein degradation AR proteins provides a benefit, for example, cancers and proliferative diseases. The therapeutic methods of the disclosure comprise administering a therapeutically effective amount of a Compound of the Disclosure to a subject, e.g., a cancer patient, in need thereof. The present methods also encompass administering a second therapeutic agent to the subject in combination with the Compound of the Disclosure. The second therapeutic agent is selected from drugs known as useful in treating the disease or condition afflicting the individual in need thereof, e.g., a chemotherapeutic agent and/or radiation known as useful in treating a particular cancer.

The present disclosure provides Compounds of the Disclosure as AR protein degraders for the treatment of a variety of diseases and conditions wherein degradation of AR proteins has a beneficial effect. Compounds of the Disclosure typically have DC₅₀ (the drug concentration that results in 50% AR protein degradation) values of less than 100 μM, e.g., less than 50 μM, less than 25 μM, and less than 5 μM, less than about 1 μM, less than about 0.5 μM, or less than about 0.1 μM. In some embodiments, Compounds of the Disclosure typically have DC₅₀ values of less than about 0.01 μM. In some embodiments, Compounds of the Disclosure typically have DC₅₀ values of less than about 0.001 μM. In one embodiment, the present disclosure relates to a method of treating an individual suffering from a disease or condition wherein degradation of AR proteins provides a benefit comprising administering a therapeutically effective amount of a Compound of the Disclosure to an individual in need thereof.

Since Compounds of the Disclosure are degraders of AR protein, a number of diseases and conditions mediated by AR can be treated by employing these compounds. The present disclosure is thus directed generally to a method for treating a condition or disorder responsive to degradation of AR in an animal, e.g., a human, suffering from, or at risk of suffering from, the condition or disorder, the method comprising administering to the animal an effective amount of one or more Compounds of the Disclosure.

The present disclosure is further directed to a method of degrading AR protein in a subject in need thereof, said method comprising administering to the subject an effective amount of at least one Compound of the Disclosure.

In another aspect, the present disclosure provides a method of treating cancer in a subject comprising administering a therapeutically effective amount of a Compound of the Disclosure. While not being limited to a specific mechanism, in some embodiments, Compounds of the Disclosure treat cancer by degrading AR. Examples of treatable cancers include, but are not limited to, any one or more of the cancers of Table 2.

TABLE 2

| | | | |
|---|---|---|---|
| adrenal cancer | acinic cell carcinoma | acoustic neuroma | acral lentigious |
| | | | melanoma |
| acrospiroma | acute eosinophilic | acute erythroid | acute lymphoblastic |
| | leukemia | leukemia | leukemia |
| acute | acute monocytic | acute promyelocytic | adenocarcinoma |
| megakaryoblastic | leukemia | leukemia | |
| leukemia | | | |
| adenoid cystic | adenoma | adenomatoid | adenosquamous |
| carcinoma | | odontogenic tumor | carcinoma |
| adipose tissue | adrenocortical | adult T-cell | aggressive NK-cell |
| neoplasm | carcinoma | leukemia/lymphoma | leukemia |
| AIDS-related | alveolar | alveolar soft part | ameloblastic |
| lymphoma | rhabdomyosarcoma | sarcoma | fibroma |
| anaplastic large cell | anaplastic thyroid | angioimmunoblastic | angiomyolipoma |
| lymphoma | cancer | T-cell lymphoma | |
| angiosarcoma | astrocytoma | atypical teratoid | B-cell chronic |
| | | rhabdoid tumor | lymphocytic |
| | | | leukemia |
| B-cell | B-cell lymphoma | basal cell carcinoma | biliary tract cancer |
| prolymphocytic | | | |
| leukemia | | | |
| bladder cancer | blastoma | bone cancer | Brenner tumor |
| Brown tumor | Burkitt's lymphoma | breast cancer | brain cancer |
| carcinoma | carcinoma in situ | carcinosarcoma | cartilage tumor |
| cementoma | myeloid sarcoma | chondroma | chordoma |
| choriocarcinoma | choroid plexus | clear-cell sarcoma of | craniopharyngioma |
| | papilloma | the kidney | |
| cutaneous T-cell | cervical cancer | colorectal cancer | Degos disease |
| lymphoma | | | |
| desmoplastic small | diffuse large B-cell | dysembryoplastic | dysgerminoma |
| round cell tumor | lymphoma | neuroepithelial | |
| | | tumor | |
| embryonal | endocrine gland | endodermal sinus | enteropathy- |
| carcinoma | neoplasm | tumor | associated T-cell |
| | | | lymphoma |
| esophageal cancer | fetus in fetu | fibroma | fibrosarcoma |
| follicular | follicular thyroid | ganglioneuroma | gastrointestinal |
| lymphoma | cancer | | cancer |
| germ cell tumor | gestational | giant cell | giant cell tumor of |
| | choriocarcinoma | fibroblastoma | the bone |
| glial tumor | glioblastoma | glioma | gliomatosis cerebri |
| | multiforme | | |
| glucagonoma | gonadoblastoma | granulosa cell tumor | gynandroblastoma |
| gallbladder cancer | gastric cancer | hairy cell leukemia | hemangioblastoma |
| head and neck | hemangiopericytoma | hematological | hepatoblastoma |
| cancer | | cancer | |
| hepatosplenic T-cell | Hodgkin's | non-Hodgkin's | invasive lobular |
| lymphoma | lymphoma | lymphoma | carcinoma |
| intestinal cancer | kidney cancer | laryngeal cancer | lentigo maligna |
| lethal midline | leukemia | leydig cell tumor | liposarcoma |
| carcinoma | | | |
| lung cancer | lymphangioma | lymphangiosarcoma | lymphoepithelioma |
| lymphoma | acute lymphocytic | acute myelogeous | chronic |
| | leukemia | leukemia | lymphocytic |
| | | | leukemia |
| liver cancer | small cell lung | non-small cell lung | MALT lymphoma |
| | cancer | cancer | |
| malignant fibrous | malignant peripheral | malignant triton | mantle cell |
| histiocytoma | nerve sheath tumor | tumor | lymphoma |
| marginal zone B- | mast cell leukemia | mediastinal germ | medullary |
| cell lymphoma | | cell tumor | carcinoma of the |
| | | | breast |
| medullary thyroid | medulloblastoma | melanoma | meningioma |
| cancer | | | |
| merkel cell cancer | mesothelioma | metastatic urothelial | mixed Mullerian |
| | | carcinoma | tumor |
| mucinous tumor | multiple myeloma | muscle tissue | mycosis fungoides |
| | | neoplasm | |
| myxoid | myxoma | myxosarcoma | nasopharyngeal |
| liposarcoma | | | carcinoma |
| neurinoma | neuroblastoma | neurofibroma | neuroma |
| nodular melanoma | ocular cancer | oligoastrocytoma | oligodendroglioma |
| oncocytoma | optic nerve sheath | optic nerve tumor | oral cancer |
| | meningioma | | |
| osteosarcoma | ovarian cancer | Pancoast tumor | papillary thyroid |
| | | | cancer |
| paraganglioma | pinealoblastoma | pineocytoma | pituicytoma |
| pituitary adenoma | pituitary tumor | plasmacytoma | polyembryoma |
| precursor T- | primary central | primary effusion | preimary peritoneal |
| lymphoblastic | nervous system | lymphoma | cancer |
| lymphoma | lymphoma | | |
| prostate cancer | pancreatic cancer | pharyngeal cancer | pseudomyxoma |
| | | | periotonei |
| renal cell carcinoma | renal medullary | retinoblastoma | rhabdomyoma |
| | carcinoma | | |
| rhabdomyosarcoma | Richter's | rectal cancer | sarcoma |
| | transformation | | |
| Schwannomatosis | seminoma | Sertoli cell tumor | sex cord-gonadal |
| | | | stromal tumor |
| signet ring cell | skin cancer | small blue round cell | small cell |
| carcinoma | | tumors | carcinoma |
| soft tissue sarcoma | somatostatinoma | soot wart | spinal tumor |
| splenic marginal | squamous cell | synovial sarcoma | Sezary's disease |
| zone lymphoma | carcinoma | | |
| small intestine | squamous carcinoma | stomach cancer | T-cell lymphoma |
| cancer | | | |
| testicular cancer | thecoma | thyroid cancer | transitional cell |
| | | | carcinoma |
| throat cancer | urachal cancer | urogenital cancer | urothelial |
| | | | carcinoma |
| uveal melanoma | uterine cancer | verrucous carcinoma | visual pathway |
| | | | glioma |
| vulvar cancer | vaginal cancer | Waldenstrom's | Warthin's tumor |
| | | macroglobulinemia | |
| Wilms' tumor | | | |

In another embodiment, the cancer is a solid tumor. In another embodiment, the cancer a hematological cancer. Exemplary hematological cancers include, but are not limited to, the cancers listed in Table 3. In another embodiment, the hematological cancer is acute lymphocytic leukemia, chronic lymphocytic leukemia (including B-cell chronic lymphocytic leukemia), or acute myeloid leukemia.

TABLE 3

| | |
|---|---|
| acute lymphocytic leukemia (ALL) | acute eosinophilic leukemia |
| acute myeloid leukemia (AML) | acute erythroid leukemia |
| chronic lymphocytic leukemia (CLL) | acute lymphoblastic leukemia |
| small lymphocytic lymphoma (SLL) | acute megakaryoblastic leukemia |
| multiple myeloma (MM) | acute monocytic leukemia |
| Hodgkins lymphoma (HL) | acute promyelocytic leukemia |
| non-Hodgkin's lymphoma (NHL) | acute myelogeous leukemia |
| mantle cell lymphoma (MCL) | B-cell prolymphocytic leukemia |
| marginal zone B-cell lymphoma | B-cell lymphoma |
| splenic marginal zone lymphoma | MALT lymphoma |
| follicular lymphoma (FL) | precursor T-lymphoblastic lymphoma |
| Waldenstrom's macroglobulinemia (WM) | T-cell lymphoma |
| diffuse large B-cell lymphoma (DLBCL) | mast cell leukemia |
| marginal zone lymphoma (MZL) | adult T cell leukemia/lymphoma |
| hairy cell leukemia (HCL) | aggressive NK-cell leukemia |
| Burkitt's lymphoma (BL) | angioimmunoblastic T-cell lymphoma |
| Richter's transformation | |

In another embodiment, the cancer is a leukemia, for example a leukemia selected from acute monocytic leukemia, acute myelogenous leukemia, chronic myelogenous leukemia, chronic lymphocytic leukemia and mixed lineage leukemia (MLL). In another embodiment the cancer is NUT-midline carcinoma. In another embodiment the cancer is multiple myeloma. In another embodiment the cancer is a lung cancer such as small cell lung cancer (SCLC). In another embodiment the cancer is a neuroblastoma. In another embodiment the cancer is Burkitt's lymphoma. In another embodiment the cancer is cervical cancer. In another embodiment the cancer is esophageal cancer. In another embodiment the cancer is ovarian cancer. In another embodiment the cancer is colorectal cancer. In another embodiment, the cancer is prostate cancer. In another embodiment, the cancer is breast cancer.

In another embodiment, the cancer is selected from the group consisting of acute monocytic leukemia, acute myelogenous leukemia, chronic myelogenous leukemia, chronic lymphocytic leukemia mixed lineage leukemia, NUT-midline carcinoma, multiple myeloma, small cell lung cancer, non-small cell lung cancer, neuroblastoma, Burkitt's lymphoma, cervical cancer, esophageal cancer, ovarian cancer, colorectal cancer, prostate cancer, breast cancer, bladder cancer, ovary cancer, glioma, sarcoma, esophageal squamous cell carcinoma, and papillary thyroid carcinoma.

In another embodiment, Compounds of the Disclosure are administered to a subject in need thereof to treat breast cancer or prostate cancer. In another embodiment, the cancer is breast cancer. In another embodiment, the cancer is prostate cancer. In another embodiment, the cancer is metastatic castration-resistant prostate cancer.

The methods of the present disclosure can be accomplished by administering a Compound of the Disclosure as the neat compound or as a pharmaceutical composition. Administration of a pharmaceutical composition, or neat Compound of the Disclosure, can be performed during or after the onset of the disease or condition of interest. Typically, the pharmaceutical compositions are sterile, and contain no toxic, carcinogenic, or mutagenic compounds that would cause an adverse reaction when administered.

In one embodiment, a Compound of the Disclosure is administered as a single agent to treat a disease or condition wherein degradation of AR protein provides a benefit. In another embodiment, a Compound of the Disclosure is administered in conjunction with a second therapeutic agent useful in the treatment of a disease or condition wherein degradation of AR protein provides a benefit. The second therapeutic agent is different from the Compound of the Disclosure. A Compound of the Disclosure and the second therapeutic agent can be administered simultaneously or sequentially to achieve the desired effect. In addition, the Compound of the Disclosure and second therapeutic agent can be administered as a single pharmaceutical composition or two separate pharmaceutical compositions.

The second therapeutic agent is administered in an amount to provide its desired therapeutic effect. The effective dosage range for each second therapeutic agent is known in the art, and the second therapeutic agent is administered to an individual in need thereof within such established ranges.

A Compound of the Disclosure and the second therapeutic agent can be administered together as a single-unit dose or separately as multi-unit doses, wherein the Compound of the Disclosure is administered before the second therapeutic agent or vice versa. One or more doses of the Compound of the Disclosure and/or one or more doses of the second therapeutic agent can be administered. The Compound of the Disclosure therefore can be used in conjunction with one or more second therapeutic agents, for example, but not limited to, anticancer agents.

In methods of the present disclosure, a therapeutically effective amount of a Compound of the Disclosure, typically formulated in accordance with pharmaceutical practice, is administered to a subject, e.g., a human cancer patient, in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

A Compound of the Disclosure can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high pressure technique.

Pharmaceutical compositions include those wherein a Compound of the Disclosure is administered in an effective amount to achieve its intended purpose. The exact formulation, route of administration, and dosage is determined by an individual physician in view of the diagnosed condition or disease. Dosage amount and interval can be adjusted individually to provide levels of a Compound of the Disclosure that is sufficient to maintain therapeutic effects.

Toxicity and therapeutic efficacy of the Compounds of the Disclosure can be determined by standard pharmaceutical procedures in cell cultures or experimental animals, e.g., for determining the maximum tolerated dose (MTD) of a compound, which defines as the highest dose that causes no toxicity in animals. The dose ratio between the maximum tolerated dose and therapeutic effects (e.g. inhibiting of tumor growth) is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of a Compound of the Disclosure required for use in therapy varies with the nature of the condition being treated, the length of time that activity is desired, and the age and the condition of the patient, and ultimately is determined by the attendant physician. Dosage amounts and intervals can be adjusted individually to provide plasma levels of the AR protein degrader that are sufficient to maintain the desired therapeutic effects. The desired dose conveniently can be administered in a single dose, or as multiple doses administered at appropriate intervals, for example as one, two, three, four or more subdoses per day. Multiple doses often are desired, or required. For example, a Compound of the Disclosure can be administered at a frequency of: four doses delivered as one dose per day at four-day intervals (q4d×4); four doses delivered as one dose per day at three-day intervals (q3d×4); one dose delivered per day at five-day intervals (qd×5); one dose per week for three weeks (qwk3); five daily doses, with two days rest, and another five daily doses (5/2/5); or, any dose regimen determined to be appropriate for the circumstance.

A Compound of the Disclosure used in a method of the present disclosure can be administered in an amount of about 0.005 to about 500 milligrams per dose, about 0.05 to about 250 milligrams per dose, or about 0.5 to about 100 milligrams per dose. For example, a Compound of the Disclosure can be administered, per dose, in an amount of about 0.005, 0.05, 0.5, 5, 10, 20, 30, 40, 50, 100, 150, 200, 250, 300, 350, 400, 450, or 500 milligrams, including all doses between 0.005 and 500 milligrams.

The dosage of a composition containing a Compound of the Disclosure, or a composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg, or more. The above dosages are exemplary of the average case, but there can be individual instances in which higher or lower dosages are merited, and such are within the scope of this disclosure. In practice, the physician determines the actual dosing regimen that is most suitable for an individual patient, which can vary with the age, weight, and response of the particular patient.

As stated above, a Compound of the Disclosure can be administered in combination with a second therapeutically active agent. In some embodiments, the second therapeutic agent is an epigenetic drug. As used herein, the term “epigenetic drug” refers to a therapeutic agent that targets an epigenetic regulator. Examples of epigenetic regulators include the histone lysine methyltransferases, histone arginine methyl transferases, histone demethylases, histone deacetylases, histone acetylases, and DNA methyltransferases. Histone deacetylase inhibitors include, but are not limited to, vorinostat.

In another embodiment, chemotherapeutic agents or other anti-proliferative agents can be combined with Compound of the Disclosure to treat proliferative diseases and cancer. Examples of therapies and anticancer agents that can be used in combination with Compounds of the Disclosure include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

Examples of antiproliferative compounds include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DMI, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary nonlimiting Flt-3 inhibitors include PKC412, midostaurin, a staurosporine derivative, SU11248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino, 17-demethoxygeldanamycin (17AAG), a geldanamycin derivative; other geldanamycin related compounds; radicicol and HDAC inhibitors.

The phrase “a compound targeting/decreasing a protein or lipid kinase activity; or a protein or lipid phosphatase activity; or any further anti-angiogenic compound” as used herein includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SUlOl, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, Cl-1033, EKB-569, GW-2016, antibodies EL.1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, exemplary chemotherapeutic compounds, one or more of which may be used in combination with a Compound of the Disclosure, include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, 1-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortexolone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

Other examples of second therapeutic agents, one or more of which a Compound of the Disclosure also can be combined, include, but are not limited to: a treatment for Alzheimer's Disease, such as donepezil and rivastigmine; a treatment for Parkinson's Disease, such as L-DOPA/carbidopa, entacapone, ropinrole, pramipexole, bromocriptine, pergolide, trihexephendyl, and amantadine; an agent for treating multiple sclerosis (MS) such as beta interferon (e.g., AVONEX® and REBIF®), glatiramer acetate, and mitoxantrone; a treatment for asthma, such as albuterol and montelukast; an agent for treating schizophrenia, such as zyprexa, risperdal, seroquel, and haloperidol; an anti-inflammatory agent, such as a corticosteroid, a TNF blocker, IL-1 RA, azathioprine, cyclophosphamide, and sulfasalazine; an immunomodulatory agent, including immunosuppressive agents, such as cyclosporin, tacrolimus, rapamycin, mycophenolate mofetil, an interferon, a corticosteroid, cyclophosphamide, azathioprine, and sulfasalazine; a neurotrophic factor, such as an acetylcholinesterase inhibitor, an MAO inhibitor, an interferon, an anti-convulsant, an ion channel blocker, riluzole, or an anti-Parkinson's agent; an agent for treating cardiovascular disease, such as a beta-blocker, an ACE inhibitor, a diuretic, a nitrate, a calcium channel blocker, or a statin; an agent for treating liver disease, such as a corticosteroid, cholestyramine, an interferon, and an anti-viral agent; an agent for treating blood disorders, such as a corticosteroid, an anti-leukemic agent, or a growth factor; or an agent for treating immunodeficiency disorders, such as gamma globulin.

In another embodiment, the second therapeutically active agent is an immune checkpoint inhibitor. Examples of immune checkpoint inhibitors include PD-1 inhibitors, PD-L1 inhibitors, CTLA-4 inhibitors, LAG3 inhibitors, TIM3 inhibitors, cd47 inhibitors, and B7-H1 inhibitors. Thus, in one embodiment, a Compound of the Disclosure is administered in combination with an immune checkpoint inhibitor is selected from the group consisting of a PD-1 inhibitor, a PD-L1 inhibitor, a CTLA-4 inhibitor, a LAG3 inhibitor, a TIM3 inhibitor, and a cd47 inhibitor.

In another embodiment, the immune checkpoint inhibitor is a programmed cell death (PD-1) inhibitor. PD-1 is a T-cell coinhibitory receptor that plays a pivotal role in the ability of tumor cells to evade the host's immune system. Blockage of interactions between PD-1 and PD-L1, a ligand of PD-1, enhances immune function and mediates antitumor activity. Examples of PD-1 inhibitors include antibodies that specifically bind to PD-1. Particular anti-PD-1 antibodies include, but are not limited to nivolumab, pembrolizumab, STI-A1014, and pidilzumab. For a general discussion of the availability, methods of production, mechanism of action, and clinical studies of anti-PD-1 antibodies, see U.S. 2013/0309250, U.S. Pat. Nos. 6,808,710, 7,595,048, 8,008,449, 8,728,474, 8,779,105, 8,952,136, 8,900,587, 9,073,994, 9,084,776, and Naido et al., British Journal of Cancer 111:2214-19 (2014).

In another embodiment, the immune checkpoint inhibitor is a PD-L1 (also known as B7-H1 or CD274) inhibitor. Examples of PD-L1 inhibitors include antibodies that specifically bind to PD-L1. Particular anti-PD-L1 antibodies include, but are not limited to, avelumab, atezolizumab, durvalumab, and BMS-936559. For a general discussion of the availability, methods of production, mechanism of action, and clinical studies, see U.S. Pat. No. 8,217,149, U.S. 2014/0341917, U.S. 2013/0071403, WO 2015036499, and Naido et al., British Journal of Cancer 111:2214-19 (2014).

In another embodiment, the immune checkpoint inhibitor is a CTLA-4 inhibitor. CTLA-4, also known as cytotoxic T-lymphocyte antigen 4, is a protein receptor that downregulates the immune system. CTLA-4 is characterized as a “brake” that binds costimulatory molecules on antigen-presenting cells, which prevents interaction with CD28 on T cells and also generates an overtly inhibitory signal that constrains T cell activation. Examples of CTLA-4 inhibitors include antibodies that specifically bind to CTLA-4. Particular anti-CTLA-4 antibodies include, but are not limited to, ipilimumab and tremelimumab. For a general discussion of the availability, methods of production, mechanism of action, and clinical studies, see U.S. Pat. Nos. 6,984,720, 6,207,156, and Naido et al., British Journal of Cancer 111:2214-19 (2014).

In another embodiment, the immune checkpoint inhibitor is a LAG3 inhibitor. LAG3, Lymphocyte Activation Gene 3, is a negative co-stimulatory receptor that modulates T cell homeostatis, proliferation, and activation. In addition, LAG3 has been reported to participate in regulatory T cells (Tregs) suppressive function. A large proportion of LAG3 molecules are retained in the cell close to the microtubule-organizing center, and only induced following antigen specific T cell activation. U.S. 2014/0286935. Examples of LAG3 inhibitors include antibodies that specifically bind to LAG3. Particular anti-LAG3 antibodies include, but are not limited to, GSK2831781. For a general discussion of the availability, methods of production, mechanism of action, and studies, see, U.S. 2011/0150892, U.S. 2014/0093511, U.S. 20150259420, and Huang et al., Immunity 21:503-13 (2004).

In another embodiment, the immune checkpoint inhibitor is a TIM3 inhibitor. TIM3, T-cell immunoglobulin and mucin domain 3, is an immune checkpoint receptor that functions to limit the duration and magnitude of T_{H}1 and T_{C}1 T-cell responses. The TIM3 pathway is considered a target for anticancer immunotherapy due to its expression on dysfunctional CD8⁺ T cells and Tregs, which are two reported immune cell populations that constitute immunosuppression in tumor tissue. Anderson, Cancer Immunology Research 2:393-98 (2014). Examples of TIM3 inhibitors include antibodies that specifically bind to TIM3. For a general discussion of the availability, methods of production, mechanism of action, and studies of TIM3 inhibitors, see U.S. 20150225457, U.S. 20130022623, U.S. Pat. No. 8,522,156, Ngiow et al., Cancer Res 71: 6567-71 (2011), Ngiow, et al., Cancer Res 71:3540-51 (2011), and Anderson, Cancer Immunology Res 2:393-98 (2014).

In another embodiment, the immune checkpoint inhibitor is a cd47 inhibitor. See Unanue, E. R., PNAS 110:10886-87 (2013).

The term “antibody” is meant to include intact monoclonal antibodies, polyclonal antibodies, multispecific antibodies formed from at least two intact antibodies, and antibody fragments, so long as they exhibit the desired biological activity. In another embodiment, “antibody” is meant to include soluble receptors that do not possess the Fc portion of the antibody. In one embodiment, the antibodies are humanized monoclonal antibodies and fragments thereof made by means of recombinant genetic engineering.

Another class of immune checkpoint inhibitors include polypeptides that bind to and block PD-1 receptors on T-cells without triggering inhibitor signal transduction. Such peptides include B7-DC polypeptides, B7-H1 polypeptides, B7-1 polypeptides and B7-2 polypeptides, and soluble fragments thereof, as disclosed in U.S. Pat. No. 8,114,845.

Another class of immune checkpoint inhibitors include compounds with peptide moieties that inhibit PD-1 signaling. Examples of such compounds are disclosed in U.S. Pat. No. 8,907,053.

Another class of immune checkpoint inhibitors include inhibitors of certain metabolic enzymes, such as indoleamine 2,3 dioxygenase (IDO), which is expressed by infiltrating myeloid cells and tumor cells. The IDO enzyme inhibits immune responses by depleting amino acids that are necessary for anabolic functions in T cells or through the synthesis of particular natural ligands for cytosolic receptors that are able to alter lymphocyte functions. Pardoll, Nature Reviews. Cancer 12:252-64 (2012); Löb, Cancer Immunol Immunother 58:153-57 (2009). Particular IDO blocking agents include, but are not limited to levo-1-methyl typtophan (L-1MT) and 1-methyl-tryptophan (1MT). Qian et al., Cancer Res 69:5498-504 (2009); and Löb et al., Cancer Immunol Immunother 58:153-7 (2009).

In one embodiment, the immune checkpoint inhibitor is nivolumab, pembrolizumab, pidilizumab, STI-A1110, avelumab, atezolizumab, durvalumab, STI-A1014, ipilimumab, tremelimumab, GSK2831781, BMS-936559 or MED14736

The above-mentioned second therapeutically active agents, one or more of which can be used in combination with a Compound of the Disclosure, are prepared and administered as described in the art.

Compounds of the Disclosure typically are administered in admixture with a pharmaceutical carrier selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of Compound of the Disclosure.

These pharmaceutical compositions can be manufactured, for example, by conventional mixing, dissolving, granulating, dragee-making, emulsifying, encapsulating, entrapping, or lyophilizing processes. Proper formulation is dependent upon the route of administration chosen. When a therapeutically effective amount of the Compound of the Disclosure is administered orally, the composition typically is in the form of a tablet, capsule, powder, solution, or elixir. When administered in tablet form, the composition additionally can contain a solid carrier, such as a gelatin or an adjuvant. The tablet, capsule, and powder contain about 0.01% to about 95%, and preferably from about 1% to about 50%, of a Compound of the Disclosure. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.1% to about 90%, and preferably about 1% to about 50%, by weight, of a Compound of the Disclosure.

When a therapeutically effective amount of a Compound of the Disclosure is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, an isotonic vehicle.

Compounds of the Disclosure can be readily combined with pharmaceutically acceptable carriers well-known in the art. Standard pharmaceutical carriers are described in Remington's Pharmaceutical Sciences, Mack Publishing Co., Easton, Pa., 19th ed. 1995. Such carriers enable the active agents to be formulated as tablets, pills, dragees, capsules, liquids, gels, syrups, slurries, suspensions and the like, for oral ingestion by a patient to be treated. Pharmaceutical preparations for oral use can be obtained by adding the Compound of the Disclosure to a solid excipient, optionally grinding the resulting mixture, and processing the mixture of granules, after adding suitable auxiliaries, if desired, to obtain tablets or dragee cores.

Suitable excipients include fillers such as saccharides (for example, lactose, sucrose, mannitol or sorbitol), cellulose preparations, calcium phosphates (for example, tricalcium phosphate or calcium hydrogen phosphate), as well as binders such as starch paste (using, for example, maize starch, wheat starch, rice starch, or potato starch), gelatin, tragacanth, methyl cellulose, hydroxypropylmethylcellulose, sodium carboxymethylcellulose, and/or polyvinyl pyrrolidone. If desired, one or more disintegrating agents can be added, such as the above-mentioned starches and also carboxymethyl-starch, cross-linked polyvinyl pyrrolidone, agar, or alginic acid or a salt thereof, such as sodium alginate. Buffers and pH modifiers can also be added to stabilize the pharmaceutical composition.

Auxiliaries are typically flow-regulating agents and lubricants such as, for example, silica, talc, stearic acid or salts thereof (e.g., magnesium stearate or calcium stearate), and polyethylene glycol. Dragee cores are provided with suitable coatings that are resistant to gastric juices. For this purpose, concentrated saccharide solutions can be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate can be used. Dye stuffs or pigments can be added to the tablets or dragee coatings, for example, for identification or in order to characterize combinations of active compound doses.

Compound of the Disclosure can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions, or emulsions in oily or aqueous vehicles, and can contain formulatory agents such as suspending, stabilizing, and/or dispersing agents.

Pharmaceutical compositions for parenteral administration include aqueous solutions of the active agent in water-soluble form. Additionally, suspensions of a Compound of the Disclosure can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle, e.g., sterile pyrogen-free water, before use.

Compounds of the Disclosure also can be formulated in rectal compositions, such as suppositories or retention enemas, e.g., containing conventional suppository bases. In addition to the formulations described previously, the Compound of the Disclosure also can be formulated as a depot preparation. Such long-acting formulations can be administered by implantation (for example, subcutaneously or intramuscularly) or by intramuscular injection. Thus, for example, the Compound of the Disclosure can be formulated with suitable polymeric or hydrophobic materials (for example, as an emulsion in an acceptable oil) or ion exchange resins.

In particular, the Compounds of the Disclosure can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. Compound of the Disclosure also can be injected parenterally, for example, intravenously, intramuscularly, subcutaneously, or intracoronarily. For parenteral administration, the Compound of the Disclosure are typically used in the form of a sterile aqueous solution which can contain other substances, for example, salts or monosaccharides, such as mannitol or glucose, to make the solution isotonic with blood.

The disclosure provides the following particular embodiments in connection with treating a disease in a subject with a Compound of the Disclosure.

Embodiment I. A method of treating a subject, the method comprising administering to the subject a therapeutically effective amount of a Compound of the Disclosure, wherein the subject has cancer, a chronic autoimmune disorder, an inflammatory condition, a proliferative disorder, sepsis, or a viral infection.

Embodiment II. The method Embodiment I, wherein the subject has cancer, e.g., any one of more of the cancers of Table 2 or Table 3.

Embodiment III. The method of Embodiment II, wherein the cancer is prostate cancer or breast cancer.

Embodiment IV. The method of Embodiment II, wherein the cancer is breast cancer.

Embodiment V. The method of Embodiment II, wherein the cancer is prostate cancer, e.g., metastatic castration-resistant prostate cancer.

Embodiment VI. The method of any one of Embodiments I-V further comprising administering a therapeutically effective amount of a second therapeutic agent useful in the treatment of the disease or condition, e.g., an immune checkpoint inhibitor or other anticancer agent.

Embodiment VII. A pharmaceutical composition comprising a Compound of the Disclosure and a pharmaceutically acceptable excipient for use in treating cancer, a chronic autoimmune disorder, an inflammatory condition, a proliferative disorder, sepsis, or a viral infection.

Embodiment VIII. The pharmaceutical composition of Embodiment VII for use in treating cancer.

Embodiment IX. The pharmaceutical composition of Embodiment VIII, wherein the cancer is prostate cancer or breast cancer.

Embodiment X. The pharmaceutical composition of Embodiment VIII, wherein the cancer is breast cancer.

Embodiment XI. The pharmaceutical composition of Embodiment VIII, wherein the cancer is prostate cancer, e.g., metastatic castration-resistant prostate cancer.

Embodiment XII. A Compound of the Disclosure for use in treatment of cancer, a chronic autoimmune disorder, an inflammatory condition, a proliferative disorder, sepsis, or a viral infection.

Embodiment XIII. The compound of Embodiment XIII for use in treating cancer.

Embodiment XIV. The compound of Embodiment XIII, wherein the cancer is breast cancer.

Embodiment XV. The compound of Embodiment XIII, wherein the cancer is prostate cancer, e.g., metastatic castration-resistant prostate cancer.

Embodiment XVI. Use of a Compound of the Disclosure for the manufacture of a medicament for treatment of cancer, a chronic autoimmune disorder, an inflammatory condition, a proliferative disorder, sepsis, or a viral infection.

Embodiment XVII. The use of Embodiment XVI for the treatment of cancer.

Embodiment XVIII. The use of Embodiment XVII, wherein the cancer is prostate cancer or breast cancer.

Embodiment XIV. The use of Embodiment XVII, wherein the cancer is breast cancer.

Embodiment XX. The use of Embodiment XVII, wherein the cancer is prostate cancer, e.g., metastatic castration-resistant prostate cancer.

Embodiment XXI. A method of reducing AR protein within a cell of a subject in need thereof, the method comprising administering to the subject a Compound of the Disclosure. In one embodiment, the AR protein is reduced by about 50% or less, e.g., 1%, about 2%, about 3%, about 4%, about 5%, about 10%, about 15%, about 20%, about 25%, about 30%, about 35%, about 40%, or about 45%. In one embodiment, the AR protein is reduced by about 51% or more, e.g., about 55%, about 60%, about 65%, about 70%, about 75%, about 80%, about 85%, about 90%, or about 95%.

### III. Kits of the Disclosure

In another embodiment, the present disclosure provides kits which comprise a Compound of the Disclosure (or a composition comprising a Compound of the Disclosure) packaged in a manner that facilitates its use to practice methods of the present disclosure. In one embodiment, the kit includes a Compound of the Disclosure (or a composition comprising a Compound of the Disclosure) packaged in a container, such as a sealed bottle or vessel, with a label affixed to the container or included in the kit that describes use of the compound or composition to practice the method of the disclosure. In one embodiment, the compound or composition is packaged in a unit dosage form. The kit further can include a device suitable for administering the composition according to the intended route of administration.

### IV. Definitions

The term “a disease or condition wherein degradation of androgen receptor (AR) provides a benefit” and the like pertains to a disease or condition in which the androgen receptor is important or necessary, e.g., for the onset, progress, expression of that disease or condition, or a disease or a condition which is known to be treated by an AR degrader. Examples of such conditions include, but are not limited to, a cancer. One of ordinary skill in the art is readily able to determine whether a compound treats a disease or condition mediated by an AR degrader for any particular cell type, for example, by assays which conveniently can be used to assess the activity of particular compounds.

The term “androgen receptor degrader,” “AR degrader,” and the like refer to a heterobifunctional small molecule that degrades AR protein. AR degraders contain a first ligand which binds to AR protein, a second ligand for an E3 ligase system, and a chemical linker that tethers the first and second ligands. Representative Compounds of the Disclosure that degrade AR protein are disclosed in Table 1.

The term “second therapeutic agent” refers to a therapeutic agent different from a Compound of the Disclosure and that is known to treat the disease or condition of interest. For example when a cancer is the disease or condition of interest, the second therapeutic agent can be a known chemotherapeutic drug, like taxol, or radiation, for example.

The term “disease” or “condition” denotes disturbances and/or anomalies that as a rule are regarded as being pathological conditions or functions, and that can manifest themselves in the form of particular signs, symptoms, and/or malfunctions. Compounds of the Disclosure are degraders of AR and can be used in treating or preventing diseases and conditions wherein degradation of AR provides a benefit.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

As used herein, the terms “prevent,” “preventing,” and “prevention” refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “therapeutically effective amount” or “effective dose” as used herein refers to an amount of the active ingredient(s) that is(are) sufficient, when administered by a method of the disclosure, to efficaciously deliver the active ingredient(s) for the treatment of condition or disease of interest to a subject in need thereof. In the case of a cancer or other proliferation disorder, the therapeutically effective amount of the agent may reduce (i.e., retard to some extent or stop) unwanted cellular proliferation; reduce the number of cancer cells; reduce the tumor size; inhibit (i.e., retard to some extent or stop) cancer cell infiltration into peripheral organs; inhibit (i.e., retard to some extent or stop) tumor metastasis; inhibit, to some extent, tumor growth; and/or relieve, to some extent, one or more of the symptoms associated with the cancer. To the extent the administered compound or composition prevents growth and/or kills existing cancer cells, it may be cytostatic and/or cytotoxic.

The term “container” means any receptacle and closure therefore suitable for storing, shipping, dispensing, and/or handling a pharmaceutical product.

The term “insert” means information accompanying a pharmaceutical product that provides a description of how to administer the product, along with the safety and efficacy data required to allow the physician, pharmacist, and patient to make an informed decision regarding use of the product. The package insert generally is regarded as the “label” for a pharmaceutical product.

“Concurrent administration,” “administered in combination,” “simultaneous administration,” and similar phrases mean that two or more agents are administered concurrently to the subject being treated. By “concurrently,” it is meant that each agent is administered either simultaneously or sequentially in any order at different points in time. However, if not administered simultaneously, it is meant that they are administered to a subject in a sequence and sufficiently close in time so as to provide the desired therapeutic effect and can act in concert. For example, a Compound of the Disclosure can be administered at the same time or sequentially in any order at different points in time as a second therapeutic agent. A Compound of the Disclosure and the second therapeutic agent can be administered separately, in any appropriate form and by any suitable route. When a Compound of the Disclosure and the second therapeutic agent are not administered concurrently, it is understood that they can be administered in any order to a subject in need thereof. For example, a Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent treatment modality (e.g., radiotherapy), to a subject in need thereof. In various embodiments, a Compound of the Disclosure and the second therapeutic agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the components of the combination therapies are administered at about 1 minute to about 24 hours apart.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “halo” as used herein by itself or as part of another group refers to —Cl, —F, —Br, or —I.

The term “nitro” as used herein by itself or as part of another group refers to —NO₂.

The term “cyano” as used herein by itself or as part of another group refers to —CN.

The term “hydroxy” as herein used by itself or as part of another group refers to —OH.

The term “alkyl” as used herein by itself or as part of another group refers to a straight- or branched-chain aliphatic hydrocarbon containing one to twelve carbon atoms, i.e., a C₁-C₁₂ alkyl, or the number of carbon atoms designated, e.g., a C₁ alkyl such as methyl, a C₂ alkyl such as ethyl, etc. In one embodiment, the alkyl is a C₁-C₁₀ alkyl. In another embodiment, the alkyl is a C₁-C₆ alkyl. In another embodiment, the alkyl is a C₁-C₄ alkyl. In another embodiment, the alkyl is a C₁-C₃ alkyl, i.e., methyl, ethyl, propyl, or isopropyl. Non-limiting exemplary C₁-C₁₂ alkyl groups include methyl, ethyl, propyl, isopropyl, butyl, sec-butyl, tert-butyl, iso-butyl, 3-pentyl, hexyl, heptyl, octyl, nonyl, and decyl.

The term “optionally substituted alkyl” as used herein by itself or as part of another group refers to an alkyl group that is either unsubstituted or substituted with one, two, or three substituents, wherein each substituent is independently nitro, haloalkoxy, aryloxy, aralkyloxy, alkylthio, sulfonamido, alkylcarbonyl, arylcarbonyl, alkylsulfonyl, arylsulfonyl, ureido, guanidino, carbamate, carboxy, alkoxycarbonyl, carboxyalkyl, —N(R^{56a})C(═O)R^{56b}, —N(R^{56c})S(═O)₂R^{56d}, —C(═O)R⁵⁷, —S(═O)R^{56e}, or —S(═O)₂R⁵⁸; wherein:

R^{56a} is hydrogen or alkyl;

R^{56b} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R^{56c} is hydrogen or alkyl;

R^{56d} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R^{56e} is alkyl, haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, optionally substituted C₆-C₁₀ aryl, or optionally substituted heteroaryl;

R⁵⁷ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl; and

R⁵⁸ is haloalkyl, optionally substituted cycloalkyl, alkoxy, (alkoxy)alkyl, (aryl)alkyl, (heteroaryl)alkyl, (amino)alkyl, (hydroxy)alkyl, (cyano)alkyl, optionally substituted alkenyl, optionally substituted alkynyl, optionally substituted cycloalkyl, optionally substituted heterocycle, or optionally substituted heteroaryl. Non-limiting exemplary optionally substituted alkyl groups include —CH(CO₂Me)CH₂CO₂Me and —CH(CH₃)CH₂N(H)C(═O)O(CH₃)₃.

The term “alkenyl” as used herein by itself or as part of another group refers to an alkyl group containing one, two, or three carbon-to-carbon double bonds. In one embodiment, the alkenyl group is a C₂-C₆ alkenyl group. In another embodiment, the alkenyl group is a C₂-C₄ alkenyl group. In another embodiment, the alkenyl group has one carbon-to-carbon double bond. Non-limiting exemplary alkenyl groups include ethenyl, propenyl, isopropenyl, butenyl, sec-butenyl, pentenyl, and hexenyl.

The term “optionally substituted alkenyl” as used herein by itself or as part of another refers to an alkenyl group that is either unsubstituted or substituted with one, two or three substituents, wherein each substituent is independently halo, nitro, cyano, hydroxy, amino (e.g., alkylamino, dialkylamino), haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkenyl groups include —CH═CHPh.

The term “alkynyl” as used herein by itself or as part of another group refers to an alkyl group containing one, two, or three carbon-to-carbon triple bonds. In one embodiment, the alkynyl is a C₂-C₆ alkynyl. In another embodiment, the alkynyl is a C₂-C₄ alkynyl. In another embodiment, the alkynyl has one carbon-to-carbon triple bond. Non-limiting exemplary alkynyl groups include ethynyl, propynyl, butynyl, 2-butynyl, pentynyl, and hexynyl groups.

The term “optionally substituted alkynyl” as used herein by itself or as part of another group refers to an alkynyl group that is either unsubstituted or substituted with one, two or three substituents, wherein each substituent is independently halo, nitro, cyano, hydroxy, amino, e.g., alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted cycloalkyl, alkenyl, alkynyl, optionally substituted aryl, optionally substituted heteroaryl, or optionally substituted heterocyclo. Non-limiting exemplary optionally substituted alkynyl groups include —C≡CPh and —CH(Ph)C≡CH.

The term “haloalkyl” as used herein by itself or as part of another group refers to an alkyl group substituted by one or more fluorine, chlorine, bromine, and/or iodine atoms. In one embodiment, the alkyl is substituted by one, two, or three fluorine and/or chlorine atoms. In another embodiment, the alkyl is substituted by one, two, or three fluorine atoms. In another embodiment, the alkyl is a C₁-C₆ alkyl. In another embodiment, the alkyl is a C₁-C₄ alkyl. In another embodiment, the alkyl group is a C₁ or C₂ alkyl. Non-limiting exemplary haloalkyl groups include fluoromethyl, difluoromethyl, trifluoromethyl, pentafluoroethyl, 1,1-difluoroethyl, 2,2-difluoroethyl, 2,2,2-trifluoroethyl, 3,3,3-trifluoropropyl, 4,4,4-trifluorobutyl, and trichloromethyl groups.

The terms “hydroxyalkyl” or “(hydroxy)alkyl” as used herein by themselves or as part of another group refer to an alkyl group substituted with one, two, or three hydroxy groups. In one embodiment, the alkyl is a C₁-C₆ alkyl. In another embodiment, the alkyl is a C₁-C₄ alkyl. In another embodiment, the alkyl is a C₁ or C₂ alkyl. In another embodiment, the hydroxyalkyl is a monohydroxyalkyl group, i.e., substituted with one hydroxy group. In another embodiment, the hydroxyalkyl group is a dihydroxyalkyl group, i.e., substituted with two hydroxy groups. Non-limiting exemplary (hydroxyl)alkyl groups include hydroxymethyl, hydroxyethyl, hydroxypropyl and hydroxybutyl groups, such as 1-hydroxyethyl, 2-hydroxyethyl, 1,2-dihydroxyethyl, 2-hydroxypropyl, 3-hydroxypropyl, 3-hydroxybutyl, 4-hydroxybutyl, 2-hydroxy-1-methylpropyl, and 1,3-dihydroxyprop-2-yl.

The term “alkoxy” as used herein by itself or as part of another group refers to an alkyl group attached to a terminal oxygen atom. In one embodiment, the alkyl is a C₁-C₆ alkyl and resulting alkoxy is thus referred to as a “C₁-C₆ alkoxy.” In another embodiment, the alkyl is a C₁-C₄ alkyl group. Non-limiting exemplary alkoxy groups include methoxy, ethoxy, and tert-butoxy.

The term “haloalkoxy” as used herein by itself or as part of another group refers to a haloalkyl group attached to a terminal oxygen atom. In one embodiment, the haloalkyl group is a C₁-C₆ haloalkyl. In another embodiment, the haloalkyl group is a C₁-C₄ haloalkyl group. Non-limiting exemplary haloalkoxy groups include fluoromethoxy, difluoromethoxy, trifluoromethoxy, and 2,2,2-trifluoroethoxy.

The term “alkylthio” as used herein by itself or as part of another group refers to an alkyl group attached to a terminal sulfur atom. In one embodiment, the alkyl group is a C₁-C₄ alkyl group. Non-limiting exemplary alkylthio groups include —SCH₃, and —SCH₂CH₃.

The terms “alkoxyalkyl” or “(alkoxy)alkyl” as used herein by themselves or as part of another group refers to an alkyl group substituted with one alkoxy group. In one embodiment, the alkoxy is a C₁-C₆ alkoxy. In another embodiment, the alkoxy is a C₁-C₄ alkoxy. In another embodiment, the alkyl is a C₁-C₆ alkyl. In another embodiment, the alkyl is a C₁-C₄ alkyl. Non-limiting exemplary alkoxyalkyl groups include methoxymethyl, methoxyethyl, methoxypropyl, methoxybutyl, ethoxymethyl, ethoxyethyl, ethoxypropyl, ethoxybutyl, propoxymethyl, iso-propoxymethyl, propoxyethyl, propoxypropyl, butoxymethyl, tert-butoxymethyl, isobutoxymethyl, sec-butoxymethyl, and pentyloxymethyl.

The term “heteroalkyl” as used by itself or part of another group refers to unsubstituted straight- or branched-chain aliphatic hydrocarbons containing from three to twenty chain atoms, i.e., 3- to 20-membered heteroalkyl, or the number of chain atoms designated, wherein at least one —CH₂— is replaced with at least one of —O—, —N(H)—, —N(C₁-C₄ alkyl)-, or —S—. The —O—, —N(H)—, —N(C₁-C₄ alkyl)-, or —S— can independently be placed at any position of the aliphatic hydrocarbon chain so long as each —O—, —N(H)—, —N(C₁-C₄ alkyl)-, and —S— group is separated by at least two —CH₂— groups. In one embodiment, one —CH₂— group is replaced with one —O— group. In another embodiment, two —CH₂— groups are replaced with two —O— groups. In another embodiment, three —CH₂— groups are replaced with three —O— groups. In another embodiment, four —CH₂— groups are replaced with four —O— groups. Non-limiting exemplary heteroalkyl groups include —CH₂OCH₃, —CH₂OCH₂CH₂CH₃, —CH₂CH₂CH₂OCH₃, —NHCH₂CH₂OCH—₂CH₂OCH₂CH₃, —CH₂CH₂OCH₂CH₂OCH₂CH₃, —CH₂CH₂OCH₂CH₂OCH—₂CH₂OCH₂CH₃.

The term “cycloalkyl” as used herein by itself or as part of another group refers to saturated and partially unsaturated, e.g., containing one or two double bonds, monocyclic, bicyclic, or tricyclic aliphatic hydrocarbons containing three to twelve carbon atoms, i.e., a C₃₋₁₂ cycloalkyl, or the number of carbons designated, e.g., a C₃ cycloalkyl such a cyclopropyl, a C₄ cycloalkyl such as cyclobutyl, etc. In one embodiment, the cycloalkyl is bicyclic, i.e., it has two rings. In another embodiment, the cycloalkyl is monocyclic, i.e., it has one ring. In another embodiment, the cycloalkyl is a C₃₋₈ cycloalkyl. In another embodiment, the cycloalkyl is a C₃₋₆ cycloalkyl, i.e., cyclopropyl, cyclobutyl, cyclopentyl, or cyclohexyl. In another embodiment, the cycloalkyl is a C₅ cycloalkyl, i.e., cyclopentyl or cyclopentenyl. In another embodiment, the cycloalkyl is a C₆ cycloalkyl, i.e., cyclohexyl or cyclohexenyl. Non-limiting exemplary C₃₋₁₂ cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

The term “optionally substituted cycloalkyl” as used herein by itself or as part of another group refers to a cycloalkyl group that is either unsubstituted or substituted with one, two, or three substituents, wherein each substituent is independently halo, nitro, cyano, hydroxy, amino (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, and R⁵⁸ are as defined in connection with the term “optionally substituted alkyl” and R⁹ is (hydroxy)alkyl or (amino)alkyl. The term optionally substituted cycloalkyl also includes cycloalkyl groups having fused optionally substituted aryl or optionally substituted heteroaryl groups such as

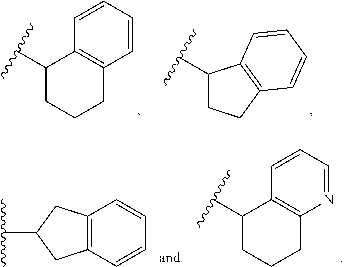 [SMILES: *C1CCCc2ccccc21 ; *C1CCc2ccccc21 ; *C1Cc2ccccc2C1 ; *C1CCCc2ncccc21]

Non-limiting exemplary optionally substituted cycloalkyl groups include:

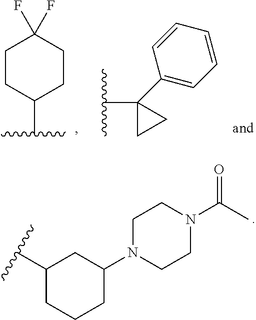

The term “heterocyclo” as used herein by itself or as part of another group refers to saturated and partially unsaturated, e.g., containing one or two double bonds, monocyclic, bicyclic, or tricyclic groups containing three to eighteen ring members, i.e., a 3- to 18-membered heterocyclo, comprising one, two, three, or four heteroatoms. Each heteroatom is independently oxygen, sulfur, or nitrogen. Each sulfur atom is independently oxidized to give a sulfoxide, i.e., S(═O), or sulfone, i.e., S(═O)₂. The term heterocyclo includes groups wherein one or more —CH₂— groups is replaced with one or more —C(═O)— groups, including cyclic ureido groups such as imidazolidinyl-2-one, cyclic amide groups such as pyrrolidin-2-one or piperidin-2-one, and cyclic carbamate groups such as oxazolidinyl-2-one. The term heterocyclo also includes groups having fused optionally substituted aryl or optionally substituted heteroaryl groups such as indoline, indolin-2-one, 2,3-dihydro-1H-pyrrolo[2,3-c]pyridine, 2,3,4,5-tetrahydro-1H-benzo[d]azepine, or 1,3,4,5-tetrahydro-2H-benzo[d]azepin-2-one.

In one embodiment, the heterocyclo group is a 4- to 8-membered cyclic group containing one ring and one or two oxygen atoms, e.g., tetrahydrofuran or tetrahydropyran, or one or two nitrogen atoms, e.g., pyrrolidine, piperidine, or piperazine, or one oxygen and one nitrogen atom, e.g., morpholine, and, optionally, one —CH₂— group is replaced with one —C(═O)— group, e.g., pyrrolidin-2-one or piperazin-2-one. In another embodiment, the heterocyclo group is a 5- to 8-membered cyclic group containing one ring and one or two nitrogen atoms and, optionally, one —CH₂— group is replaced with one —C(═O)— group. In another embodiment, the heterocyclo group is a 5- or 6-membered cyclic group containing one ring and one or two nitrogen atoms and, optionally, one —CH₂— group is replaced with one —C(═O)— group. In another embodiment, the heterocyclo group is a 8- to 12-membered cyclic group containing two rings and one or two nitrogen atoms. The heterocyclo can be linked to the rest of the molecule through any available carbon or nitrogen atom. Non-limiting exemplary heterocyclo groups include:

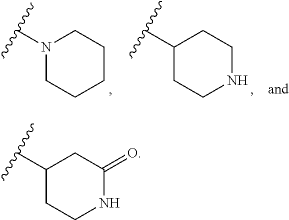

The term “optionally substituted heterocyclo” as used herein by itself or part of another group refers to a heterocyclo group that is either unsubstituted or substituted with one to four substituents, wherein each substituent is independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.” Substitution may occur on any available carbon or nitrogen atom of the heterocyclo group. Non-limiting exemplary optionally substituted heterocyclo groups include:

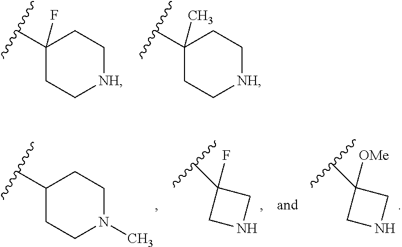

In one embodiment, the heterocyclo group is a spiroheterocyclo. The term “spiroheterocyclo” as used herein by itself or part of another group refers to an optionally substituted heterocyclo group containing seven to eighteen ring members, wherein:

(i) a first and second ring are connected through a quaternary carbon atom, i.e., a spirocarbon;

(ii) the first ring is an optionally substituted mono- or bicyclic heterocyclo containing a nitrogen atom; and

(iii) the second ring is either:

(a) an optionally substituted mono- or bicyclic cycloalkyl; or

(b) an optionally substituted mono- or bicyclic heterocyclo containing a nitrogen atom.

In one embodiment, the first ring is an optionally substituted monocyclic 4- to 9-membered heterocyclo containing a nitrogen atom. In another embodiment, the second ring is an optionally substituted monocyclic Cm cycloalkyl. In another embodiment, the second ring is a monocyclic Cm cycloalkyl substituted with a hydroxy group. In another embodiment, the second ring is an optionally substituted monocyclic 4- to 9-membered heterocyclo containing a nitrogen atom. Non-limiting exemplary spiroheterocyclo groups include:

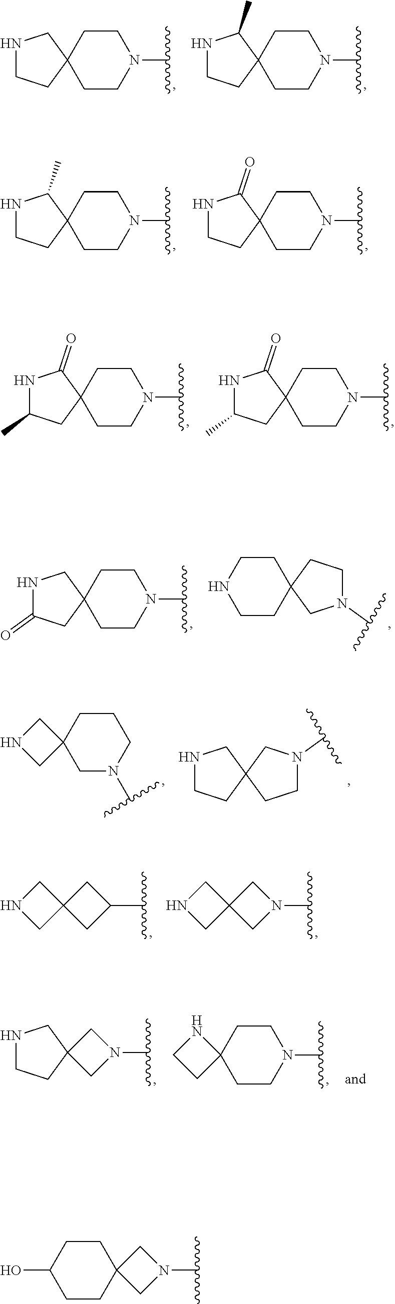

The term “aryl” as used herein by itself or as part of another group refers to an aromatic ring system having six to fourteen carbon atoms, i.e., C₆-C₁₄ aryl. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is phenyl or naphthyl. In another embodiment, the aryl group is phenyl.

The term “optionally substituted aryl” as used herein by itself or as part of another group refers to aryl that is either unsubstituted or substituted with one to five substituents, wherein the substituents are each independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted aryl is an optionally substituted phenyl. In another embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary optionally substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, 3-chloro-4-fluorophenyl, and 2-phenylpropan-2-amine. The term optionally substituted aryl includes aryl groups having fused optionally substituted cycloalkyl groups and fused optionally substituted heterocyclo groups. Non-limiting examples include: 2,3-dihydro-1H-inden-1-yl, 1,2,3,4-tetrahydronaphthalen-1-yl, 1,3,4,5-tetrahydro-2H-benzo[c]azepin-2-yl, 1,2,3,4-tetrahydroisoquinolin-1-yl, and 2-oxo-2,3,4,5-tetrahydro-1H-benzo[d]azepin-1-yl.

The term “heteroaryl” as used herein by itself or as part of another group refers to monocyclic and bicyclic aromatic ring systems having five to 14 fourteen ring members, i.e., a 5- to 14-membered heteroaryl, comprising one, two, three, or four heteroatoms. Each heteroatom is independently oxygen, sulfur, or nitrogen. In one embodiment, the heteroaryl has three heteroatoms. In another embodiment, the heteroaryl has two heteroatoms. In another embodiment, the heteroaryl has one heteroatom. In another embodiment, the heteroaryl is a 5- to 10-membered heteroaryl. In another embodiment, the heteroaryl has 5 ring atoms, e.g., thienyl, a 5-membered heteroaryl having four carbon atoms and one sulfur atom. In another embodiment, the heteroaryl has 6 ring atoms, e.g., pyridyl, a 6-membered heteroaryl having five carbon atoms and one nitrogen atom. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term heteroaryl also includes N-oxides. A non-limiting exemplary N-oxide is pyridyl N-oxide.

The term “optionally substituted heteroaryl” as used herein by itself or as part of another group refers to a heteroaryl that is either unsubstituted or substituted with one to four substituents, wherein the substituents are independently halo, nitro, cyano, hydroxy, amino, (e.g., —NH₂, alkylamino, dialkylamino, aralkylamino, hydroxyalkylamino, or (heterocyclo)alkylamino), heteroalkyl, haloalkyl, hydroxyalkyl, alkoxy, haloalkoxy, aryloxy, aralkyl, aralkyloxy, alkylthio, carboxamido, sulfonamido, alkylcarbonyl, arylcarbonyl, alkylsulfonyl, arylsulfonyl, ureido, guanidino, carboxy, carboxyalkyl, optionally substituted alkyl, optionally substituted cycloalkyl, alkenyl, alkynyl, optionally substituted aryl, optionally substituted heteroaryl, optionally substituted heterocyclo, alkoxyalkyl, (amino)alkyl, (cyano)alkyl, (carboxamido)alkyl, mercaptoalkyl, (heterocyclo)alkyl, (heteroaryl)alkyl, —N(R^{56a})C(═O)R^{56b}, —N(R^{56c})S(═O)₂R^{56d}, —C(═O)R⁵⁷, —S(═O)R^{56e}, —S(═O)₂R⁵⁸, or —OR⁵⁹, wherein R^{56a}, R^{56b}, R^{56c}, R^{56d}, R^{56e}, R⁵⁷, R⁵⁸, and R⁵⁹ are as defined in connection with the term “optionally substituted cycloalkyl.”

In one embodiment, the optionally substituted heteroaryl has two substituents. In another embodiment, the optionally substituted heteroaryl has one substituent. Any available carbon or nitrogen atom can be substituted.

The term “aryloxy” as used herein by itself or as part of another group refers to an optionally substituted aryl attached to a terminal oxygen atom. A non-limiting exemplary aryloxy group is PhO—.

The term “heteroaryloxy” as used herein by itself or as part of another group refers to an optionally substituted heteroaryl attached to a terminal oxygen atom. A non-limiting exemplary aryloxy group is pyridyl-O—.

The term “aralkyloxy” as used herein by itself or as part of another group refers to an aralkyl attached to a terminal oxygen atom. A non-limiting exemplary aralkyloxy group is PhCH₂O—.

The term “(cyano)alkyl” as used herein by itself or as part of another group refers to an alkyl substituted with one, two, or three cyano groups. In one embodiment, the alkyl is substituted with one cyano group. In another embodiment, the alkyl is a C₁-C₆ alkyl In another embodiment, the alkyl is a C₁-C₄ alkyl. Non-limiting exemplary (cyano)alkyl groups include —CH₂CH₂CN and —CH₂CH₂CH₂CN.

The term “(cycloalkyl)alkyl” as used herein by itself or as part of another group refers to an alkyl substituted with one or two optionally substituted cycloalkyl groups. In one embodiment, the cycloalkyl group(s) is an optionally substituted C₃-C₆ cycloalkyl. In another embodiment, the alkyl is a C₁-C₆ alkyl. In another embodiment, the alkyl is a C₁-C₄ alkyl. In another embodiment, the alkyl is a C₁ or C₂ alkyl. In another embodiment, the alkyl is substituted with one optionally substituted cycloalkyl group. In another embodiment, the alkyl is substituted with two optionally substituted cycloalkyl groups. Non-limiting exemplary (cycloalkyl)alkyl groups include:

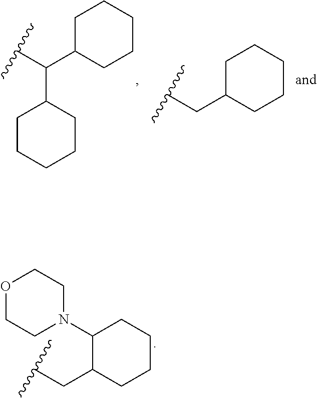 [SMILES: *C(C1CCCCC1)C1CCCCC1 ; *CC1CCCCC1 ; *CC1CCCCC1N1CCOCC1]

The term “sulfonamido” as used herein by itself or as part of another group refers to a radical of the formula —SO₂NR^{50a}R^{50b}, wherein R^{50a} and R^{50b} are each independently hydrogen, alkyl, optionally substituted cycloalkyl, optionally substituted heterocyclo, optionally substituted aryl, or optionally substituted heteroaryl; or R^{50a} and R^{50b} taken together with the nitrogen to which they are attached form a 3- to 8-membered optionally substituted heterocyclo group. Non-limiting exemplary sulfonamido groups include —SO₂NH₂, —SO₂N(H)CH₃, and —SO₂N(H)Ph.

The term “alkylcarbonyl” as used herein by itself or as part of another group refers to a carbonyl group, i.e., —C(═O)—, substituted by an alkyl group. In one embodiment, the alkyl is a C₁-C₄ alkyl. A non-limiting exemplary alkylcarbonyl group is —COCH₃.

The term “arylcarbonyl” as used herein by itself or as part of another group refers to a carbonyl group, i.e., —C(═O)—, substituted by an optionally substituted aryl group. A non-limiting exemplary arylcarbonyl group is —COPh.

The term “alkylsulfonyl” as used herein by itself or as part of another group refers to a sulfonyl group, i.e., —SO₂—, substituted by an alkyl group. A non-limiting exemplary alkylsulfonyl group is —SO₂CH₃.

The term “arylsulfonyl” as used herein by itself or as part of another group refers to a sulfonyl group, i.e., —SO₂—, substituted by an optionally substituted aryl group. A non-limiting exemplary arylsulfonyl group is —SO₂Ph.

The term “mercaptoalkyl” as used herein by itself or as part of another group refers to an alkyl substituted by a —SH group.

The term “carboxy” as used by itself or as part of another group refers to a radical of the formula —C(═O)OH.

The term “ureido” as used herein by itself or as part of another group refers to a radical of the formula —NR^{51a}—C(═O)—NR^{51b}R^{51c}, wherein R^{51a} is hydrogen or alkyl; and R^{51b} and R^{51c} are each independently hydrogen, alkyl, optionally substituted cycloalkyl, optionally substituted heterocyclo, optionally substituted aryl, or optionally substituted heteroaryl, or R^{51b} and R^{51c} taken together with the nitrogen to which they are attached form a 4- to 8-membered optionally substituted heterocyclo group. Non-limiting exemplary ureido groups include —NH—C(C═O)—NH₂ and —NH—C(C═O)—NHCH₃.

The term “guanidino” as used herein by itself or as part of another group refers to a radical of the formula —NR^{52a}—C(═NR⁵³)—NR^{52b}R^{52c}, wherein R^{52a} is hydrogen or alkyl; R^{52b} and R^{53c} are each independently hydrogen, alkyl, optionally substituted cycloalkyl, optionally substituted heterocyclo, optionally substituted aryl, or optionally substituted heteroaryl; or R^{52b} and R^{52c} taken together with the nitrogen to which they are attached form a 4- to 8-membered optionally substituted heterocyclo group; and R⁵³ is hydrogen, alkyl, cyano, alkylsulfonyl, alkylcarbonyl, carboxamido, or sulfonamido. Non-limiting exemplary guanidino groups include —NH—C(C═NH)—NH₂, —NH—C(C═NCN)—NH₂, and —NH—C(C═NH)—NHCH₃.

The term “(heterocyclo)alkyl” as used herein by itself or as part of another group refers to an alkyl substituted with one, two, or three optionally substituted heterocyclo groups. In one embodiment, the alkyl is substituted with one optionally substituted 5- to 8-membered heterocyclo group. In another embodiment, alkyl is a C₁-C₆ alkyl. In another embodiment, alkyl is a C₁-C₄ alkyl. The heterocyclo group can be linked to the alkyl group through a carbon or nitrogen atom. Non-limiting exemplary (heterocyclo)alkyl groups include:

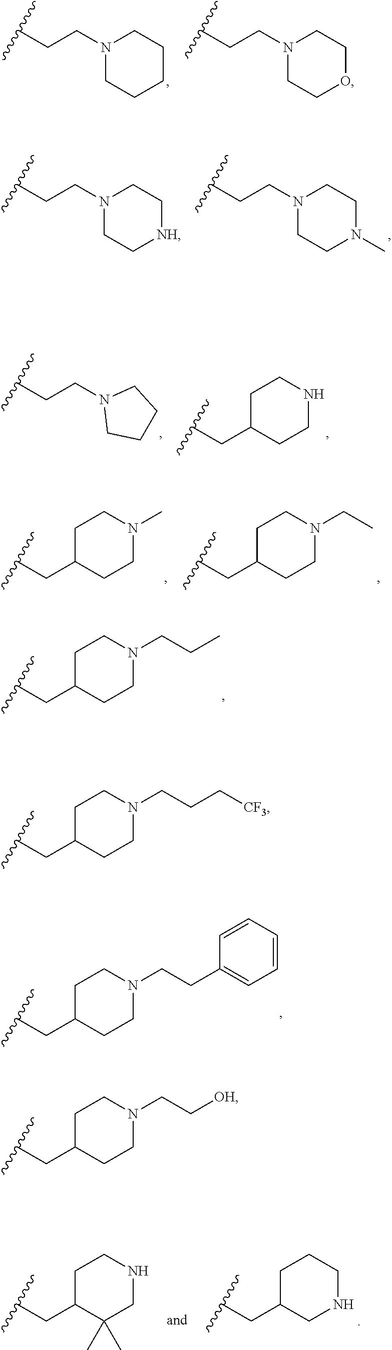

The term “carbamate” as used herein by itself or as part of another group refers to a radical of the formula —NR^{54a}—C(═O)—OR^{54b}, wherein R^{54a} is hydrogen or alkyl, and R^{54b} is hydrogen, alkyl, optionally substituted cycloalkyl, optionally substituted heterocyclo, optionally substituted aryl, or optionally substituted heteroaryl. A non-limiting exemplary carbamate group is —NH—(C═O)—OtBu.

The term “(heteroaryl)alkyl” as used herein by itself or as part of another group refers to an alkyl substituted with one or two optionally substituted heteroaryl groups. In one embodiment, the alkyl group is substituted with one optionally substituted 5- to 14-membered heteroaryl group. In another embodiment, the alkyl group is substituted with two optionally substituted 5- to 14-membered heteroaryl groups. In another embodiment, the alkyl group is substituted with one optionally substituted 5- to 9-membered heteroaryl group. In another embodiment, the alkyl group is substituted with two optionally substituted 5- to 9-membered heteroaryl groups. In another embodiment, the alkyl group is substituted with one optionally substituted 5- or 6-membered heteroaryl group. In another embodiment, the alkyl group is substituted with two optionally substituted 5- or 6-membered heteroaryl groups. In one embodiment, the alkyl group is a C₁-C₆ alkyl. In another embodiment, the alkyl group is a C₁-C₄ alkyl. In another embodiment, the alkyl group is a C₁ or C₂ alkyl. Non-limiting exemplary (heteroaryl)alkyl groups include:

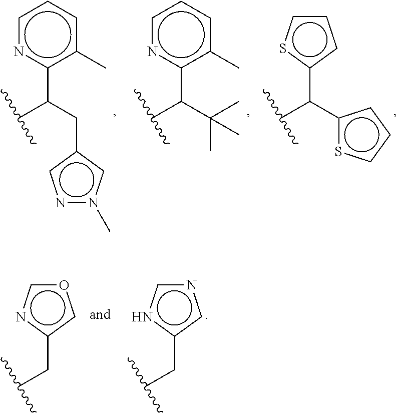

The term “(amino)(heteroaryl)alkyl” as used herein by itself or as part of another group refers to an alkyl group substituted with one optionally substituted heteroaryl group and one amino group. In one embodiment, the heteroaryl is an optionally substituted 5- to 9-membered heteroaryl group. In another embodiment, the heteroaryl is an optionally substituted 5- or 6-membered heteroaryl group. In one embodiment, the alkyl is a C₁-C₆ alkyl. In another embodiment, the alkyl is a C₁-C₄ alkyl. In another embodiment, the alkyl is a C₁ or C₂ alkyl. A non-limiting exemplary (amino)(heteroaryl)alkyl group is:

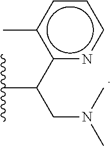

The terms “aralkyl” or “(aryl)alkyl” as used herein by themselves or as part of another group refers to an alkyl substituted with one, two, or three optionally substituted aryl groups. In one embodiment, the alkyl is substituted with one optionally substituted aryl group. In another embodiment, the alkyl is substituted with two optionally substituted aryl groups. In one embodiment, the aryl is an optionally substituted phenyl or optionally substituted naphthyl. In another embodiment, the aryl is an optionally substituted phenyl. In one embodiment, the alkyl is a C₁-C₆ alkyl. In another embodiment, the alkyl is a C₁-C₄ alkyl. In another embodiment, the alkyl is a C₁ or C₂ alkyl. Non-limiting exemplary (aryl)alkyl groups include benzyl, phenethyl, —CHPh₂, and —CH(4-F-Ph)₂.

The term “amido” as used herein by itself or as part of another group refers to a radical of formula —C(═O)NR^{60a}R^{60b}, wherein R^{60a} and R^{60b} are each independently hydrogen, optionally substituted alkyl, optionally substituted alkenyl, optionally substituted alkynyl, haloalkyl, (alkoxy)alkyl, (hydroxy)alkyl, (cyano)alkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl; or R^{60a} and R^{60b} taken together with the nitrogen to which they are attached from a 4- to 8-membered optionally substituted heterocyclo group. In one embodiment, R^{60a} and R^{60b} are each independently hydrogen or C₁-C₆ alkyl.

The term “(amido)(aryl)alkyl” as used herein by itself or as part of another group refers to an alkyl group substituted with one amido group and one optionally substituted aryl group. In one embodiment, the aryl group is an optionally substituted phenyl. In one embodiment, the alkyl is a C₁-C₆ alkyl. In another embodiment, the alkyl is a C₁-C₄ alkyl. Non-limiting exemplary (amido)(aryl)alkyl groups include:

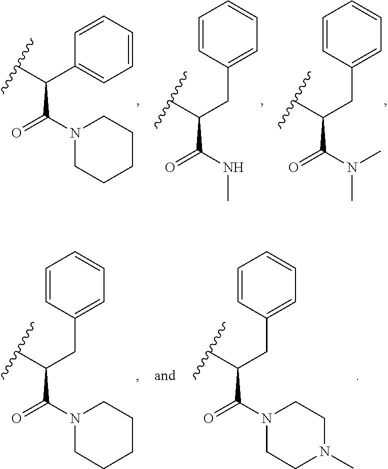

The term “(amino)(aryl)alkyl” as used herein by itself or as part of another group refers to an alkyl group substituted with one amino group and one optionally substituted aryl group. In one embodiment, the amino group is —NH₂, alkylamino, or dialkylamino. In one embodiment, the aryl group is an optionally substituted phenyl. In one embodiment, the alkyl is a C₁-C₆ alkyl. In another embodiment, the alkyl is a C₁-C₄ alkyl. Non-limiting exemplary (amino)(aryl)alkyl groups include:

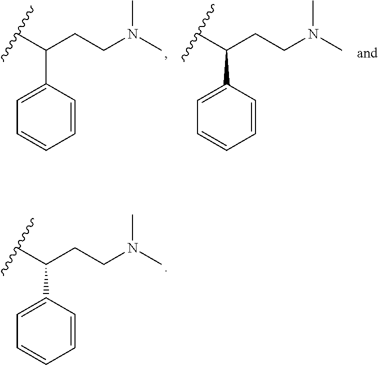

The term “amino” as used by itself or as part of another group refers to a radical of the formula —NR^{55a}R^{55b}, wherein R^{55a} and R^{55b} are independently hydrogen, optionally substituted alkyl, haloalkyl, (hydroxy)alkyl, (alkoxy)alkyl, (amino)alkyl, heteroalkyl, optionally substituted cycloalkyl, optionally substituted heterocyclo, optionally substituted aryl, optionally substituted heteroaryl, (aryl)alkyl, (cycloalkyl)alkyl, (heterocyclo)alkyl, or (heteroaryl)alkyl.

In one embodiment, the amino is —NH₂.

In another embodiment, the amino is an “alkylamino,” i.e., an amino group wherein R^{55a} is C₁₋₆ alkyl and R^{55b} is hydrogen. In one embodiment, R^{55a} is C₁-C₄ alkyl. Non-limiting exemplary alkylamino groups include —N(H)CH₃ and —N(H)CH₂CH₃.

In another embodiment, the amino is a “dialkylamino,” i.e., an amino group wherein R^{55a} and R^{55b} are each independently C₁₋₆alkyl. In one embodiment, R^{55a} and R^{55b} are each independently C₁-C₄ alkyl. Non-limiting exemplary dialkylamino groups include —N(CH₃)₂ and —N(CH₃)CH₂CH(CH₃)₂.

In another embodiment, the amino is a “hydroxyalkylamino,” i.e., an amino group wherein R^{55a} is (hydroxyl)alkyl and R^{55b} is hydrogen or C₁-C₄ alkyl.

In another embodiment, the amino is a “cycloalkylamino,” i.e., an amino group wherein R^{55a} is optionally substituted cycloalkyl and R^{55b} is hydrogen or C₁-C₄ alkyl.

In another embodiment, the amino is a “aralkylamino,” i.e., an amino group wherein R^{55a} is aralkyl and R^{55b} is hydrogen or C₁-C₄ alkyl. Non-limiting exemplary aralkylamino groups include —N(H)CH₂Ph, —N(H)CHPh₂, and —N(CH₃)CH₂Ph.

In another embodiment, the amino is a “(cycloalkyl)alkylamino,” i.e., an amino group wherein R^{55a} is (cycloalkyl)alkyl and R^{55b} is hydrogen or C₁-C₄ alkyl. Non-limiting exemplary (cycloalkyl)alkylamino groups include:

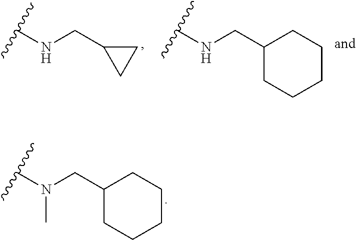

In another embodiment, the amino is a “(heterocyclo)alkylamino,” i.e., an amino group wherein R^{55a} is (heterocyclo)alkyl and R^{55b} is hydrogen or C₁-C₄ alkyl. Non-limiting exemplary (heterocyclo)alkylamino groups include:

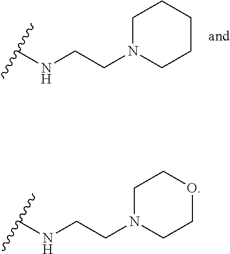 [SMILES: *NCCN1CCCCC1 ; *NCCN1CCOCC1]

The term “(amino)alkyl” as used herein by itself or as part of another group refers to an alkyl substituted with one amino group. In one embodiment, the amino group is —NH₂. In one embodiment, the amino group is an alkylamino. In another embodiment, the amino group is a dialkylamino. In another embodiment, the alkyl is a C₁-C₆ alkyl. In another embodiment, the alkyl is a C₁-C₄ alkyl. Non-limiting exemplary (amino)alkyl groups include —CH₂NH₂, CH₂CH₂N(H)CH₃, —CH₂CH₂N(CH₃)₂, CH₂N(H)cyclopropyl, —CH₂N(H)cyclobutyl, and —CH₂N(H)cyclohexyl, and —CH₂CH₂CH₂N(H)CH₂Ph and —CH₂CH₂CH₂N(H)CH₂(4-CF₃-Ph).

The term “heteroarylenyl” as used herein by itself or part of another group refers to a divalent form of an optionally substituted 5- to 9-membered heteroaryl group. In one embodiment, the heteroarylenyl is a 6-membered heteroarylenyl, e.g., heteroarylenyl derived from pyridine. In one embodiment, the heteroarylenyl is a bicyclic 9-membered heteroarylenyl. Exemplary non-limiting exemplary bicyclic 9-membered heteroarylenyl groups include:

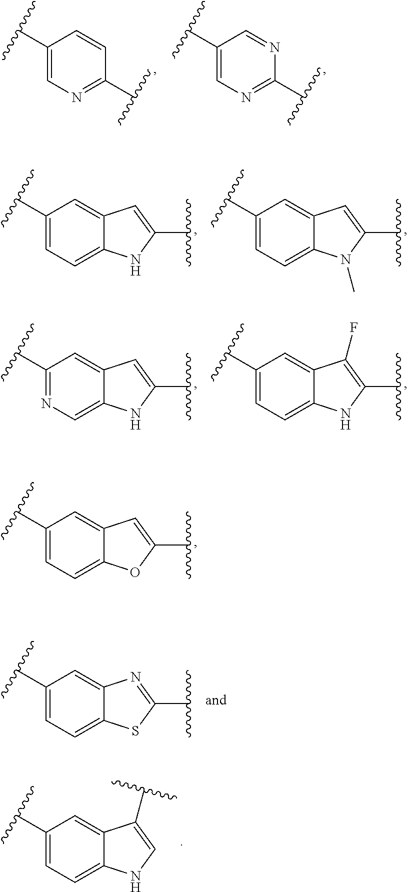

In the present disclosure, the term “alkylenyl” as used herein by itself or part of another group refers to a divalent form of an alkyl group, wherein the alkyl group is either unsubstituted or substituted with one or two groups independently selected from the group consisting of optionally substituted phenyl and optionally substituted 5- or 6-membered heteroaryl. In one embodiment, the alkylenyl is a divalent form of a C₁₋₁₂ alkyl, i.e., a C₁-C₁₂ alkylenyl. In one embodiment, the alkylenyl is a divalent form of a C₁₋₁₀ alkyl, i.e., a C₁-C₁₀ alkylenyl. In one embodiment, the alkylenyl is a divalent form of a C₁₋₈ alkyl, i.e., a C₁-C₈ alkylenyl. In one embodiment, the alkylenyl is a divalent form of an unsubstituted C₁₋₆ alkyl, i.e., a C₁-C₆ alkylenyl. In another embodiment, the alkylenyl is a divalent form of an unsubstituted C₁₋₄ alkyl, i.e., a C₁-C₄ alkylenyl. In another embodiment, the alkylenyl is a divalent form of a C₁₋₄ alkyl substituted with one or two optionally substituted phenyl groups. Non-limiting exemplary alkylenyl groups include —CH₂—, —CH₂CH₂—, —CH(Ph)-, —CH(Ph)CH₂—, —CH₂CH₂CH₂—, —CH(Ph)CH₂CH₂—, —CH₂(CH₂)₂CH₂—, —CH(CH₂)₃CH₂—, and —CH₂(CH₂)₄CH₂—.

The term “heteroalkylenyl” as used herein by itself or part of another group refers to a divalent form of a heteroalkyl group. In one embodiment, the heteroalkylenyl is a divalent form of a 3- to 20-membered heteroalkyl, i.e., a 3- to 20-membered heteroalkylenyl. In another embodiment, the heteroalkylenyl is a divalent form of a 3- to 10-membered heteroalkyl, i.e., a 3- to 10-membered heteroalkylenyl. In another embodiment, the heteroalkylenyl is a divalent form of a 3- to 8-membered heteroalkyl, i.e., a 3- to 8-membered heteroalkylenyl. In another embodiment, the heteroalkylenyl is a divalent form of a 3- to 6-membered heteroalkyl, i.e., a 3- to 6-membered heteroalkylenyl. In another embodiment, the heteroalkylenyl is a divalent form of a 3- or 4-membered heteroalkyl, i.e., a 3- or 4-membered heteroalkylenyl. In another embodiment, the heteroalkylenyl is a radical of the formula —(CH₂CH₂O)ᵤ₁— wherein u₁ is 1, 2, 3, 4, 5, or 6. Non-limiting exemplary heteroalkylenyl groups include —CH₂OCH₂—, —CH₂CH₂OCH₂CH₂O—, —CH₂OCH₂CH₂CH₂—, and —CH₂CH₂OCH₂CH₂OCH₂CH₂O—.

The term “heterocyclenyl” as used herein by itself or part of another group refers to a divalent form of an optionally substituted heterocyclo. In another embodiment, the heterocyclenyl is a divalent form of a 4- to 14-membered heterocyclo group, i.e., a 4- to 14-membered heterocyclenyl. In another embodiment, the heterocyclenyl is a divalent form of a 4- to 10-membered heterocyclo group, i.e., a 4- to 10-membered heterocyclenyl. In another embodiment, the heterocyclenyl is a divalent form of a 4- to 8-membered heterocyclo group, i.e., a 4- to 8-membered heterocyclenyl. In one embodiment, the heterocyclenyl is a divalent form of an optionally substituted azetidine. In another embodiment, the heterocyclenyl is a divalent form of an optionally substituted piperidinyl. In another embodiment, the heterocyclenyl is a divalent form of an optionally substituted piperazinyl. Non-limiting exemplary heterocyclenyl groups include:

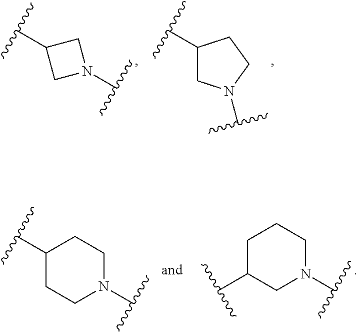 [SMILES: *C1CN(*)C1 ; *C1CCN(*)C1 ; *C1CCN(*)CC1 ; *C1CCCN(*)C1]

In another embodiment, the heterocyclenyl is a spiroheterocyclenyl.

The term “spiroheterocyclenyl” as used herein by itself or part of another group refers to a divalent form of a spiroheterocyclo. Non-limiting exemplary spiroheterocyclenyl groups include:

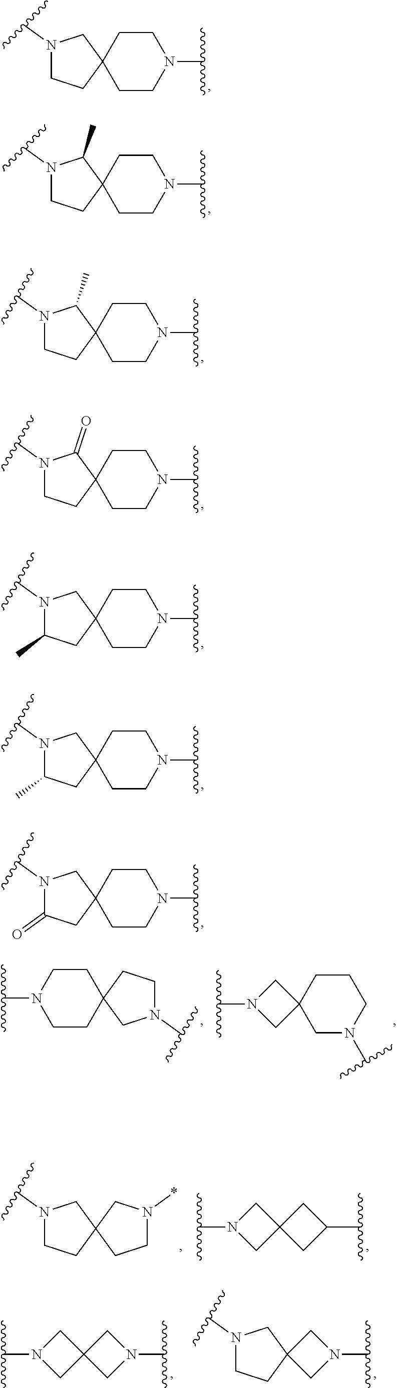

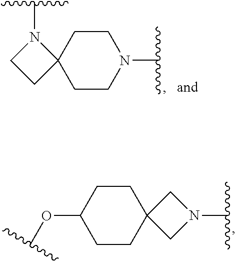 [SMILES: *N1CCC2(CC1)CCN2* ; *OC1CCC2(CC1)CN(*)C2]

The term “cycloalkylenyl” as used herein by itself or part of another group refers to a divalent form of an optionally substituted C₄-C₆ cycloalkyl group. In one embodiment, the cycloalkylenyl is a 4-membered cycloalkylenyl. In another embodiment, the cycloalkylenyl is a 5-membered cycloalkylenyl. In another embodiment, the cycloalkylenyl is a 6-membered cycloalkylenyl. Non-limiting exemplary groups include:

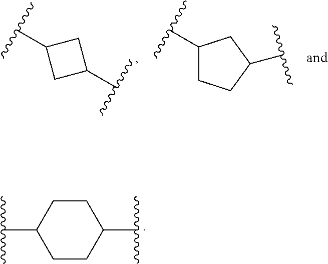 [SMILES: *C1CC(*)C1 ; *C1CCC(*)C1 ; *C1CCC(*)CC1]

The term “phenylenyl” as used herein by itself or part of another group refers to a divalent form of an optionally substituted phenyl group. Non-limiting examples include:

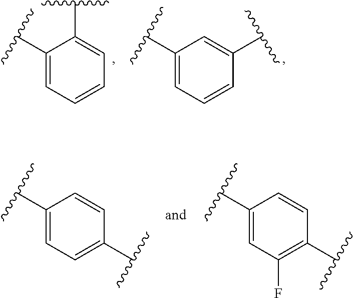

The present disclosure encompasses any of the Compounds of the Disclosure being isotopically-labelled (i.e., radiolabeled) by having one or more atoms replaced by an atom having a different atomic mass or mass number. Examples of isotopes that can be incorporated into the disclosed compounds include isotopes of hydrogen, carbon, nitrogen, oxygen, phosphorous, fluorine and chlorine, such as ²H (or deuterium (D)), ³H, ¹¹C, ¹³C, ¹⁴C, ¹⁵N, ¹⁸O, ¹⁷O, ³¹P, ³²P, ³⁵S, ¹⁸F, and ³⁶Cl, respectively, e.g., ³H, ¹¹C, and ¹⁴C. In one embodiment, provided is a composition wherein substantially all of the atoms at a position within the Compound of the Disclosure are replaced by an atom having a different atomic mass or mass number. In another embodiment, provided is a composition wherein a portion of the atoms at a position within the Compound of the disclosure are replaced, i.e., the Compound of the Disclosure is enriched at a position with an atom having a different atomic mass or mass number.” Isotopically-labelled Compounds of the Disclosure can be prepared by methods known in the art.

As noted above, Compounds of the Disclosure contain one or more asymmetric carbon atoms and may thus give rise to enantiomers, diastereomers, and other stereoisomeric forms. The present disclosure encompasses the use of all such possible forms, as well as their racemic and resolved forms and mixtures thereof. The individual enantiomers can be separated according to methods known in the art in view of the present disclosure. When the compounds described herein contain olefinic double bonds or other centers of geometric asymmetry, and unless specified otherwise, it is intended that they include both E and Z geometric isomers. All tautomers are also encompassed by the present disclosure.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive. In one embodiment, Compounds of the Disclosure are racemic.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The term “about,” as used herein, includes the recited number±10%. Thus, “about 10” means 9 to 11.

### EXAMPLES

### Example 1

### Synthesis of 2-Chloro-4-((1r,3r)-3-(5-(4-(1-(2-(2,6-dioxopiperidin-3-yl)-1,3-dioxoisoindolin-5-yl)piperidin-4-yl)piperazine-1-carbonyl)-1-oxoisoindolin-2-yl)-2,2,4,4-tetramethylcyclobutoxy)benzonitrile (Cpd. No. 141)

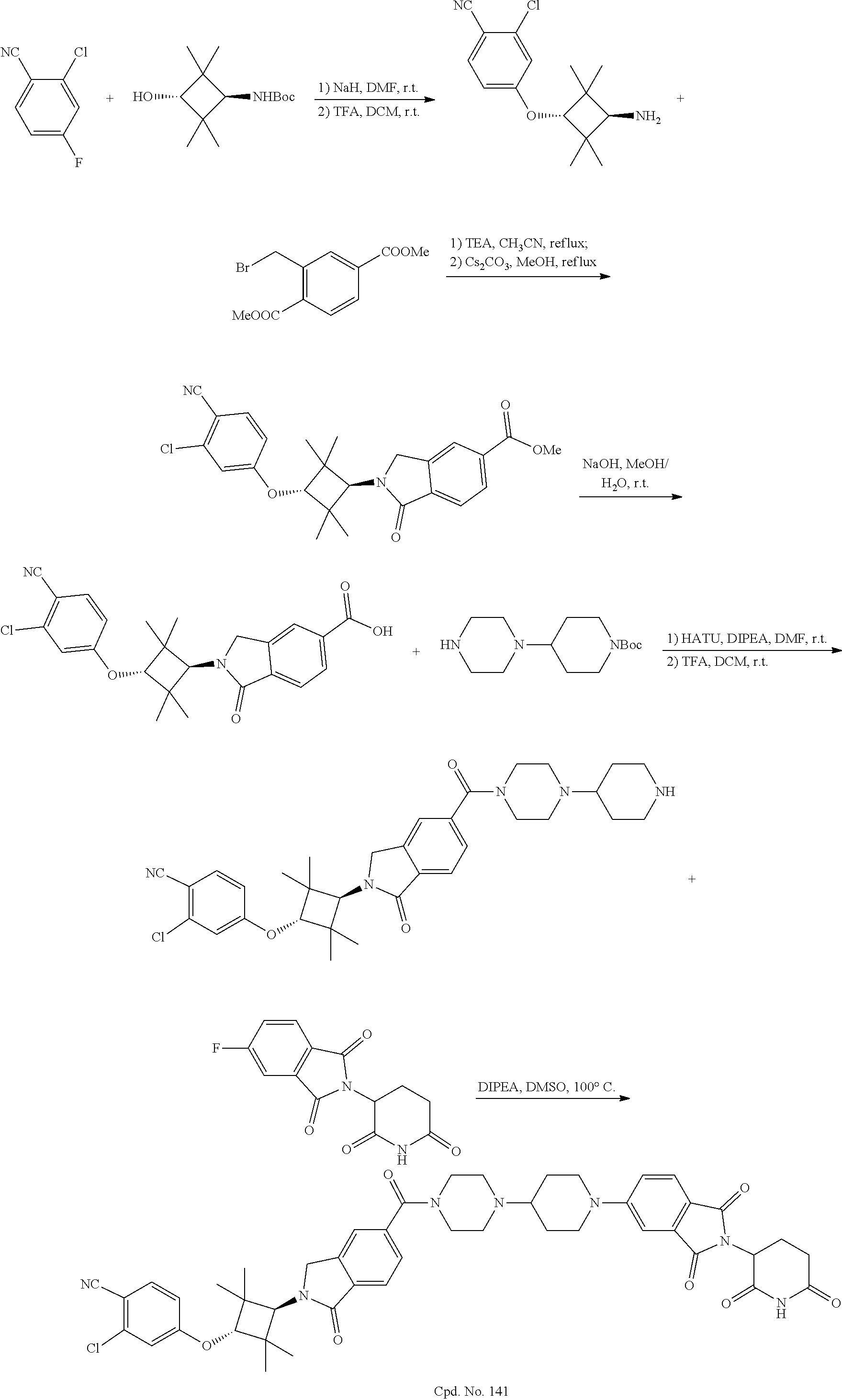

### Step 1: Synthesis of 4-((1r,3r)-3-Amino-2,2,4,4-tetramethylcyclobutoxy)-2-chlorobenzonitrile

To a solution of tert-butyl ((1r,3r)-3-hydroxy-2,2,4,4-tetramethylcyclobutyl)carbamate (2.43 g, 10 mmol) in dry DMF was added NaH (1.2 eq.) at 0° C. After stirring the mixture at 0° C. for 20 min, 2-chloro-4-fluorobenzonitrile was added and the mixture was stirred at room temperature for 4 h. After UPLC-MS demonstrated the full conversion of starting materials, H₂O was added, the mixture was extracted with EtOAc, and the combined organic layers were washed with brine and dried over anhydrous Na₂SO₄. The solvent was removed on a rotary evaporator. The desired intermediate 4-((1r,3r)-3-amino-2,2,4,4-tetramethylcyclobutoxy)-2-chlorobenzonitrile was obtained by deprotection with TFA in DCM in 88% yield. ESI-MS: 278.12.

### Step 2: Synthesis of methyl 2-((1r,3r)-3-(3-chloro-4-cyanophenoxy)-2,2,4,4-tetramethylcyclobutyl)-1-oxoisoindoline-5-carboxylate

Triethylamine (3 eq.) was added the mixture of 4-((1r,3r)-3-amino-2,2,4,4-tetramethylcyclobutoxy)-2-chlorobenzonitrile and dimethyl 2-(bromomethyl)terephthalate in CH₃CN. After the mixture was stirred at 100° C. for 10 h, MeOH and Cs₂CO₃ were added. After the mixture was stirred at 100° C. for another 10 h, the solvents were evaporated under reduced pressure to afford the corresponding crude methyl 2-((1r,3r)-3-(3-chloro-4-cyanophenoxy)-2,2,4,4-tetramethylcyclobutyl)-1-oxoisoindoline-5-carboxylate that was purified by flash column chromatography (DCM:MeOH=20:1) with 70% yield. ESI-MS: 452.15.

### Step 3: Synthesis of 2-((1r,3r)-3-(3-chloro-4-cyanophenoxy)-2,2,4,4-tetramethylcyclobutyl)-1-oxoisoindoline-5-carboxylic Acid

NaOH (2 eq.) was added to a solution of methyl 2-((1r,3r)-3-(3-chloro-4-cyanophenoxy)-2,2,4,4-tetramethylcyclobutyl)-1-oxoisoindoline-5-carboxylate in MeOH/H₂O and stirred at room temperature for 2 h. Then the MeOH was removed under reduced pressure, the pH was adjusted to acidity with 2M HCl, and the mixture was extracted with EtOAc. The solvent was removed to afford the product 2-((1r,3r)-3-(3-chloro-4-cyanophenoxy)-2,2,4,4-tetramethylcyclobutyl)-1-oxoisoindoline-5-carboxylic acid which was used without further purification. ESI-MS: 438.13.

### Step 4: Synthesis of 2-chloro-4-((1r,3r)-2,2,4,4-tetramethyl-3-(1-oxo-5-(4-(piperidin-4-yl)piperazine-1-carbonyl)isoindolin-2-yl)cyclobutoxy)benzonitrile

2-((1r,3r)-3-(3-Chloro-4-cyanophenoxy)-2,2,4,4-tetramethylcyclobutyl)-1-oxoisoindoline-5-carboxylic acid and tert-butyl 4-(piperazin-1-yl)piperidine-1-carboxylate were dissolved in DMF. To the solution was added DIPEA (5 eq.) and HATU (1.2 eq.), and the reaction mixture was stirred at room temperature for 1 h. The reaction mixture was extracted by EA, washed by water and organic phase was dried by Na₂SO₄. tert-Butyl 4-(4-(2-((1r,3r)-3-(3-chloro-4-cyanophenoxy)-2,2,4,4-tetramethylcyclobutyl)-1-oxoisoindoline-5-carbonyl)piperazin-1-yl)piperidine-1-carboxylate was obtained by removing the solvent under vacuum and purifying by flash column chromatography on silica gel. The desired intermediate 2-chloro-4-((1r,3r)-2,2,4,4-tetramethyl-3-(1-oxo-5-(4-(piperidin-4-yl)piperazine-1-carbonyl)isoindolin-2-yl)cyclobutoxy)benzonitrile was obtained by deprotection with TFA in DCM in 89% yield. ESI-MS: 589.28.

### Step 5: Synthesis of 2-chloro-4-((1r,3r)-3-(5-(4-(1-(2-(2,6-dioxopiperidin-3-yl)-1,3-dioxoisoindolin-5-yl)piperidin-4-yl)piperazine-1-carbonyl)-1-oxoisoindolin-2-yl)-2,2,4,4-tetramethylcyclobutoxy)benzonitrile (Cpd. No. 141)

DIPEA (5 eq.) was added to a solution of 2-chloro-4-((1r,3r)-2,2,4,4-tetramethyl-3-(1-oxo-5-(4-(piperidin-4-yl)piperazine-1-carbonyl)isoindolin-2-yl)cyclobutoxy)benzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 141 with 90% yield. ESI-MS: 845.33.

### Example 2

### Synthesis of 2-chloro-4-((1r,3r)-3-(5-(4-(1-(2-(2,6-dioxopiperidin-3-yl)-1,3-dioxoisoindolin-5-yl)azetidin-3-yl)piperazine-1-carbonyl)-1-oxoisoindolin-2-yl)-2,2,4,4-tetramethylcyclobutoxy)benzonitrile (Cpd. No. 140)

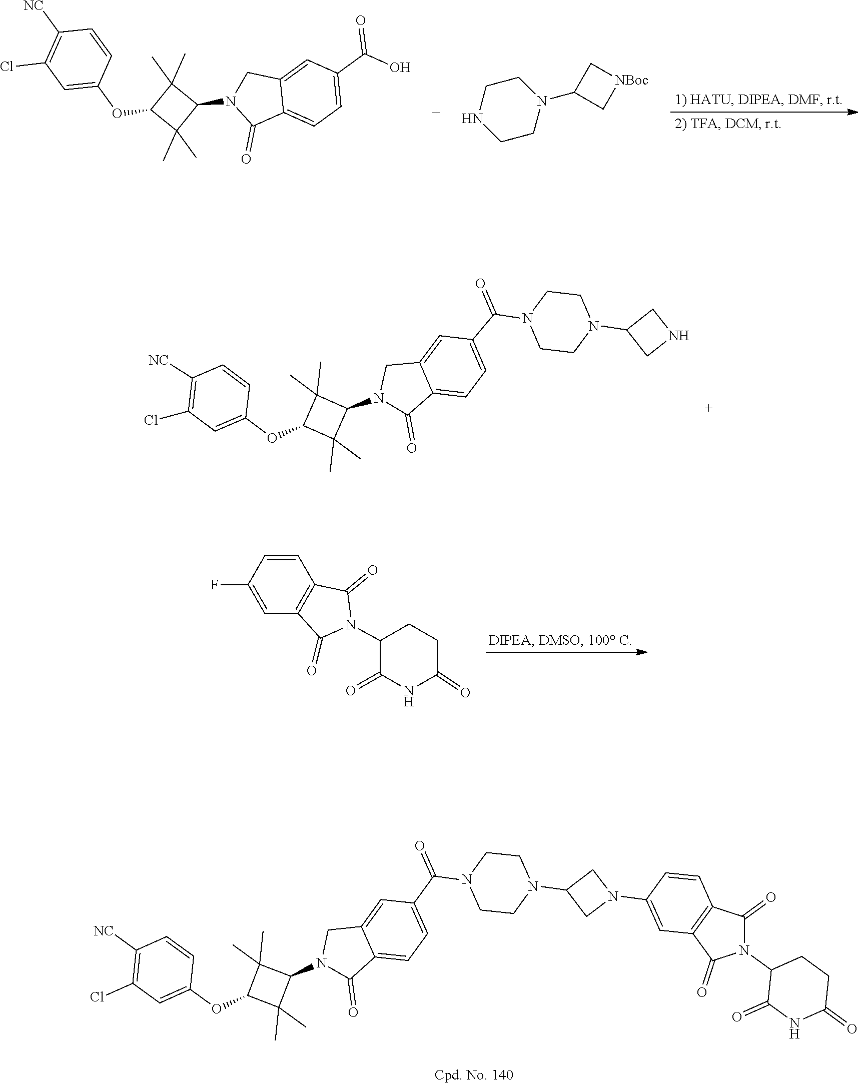 [SMILES: CC1(C)[C@H](Oc2ccc(C#N)c(Cl)c2)C(C)(C)[C@H]1N1Cc2cc(C(=O)O)ccc2C1=O ; CC(C)(C)OC(=O)N1CC(N2CCNCC2)C1 ; CC1(C)[C@H](Oc2ccc(C#N)c(Cl)c2)C(C)(C)[C@H]1N1Cc2cc(C(=O)N3CCN(C4CNC4)CC3)ccc2C1=O ; O=C1CCC(N2C(=O)c3ccc(F)cc3C2=O)C(=O)N1 ; CC1(C)[C@H](Oc2ccc(C#N)c(Cl)c2)C(C)(C)[C@H]1N1Cc2cc(C(=O)N3CCN(C4CN(c5ccc6c(c5)C(=O)N(C5CCC(=O)NC5=O)C6=O)C4)CC3)ccc2C1=O]

### Step 1: Synthesis of 4-((1r,3r)-3-(5-(4-(azetidin-3-yl)piperazine-1-carbonyl)-1-oxoisoindolin-2-yl)-2,2,4,4-tetramethylcyclobutoxy)-2-chlorobenzonitrile

2-((1r,3r)-3-(3-Chloro-4-cyanophenoxy)-2,2,4,4-tetramethylcyclobutyl)-1-oxoisoindoline-5-carboxylic acid and tert-butyl 3-(piperazin-1-yl)azetidine-1-carboxylate were dissolved in DMF. To the solution was added DIPEA (5 eq.) and HATU (1.2 eq.), and the reaction mixture was stirred at room temperature for 1 h. The reaction mixture was extracted by EA, washed by water and the organic phase was dried with Na₂SO₄. tert-Butyl 3-(4-(2-((1r,3r)-3-(3-chloro-4-cyanophenoxy)-2,2,4,4-tetramethylcyclobutyl)-1-oxoisoindoline-5-carbonyl)piperazin-1-yl)azetidine-1-carboxylate was obtained by removing the solvent under vacuum and purifying by flash column chromatography on silica gel. The desired intermediate 4-((1r,3r)-3-(5-(4-(azetidin-3-yl)piperazine-1-carbonyl)-1-oxoisoindolin-2-yl)-2,2,4,4-tetramethylcyclobutoxy)-2-chlorobenzonitrile was obtained by deprotection with TFA in DCM in 89% yield. ESI-MS: 561.25.

### Step 2: Synthesis of 2-chloro-4-((1r,3r)-3-(5-(4-(1-(2-(2,6-dioxopiperidin-3-yl)-1,3-dioxoisoindolin-5-yl)azetidin-3-yl)piperazine-1-carbonyl)-1-oxoisoindolin-2-yl)-2,2,4,4-tetramethylcyclobutoxy)benzonitrile (Cpd. No. 140)

DIPEA (5 eq.) was added to a solution of 4-((1r,3r)-3-(5-(4-(azetidin-3-yl)piperazine-1-carbonyl)-1-oxoisoindolin-2-yl)-2,2,4,4-tetramethylcyclobutoxy)-2-chlorobenzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 140 with 91% yield. ESI-MS: 845.33.

### Example 3

### Synthesis of 2-chloro-4-((1r,3r)-3-(5-((1′-(2-(2,6-dioxopiperidin-3-yl)-1,3-dioxoisoindolin-5-yl)-[1,3′-biazetidin]-3-yl)ethynyl)-1-oxoisoindolin-2-yl)-2,2,4,4-tetramethylcyclobutoxy)benzonitrile (Cpd. No. 154)

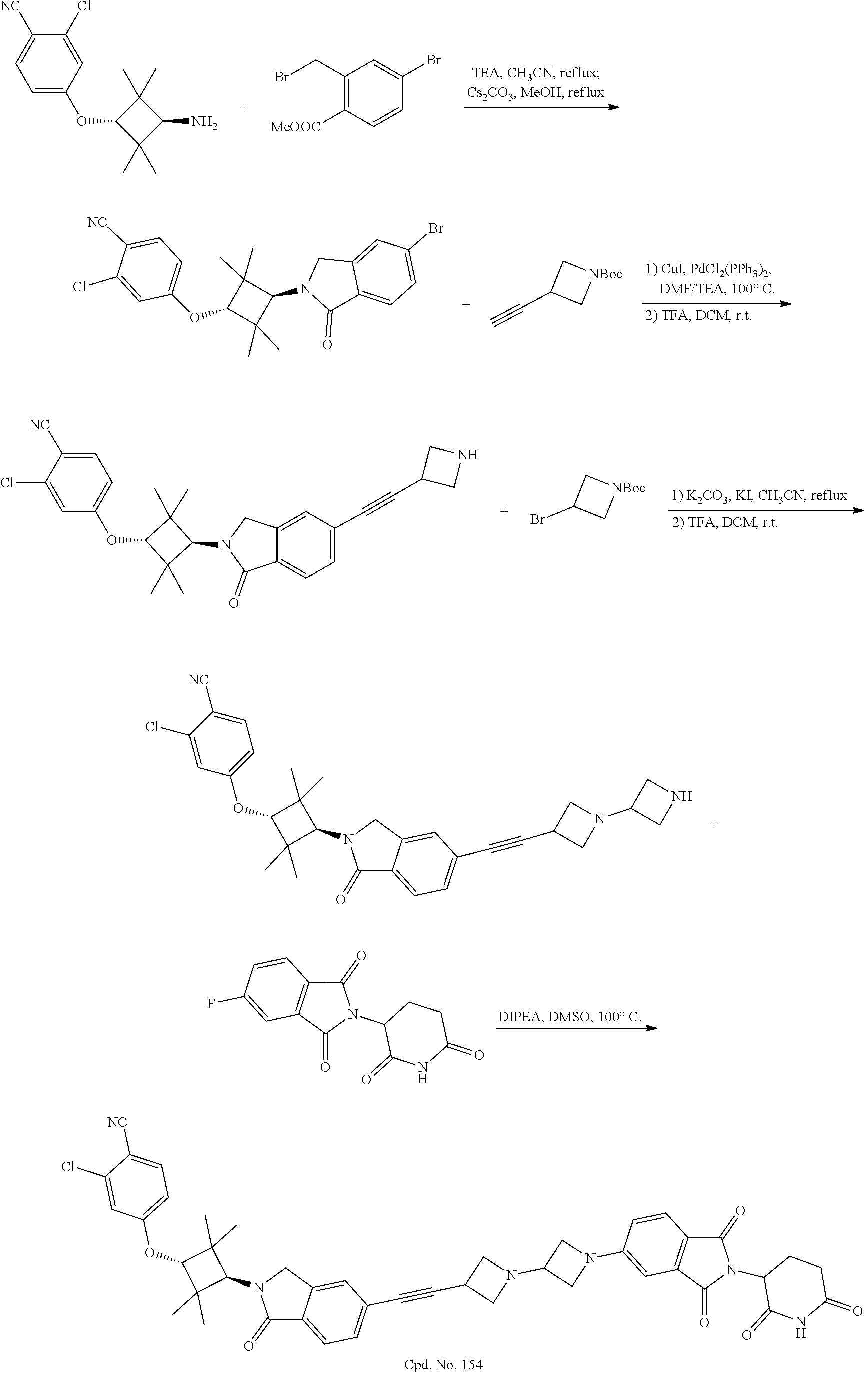

### Step 1: Synthesis of 4-((1r,3r)-3-(5-bromo-1-oxoisoindolin-2-yl)-2,2,4,4-tetramethylcyclobutoxy)-2-chlorobenzonitrile

Triethylamine (3 eq.) was added to a mixture of 4-((1r,3r)-3-amino-2,2,4,4-tetramethylcyclobutoxy)-2-chlorobenzonitrile and methyl 4-bromo-2-(bromomethyl)benzoate in CH₃CN. After the mixture was stirred at 100° C. for 10 h, MeOH and Cs₂CO₃ were added. After the mixture was stirred at 100° C. for another 10 h, the solvents were evaporated under reduced pressure to afford the corresponding crude 4-((1r,3r)-3-(5-bromo-1-oxoisoindolin-2-yl)-2,2,4,4-tetramethylcyclobutoxy)-2-chlorobenzonitrile that was purified by flash column chromatography (DCM:MeOH=20:1) with 70% yield. ESI-MS: 472.06.

### Step 2: Synthesis of 4-((1r,3r)-3-(5-(azetidin-3-ylethynyl)-1-oxoisoindolin-2-yl)-2,2,4,4-tetramethylcyclobutoxy)-2-chlorobenzonitrile

4-((1r,3r)-3-(5-Bromo-1-oxoisoindolin-2-yl)-2,2,4,4-tetramethylcyclobutoxy)-2-chlorobenzonitrile, tert-butyl 3-ethynylazetidine-1-carboxylate (1.1 eq.), CuI (0.2 eq.), PdCl₂(PPh₃)₂(0.1 eq.) in DMF/TEA were placed in a 25 mL round bottom flask under Ar. The mixture was stirred for 4 h at 100° C. Then H₂O was added into the resulting complex which was extracted with EtOAc three times. The organic layer was again washed with H₂O before being dried over MgSO₄ and the solvent was removed under vacuum leaving the crude product. The pure product was obtained by flash column chromatography (DCM:MeOH=20:1). 4-((1r,3r)-3-(5-(Azetidin-3-ylethynyl)-1-oxoisoindolin-2-yl)-2,2,4,4-tetramethylcyclobutoxy)-2-chlorobenzonitrile was obtained through the deprotection by TFA in DCM (85% yield). ESI-MS: 473.19.

### Step 3: Synthesis of 4-((1r,3r)-3-(5-([1,3′-biazetidin]-3-ylethynyl)-1-oxoisoindolin-2-yl)-2,2,4,4-tetramethylcyclobutoxy)-2-chlorobenzonitrile

K₂CO₃ (1.2 equiv) and KI (0.2 equiv) were added to a solution of the intermediate 4-((1r,3r)-3-(5-(azetidin-3-ylethynyl)-1-oxoisoindolin-2-yl)-2,2,4,4-tetramethylcyclobutoxy)-2-chlorobenzonitrile and tert-butyl 3-bromoazetidine-1-carboxylate (1.2 eq.) in CH₃CN. After stirring the mixture overnight at 100° C., the solvents were evaporated under reduced pressure to afford the corresponding crude compound that was purified by flash column chromatography (DCM:MeOH=20:1). Then, 4-((1r,3r)-3-(5-([1,3′-biazetidin]-3-ylethynyl)-1-oxoisoindolin-2-yl)-2,2,4,4-tetramethylcyclobutoxy)-2-chlorobenzonitrile was obtained through the deprotection by TFA in DCM (85% yield). ESI-MS: 528.23.

### Step 4: Synthesis of 2-chloro-4-((1r,3r)-3-(5-((1′-(2-(2,6-dioxopiperidin-3-yl)-1,3-dioxoisoindolin-5-yl)-[1,3′-biazetidin]-3-yl)ethynyl)-1-oxoisoindolin-2-yl)-2,2,4,4-tetramethylcyclobutoxy)benzonitrile (Cpd. No. 154)

DIPEA (5 eq.) was added to a solution of 4-((1r,3r)-3-(5-([1,3′-biazetidin]-3-ylethynyl)-1-oxoisoindolin-2-yl)-2,2,4,4-tetramethylcyclobutoxy)-2-chlorobenzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 154 with 90% yield. ESI-MS: 784.28.

### Example 4

### Synthesis of 2-chloro-4-((1r,3r)-3-(5-((1-(1-(2-(2,6-dioxopiperidin-3-yl)-1,3-dioxoisoindolin-5-yl)piperidin-4-yl)azetidin-3-yl)ethynyl)-1-oxoisoindolin-2-yl)-2,2,4,4-tetramethylcyclobutoxy)benzonitrile (Cpd. No. 150)

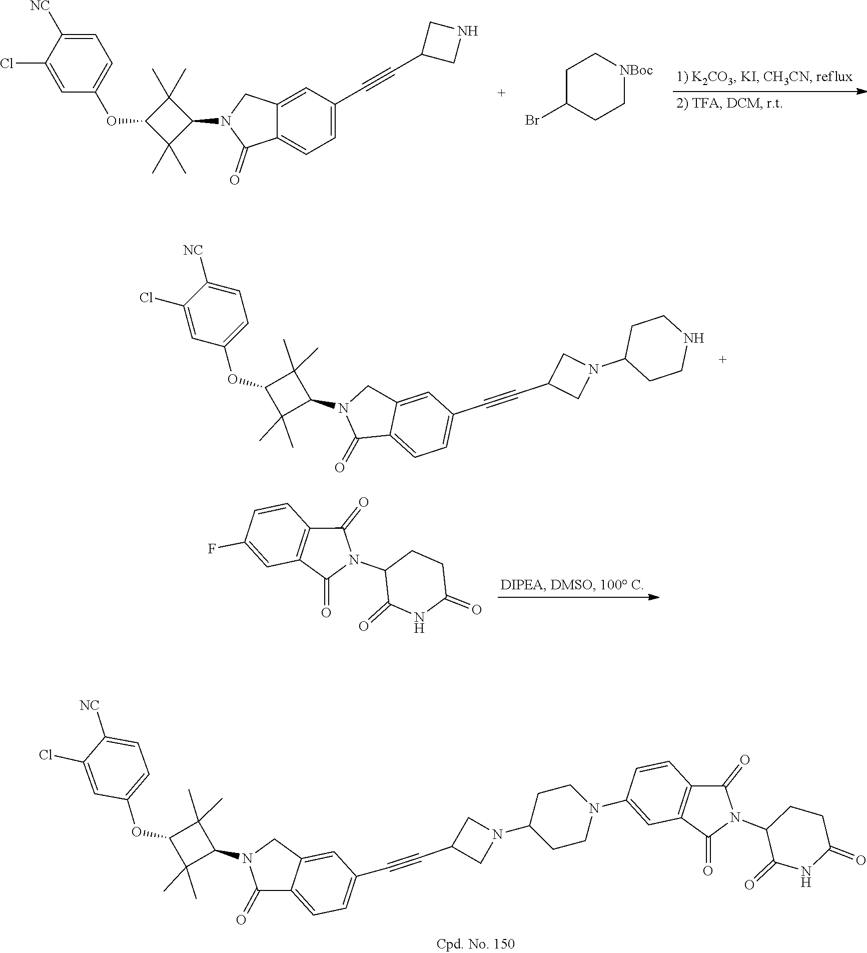

### Step 1: Synthesis of 2-chloro-4-((1r,3r)-2,2,4,4-tetramethyl-3-(1-oxo-5-((1-(piperidin-4-yl)azetidin-3-yl)ethynyl)isoindolin-2-yl)cyclobutoxy)benzonitrile

K₂CO₃ (1.2 equiv) and KI (0.2 equiv) were added to a solution of the intermediate 4-((1r,3r)-3-(5-(azetidin-3-ylethynyl)-1-oxoisoindolin-2-yl)-2,2,4,4-tetramethylcyclobutoxy)-2-chlorobenzonitrile and tert-butyl 4-bromopiperidine-1-carboxylate (1.2 eq.) in CH₃CN. After stirring the mixture overnight at 100° C., the solvents were evaporated under reduced pressure to afford the corresponding crude compound that was purified by flash column chromatography (DCM:MeOH=20:1). Then, 2-chloro-4-((1r,3r)-2,2,4,4-tetramethyl-3-(1-oxo-5-((1-(piperidin-4-yl)azetidin-3-yl)ethynyl)isoindolin-2-yl)cyclobutoxy)benzonitrile was obtained through the deprotection by TFA in DCM (88% yield). ESI-MS: 556.26.

### Step 2: Synthesis of 2-chloro-4-((1r,3r)-3-(5-((1-(1-(2-(2,6-dioxopiperidin-3-yl)-1,3-dioxoisoindolin-5-yl)piperidin-4-yl)azetidin-3-yl)ethynyl)-1-oxoisoindolin-2-yl)-2,2,4,4-tetramethylcyclobutoxy)benzonitrile (Cpd. No. 150)

DIPEA (5 eq.) was added to a solution of 2-chloro-4-((1r,3r)-2,2,4,4-tetramethyl-3-(1-oxo-5-((1-(piperidin-4-yl)azetidin-3-yl)ethynyl)isoindolin-2-yl)cyclobutoxy)benzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 150 with 80% yield. ESI-MS: 812.31.

### Example 5

### Synthesis of 2-chloro-4-((1r,3r)-3-(5-((1-(1-(2-(2,6-dioxopiperidin-3-yl)-1,3-dioxoisoindolin-5-yl)azetidin-3-yl)piperidin-4-yl)ethynyl)-1-oxoisoindolin-2-yl)-2,2,4,4-tetramethylcyclobutoxy)benzonitrile (Cpd. No. 142)

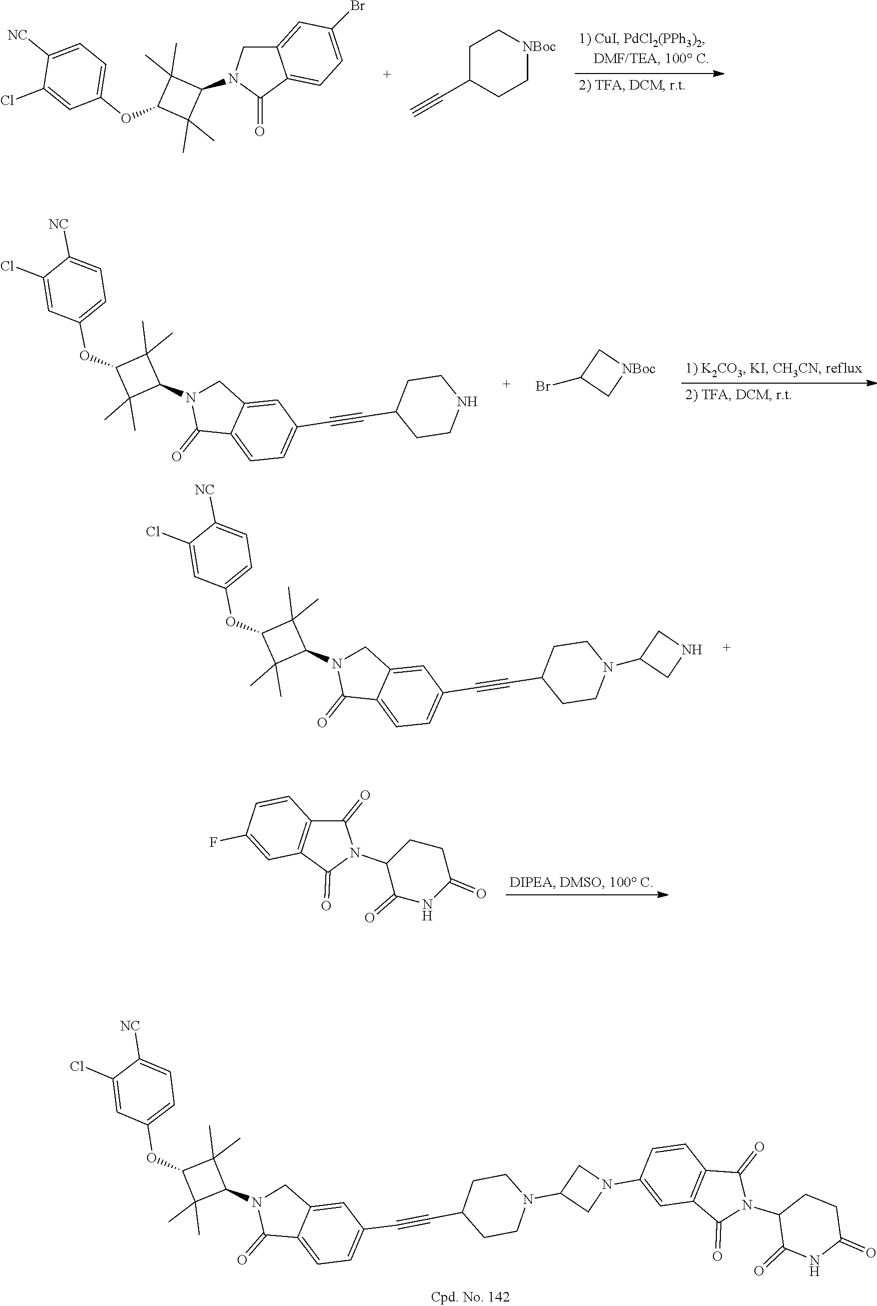

### Step 1: Synthesis of 2-chloro-4-((1r,3r)-2,2,4,4-tetramethyl-3-(1-oxo-5-(piperidin-4-ylethynyl)isoindolin-2-yl)cyclobutoxy)benzonitrile

4-((1r,3r)-3-(5-Bromo-1-oxoisoindolin-2-yl)-2,2,4,4-tetramethylcyclobutoxy)-2-chlorobenzonitrile and tert-butyl 4-ethynylpiperidine-1-carboxylate (1.1 eq.), CuI (0.2 eq.), PdCl₂(PPh₃)₂(0.1 eq.) in DMF and TEA solvent were placed in a 25 mL round bottom flask under Ar. Then the mixture was stirred for 4 h at 100° C. Then H₂O was added into the resulting complex which was extracted with EtOAc three times. The organic layer was again washed with H₂O before being dried over MgSO₄ and the solvent was removed under vacuum leaving the crude product. The pure product was obtained by flash column chromatography (DCM:MeOH=20:1). Then, 2-chloro-4-((1r,3r)-2,2,4,4-tetramethyl-3-(1-oxo-5-(piperidin-4-ylethynyl)isoindolin-2-yl)cyclobutoxy)benzonitrile was obtained through the deprotection by TFA in DCM (80% yield). ESI-MS: 501.22.

### Step 2: Synthesis of 4-((1r,3r)-3-(5-((1-(azetidin-3-yl)piperidin-4-yl)ethynyl)-1-oxoisoindolin-2-yl)-2,2,4,4-tetramethylcyclobutoxy)-2-chlorobenzonitrile

K₂CO₃ (1.2 equiv) and KI (0.2 equiv) were added to a solution of the intermediate 2-chloro-4-((1r,3r)-2,2,4,4-tetramethyl-3-(1-oxo-5-(piperidin-4-ylethynyl)isoindolin-2-yl)cyclobutoxy)benzonitrile and tert-butyl 3-bromoazetidine-1-carboxylate (1.2 eq.) in CH₃CN. After stirring the mixture overnight at 100° C., the solvents were evaporated under reduced pressure to afford the corresponding crude compound that was purified by flash column chromatography (DCM:MeOH=20:1). Then, 4-((1r,3r)-3-(5-((1-(azetidin-3-yl)piperidin-4-yl)ethynyl)-1-oxoisoindolin-2-yl)-2,2,4,4-tetramethylcyclobutoxy)-2-chlorobenzonitrile was obtained through the deprotection by TFA in DCM (87% yield). ESI-MS: 556.26.

### Step 3: Synthesis of 2-chloro-4-((1r,3r)-3-(5-((1-(1-(2-(2,6-dioxopiperidin-3-yl)-1,3-dioxoisoindolin-5-yl)azetidin-3-yl)piperidin-4-yl)ethynyl)-1-oxoisoindolin-2-yl)-2,2,4,4-tetramethylcyclobutoxy)benzonitrile (Cpd. No. 142)

DIPEA (5 eq.) was added to a solution of 4-((1r,3r)-3-(5-((1-(azetidin-3-yl)piperidin-4-yl)ethynyl)-1-oxoisoindolin-2-yl)-2,2,4,4-tetramethylcyclobutoxy)-2-chlorobenzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 142 with 80% yield. ESI-MS: 812.31.

### Example 6

### Synthesis of 2-chloro-4-((1r,3r)-3-(5-((1-(2-(2,6-dioxopiperidin-3-yl)-1,3-dioxoisoindolin-5-yl)piperidin-4-yl)ethynyl)-1-oxoisoindolin-2-yl)-2,2,4,4-tetramethylcyclobutoxy)benzonitrile (Cpd. No. 155)

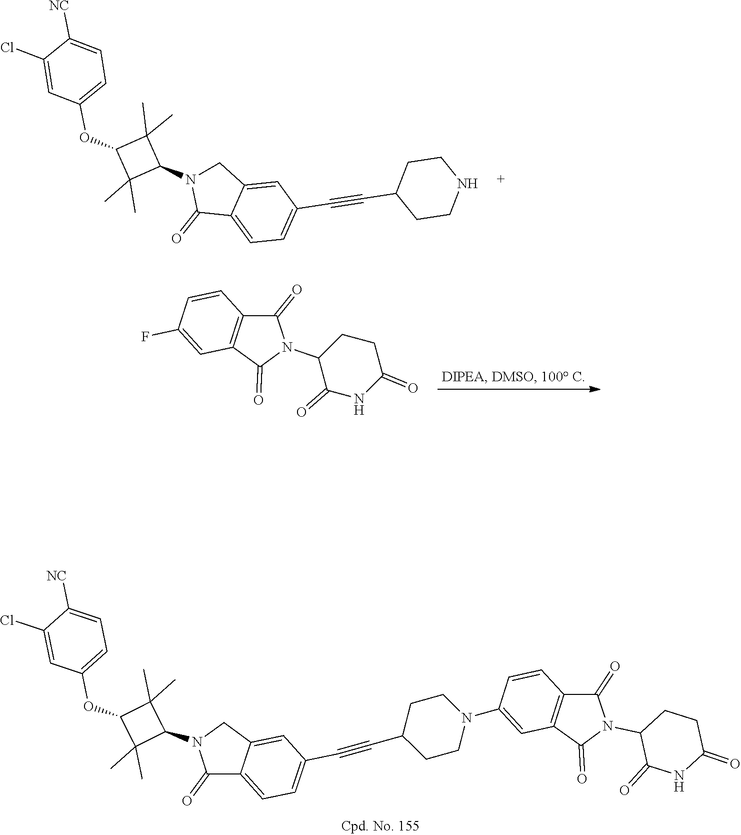 [SMILES: CC1(C)[C@H](Oc2ccc(C#N)c(Cl)c2)C(C)(C)[C@H]1N1Cc2cc(C#CC3CCNCC3)ccc2C1=O ; O=C1CCC(N2C(=O)c3ccc(F)cc3C2=O)C(=O)N1 ; CC1(C)[C@H](Oc2ccc(C#N)c(Cl)c2)C(C)(C)[C@H]1N1Cc2cc(C#CC3CCN(c4ccc5c(c4)C(=O)N(C4CCC(=O)NC4=O)C5=O)CC3)ccc2C1=O]

DIPEA (5 eq.) was added to a solution of 2-chloro-4-((1r,3r)-2,2,4,4-tetramethyl-3-(1-oxo-5-(piperidin-4-ylethynyl)isoindolin-2-yl)cyclobutoxy)benzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 155 with 80% yield. ESI-MS: 757.27.

### Example 7

### Synthesis of 2-chloro-4-((1r,3r)-3-(5-(4-((1-(2-(2,6-dioxopiperidin-3-yl)-1,3-dioxoisoindolin-5-yl)piperidin-4-yl)methyl)piperazine-1-carbonyl)-1-oxoisoindolin-2-yl)-2,2,4,4-tetramethylcyclobutoxy)benzonitrile (Cpd. No. 143)

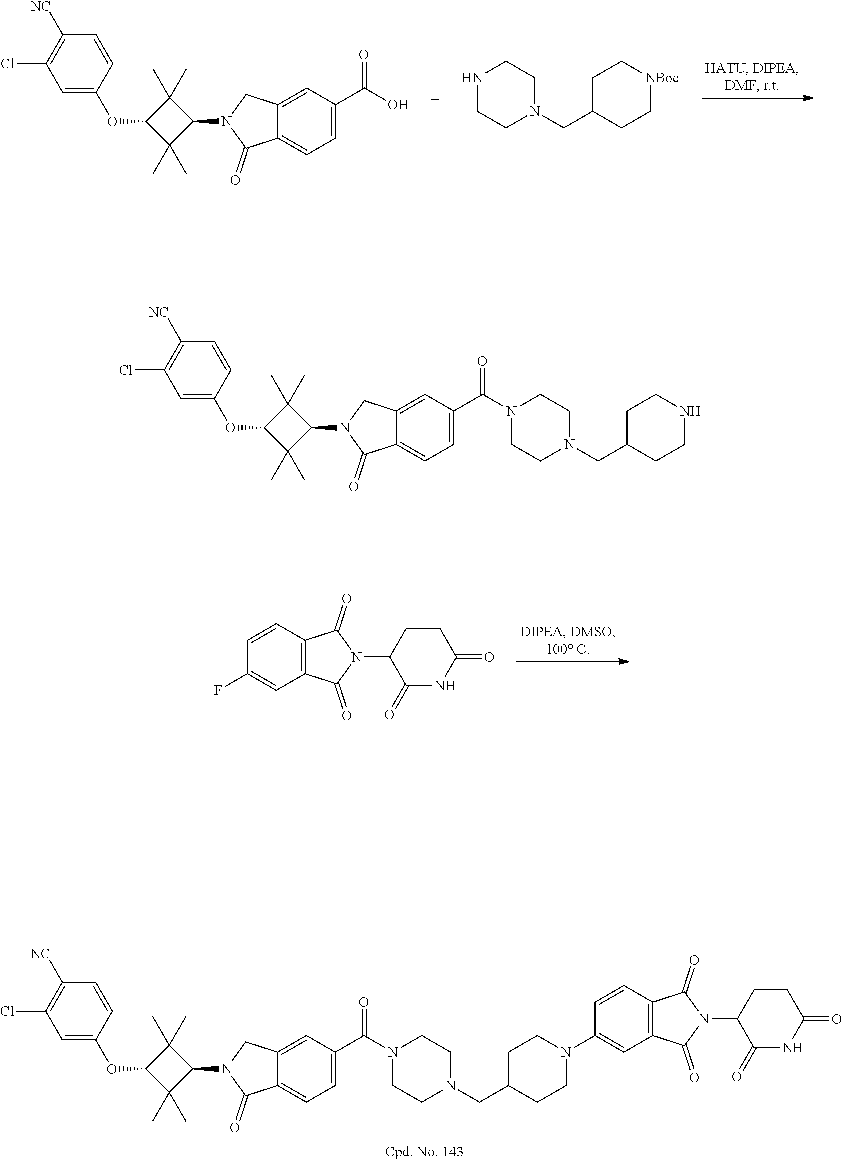 [SMILES: CC1(C)[C@H](Oc2ccc(C#N)c(Cl)c2)C(C)(C)[C@H]1N1Cc2cc(C(=O)O)ccc2C1=O ; CC(C)(C)OC(=O)N1CCC(CN2CCNCC2)CC1 ; CC1(C)[C@H](Oc2ccc(C#N)c(Cl)c2)C(C)(C)[C@H]1N1Cc2cc(C(=O)N3CCN(CC4CCNCC4)CC3)ccc2C1=O ; O=C1CCC(N2C(=O)c3ccc(F)cc3C2=O)C(=O)N1 ; CC1(C)[C@H](Oc2ccc(C#N)c(Cl)c2)C(C)(C)[C@H]1N1Cc2cc(C(=O)N3CCN(CC4CCN(c5ccc6c(c5)C(=O)N(C5CCC(=O)NC5=O)C6=O)CC4)CC3)ccc2C1=O]

### Step 1: Synthesis of 2-chloro-4-((1r,3r)-2,2,4,4-tetramethyl-3-(1-oxo-5-(4-(piperidin-4-ylmethyl)piperazine-1-carbonyl)isoindolin-2-yl)cyclobutoxy)benzonitrile

2-((1r,3r)-3-(3-Chloro-4-cyanophenoxy)-2,2,4,4-tetramethylcyclobutyl)-1-oxoisoindoline-5-carboxylic acid and tert-butyl 4-(piperazin-1-ylmethyl)piperidine-1-carboxylate were dissolved in DMF. The solution was added DIPEA (5 eq.) and HATU (1.2 eq.), the reaction mixture was stirred at room temperature for 1 h. The reaction mixture was extracted by EA, washed by water and organic phase was dried by Na₂SO₄. tert-Butyl 3-(4-(2-((1r,3r)-3-(3-chloro-4-cyanophenoxy)-2,2,4,4-tetramethylcyclobutyl)-1-oxoisoindoline-5-carbonyl)piperazin-1-yl)azetidine-1-carboxylate was obtained by removing the solvent under vacuum and purifying by flash column chromatography on silica gel. The desired intermediate 2-chloro-4-((1r,3r)-2,2,4,4-tetramethyl-3-(1-oxo-5-(4-(piperidin-4-ylmethyl)piperazine-1-carbonyl)isoindolin-2-yl)cyclobutoxy)benzonitrile was obtained by deprotection with TFA in DCM in 89% yield. ESI-MS: 603.30.

### Step 2: Synthesis of 2-chloro-4-((1r,3r)-3-(5-(4-((1-(2-(2,6-dioxopiperidin-3-yl)-1,3-dioxoisoindolin-5-yl)piperidin-4-yl)methyl)piperazine-1-carbonyl)-1-oxoisoindolin-2-yl)-2,2,4,4-tetramethylcyclobutoxy)benzonitrile (Cpd. No. 143)

DIPEA (5 eq.) was added to a solution of 2-chloro-4-((1r,3r)-2,2,4,4-tetramethyl-3-(1-oxo-5-(4-(piperidin-4-ylmethyl)piperazine-1-carbonyl)isoindolin-2-yl)cyclobutoxy)benzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 143 with 86% yield. ESI-MS: 859.35.

### Example 8

### Synthesis of 2-chloro-4-((1r,3r)-3-(5-(4-((1-(2-(2,6-dioxopiperidin-3-yl)-1,3-dioxoisoindolin-5-yl)azetidin-3-yl)methyl)piperazine-1-carbonyl)-1-oxoisoindolin-2-yl)-2,2,4,4-tetramethylcyclobutoxy)benzonitrile (Cpd. No. 144)

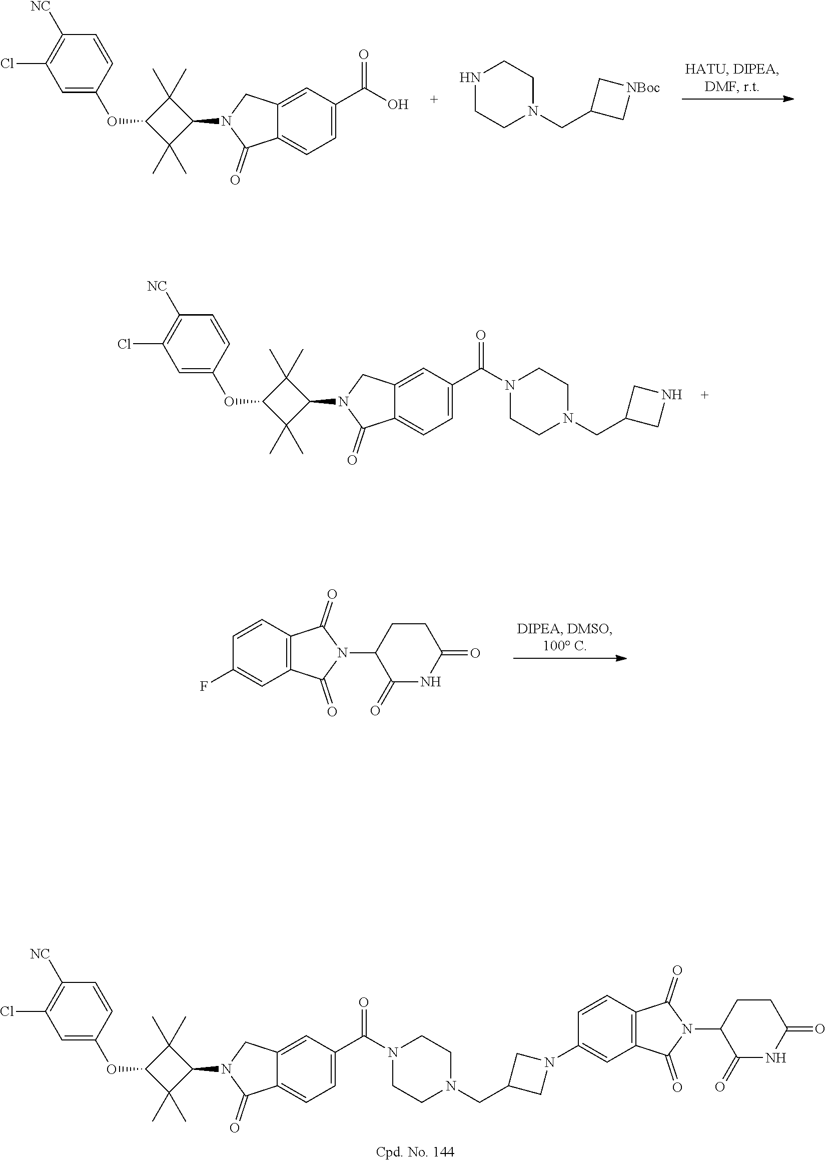 [SMILES: CC1(C)[C@H](Oc2ccc(C#N)c(Cl)c2)C(C)(C)[C@H]1N1Cc2cc(C(=O)O)ccc2C1=O ; CC(C)(C)OC(=O)N1CC(CN2CCNCC2)C1 ; CC1(C)[C@H](Oc2ccc(C#N)c(Cl)c2)C(C)(C)[C@H]1N1Cc2cc(C(=O)N3CCN(CC4CNC4)CC3)ccc2C1=O ; O=C1CCC(N2C(=O)c3ccc(F)cc3C2=O)C(=O)N1 ; CC1(C)[C@H](Oc2ccc(C#N)c(Cl)c2)C(C)(C)[C@H]1N1Cc2cc(C(=O)N3CCN(CC4CN(c5ccc6c(c5)C(=O)N(C5CCC(=O)NC5=O)C6=O)C4)CC3)ccc2C1=O]

### Step 1: Synthesis of 4-((1r,3r)-3-(5-(4-(azetidin-3-ylmethyl)piperazine-1-carbonyl)-1-oxoisoindolin-2-yl)-2,2,4,4-tetramethylcyclobutoxy)-2-chlorobenzonitrile

2-((1r,3r)-3-(3-Chloro-4-cyanophenoxy)-2,2,4,4-tetramethylcyclobutyl)-1-oxoisoindoline-5-carboxylic acid and tert-butyl 3-(piperazin-1-ylmethyl)azetidine-1-carboxylate were dissolved in DMF. The solution was added DIPEA (5 eq.) and HATU (1.2 eq.), the reaction mixture was stirred at room temperature for 1 h. The reaction mixture was extracted by EA, washed by water and organic phase was dried by Na₂SO₄. tert-Butyl 3-(4-(2-((1r,3r)-3-(3-chloro-4-cyanophenoxy)-2,2,4,4-tetramethylcyclobutyl)-1-oxoisoindoline-5-carbonyl)piperazin-1-yl)azetidine-1-carboxylate was obtained by removing the solvent under vacuum and purifying by flash column chromatography on silica gel. The desired intermediate 4-((1r,3r)-3-(5-(4-(azetidin-3-ylmethyl)piperazine-1-carbonyl)-1-oxoisoindolin-2-yl)-2,2,4,4-tetramethylcyclobutoxy)-2-chlorobenzonitrile was obtained by deprotection with TFA in DCM in 92% yield. ESI-MS: 575.27.

### Step 2: Synthesis of 2-chloro-4-((1r,3r)-3-(5-(4-((1-(2-(2,6-dioxopiperidin-3-yl)-1,3-dioxoisoindolin-5-yl)azetidin-3-yl)methyl)piperazine-1-carbonyl)-1-oxoisoindolin-2-yl)-2,2,4,4-tetramethylcyclobutoxy)benzonitrile (Cpd. No. 144)

DIPEA (5 eq.) was added to a solution of 4-((1r,3r)-3-(5-(4-(azetidin-3-ylmethyl)piperazine-1-carbonyl)-1-oxoisoindolin-2-yl)-2,2,4,4-tetramethylcyclobutoxy)-2-chlorobenzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 144 with 80% yield. ESI-MS: 831.31.

### Example 9

### Synthesis of 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)benzonitrile (Cpd. No. 190)

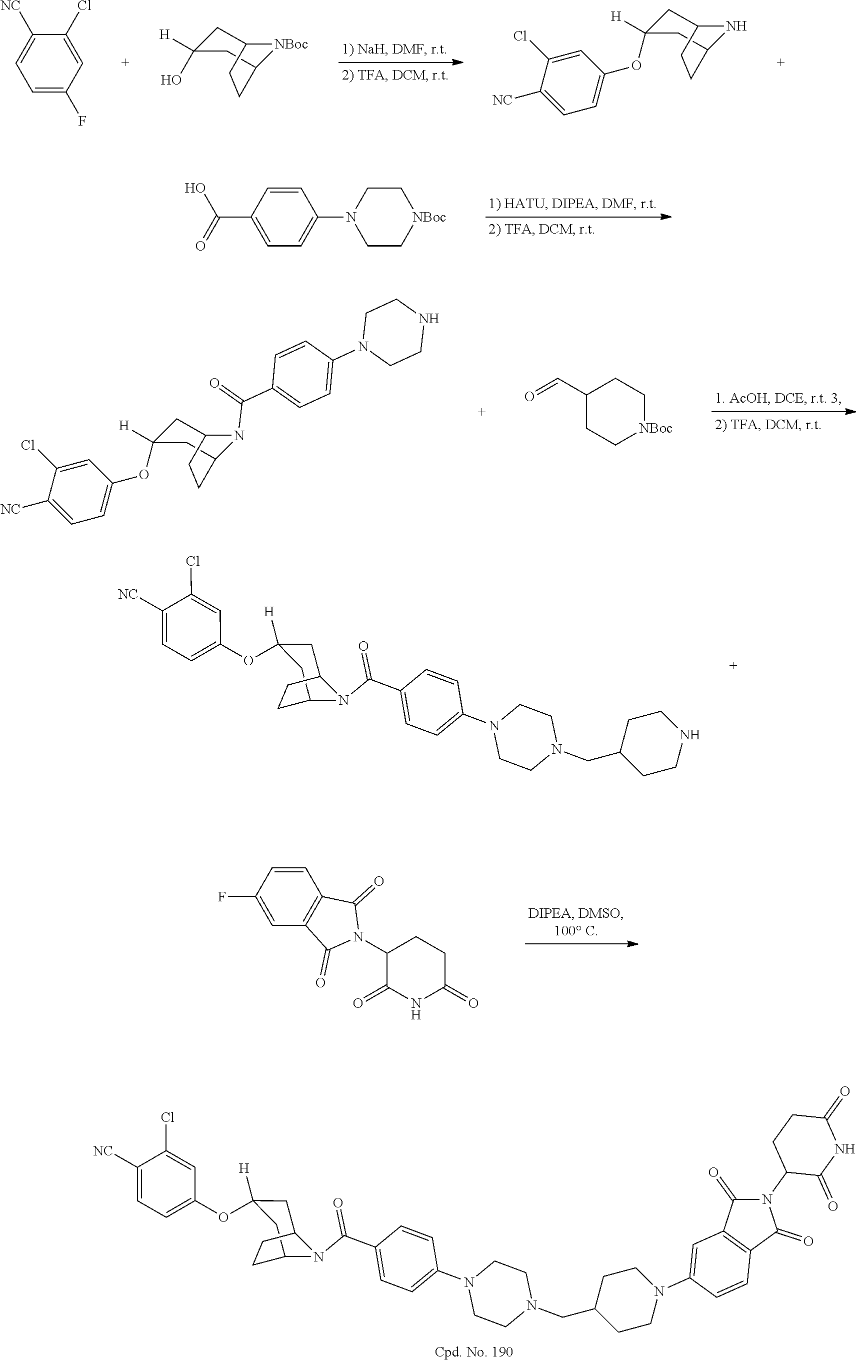

### Step 1: Synthesis of 4-(((1R,3r,5S)-8-azabicyclo[3.2.1]octan-3-yl)oxy)-2-chlorobenzonitrile

To a solution of tert-butyl (1R,3r,5S)-3-hydroxy-8-azabicyclo[3.2.1]octane-8-carboxylate (2.27 g, 10 mmol) in dry DMF was added NaH (1.2 eq.) at 0° C. After stirring the mixture at 0° C. for 20 min, 2-chloro-4-fluorobenzonitrile was added and the mixture was stirred at room temperature for 4 h. After UPLC-MS demonstrated the full conversion of starting materials, H₂O was added and the mixture was extracted with EtOAc, the combined organic layers were washed with brine, then dried over anhydrous Na₂SO₄. The solvent was removed on a rotary evaporator. The desired intermediate 4-(((1R,3r,5S)-8-azabicyclo[3.2.1]octan-3-yl)oxy)-2-chlorobenzonitrile was obtained by deprotection with TFA in DCM in 90% yield. ESI-MS: 262.09.

### Step 2: Synthesis of 2-chloro-4-(((1R,3r,5S)-8-(4-(piperazin-1-yl)benzoyl)-8-azabicyclo[3.2.1]octan-3-yl)oxy)benzonitrile

4-(((1R,3r,5S)-8-Azabicyclo[3.2.1]octan-3-yl)oxy)-2-chlorobenzonitrile and 4-(4-(tert-butoxycarbonyl)piperazin-1-yl)benzoic acid were dissolved in DMF. The solution was added DIPEA (5 eq.) and HATU (1.2 eq.), the reaction mixture was stirred at room temperature for 1 h. The reaction mixture was extracted by EA, washed by water and organic phase was dried by Na₂SO₄. tert-Butyl 4-(4-(2-((1r,3r)-3-(3-chloro-4-cyanophenoxy)-2,2,4,4-tetramethylcyclobutyl)-1-oxoisoindoline-5-carbonyl)piperazin-1-yl)piperidine-1-carboxylate was obtained by removing the solvent under vacuum and purifying by flash column chromatography on silica gel. The desired intermediate 2-chloro-4-(((1R,3r,5S)-8-(4-(piperazin-1-yl)benzoyl)-8-azabicyclo[3.2.1]octan-3-yl)oxy)benzonitrile was obtained by deprotection with TFA in DCM in 87% yield. ESI-MS: 450.18.

### Step 3: Synthesis of 2-chloro-4-(((1R,3r,5S)-8-(4-(4-(piperidin-4-ylmethyl)piperazin-1-yl)benzoyl)-8-azabicyclo[3.2.1]octan-3-yl)oxy)benzonitrile

To a solution of 2-chloro-4-(((1R,3r,5S)-8-(4-(piperazin-1-yl)benzoyl)-8-azabicyclo[3.2.1]octan-3-yl)oxy)benzonitrile and tert-butyl 4-formylpiperidine-1-carboxylate in DCE was added NaBH(OAc)₃ (1.5 eq.), AcOH and TEA. The reaction mixture was stirred at room temperature for 6 h. All volatiles were removed and the residue was chromatographed on silica gel to afford intermediate 2-chloro-4-(((1R,3r,5S)-8-(4-(4-(piperidin-4-ylmethyl)piperazin-1-yl)benzoyl)-8-azabicyclo[3.2.1]octan-3-yl)oxy)benzonitrile after removing the Boc group. ESI-MS: 547.27.

### Step 4: Synthesis of 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)benzonitrile (Cpd. No. 190)

DIPEA (5 eq.) was added to a solution of 2-chloro-4-((1r,3r)-2,2,4,4-tetramethyl-3-(1-oxo-5-(4-(piperidin-4-yl)piperazine-1-carbonyl)isoindolin-2-yl)cyclobutoxy)benzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 190 with 80% yield. ESI-MS: 803.32.

### Example 10

### Synthesis of 5-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)-3-(trifluoromethyl)picolinonitrile (Cpd. No. 7)

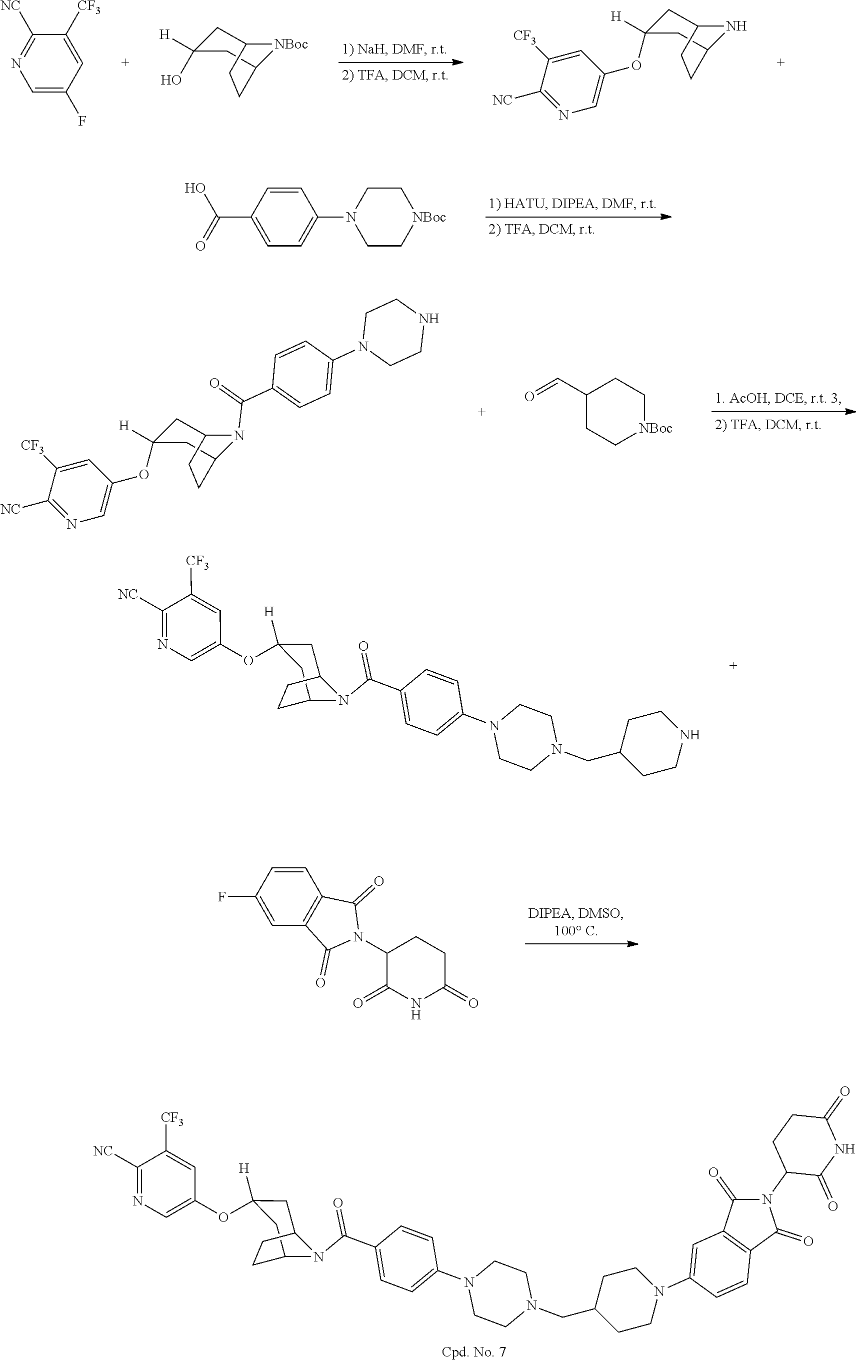

### Step 1: Synthesis of 5-(((1R,3r,5S)-8-azabicyclo[3.2.1]octan-3-yl)oxy)-3-(trifluoromethyl)picolinonitrile

To a solution of tert-butyl (1R,3r,5S)-3-hydroxy-8-azabicyclo[3.2.1]octane-8-carboxylate (2.27 g, 10 mmol) in dry DMF was added NaH (1.2 eq.) at 0° C. After stirring the mixture at 0° C. for 20 min, 5-fluoro-3-(trifluoromethyl)picolinonitrile was added and the mixture was stirred at room temperature for 4 h. After UPLC-MS demonstrated the full conversion of starting materials, H₂O was added and the mixture was extracted with EtOAc, the combined organic layers were washed with brine, then dried over anhydrous Na₂SO₄. The solvent was removed on a rotary evaporator. The desired intermediate 5-(((1R,3r,5S)-8-azabicyclo[3.2.1]octan-3-yl)oxy)-3-(trifluoromethyl)picolinonitrile was obtained by deprotection with TFA in DCM in 90% yield. ESI-MS: 297.11.

### Step 2: Synthesis of 5-(((1R,3r,5S)-8-(4-(piperazin-1-yl)benzoyl)-8-azabicyclo[3.2.1]octan-3-yl)oxy)-3-(trifluoromethyl)picolinonitrile

5-(((1R,3r,5S)-8-Azabicyclo[3.2.1]octan-3-yl)oxy)-3-(trifluoromethyl)picolinonitrile and 4-(4-(tert-butoxycarbonyl)piperazin-1-yl)benzoic acid were dissolved in DMF. The solution was added DIPEA (5 eq.) and HATU (1.2 eq.), the reaction mixture was stirred at room temperature for 1 h. The reaction mixture was extracted by EA, washed by water and organic phase was dried by Na₂SO₄. tert-Butyl 4-(4-(2-((1r,3r)-3-(3-chloro-4-cyanophenoxy)-2,2,4,4-tetramethylcyclobutyl)-1-oxoisoindoline-5-carbonyl)piperazin-1-yl)piperidine-1-carboxylate was obtained by removing the solvent under vacuum and purifying by flash column chromatography on silica gel. The desired intermediate 5-(((1R,3r,5S)-8-(4-(piperazin-1-yl)benzoyl)-8-azabicyclo[3.2.1]octan-3-yl)oxy)-3-(trifluoromethyl)picolinonitrile was obtained by deprotection with TFA in DCM in 86% yield. ESI-MS: 485.20.

### Step 3: Synthesis of 5-(((1R,3r,5S)-8-(4-(4-(piperidin-4-ylmethyl)piperazin-1-yl)benzoyl)-8-azabicyclo[3.2.1]octan-3-yl)oxy)-3-(trifluoromethyl)picolinonitrile

To a solution of 5-(((1R,3r,5S)-8-(4-(piperazin-1-yl)benzoyl)-8-azabicyclo[3.2.1]octan-3-yl)oxy)-3-(trifluoromethyl)picolinonitrile and tert-butyl 4-formylpiperidine-1-carboxylate in DCE was added NaBH(OAc)₃ (1.5 eq.), AcOH and TEA. The reaction mixture was stirred at room temperature for 6 h. All volatiles were removed and the residue was chromatographed on silica gel to afford intermediate 5-(((1R,3r,5S)-8-(4-(4-(piperidin-4-ylmethyl)piperazin-1-yl)benzoyl)-8-azabicyclo[3.2.1]octan-3-yl)oxy)-3-(trifluoromethyl)picolinonitrile after removing the Boc group. ESI-MS: 582.29.

### Step 4: Synthesis of 5-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)-3-(trifluoromethyl)picolinonitrile (Cpd. No. 7)

DIPEA (5 eq.) was added to a solution of 5-(((1R,3r,5S)-8-(4-(4-(piperidin-4-ylmethyl)piperazin-1-yl)benzoyl)-8-azabicyclo[3.2.1]octan-3-yl)oxy)-3-(trifluoromethyl)picolinonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 7 with 82% yield. ESI-MS: 838.34.

### Example 11

### Synthesis of 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazine-1-carbonyl)benzoyl)-8-azabicyclo[3.2.1]octan-3-yl)oxy)benzonitrile (Cpd. No. 12)

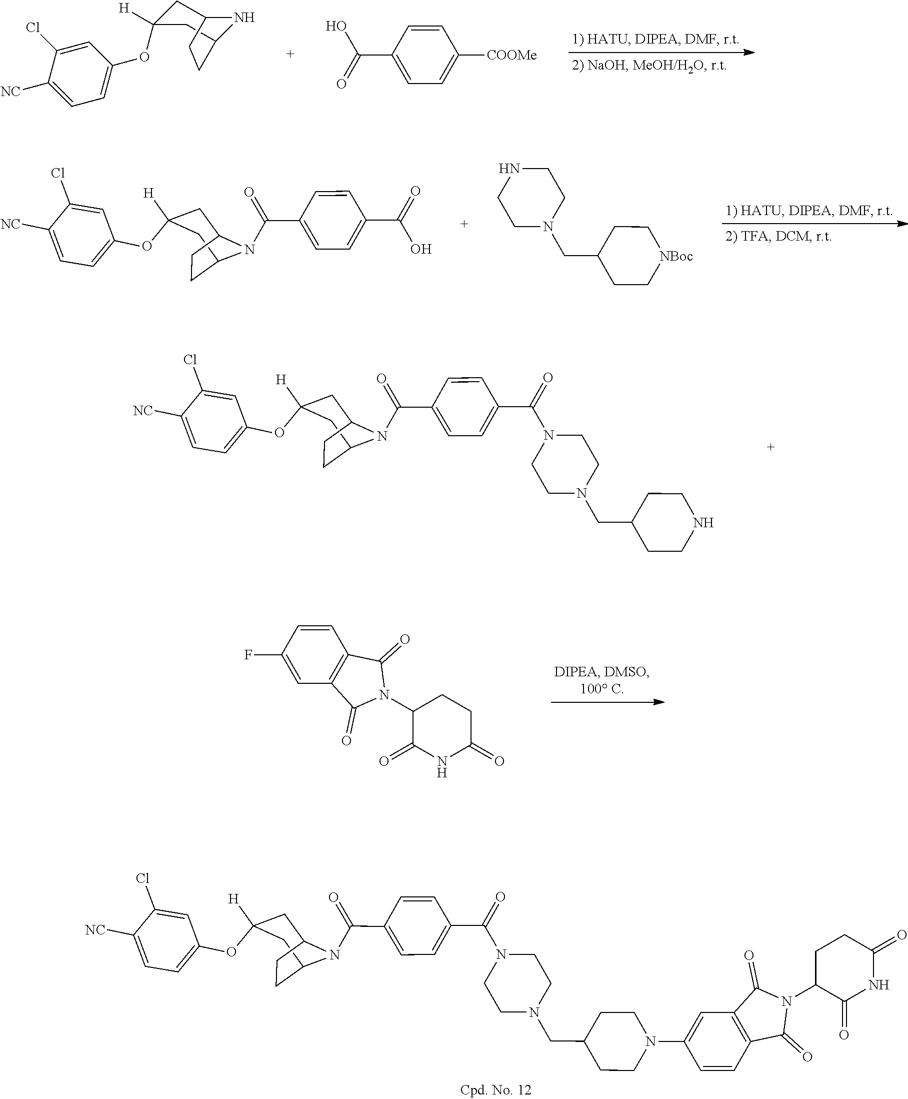

### Step 1: Synthesis of 4-((1R,3r,5S)-3-(3-chloro-4-cyanophenoxy)-8-azabicyclo[3.2.1]octane-8-carbonyl)benzoic Acid

4-(((1R,3r,5S)-8-Azabicyclo[3.2.1]octan-3-yl)oxy)-2-chlorobenzonitrile and 4-(methoxycarbonyl)benzoic acid were dissolved in DMF. The solution was added DIPEA (5 eq.) and HATU (1.2 eq.), the reaction mixture was stirred at room temperature for 1 h. The reaction mixture was extracted by EA, washed by water and organic phase was dried by Na₂SO₄. tert-Butyl 4-(4-(2-((1r,3r)-3-(3-chloro-4-cyanophenoxy)-2,2,4,4-tetramethylcyclobutyl)-1-oxoisoindoline-5-carbonyl)piperazin-1-yl)piperidine-1-carboxylate was obtained by removing the solvent under vacuum and purifying by flash column chromatography on silica gel. The desired intermediate 4-((1R,3r,5S)-3-(3-chloro-4-cyanophenoxy)-8-azabicyclo[3.2.1]octane-8-carbonyl)benzoic acid was obtained by hydrolyzing methyl ester in 86% yield. ESI-MS: 410.10.

### Step 2: Synthesis of 2-chloro-4-(((1R,3r,5S)-8-(4-(4-(piperidin-4-ylmethyl)piperazine-1-carbonyl)benzoyl)-8-azabicyclo[3.2.1]octan-3-yl)oxy)benzonitrile

4-((1R,3r,5S)-3-(3-Chloro-4-cyanophenoxy)-8-azabicyclo[3.2.1]octane-8-carbonyl)benzoic acid and tert-butyl 4-(piperazin-1-ylmethyl)piperidine-1-carboxylate were dissolved in DMF. The solution was added DIPEA (5 eq.) and HATU (1.2 eq.), the reaction mixture was stirred at room temperature for 1 h. The reaction mixture was extracted by EA, washed by water and organic phase was dried by Na₂SO₄. The desired intermediate 2-chloro-4-(((1R,3r,5S)-8-(4-(4-(piperidin-4-ylmethyl)piperazine-1-carbonyl)benzoyl)-8-azabicyclo[3.2.1]octan-3-yl)oxy)benzonitrile was obtained by deprotection with TFA in DCM in 86% yield. ESI-MS: 575.27.

### Step 3: Synthesis of 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazine-1-carbonyl)benzoyl)-8-azabicyclo[3.2.1]octan-3-yl)oxy)benzonitrile (Cpd. No. 12)

DIPEA (5 eq.) was added to a solution of 2-chloro-4-(((1R,3r,5S)-8-(4-(4-(piperidin-4-ylmethyl)piperazine-1-carbonyl)benzoyl)-8-azabicyclo[3.2.1]octan-3-yl)oxy)benzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 12 with 82% yield. ESI-MS: 831.31.

### Example 12

### Synthesis of 2-chloro-4-(((1R,3r,5S)-8-(4-(1-((1-(2-(2,6-dioxopiperidin-3-yl)-1,3-dioxoisoindolin-5-yl)piperidin-4-yl)methyl)piperidin-4-yl)benzoyl)-8-azabicyclo[3.2.1]octan-3-yl)oxy)benzonitrile (Cpd. No. 15)

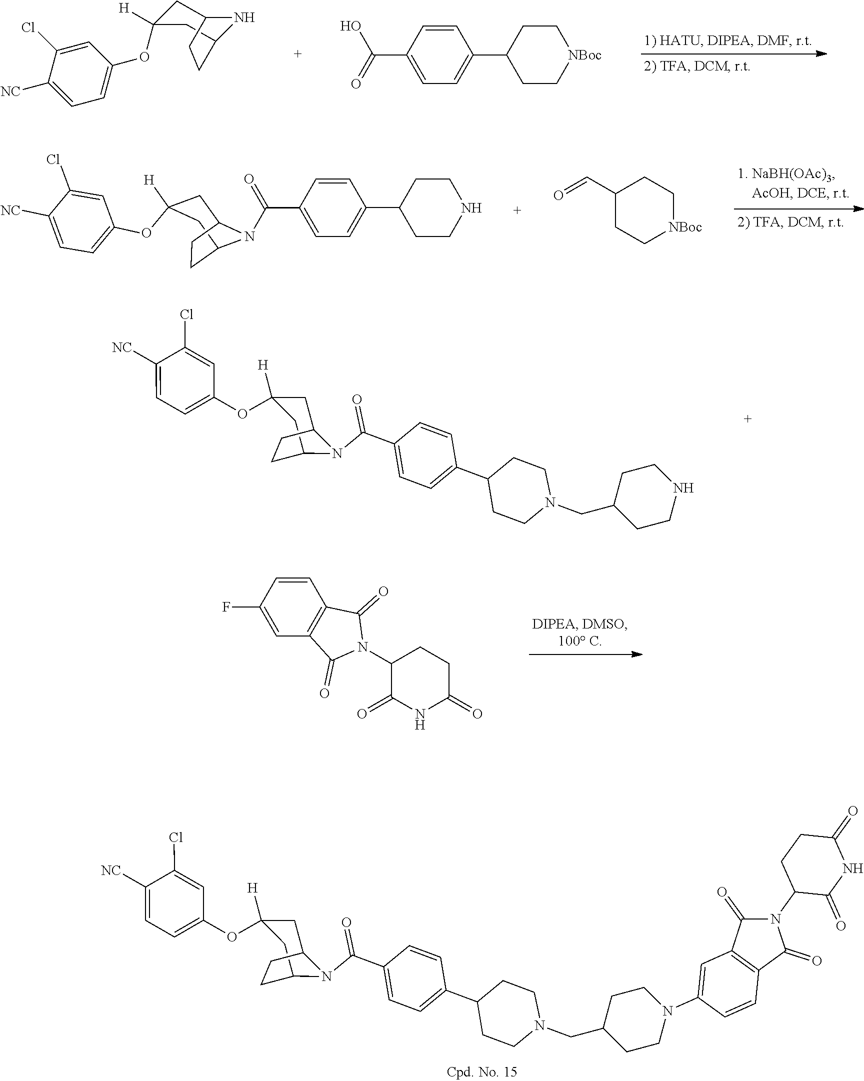

### Step 1: Synthesis of 2-chloro-4-(((1R,3r,5S)-8-(4-(piperidin-4-yl)benzoyl)-8-azabicyclo[3.2.1]octan-3-yl)oxy)benzonitrile

4-(((1R,3r,5S)-8-Azabicyclo[3.2.1]octan-3-yl)oxy)-2-chlorobenzonitrile and 4-(1-(tert-butoxycarbonyl)piperidin-4-yl)benzoic acid were dissolved in DMF. The solution was added DIPEA (5 eq.) and HATU (1.2 eq.), the reaction mixture was stirred at room temperature for 1 h. The reaction mixture was extracted by EA, washed by water and organic phase was dried by Na₂SO₄. tert-Butyl 4-(4-(2-((1r,3r)-3-(3-chloro-4-cyanophenoxy)-2,2,4,4-tetramethylcyclobutyl)-1-oxoisoindoline-5-carbonyl)piperazin-1-yl)piperidine-1-carboxylate was obtained by removing the solvent under vacuum and purifying by flash column chromatography on silica gel. The desired intermediate 2-chloro-4-(((1R,3r,5S)-8-(4-(piperidin-4-yl)benzoyl)-8-azabicyclo[3.2.1]octan-3-yl)oxy)benzonitrile was obtained by deprotection with TFA in DCM in 87% yield. ESI-MS: 449.19.

### Step 2: Synthesis of 2-chloro-4-(((1R,3r,5S)-8-(4-(1-(piperidin-4-ylmethyl)piperidin-4-yl)benzoyl)-8-azabicyclo[3.2.1]octan-3-yl)oxy)benzonitrile

To a solution of 2-chloro-4-(((1R,3r,5S)-8-(4-(piperidin-4-yl)benzoyl)-8-azabicyclo[3.2.1]octan-3-yl)oxy)benzonitrile and tert-butyl 4-formylpiperidine-1-carboxylate in DCE was added NaBH(OAc)₃ (1.5 eq.), AcOH and TEA. The reaction mixture was stirred at room temperature for 6 h. All volatiles were removed and the residue was chromatographed on silica gel to afford intermediate 2-chloro-4-(((1R,3r,5S)-8-(4-(1-(piperidin-4-ylmethyl)piperidin-4-yl)benzoyl)-8-azabicyclo[3.2.1]octan-3-yl)oxy)benzonitrile after removing the Boc group. ESI-MS: 546.28.

### Step 3: Synthesis of 2-chloro-4-(((1R,3r,5S)-8-(4-(1-((1-(2-(2,6-dioxopiperidin-3-yl)-1,3-dioxoisoindolin-5-yl)piperidin-4-yl)methyl)piperidin-4-yl)benzoyl)-8-azabicyclo[3.2.1]octan-3-yl)oxy)benzonitrile (Cpd. No. 15)

DIPEA (5 eq.) was added to a solution of 2-chloro-4-(((1R,3r,5S)-8-(4-(1-(piperidin-4-ylmethyl)piperidin-4-yl)benzoyl)-8-azabicyclo[3.2.1]octan-3-yl)oxy)benzonitrile and 2-(2,6-dioxopiperidin-3-yl)-5-fluoroisoindoline-1,3-dione (1.1 eq.) in DMSO (2 mL). After 4 h at 80° C., the mixture was subjected to HPLC purification to afford Cpd. No. 15 in 80% yield. ESI-MS: 802.32.

### Example 13

### Synthesis of 2-chloro-4-((1-(4-(4-((1-(2-(2,6-dioxopiperidin-3-yl)-1,3-dioxoisoindolin-5-yl)piperidin-4-yl)methyl)piperazin-1-yl)benzoyl)piperidin-4-yl)oxy)-3-methylbenzonitrile (Cpd. No. 70)

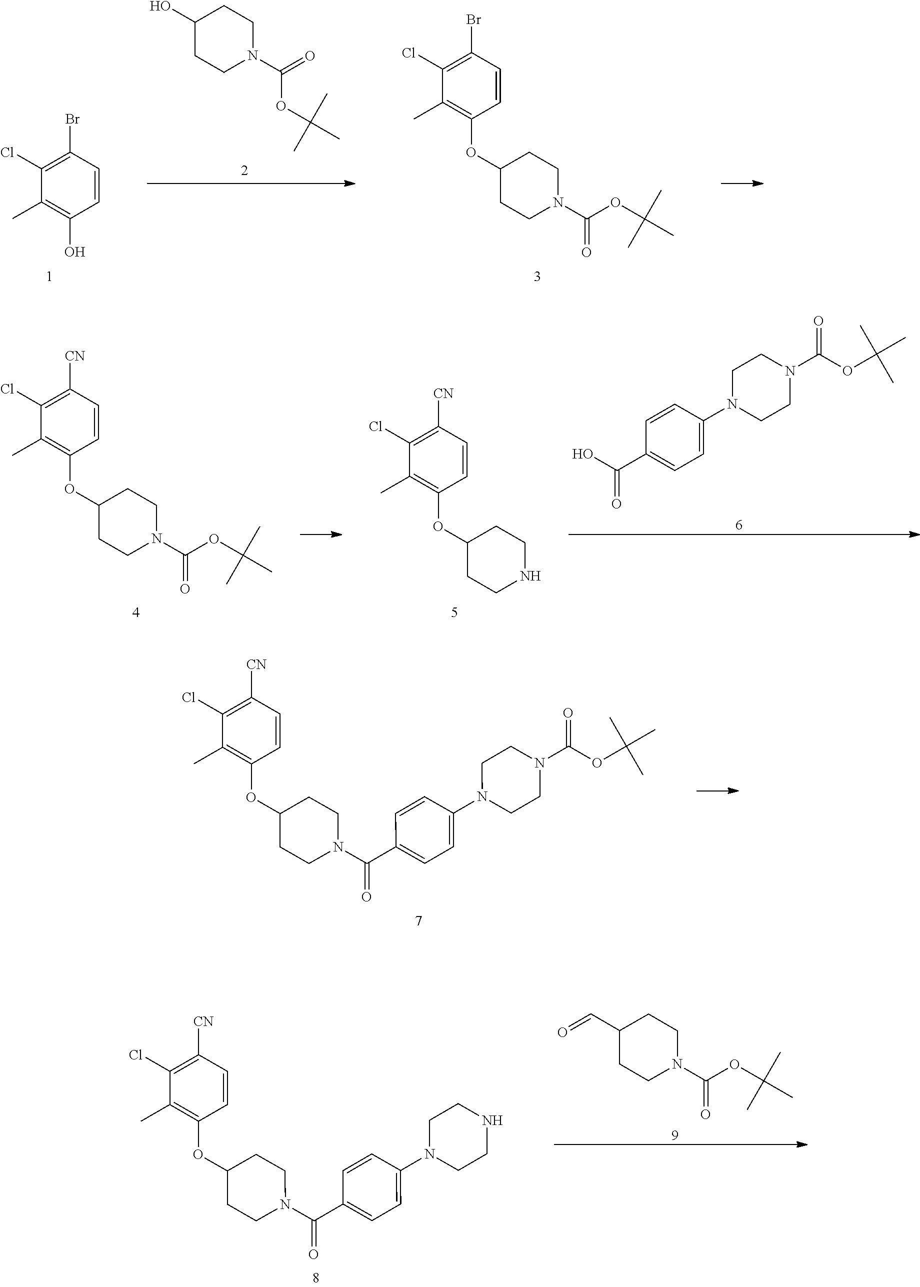

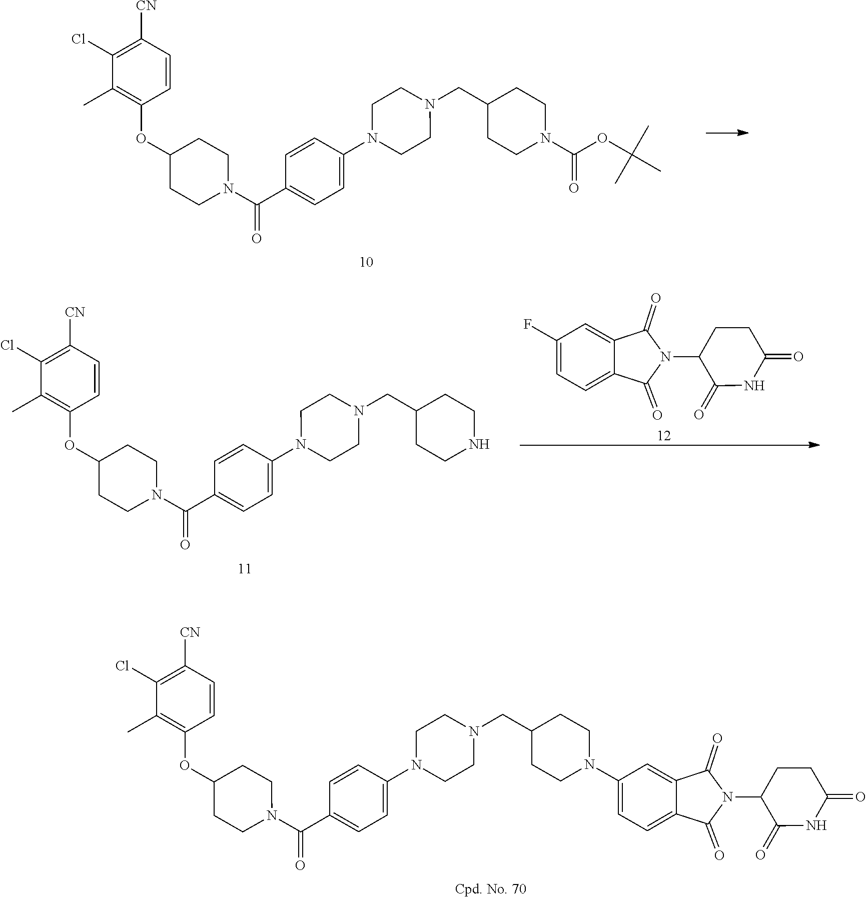

Compound 2 (1.0 eq) was dissolved in THF, and PPh₃ (1.1 eq) and DIAD (1.1 eq) were added at room temperature. After 15 min, compound 1 (1.0 eq) was added, and the reaction was stirred overnight. The reaction mixture was purified using a Combiflash chromatography system with hexane/EtOAc as the eluent to provide compound 3 in 80% yield.

Compound 3 (1.0 eq) was dissolved in DMF, and Pd(PPh₃)₄Cl₂ (0.2 eq) and CuCN (1.5 eq) were added to the degassed vial. The vial was placed in a microwave initiator at 140° C. for 1 h. The reaction mixture was cooled to room temperature and H₂O and EtOAC were added. The organic layer was separated, concentrated, and purified using a Combiflash chromatography system with hexane/EtOAc as the eluent to provide compound 4 in 75% yield.

Compound 4 was dissolved in DCM and TFA (10×) was added. All the solvent and TFA were removed to give compound 5.

Compound 6 was dissolved in DCM, and DIPEA (3 eq) and HATU (1.3 eq) were added. Compound 2 was dissolved in DCM and DIPEA (3 eq) was added. The compound 2 solution was poured into the compound 6 solution. The reaction was complete in 0.5 h. The reaction mixture was directly purified using a Combiflash chromatography system with liquid loading, and eluted with hexane/EtOAc to afford compound 7.

Compound 7 was dissolved in DCM and TFA (10×) was added. All the solvent and TFA were removed to give compound 8.

Compound 8 (1.5 eq) was dissolved in DCE, and compound 9 and AcOH (4 eq) were added. The mixture was stirred overnight. NaB(OAc)₃H (3 eq) was added and the reaction was complete in about 3 h. The reaction mixture was concentrated with silica gel and purified using a Combiflash chromatography system with DCM/MeOH (5%) as the eluent.

Compound 10 was dissolved in DCM and TFA (20×) was added. All the solvent and TFA were removed to give compound 11.

Compound 11 was dissolved in DMF, and DIPEA (3 eq) and compound 12 (1.3 eq) were added. The reaction mixture was stirred at 90° C. overnight. H₂O and TFA (15×) were added to the mixture. The mixture was purified using preparative HPLC to give Cpd. No. 70 in 42% yield. UPLC-MS 4. 2 min, 792.27.

### Example 14

### Synthesis of 2-chloro-4-((1-(4-(1-((1-(2-(2,6-dioxopiperidin-3-yl)-1,3-dioxoisoindolin-5-yl)piperidin-4-yl)methyl)piperidin-4-yl)benzoyl)piperidin-4-yl)oxy)-3-methylbenzonitrile (Cpd. No. 69)

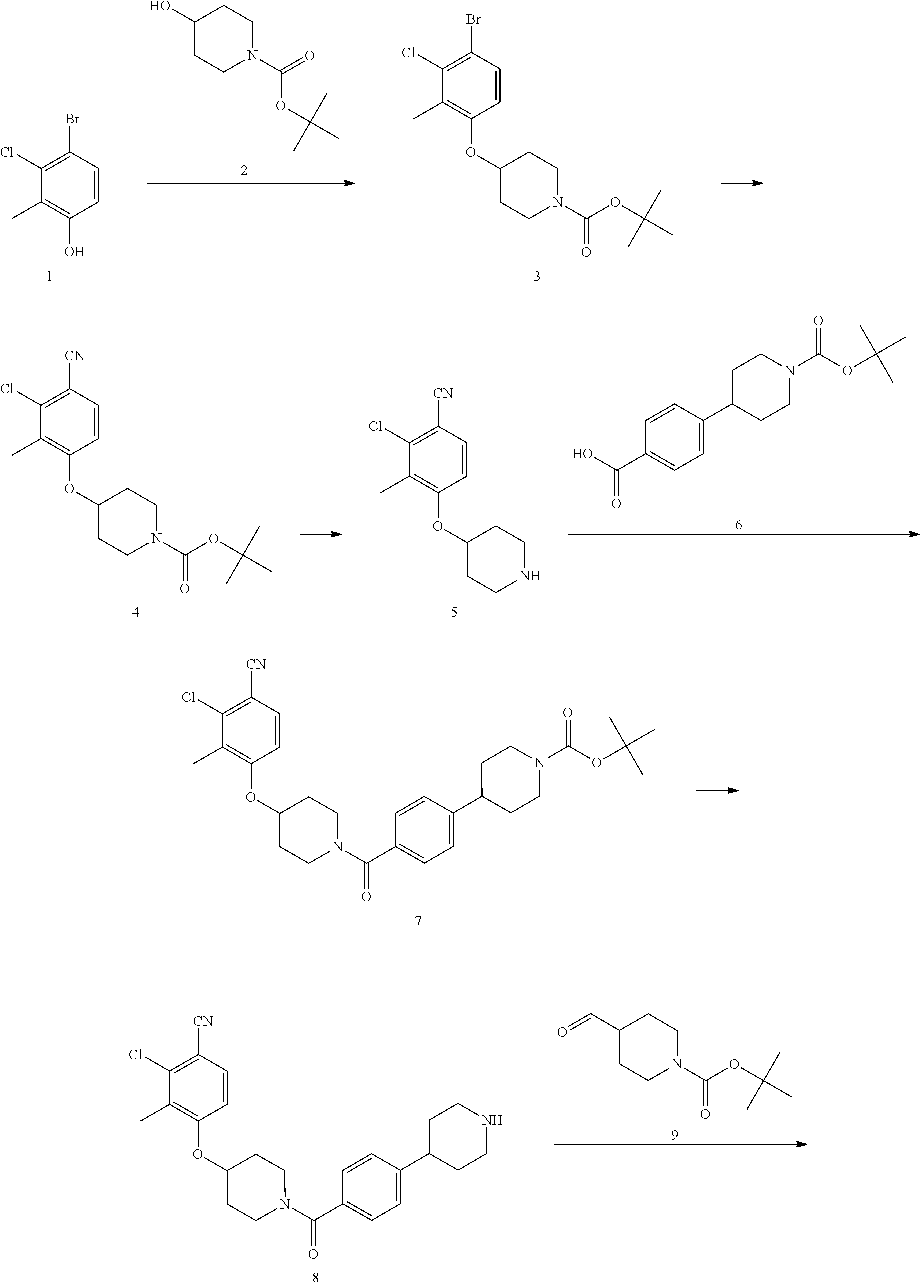

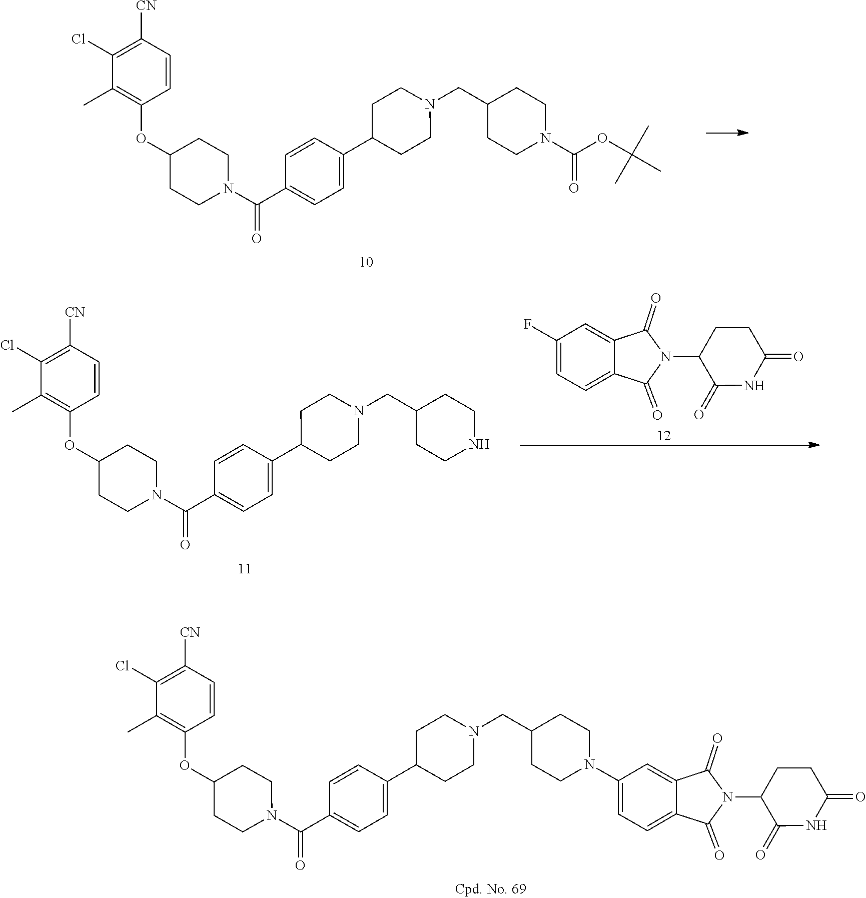

Cpd. No. 69 was synthesized in 43% yield following the procedure of EXAMPLE 13 from corresponding starting materials. UPLC-MS 4.3 min, 791.39.

### Example 15

### Synthesis of 2-chloro-4-((1-(4-(4-((4-(2-(2,6-dioxopiperidin-3-yl)-1,3-dioxoisoindolin-5-yl)piperazin-1-yl)methyl)piperidin-1-yl)benzoyl)piperidin-4-yl)oxy)benzonitrile (Cpd. No. 81)

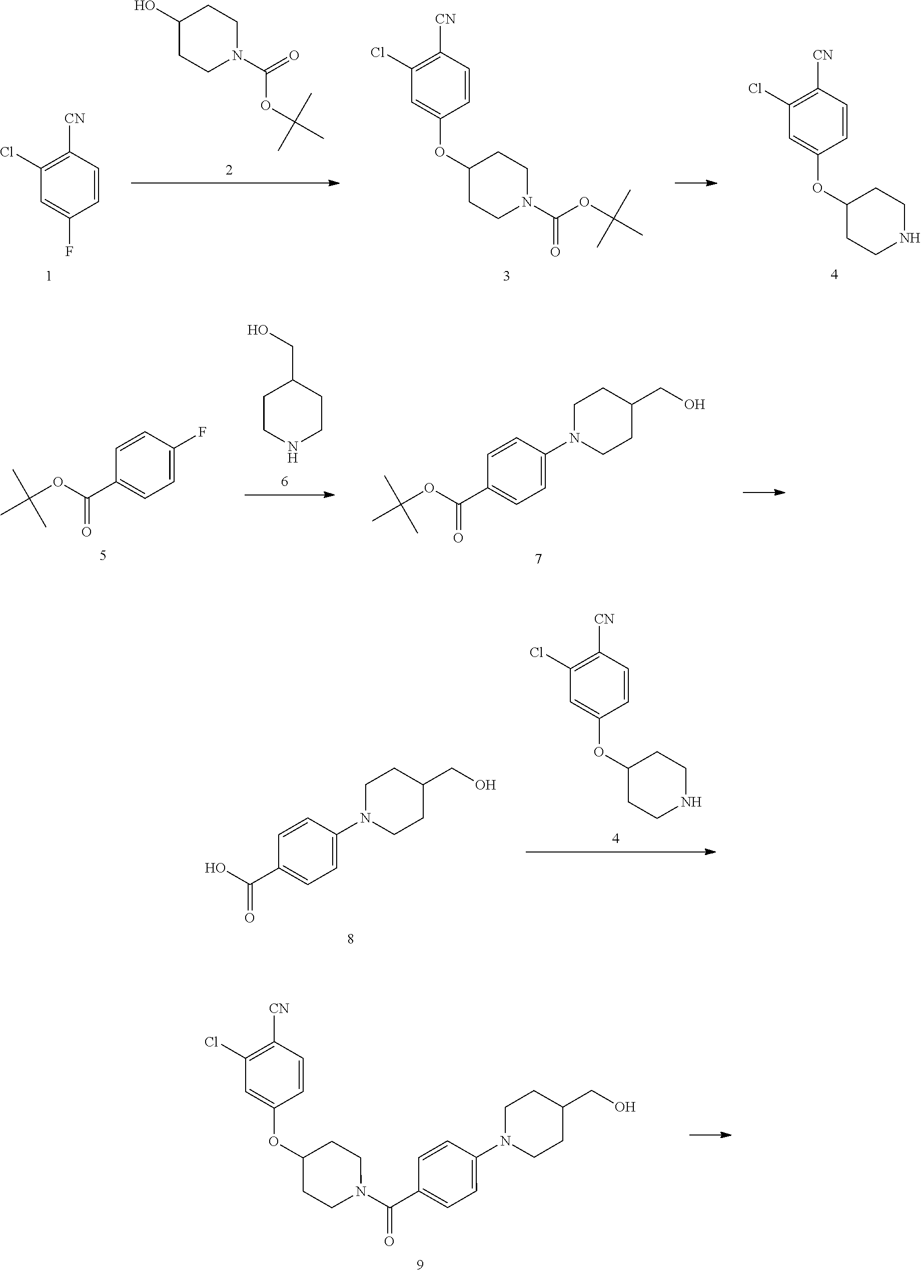

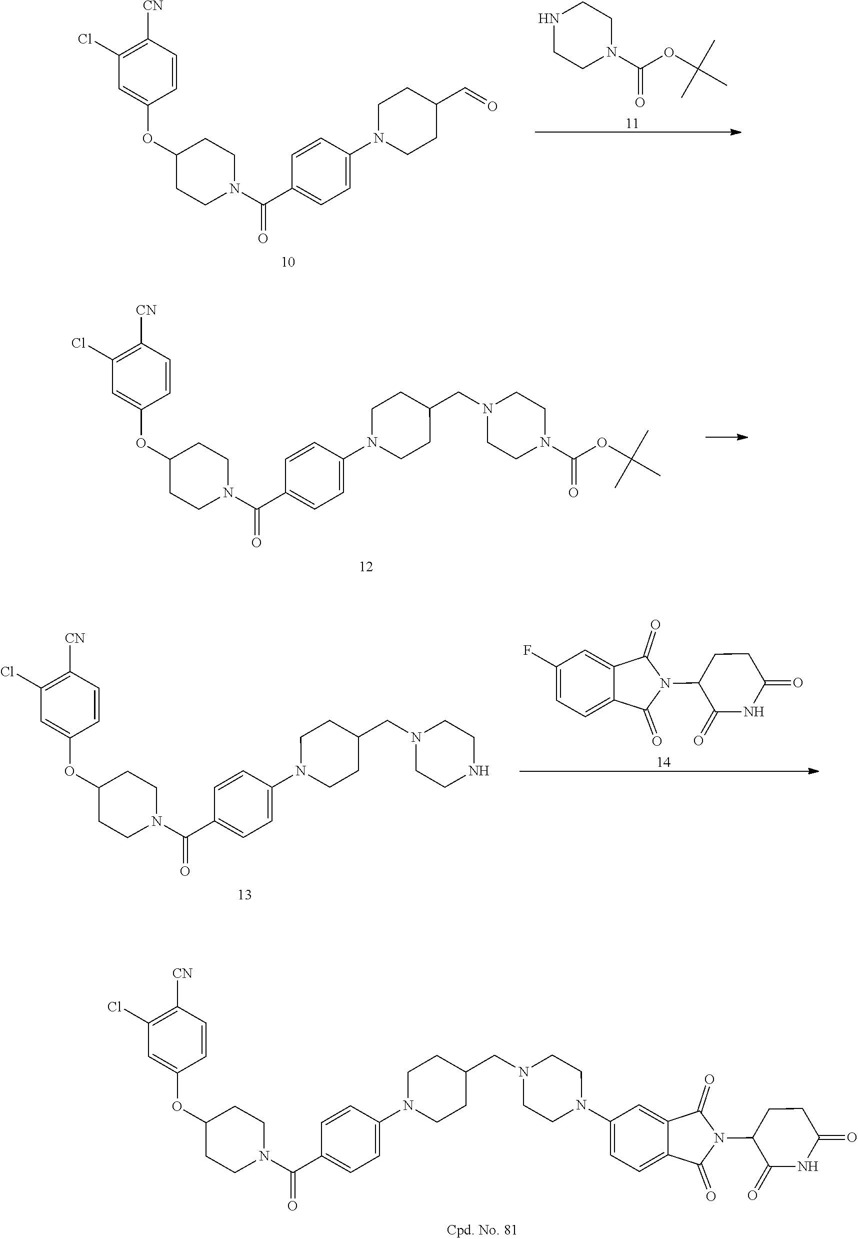

Compound 2 (1 eq) was dissolved in THF and NaH (3.0 eq) was added at 0° C. After 15 min, Compound 1 (1.1 eq) was added and the reaction mixture was stirred at room temperature for 4 h. The reaction was quenched with H₂O and extracted with EtOAc. The crude product was purified using a Combiflash chromatography system with hexane/EtOAc as the eluent to afford compound 3 in 70 yield %.

Compound 3 was dissolved in DCM and TFA (10×) was added. All the solvent and TFA were removed to give compound 4.

Compound 5 (1.0 eq), compound 6 (1.5 eq) and Cs₂CO₃ (3 eq) were dissolved in DMF. The reaction was stirred at 120° C. overnight. The reaction was cooled, and H₂O and EtOAc were added. The organic layer was concentrated and purified using a Combiflash chromatography system with hexane/EtOAc as the eluent to provide compound 7.

Compound 7 was dissolved in DCM and TFA (10×) was added. All the solvent and TFA were removed to give compound 8.

Compound 8 was dissolved in DCM and DIPEA (3 eq) and HATU (1.3 eq) were added. Compound 4 was dissolved in DCM and DIPEA (3 eq) was added. The compound 4 solution was poured into compound 8 solution. The reaction was complete in 0.5 h. The reaction mixture was directly purified using a Combiflash chromatography system with liquid loading, and eluted with hexane/EtOAc to afford compound 9.

Compound 9 (1.0 eq) was dissolved in DCM, and Dess Martin reagent (1.3 eq) was added. After 0.5 h at room temperature, the reaction was concentrated and purified using a Combiflash chromatography system with hexane/EtOAc as the eluent to give compound 10.

Compound 10 (1.0 eq) was dissolved in DCE, and compound 11 (1.5 eq) and AcOH (4 eq) were added. The mixture was stirred overnight. NaB(OAc)₃H (3 eq) was added and the reaction was complete in about 3 h. The reaction mixture was concentrated with silica gel and purified using a Combiflash chromatography system with DCM/MeOH (5%) as the eluent.

Compound 12 was dissolved in DCM and TFA (20×) was added. All the solvent and TFA were removed to give compound 13.

Compound 13 (1.0 eq) was dissolved in DMF, and DIPEA (3 eq) and compound 14 (1.3 eq) were added. The reaction mixture was stirred at 90° C. overnight. H₂O and TFA (15×) were added to the mixture. The mixture was purified using preparative HPLC to give Cpd. No. 81 in 35% yield. UPLC-MS 4.0 min, 778.36.

### Example 16

### Synthesis of 2-chloro-4-((1-(4-(1-((1-(2-(2,6-dioxopiperidin-3-yl)-1,3-dioxoisoindolin-5-yl)piperidin-4-yl)methyl)piperidin-4-yl)benzoyl)piperidin-4-yl)oxy)benzonitrile (Cpd. No. 259)

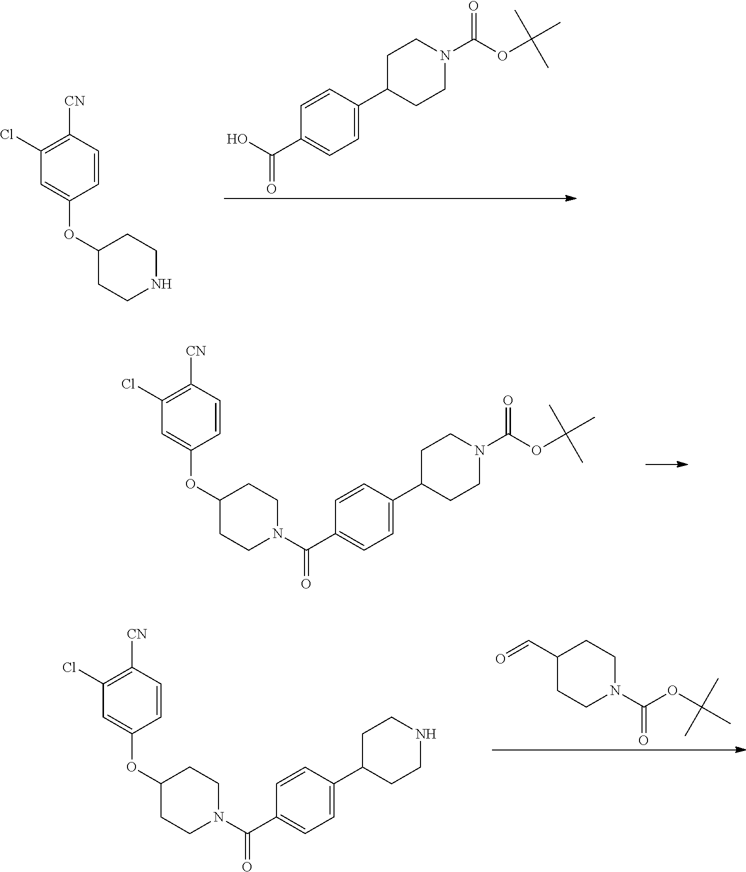 [SMILES: N#Cc1ccc(OC2CCNCC2)cc1Cl ; CC(C)(C)OC(=O)N1CCC(c2ccc(C(=O)O)cc2)CC1 ; CC(C)(C)OC(=O)N1CCC(c2ccc(C(=O)N3CCC(Oc4ccc(C#N)c(Cl)c4)CC3)cc2)CC1 ; N#Cc1ccc(OC2CCN(C(=O)c3ccc(C4CCNCC4)cc3)CC2)cc1Cl ; CC(C)(C)OC(=O)N1CCC(C=O)CC1]

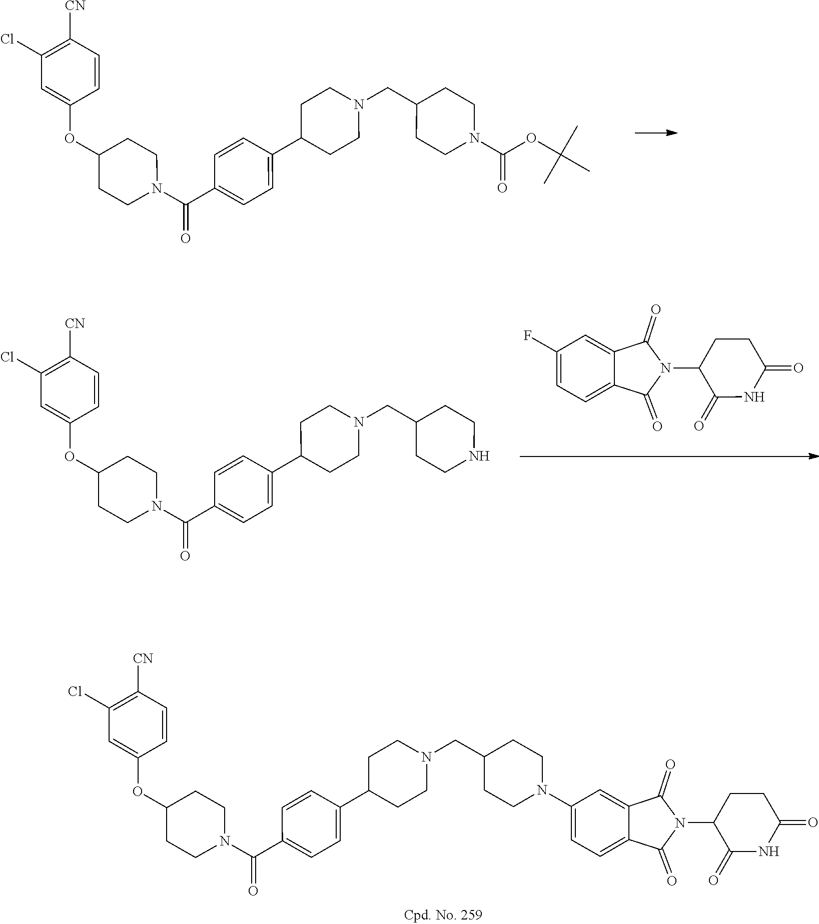 [SMILES: CC(C)(C)OC(=O)N1CCC(CN2CCC(c3ccc(C(=O)N4CCC(Oc5ccc(C#N)c(Cl)c5)CC4)cc3)CC2)CC1 ; N#Cc1ccc(OC2CCN(C(=O)c3ccc(C4CCN(CC5CCNCC5)CC4)cc3)CC2)cc1Cl ; O=C1CCC(N2C(=O)c3ccc(F)cc3C2=O)C(=O)N1 ; N#Cc1ccc(OC2CCN(C(=O)c3ccc(C4CCN(CC5CCN(c6ccc7c(c6)C(=O)N(C6CCC(=O)NC6=O)C7=O)CC5)CC4)cc3)CC2)cc1Cl]

Cpd. No. 81 was synthesized following the procedure of EXAMPLE 13 from corresponding starting materials.

### Example 17

### Synthesis of 4-(4-((1-(2-(2,6-dioxopiperidin-3-yl)-1,3-dioxoisoindolin-5-yl)piperidin-4-yl)methyl)piperazin-1-yl)phenyl (1R,3s,5S)-3-(3-chloro-4-cyanophenoxy)-8-azabicyclo[3.2.1]octane-8-carboxylate (Cpd. No. 86)

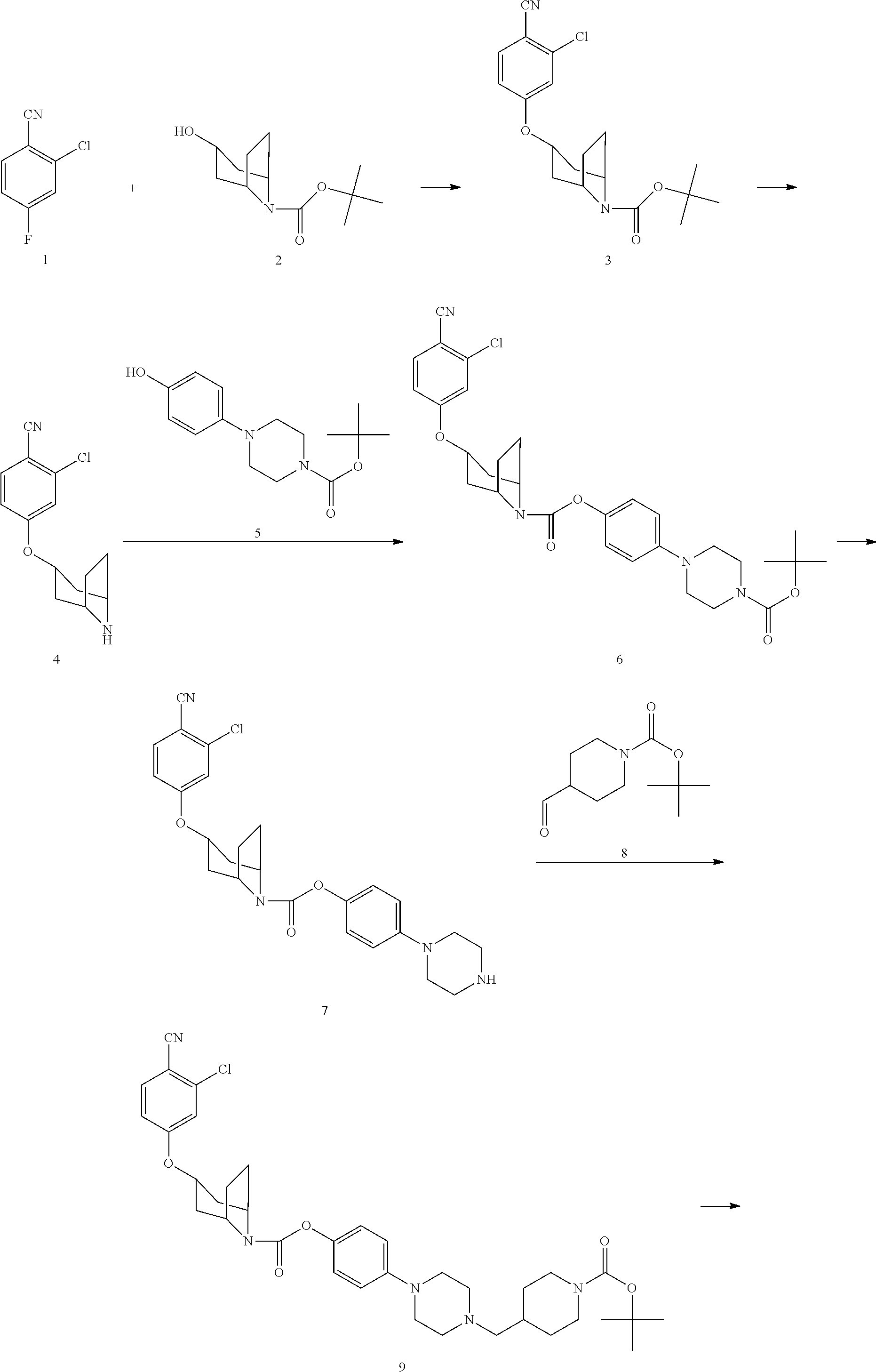

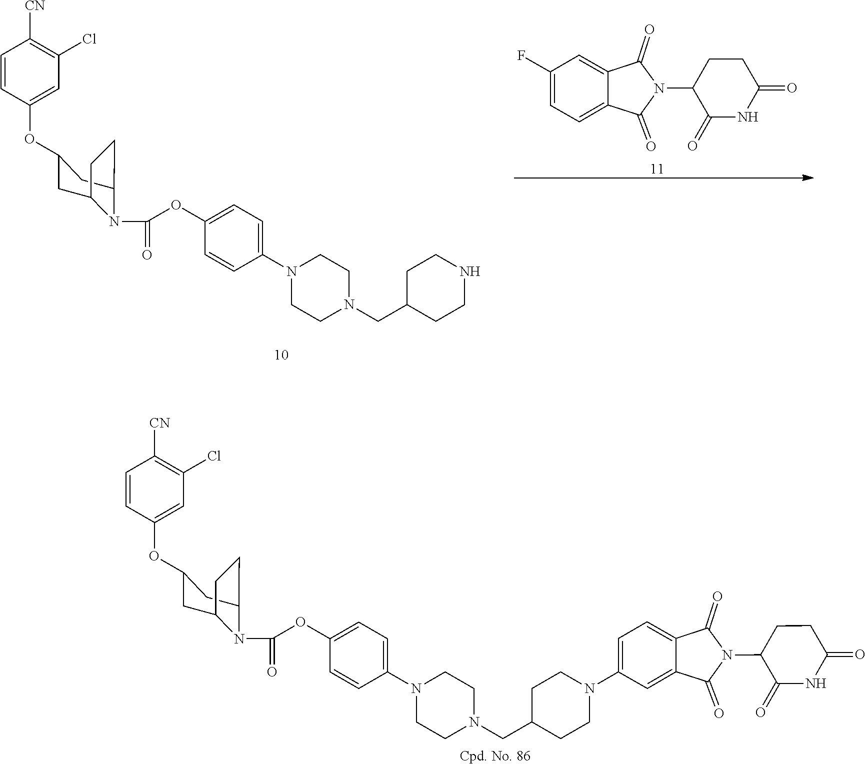

Compound 2 (1 eq) was dissolved in THF, and NaH (3.0 eq) was added at 0° C. After 15 min, Compound 1 (1.1 eq) was added, and the reaction mixture was stirred at ambient temperature for 4 h. The reaction was quenched with H₂O and extracted with EtOAc. The crude product was purified using a Combiflash chromatography system with hexane/EtOAc as the eluent to afford compound 3 in 75% yield.

Compound 3 was dissolved in DCM and TFA (10×) was added. All the solvent and TFA were removed to give compound 4.

Compound 5 (1.0 eq) and CDI (1.0 eq) were dissolved in THF and stirred at room temperature overnight. Compound 4 (1.0 eq) was added and stirred at room temperature for 12 h. The reaction mixture was concentrated and purified using a Combiflash chromatography system with hexane/EtOAc as the eluent to provide compound 6 in 65% yield.

Compound 6 was dissolved in DCM and TFA (10×) was added. All the solvent and TFA were removed to give compound 7.

Compound 7 (1.0 eq) was dissolved in DCE, and compound 8 (1.5 eq) and AcOH (4 eq) were added. The mixture was stirred overnight. NaB(OAc)₃H (3 eq) was added and the reaction was complete in about 3 h. The reaction mixture was concentrated with silica gel and purified using a Combiflash chromatography system with DCM/MeOH (5%) as the eluent.

Compound 9 was dissolved in DCM and TFA (10×) was added. All the solvent and TFA were removed to give compound 10.

Compound 10 (1.0 eq) was dissolved in DMF, and DIPEA (3 eq) and compound 11 (1.3 eq) were added. The reaction mixture was stirred at 90° C. overnight. H₂O and TFA (15×) were added to the mixture. The mixture was purified using preparative HPLC to give Cpd. No. 86 in 47% yield. UPLC-MS 4.8 min, 820.47.

### Example 18

### Synthesis of 2-chloro-4-(((1r,4r)-4-(5-(4-((1-(2-(2,6-dioxopiperidin-3-yl)-1,3-dioxoisoindolin-5-yl)piperidin-4-yl)methyl)piperazin-1-yl)-1-oxoisoindolin-2-yl)cyclohexyl)oxy)benzonitrile (Cpd. No. 88)

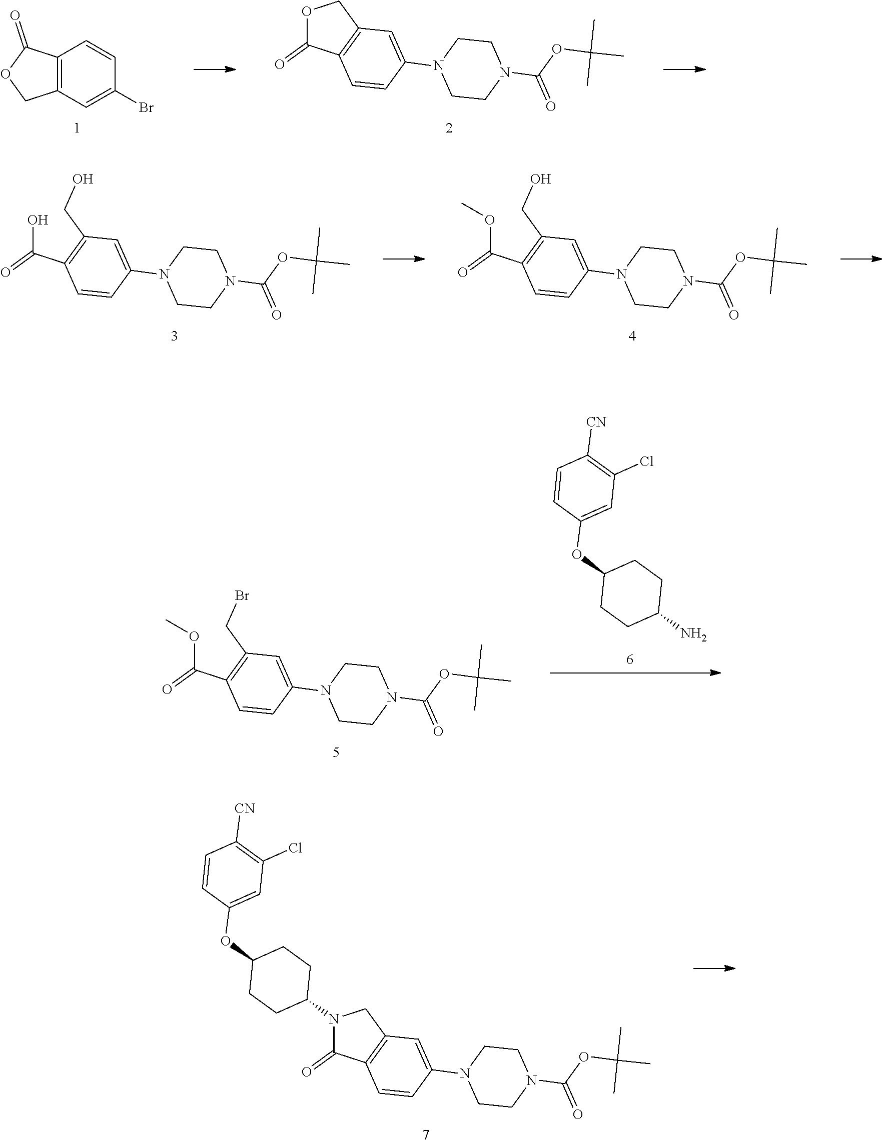

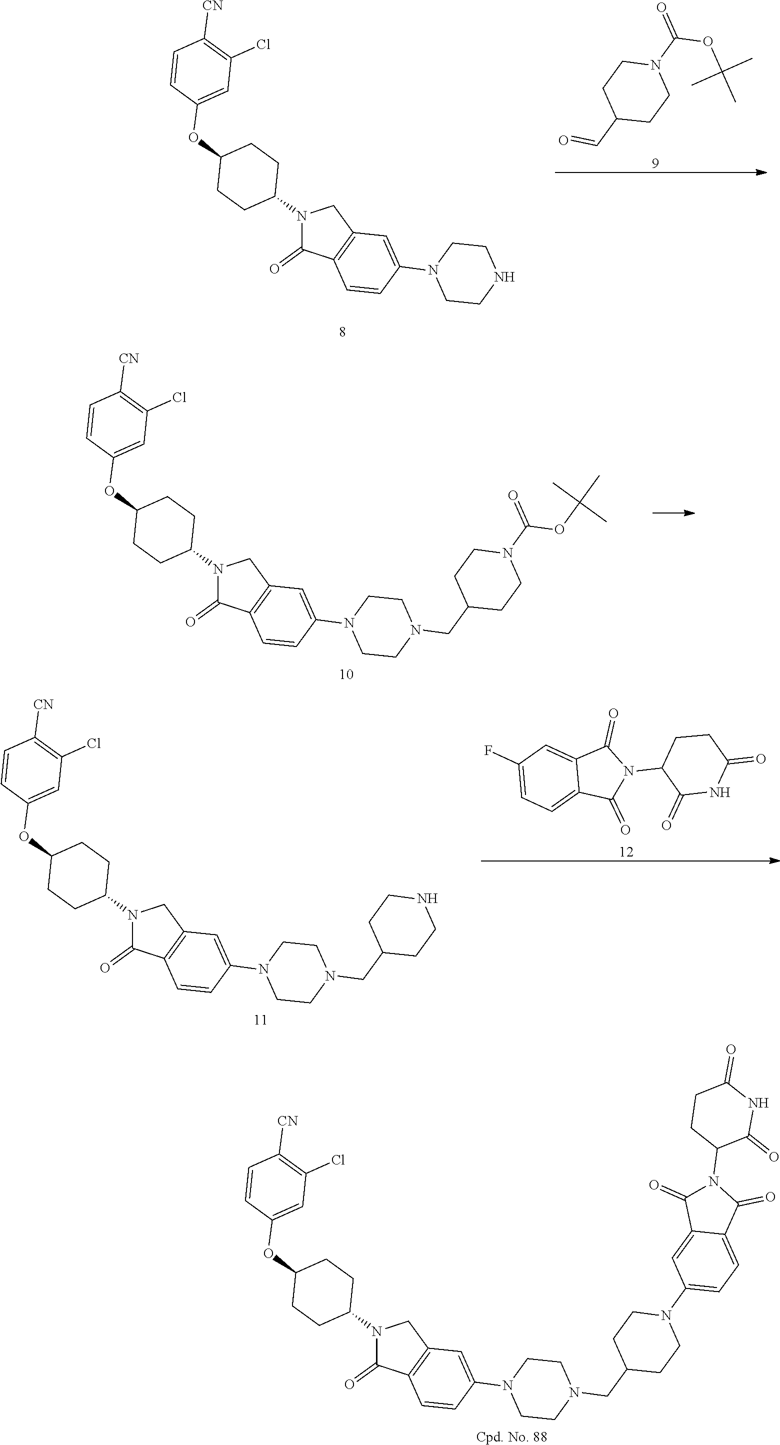

Compound 1 (1.0 eq), Pd₃(dba)₂ (0.1 eq), Xanphose (0.1 eq), Boc-piperzione (1.5 eq) and Cs₂CO₃ (3.0 eq) were dissolved in dioxane. After degassing, the reaction mixture was stirred at 90° C. overnight. The reaction was cooled, partitioned with EtOAc and H₂O, separated, and concentrated. The crude product was purified using a Combiflash chromatography system with hexane/EtOAc as the eluent to provide compound 2.

Compound 2 (1.0 eq) was dissolved in THF, MeOH, and H₂O (1:1:1), and NaOH (3 N, 10×) was added. After stirring at room temperature overnight, the mixture was acidified to pH 1, extracted with EtOAc, and purified using a Combiflash chromatography system with hexane/EtOAc as the eluent to give compound 3.

Compound 3 (1.0 eq) was dissolved in MeOH/EtOAc (1:1) at 0° C. TMSCH₂N₂ (2N, 3 eq) was added dropwise. The reaction was complete in 0.5 h. The mixture was warmed to room temperature, partitioned between EtOAc and H₂O, separated, concentrated, and purified using a Combiflash chromatography system with hexane/EtOAc as the eluent to give compound 4 in 85% yield.

Compound 4 (1.0 eq) was dissolved in THF, and PPh₃ (1.2 eq) and CBr₄ (1.2 eq) were added at room temperature. After 1 h, the mixture was partitioned between EtOAc and H₂O, separated, concentrated, and purified using a Combiflash chromatography system with hexane/EtOAc as the eluent to give compound 5 in 65% yield.

Compound 5 (1.0 eq), compound 6 (1.1 eq) and DIPEA (3.0 eq) were dissolved in DMF and stirred at 120° C. overnight. The mixture was cooled to room temperature, partitioned between EtOAc and H₂O, separated, concentrated, and purified using a Combiflash chromatography system with hexane/EtOAc as the eluent to give compound 7 in 73% yield.

Compound 7 was dissolved in DCM and TFA (10×) was added. All the solvent and TFA were removed to give compound 8.

Compound 8 (1.0 eq) was dissolved in DCE, and compound 9 (1.5 eq) and AcOH (4 eq) were added. The mixture was stirred overnight. NaB(OAc)₃H (3 eq) was added and the reaction was compete in about 3 h. The reaction mixture was concentrated with silica gel and purified using a Combiflash chromatography system with DCM/MeOH (5%) as the eluent.

Compound 10 was dissolved in DCM and TFA (10×) was added. All the solvent and TFA were removed to give compound 11.

Compound 11 (1.0 eq) was dissolved in DMF, and DIPEA (3 eq) and compound 12 (1.3 eq) were added. The reaction mixture was stirred at 90° C. overnight. H₂O and TFA (15×) was added to the mixture. The mixture was purified using preparative HPLC to give Cpd. No. 88 in 42% yield. UPLC-MS 4.2 min, 804.32.

### Example 19

### Synthesis of 2-chloro-4-((1r,3r)-3-(5-(4-((1-(2-(2,6-dioxopiperidin-3-yl)-1,3-dioxoisoindolin-5-yl)piperidin-4-yl)methyl)piperazin-1-yl)-1-oxoisoindolin-2-yl)-2,2,4,4-tetramethylcyclobutoxy)benzonitrile (Cpd. No. 89)

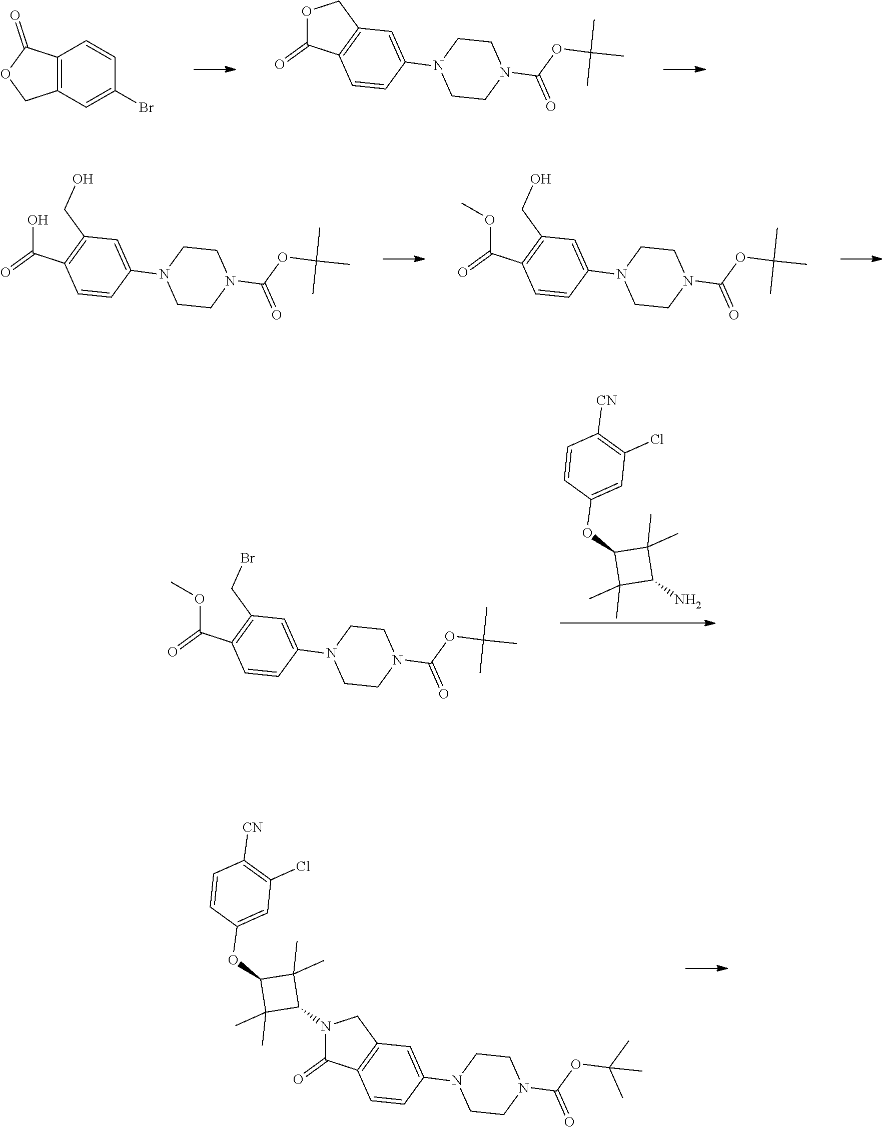

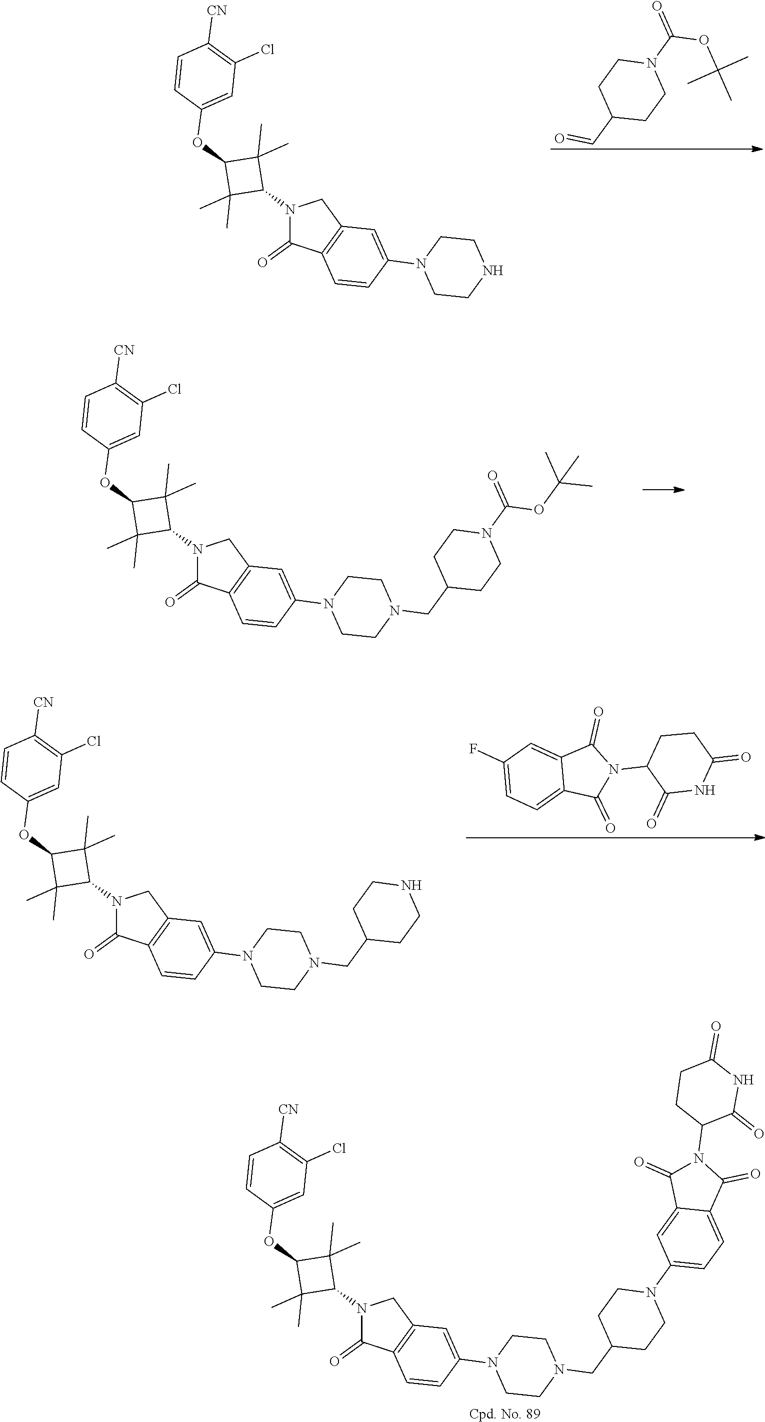

Cpd. No. 89 was synthesized in 47% yield following the procedure of EXAMPLE 18 from corresponding starting materials. UPLC-MS 4.4 min, 832.47.

### Example 20

### Synthesis of 2-chloro-4-(1-(4-((4-((3-((2-(2,6-dioxopiperidin-3-yl)-3-oxoisoindolin-5-yl)ethynyl)azetidin-1l-yl)methyl)cyclohexyl)ethynyl)benzyl)-4,5-dimethyl-1H-pyrazol-3-yl)benzonitrile (Cpd. No. 211)

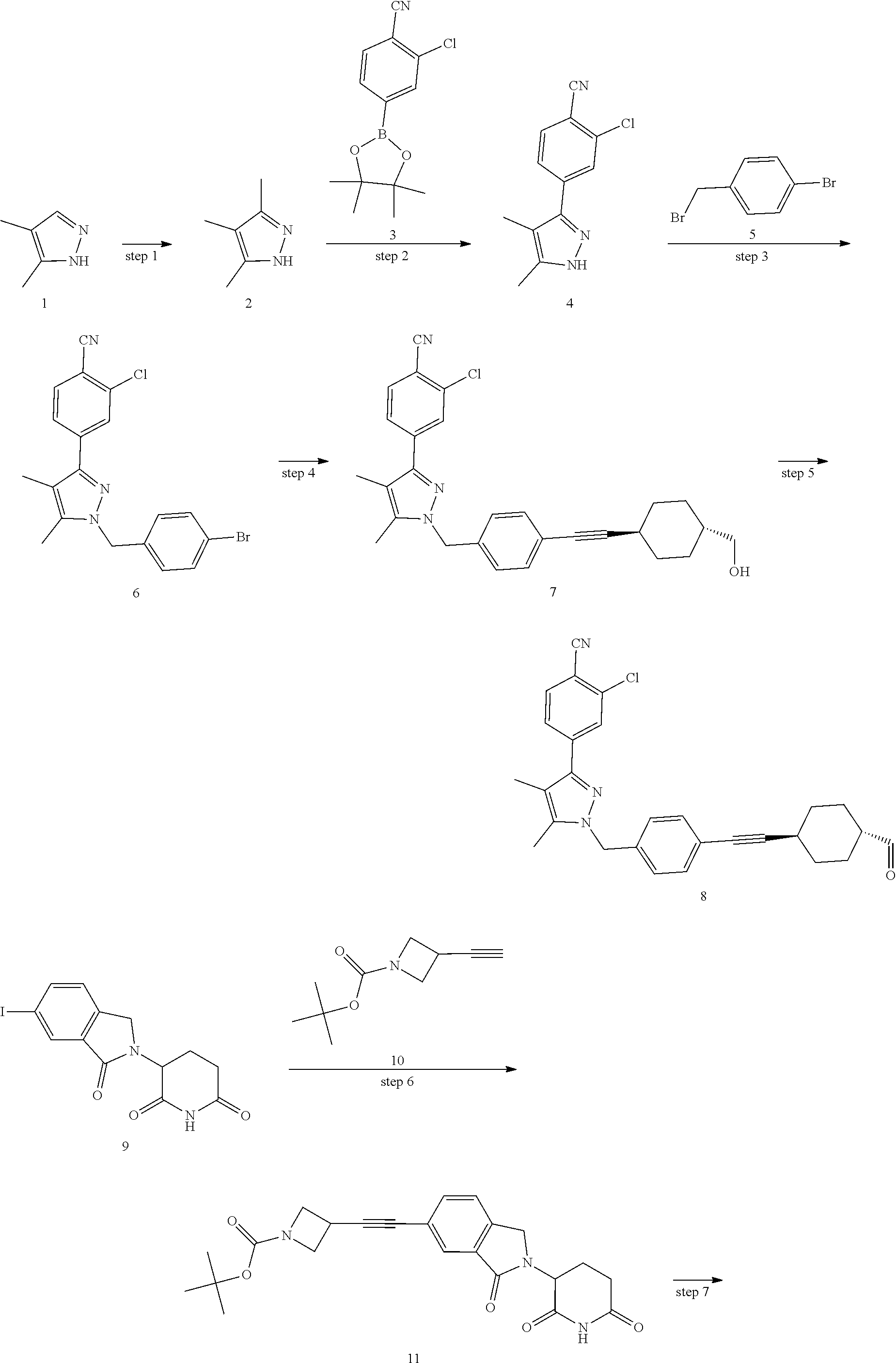

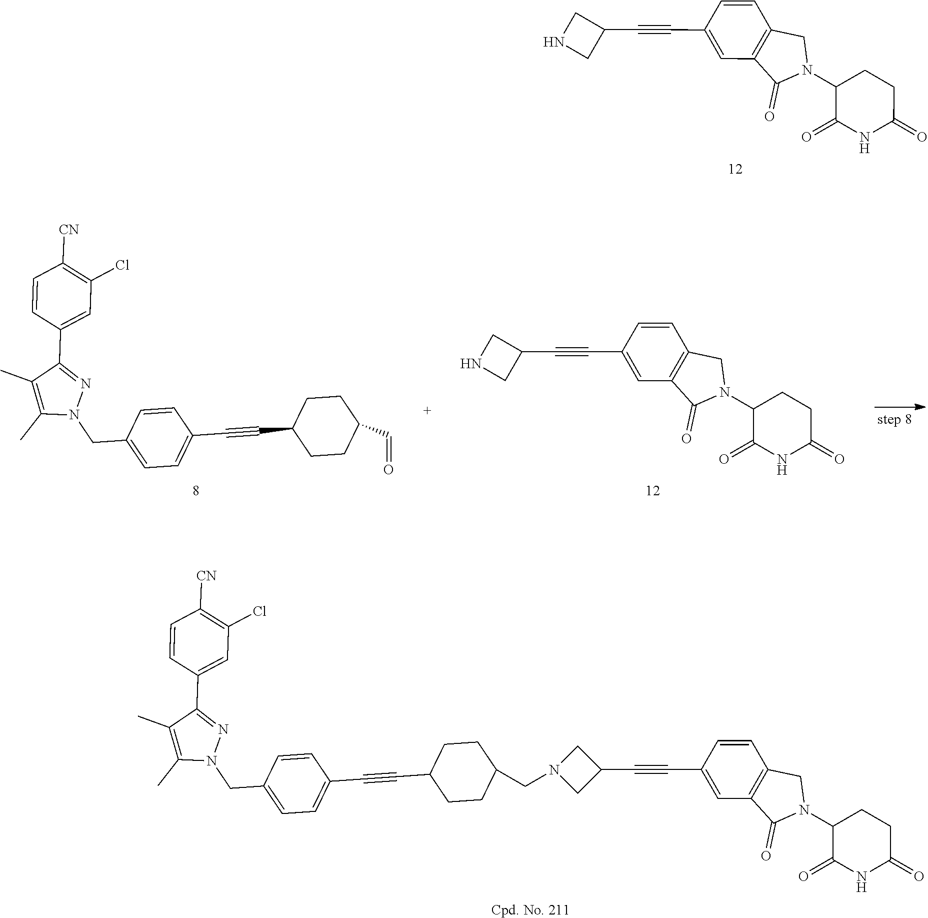

Compound 1 (1.0 eq) was dissolved in DMF, NIS (2.0 eq) was added, and the reaction mixture was stirred at room temperature for 4 h. The mixture was partitioned between H₂O and EtOAc, separated, and concentrated. Compound 2 was obtained in 90% yield using a Combiflash chromatography system with hexane/EtOAc as the eluent.

Compound 2 (1.0 eq), compound 3 (1.5 eq), Pd(dppf)₂Cl₂, and Cs₂CO₃ were dissolved in dioxane and water and stirred at 90° C. for 6 h. The mixture was cooled to room temperature, partitioned between H₂O and EtOAc, separated, and concentrated. Compound 4 was obtained in 70% yield using a Combiflash chromatography system with hexane/EtOAc as the eluent.

Compound 4 (1.0 eq) was dissolved in DMF, compound 5 (1.3.0 eq) and Cs₂CO₃ (3.0 eq) were added, and the reaction mixture was stirred at room temperature for 4 h. The mixture was partitioned between H₂O and EtOAc, separated, and concentrated. Compound 6 was obtained in 45% yield using a Combiflash chromatography system with hexane/EtOAc as the eluent.

Compound 6 (1.0 eq), 4-hydroxy methyl cyclohexyl acetylene (1.5 eq), Pd(PPh₃)₄Cl₂, CuI, and DIPEA were dissolved in DMF and stirred at 90° C. for 6 h. The mixture was cooled to room temperature, partitioned between H₂O and EtOAc, separated, and concentrated. Compound 3 in 80% yield was obtained using a Combiflash chromatography system with hexane/EtOAc as the eluent.

Compound 7 (1.0 eq) was dissolved in DCM, and Dess Martin reagent (1.3 eq) was added. The reaction mixture was stirred at room temperature for 4 h. The reaction was partitioned between EtOAc and H₂O, and the organic layer was washed with brine. The concentrated crude product was purified using a Combiflash chromatography system with hexane/EtOAc as the eluent to give compound 8 in about 75% yield.

Compound 9 (1.0 eq), compound 10 (1.5 eq), Pd(PPh₃)₄Cl₂, CuI, and DIPEA were dissolved in DMF and stirred at 90° C. for 4 h. The mixture was cooled to room temperature, partitioned between H₂O and EtOAc, separated, and concentrated. Compound 11 was obtained in 70% yield using a Combiflash chromatography system with hexane/EtOAc as the eluent.

Compound 11 was dissolved in DCM and TFA (10×) was added. All the solvent and TFA were removed to give compound 12.

Compound 12 (1.0 eq) was dissolved in DCE, and compound 8 (1.5 eq) and AcOH (4 eq) were added. The mixture was stirred overnight. NaB(OAc)₃H (3 eq) was added and the reaction was complete in about 3 h. H₂O and TFA (15×) was added to the mixture. The mixture was purified using preparative HPLC to give Cpd. No. 211 in 54% yield. UPLC-MS 5.3 min, 763.25.

### Example 21

### Synthesis of 2-chloro-4-(4-(4-(1-((1-(2-(2,6-dioxopiperidin-3-yl)-1,3-dioxoisoindolin-5-yl)piperidin-4-yl)methyl)piperidin-4-yl)phenoxy)piperidin-1-yl)benzonitrile (Cpd. No. 255)

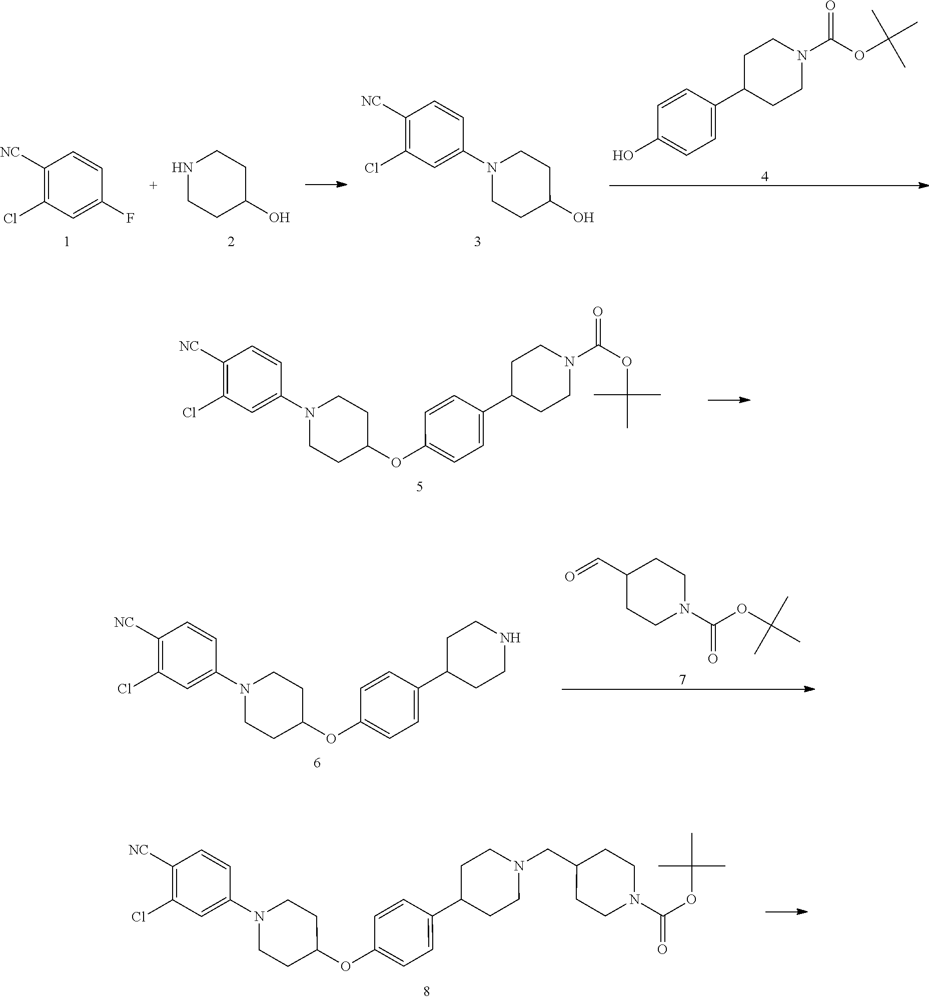

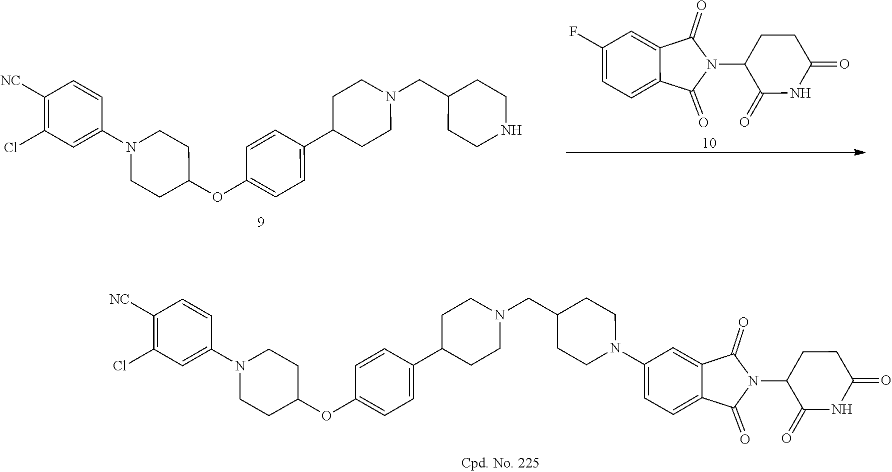

Compound 1 (1.0 eq), compound 2 (1.5 eq), and Cs₂CO₃ (3 eq) were dissolved in DMF. The reaction was stirred at 120° C. overnight. The reaction was cooled to room temperature and partitioned between H₂O and EtOAc. The organic layer was concentrated and purified using a Combiflash chromatography system with hexane/EtOAc as the eluent to provide compound 3.

Compound 3 (1.0 eq) was dissolved in THF, and PPh₃ (1.1 eq) and DIAD (1.1 eq) were added at room temperature. After 15 min, compound 4 (1.0) was added and the reaction was stirred overnight. The reaction mixture was purified using a Combiflash chromatography system with hexane/EtOAc as the eluent to provide compound 5 in 80% yield.

Compound 5 was dissolved in DCM and TFA (10×) was added. All the solvent and TFA were removed to give compound 6.

Compound 6 (1.0 eq) was dissolved in DCE, and Compound 7 (1.5 eq) and AcOH (4 eq) were added. The mixture was stirred overnight. NaB(OAc)₃H (3 eq) was added and the reaction was complete in about 3 h. The reaction mixture was concentrated with silica gel and purified using a Combiflash chromatography system with DCM/MeOH (5%) as the eluent to give compound 8.

Compound 8 was dissolved in DCM and TFA (10×) was added. All the solvent and TFA were removed to give compound 9.

Compound 9 (1.0 eq) was dissolved in DMF, and DIPEA (3 eq) and compound 10 (1.3 eq) were added. The reaction mixture was stirred at 90° C. overnight. H₂O and TFA (15×) were added to the mixture. The mixture was purified using preparative HPLC to give Cpd. No. 255 in 48% yield. UPLC-MS 4.6 min, 749.42.

### Example 22

### Representative Compounds of the Disclosure

The following representative Compounds of the Disclosure were prepared using the synthetic methods described in EXAMPLES 1-21.

Cpd. No. 1: 2-chloro-4-(((1R,3r,5S)-8-(4-(4-((4-(2-(2,6-dioxopiperidin-3-yl)-1,3-dioxoisoindolin-5-yl)piperazin-1-yl)methyl)piperidin-1-yl)benzoyl)-8-azabicyclo[3.2.1]octan-3-yl)oxy)benzonitrile. LC-MS(ESI) m/z (M+H)⁺: 804.35; calcd: 804.33; >95% purity.

Cpd. No. 2: 2-chloro-4-(((1R,3r,5S)-8-(6-(4-((1-(2-(2,6-dioxopiperidin-3-yl)-1,3-dioxoisoindolin-5-yl)piperidin-4-yl)methyl)piperazin-1-yl)nicotinoyl)-8-azabicyclo[3.2.1]octan-3-yl)oxy)benzonitrile. LC-MS(ESI) m/z (M+H)⁺: 805.32; calcd: 805.33; >95% purity.

Cpd. No. 3: 2-chloro-4-(((1R,3r,5S)-8-(6-(4-((1-(2-(2,6-dioxopiperidin-3-yl)-1,3-dioxoisoindolin-4-yl)piperidin-4-yl)methyl)piperazin-1-yl)nicotinoyl)-8-azabicyclo[3.2.1]octan-3-yl)oxy)benzonitrile. LC-MS(ESI) m/z (M+H)⁺: 805.34; calcd: 805.33; >95% purity.

Cpd. No. 4: 2-chloro-4-(((1R,3r,5S)-8-(4-(4-((1-(2-(2,6-dioxopiperidin-3-yl)-1,3-dioxoisoindolin-5-yl)azetidin-3-yl)methyl)piperazin-1-yl)benzoyl)-8-azabicyclo[3.2.1]octan-3-yl)oxy)benzonitrile. LC-MS(ESI) m/z (M+H)⁺: 776.33; calcd: 776.30; >95% purity.

Cpd. No. 5: 2-chloro-4-(((1R,3r,5S)-8-(4-(4-((1-(2-(2,6-dioxopiperidin-3-yl)-1,3-dioxoisoindolin-4-yl)azetidin-3-yl)methyl)piperazin-1-yl)benzoyl)-8-azabicyclo[3.2.1]octan-3-yl)oxy)benzonitrile. LC-MS(ESI) m/z (M+H)⁺: 776.31; calcd: 776.30; >95% purity.

Cpd. No. 6: 4-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)-2-(trifluoromethyl)benzonitrile. LC-MS(ESI) m/z (M+H)⁺: 838.38; calcd: 838.36; >95% purity.

Cpd. No. 7: 5-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)-3-(trifluoromethyl)picolinonitrile. LC-MS(ESI) m/z (M+H)⁺: 839.37; calcd: 839.35; >95% purity.

Cpd. No. 8: 2-chloro-4-(((1R,3r,5S)-8-(4-(4-(7-(2-(2,6-dioxopiperidin-3-yl)-1,3-dioxoisoindolin-5-yl)-7-azaspiro[3.5]nonan-2-yl)piperazin-1-yl)benzoyl)-8-azabicyclo[3.2.1]octan-3-yl)oxy)benzonitrile. LC-MS(ESI) m/z (M+H)⁺: 830.34; calcd: 830.35; >95% purity.

Cpd. No. 9: 2-chloro-4-(((1R,3r,5S)-8-(4-(4-(7-(2-(2,6-dioxopiperidin-3-yl)-1,3-dioxoisoindolin-4-yl)-7-azaspiro[3.5]nonan-2-yl)piperazin-1-yl)benzoyl)-8-azabicyclo[3.2.1]octan-3-yl)oxy)benzonitrile. LC-MS(ESI) m/z (M+H)⁺: 830.37; calcd: 830.35; >95% purity.

Cpd. No. 10: 2-chloro-4-(((1R,3r,5S)-8-(4-(4-(2-(1-(2-(2,6-dioxopiperidin-3-yl)-1,3-dioxoisoindolin-5-yl)azetidin-3-yl)ethyl)piperazin-1-yl)benzoyl)-8-azabicyclo[3.2.1]octan-3-yl)oxy)benzonitrile. LC-MS(ESI) m/z (M+H)⁺: 790.33; calcd: 790.31; >95% purity.

Cpd. No. 11: 2-chloro-4-(((1R,3r,5S)-8-(4-(7-(1-(2-(2,6-dioxopiperidin-3-yl)-1,3-dioxoisoindolin-5-yl)piperidin-4-yl)-2,7-diazaspiro[3.5]nonan-2-yl)benzoyl)-8-azabicyclo[3.2.1]octan-3-yl)oxy)benzonitrile. LC-MS(ESI) m/z (M+H)⁺: 830.34; calcd: 830.35; >95% purity.

Cpd. No. 12: 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazine-1-carbonyl)benzoyl)-8-azabicyclo[3.2.1]octan-3-yl)oxy)benzonitrile. LC-MS(ESI) m/z (M+H)⁺: 832.32; calcd: 832.32; >95% purity.

Cpd. No. 13: 2-chloro-4-(((1R,3r,5S)-8-(4-(4-(1-(2-(2,6-dioxopiperidin-3-yl)-1,3-dioxoisoindolin-5-yl)piperidin-4-yl)piperazine-1-carbonyl)benzoyl)-8-azabicyclo[3.2.1]octan-3-yl)oxy)benzonitrile. LC-MS(ESI) m/z (M+H)⁺: 818.33; calcd: 818.31; >95% purity.

Cpd. No. 14: 2-chloro-4-(((1R,3r,5S)-8-(4-(7-((1-(2-(2,6-dioxopiperidin-3-yl)-1,3-dioxoisoindolin-5-yl)piperidin-4-yl)methyl)-2,7-diazaspiro[3.5]nonan-2-yl)benzoyl)-8-azabicyclo[3.2.1]octan-3-yl)oxy)benzonitrile. LC-MS(ESI) m/z (M+H)⁺: 844.35; calcd: 844.36; >95% purity.

Cpd. No. 15: 2-chloro-4-(((1R,3r,5S)-8-(4-(1-((1-(2-(2,6-dioxopiperidin-3-yl)-1,3-dioxoisoindolin-5-yl)piperidin-4-yl)methyl)piperidin-4-yl)benzoyl)-8-azabicyclo[3.2.1]octan-3-yl)oxy)benzonitrile. LC-MS(ESI) m/z (M+H)⁺: 803.34; calcd: 803.33; >95% purity.

Cpd. No. 16: 2-chloro-4-((1R,3s,5S)-3-(4-(1-((1-(2-(2,6-dioxopiperidin-3-yl)-1,3-dioxoisoindolin-5-yl)piperidin-4-yl)methyl)piperidin-4-yl)phenoxy)-8-azabicyclo[3.2.1]octan-8-yl)benzonitrile. LC-MS(ESI) m/z (M+H)⁺: 775.33; calcd: 775.34; >95% purity.

Cpd. No. 17: 2-chloro-4-(((1R,3r,5S)-8-(4-((1-((1-(2-(2,6-dioxopiperidin-3-yl)-1,3-dioxoisoindolin-5-yl)piperidin-4-yl)methyl)piperidin-4-yl)oxy)benzoyl)-8-azabicyclo[3.2.1]octan-3-yl)oxy)benzonitrile. LC-MS(ESI) m/z (M+H)⁺: 819.35; calcd: 819.33; >95% purity.

Cpd. No. 18: 2-chloro-4-(((1R,3r,5S)-8-(4-((1′-(2-(2,6-dioxopiperidin-3-yl)-1,3-dioxoisoindolin-5-yl)-[1,4′-bipiperidin]-4-yl)oxy)benzoyl)-8-azabicyclo[3.2.1]octan-3-yl)oxy)benzonitrile. LC-MS(ESI) m/z (M+H)⁺: 805.33; calcd: 805.31; >95% purity.

Cpd. No. 19 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-3-methyl-8-azabicyclo[3.2.1]octan-3-yl)oxy)benzonitrile. LC-MS(ESI) m/z (M+H)⁺: 818.36; calcd: 818.35; >95% purity.

Cpd. No. 20: 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)-3-fluorobenzoyl)-8-azabicyclo[3.2.1]octan-3-yl)oxy)benzonitrile. LC-MS(ESI) m/z (M+H)⁺: 822.34; calcd: 822.32; >95% purity.

Cpd. No. 21: 2-chloro-4-(((1R,3r,5S)-8-(4-(6-(1-(2-(2,6-dioxopiperidin-3-yl)-1,3-dioxoisoindolin-5-yl)piperidin-4-yl)-2,6-diazaspiro[3.3]heptan-2-yl)benzoyl)-8-azabicyclo[3.2.1]octan-3-yl)oxy)benzonitrile. LC-MS(ESI) m/z (M+H)⁺: 802.34; calcd: 802.31; >95% purity.

Cpd. No. 22: 2-chloro-4-(((1R,3r,5S)-8-(4-(6-((1-(2-(2,6-dioxopiperidin-3-yl)-1,3-dioxoisoindolin-5-yl)azetidin-3-yl)methyl)-2,6-diazaspiro[3.3]heptan-2-yl)benzoyl)-8-azabicyclo[3.2.1]octan-3-yl)oxy)benzonitrile. LC-MS(ESI) m/z (M+H)⁺: 788.32; calcd: 788.30; >95% purity.

Cpd. No. 23: 2-chloro-4-(((1R,3r,5S)-8-(4-(4-(2-(2-(2,6-dioxopiperidin-3-yl)-1,3-dioxoisoindolin-5-yl)-2-azaspiro[3.3]heptane-6-carbonyl)piperazin-1-yl)benzoyl)-8-azabicyclo[3.2.1]octan-3-yl)oxy)benzonitrile. LC-MS(ESI) m/z (M+H)⁺: 830.33; calcd: 830.31; >95% purity.

Cpd. No. 24: 2-chloro-4-(((1R,3r,5S)-8-(4-(4-((2-(2-(2,6-dioxopiperidin-3-yl)-1,3-dioxoisoindolin-5-yl)-2-azaspiro[3.3]heptan-6-yl)methyl)piperazin-1-yl)benzoyl)-8-azabicyclo[3.2.1]octan-3-yl)oxy)benzonitrile. LC-MS(ESI) m/z (M+H)⁺: 816.32; calcd: 816.33; >95% purity.

Cpd. No. 25: 2-chloro-4-(((1R,3r,5S)-8-(4-(6-((1-(2-(2,6-dioxopiperidin-3-yl)-1,3-dioxoisoindolin-5-yl)piperidin-4-yl)methyl)-2,6-diazaspiro[3.3]heptan-2-yl)benzoyl)-8-azabicyclo[3.2.1]octan-3-yl)oxy)benzonitrile. LC-MS(ESI) m/z (M+H)⁺: 816.35; calcd: 816.33; >95% purity.

Cpd. No. 26: 6-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)-4-(trifluoromethyl)nicotinonitrile. LC-MS(ESI) m/z (M+H)⁺: 839.34; calcd: 839.35; >95% purity.

Cpd. No. 27: 2-chloro-4-(((1R,3r,5S)-8-(4-(1-((1-(2-(2,6-dioxopiperidin-3-yl)-1,3-dioxoisoindolin-5-yl)piperidin-4-yl)methyl)piperidin-4-yl)benzoyl)-8-azabicyclo[3.2.1]octan-3-yl)oxy)-3-methylbenzonitrile. LC-MS(ESI) m/z (M+H)⁺: 817.36; calcd: 817.35; >95% purity.

Cpd. No. 28: 2-chloro-4-((1-(4-(4-(1-(2-(2,6-dioxopiperidin-3-yl)-1,3-dioxoisoindolin-5-yl)piperidin-4-yl)piperazin-1-yl)benzoyl)piperidin-4-yl)oxy)benzonitrile. LC-MS(ESI) m/z (M+H)⁺: 764.32; calcd: 764.30; >95% purity.

Cpd. No. 29: 2-chloro-4-(((1R,3r,5S)-8-(4-(4-(1-(2-(2,6-dioxopiperidin-3-yl)-1,3-dioxoisoindolin-5-yl)piperidin-4-yl)piperazin-1-yl)benzoyl)-8-azabicyclo[3.2.1]octan-3-yl)oxy)benzonitrile. LC-MS(ESI) m/z (M+H)⁺: 790.33; calcd: 790.31; >95% purity.

Cpd. No. 30: 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)-3-methylbenzonitrile. LC-MS(ESI) m/z (M+H)⁺: 818.33; calcd: 818.35; >95% purity.

Cpd. No. 31: 2-chloro-4-(1-(4-((1′-(2-(2,6-dioxopiperidin-3-yl)-1,3-dioxoisoindolin-5-yl)-[1,4′-bipiperidin]-4-yl)ethynyl)-3-fluorobenzyl)-4,5-dimethyl-1H-pyrazol-3-yl)benzonitrile. LC-MS(ESI) m/z (M+H)⁺: 786.30; calcd: 786.30; >95% purity.

Cpd. No. 32: 2-chloro-4-(1-(4-((1′-(2-(2,6-dioxopiperidin-3-yl)-1,3-dioxoisoindolin-5-yl)-[1,4′-bipiperidin]-4-yl)ethynyl)-2-fluorobenzyl)-4,5-dimethyl-1H-pyrazol-3-yl)benzonitrile. LC-MS(ESI) m/z (M+H)⁺: 786.31; calcd: 786.30; >95% purity.

Cpd. No. 33: 2-chloro-4-(1-(4-((1′-(2-(2,6-dioxopiperidin-3-yl)-1,3-dioxoisoindolin-5-yl)-[1,4′-bipiperidin]-4-yl)ethynyl)benzyl)-1,4,5,6-tetrahydrocyclopenta[c]pyrazol-3-yl)benzonitrile. LC-MS(ESI) m/z (M+H)⁺: 780.29; calcd: 780.31; >95% purity.

Cpd. No. 34: 2-chloro-4-(1-(4-(4-((1-(2-(2,6-dioxopiperidin-3-yl)-1,3-dioxoisoindolin-5-yl)piperidin-4-yl)methyl)piperazine-1-carbonyl)benzyl)-4,5-dimethyl-1H-pyrazol-3-yl)benzonitrile. LC-MS(ESI) m/z (M+H)⁺: 787.32; calcd: 787.31; >95% purity.

Cpd. No. 35: 2-chloro-4-(1-(4-(4-(1-(2-(2,6-dioxopiperidin-3-yl)-1,3-dioxoisoindolin-5-yl)piperidin-4-yl)piperazine-1-carbonyl)benzyl)-4,5-dimethyl-1H-pyrazol-3-yl)benzonitrile. LC-MS(ESI) m/z (M+H)⁺: 773.31; calcd: 773.30; >95% purity.

Cpd. No. 54: 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazine-1-carbonyl)phenyl)-8-azabicyclo[3.2.1]octan-3-yl)oxy)benzonitrile. LC-MS(ESI) m/z (M+H)⁺: 804.31; calcd: 804.33; >95% purity.

Cpd. No. 55: 2-chloro-4-(((1R,3r,5S)-8-(4-(4-(1-(2-(2,6-dioxopiperidin-3-yl)-1,3-dioxoisoindolin-5-yl)piperidin-4-yl)piperazine-1-carbonyl)phenyl)-8-azabicyclo[3.2.1]octan-3-yl)oxy)benzonitrile. LC-MS(ESI) m/z (M+H)⁺: 790.29; calcd: 790.31; >95% purity.

Cpd. No. 56: 2-chloro-4-((1-(4-(4-((1-(2-(2,6-dioxopiperidin-3-yl)-1,3-dioxoisoindolin-5-yl)piperidin-4-yl)methyl)piperazine-1-carbonyl)phenyl)piperidin-4-yl)oxy)benzonitrile. LC-MS(ESI) m/z (M+H)⁺: 778.33; calcd: 778.31; >95% purity.

Cpd. No. 57: 2-chloro-4-((1-(4-(4-(1-(2-(2,6-dioxopiperidin-3-yl)-1,3-dioxoisoindolin-5-yl)piperidin-4-yl)piperazine-1-carbonyl)phenyl)piperidin-4-yl)oxy)benzonitrile. LC-MS(ESI) m/z (M+H)⁺: 764.32; calcd: 764.30; >95% purity.

Cpd. No. 129: 2-chloro-4-((6-(4-(4-((1-(2-(2,6-dioxopiperidin-3-yl)-1,3-dioxoisoindolin-5-yl)piperidin-4-yl)methyl)piperazin-1-yl)benzoyl)-6-azaspiro[3.5]nonan-9-yl)oxy)benzonitrile. LC-MS(ESI) m/z (M+H)⁺: 818.37; calcd: 818.35; >95% purity.

Cpd. No. 130: 2-chloro-4-((6-(4-(4-(1-(2-(2,6-dioxopiperidin-3-yl)-1,3-dioxoisoindolin-5-yl)piperidin-4-yl)piperazin-1-yl)benzoyl)-6-azaspiro[3.5]nonan-9-yl)oxy)benzonitrile. LC-MS(ESI) m/z (M+H)⁺: 804.35; calcd: 804.33; >95% purity.

Cpd. No. 131: 2-chloro-4-((5-(4-(4-((1-(2-(2,6-dioxopiperidin-3-yl)-1,3-dioxoisoindolin-5-yl)piperidin-4-yl)methyl)piperazin-1-yl)benzoyl)-5-azaspiro[2.5]octan-8-yl)oxy)benzonitrile. LC-MS(ESI) m/z (M+H)⁺: 804.32; calcd: 804.33; >95% purity.

Cpd. No. 132: 2-chloro-4-((5-(4-(4-(1-(2-(2,6-dioxopiperidin-3-yl)-1,3-dioxoisoindolin-5-yl)piperidin-4-yl)piperazin-1-yl)benzoyl)-5-azaspiro[2.5]octan-8-yl)oxy)benzonitrile. LC-MS(ESI) m/z (M+H)⁺: 790.30; calcd: 790.31; >95% purity.

Cpd. No. 133: 2-chloro-4-((3-(4-(4-((1-(2-(2,6-dioxopiperidin-3-yl)-1,3-dioxoisoindolin-5-yl)piperidin-4-yl)methyl)piperazin-1-yl)benzoyl)-3-azabicyclo[3.2.1]octan-8-yl)oxy)benzonitrile. LC-MS(ESI) m/z (M+H)⁺: 804.34; calcd: 804.33; >95% purity.

Cpd. No. 134: 2-chloro-4-((7-(4-(4-((1-(2-(2,6-dioxopiperidin-3-yl)-1,3-dioxoisoindolin-5-yl)piperidin-4-yl)methyl)piperazin-1-yl)benzoyl)-3-oxa-7-azabicyclo[3.3.1]nonan-9-yl)oxy)benzonitrile. LC-MS(ESI) m/z (M+H)⁺: 820.34; calcd: 820.32; >95% purity.

Cpd. No. 135: 2-chloro-4-((7-(4-(4-(1-(2-(2,6-dioxopiperidin-3-yl)-1,3-dioxoisoindolin-5-yl)piperidin-4-yl)piperazin-1-yl)benzoyl)-3-oxa-7-azabicyclo[3.3.1]nonan-9-yl)oxy)benzonitrile. LC-MS(ESI) m/z (M+H)⁺: 806.32; calcd: 806.31; >95% purity.

Cpd. No. 136: 2-chloro-4-((3-(4-(4-(1-(2-(2,6-dioxopiperidin-3-yl)-1,3-dioxoisoindolin-5-yl)piperidin-4-yl)piperazin-1-yl)benzoyl)-3-azabicyclo[3.2.1]octan-8-yl)oxy)benzonitrile. LC-MS(ESI) m/z (M+H)⁺: 790.33; calcd: 790.31; >95% purity.

Cpd. No. 187: 2-chloro-4-((1R,3s,5S)-3-(4-((1′-(2-(2,6-dioxopiperidin-3-yl)-1,3-dioxoisoindolin-5-yl)-[1,4′-bipiperidin]-4-yl)ethynyl)phenoxy)-8-azabicyclo[3.2.1]octan-8-yl)benzonitrile. LC-MS(ESI) m/z (M+H)⁺: 785.33; calcd: 785.32; >95% purity.

Cpd. No. 188: 2-chloro-4-(((1R,3r,5S)-8-(4-((1′-(2-(2,6-dioxopiperidin-3-yl)-1,3-dioxoisoindolin-5-yl)-[1,4′-bipiperidin]-4-yl)ethynyl)benzoyl)-8-azabicyclo[3.2.1]octan-3-yl)oxy)benzonitrile. LC-MS(ESI) m/z (M+H)⁺: 813.33; calcd: 813.32; >95% purity.

Cpd. No. 190: 2-chloro-4-(((1R,3r,5S)-8-(4-(4-((1-(2-(2,6-dioxopiperidin-3-yl)-1,3-dioxoisoindolin-5-yl)piperidin-4-yl)methyl)piperazin-1-yl)benzoyl)-8-azabicyclo[3.2.1]octan-3-yl)oxy)benzonitrile. LC-MS(ESI) m/z (M+H)⁺: 804.34; calcd: 804.33; >95% purity.

Cpd. No. 191: 2-chloro-4-(((1R,3r,5S)-8-(4-((1-((1-(2-(2,6-dioxopiperidin-3-yl)-1,3-dioxoisoindolin-5-yl)piperidin-4-yl)methyl)piperidin-4-yl)ethynyl)benzoyl)-8-azabicyclo[3.2.1]octan-3-yl)oxy)benzonitrile. LC-MS(ESI) m/z (M+H)⁺: 827.32; calcd: 827.33; >95% purity.

Cpd. No. 192: 2-chloro-4-(((1R,3r,5S)-8-(4-(4-(2-(4-(2-(2,6-dioxopiperidin-3-yl)-1,3-dioxoisoindolin-5-yl)piperazin-1-yl)ethyl)piperazin-1-yl)benzoyl)-8-azabicyclo[3.2.1]octan-3-yl)oxy)benzonitrile. LC-MS(ESI) m/z (M+H)⁺: 819.35; calcd: 819.34; >95% purity.

Cpd. No. 194: 2-chloro-4-(((1R,3r,5S)-8-(4-(4-(2-(1-(2-(2,6-dioxopiperidin-3-yl)-1,3-dioxoisoindolin-5-yl)piperidin-4-yl)ethyl)piperazin-1-yl)benzoyl)-8-azabicyclo[3.2.1]octan-3-yl)oxy)benzonitrile. LC-MS(ESI) m/z (M+H)⁺: 818.33; calcd: 818.35; >95% purity.

Cpd. No. 195: 2-chloro-4-(((1R,3r,5S)-8-(4-((1′-(2-(2,6-dioxopiperidin-3-yl)-1,3-dioxoisoindolin-4-yl)-[1,4′-bipiperidin]-4-yl)ethynyl)benzoyl)-8-azabicyclo[3.2.1]octan-3-yl)oxy)benzonitrile. LC-MS(ESI) m/z (M+H)⁺: 813.33; calcd: 813.32; >95% purity.

Cpd. No. 196: 2-chloro-4-(((1R,3r,5S)-8-(4-((1′-(2-(2,6-dioxopiperidin-3-yl)-1,3-dioxoisoindolin-4-yl)-[1,4′-bipiperidin]-4-yl)ethynyl)benzoyl)-8-azabicyclo[3.2.1]octan-3-yl)oxy)benzonitrile. LC-MS(ESI) m/z (M+H)⁺: 813.34; calcd: 813.32; >95% purity.

Cpd. No. 43: 2-chloro-4-(1-(4-((1′-(2-(2,6-dioxopiperidin-3-yl)-6-fluoro-1,3-dioxoisoindolin-5-yl)-[1,4′-bipiperidin]-4-yl)ethynyl)benzyl)-4,5-dimethyl-1H-pyrazol-3-yl)benzonitrile. LC-MS(ESI) m/z (M+H)⁺: 786.29; calcd: 786.30; >95% purity.

Cpd. No. 44: 2-chloro-4-(1-(4-((1′-(2-(2,6-dioxopiperidin-3-yl)-1,3-dioxoisoindolin-5-yl)-[1,4′-bipiperidin]-4-yl)ethynyl)benzyl)-4,5,6,7-tetrahydro-1H-indazol-3-yl)benzonitrile. LC-MS(ESI) m/z (M+H)⁺: 794.33; calcd: 794.32; >95% purity.

Cpd. No. 45: 2-chloro-4-(1-(4-((1′-(2-(2,6-dioxopiperidin-3-yl)-1,3-dioxoisoindolin-5-yl)-[1,4′-bipiperidin]-4-yl)ethynyl)benzyl)-4-ethyl-5-methyl-1H-pyrazol-3-yl)benzonitrile. LC-MS(ESI) m/z (M+H)⁺: 782.34; calcd: 782.32; >95% purity.

Cpd. No. 46: 4-(1-(4-((1′-(2-(2,6-dioxopiperidin-3-yl)-1,3-dioxoisoindolin-5-yl)-[1,4′-bipiperidin]-4-yl)ethynyl)benzyl)-4,5-dimethyl-1H-pyrazol-3-yl)-2-(trifluoromethyl)benzonitrile. LC-MS(ESI) m/z (M+H)⁺: 802.32; calcd: 802.34; >95% purity.

Cpd. No. 47: 2-chloro-4-(1-(4-(((1r,4r)-4-(4-((2-(2,6-dioxopiperidin-3-yl)-3-oxoisoindolin-5-yl)ethynyl)piperidine-1-carbonyl)cyclohexyl)ethynyl)benzyl)-4,5-dimethyl-1H-pyrazol-3-yl)benzonitrile. LC-MS(ESI) m/z (M+H)⁺: 805.34; calcd: 805.33; >95% purity.

Cpd. No. 53: 2-chloro-4-(1-(4-(4-(2-(2-(2,6-dioxopiperidin-3-yl)-1,3-dioxoisoindolin-5-yl)-2-azaspiro[3.3]heptan-6-yl)piperazine-1-carbonyl)benzyl)-4,5-dimethyl-1H-pyrazol-3-yl)benzonitrile. LC-MS(ESI) m/z (M+H)⁺: 785.33; calcd: 785.31; >95% purity.

Cpd. No. 137: 2-chloro-4-(1-(4-(4-(1-(2-(2,6-dioxopiperidin-3-yl)-1,3-dioxoisoindolin-5-yl)azetidin-3-yl)piperazine-1-carbonyl)benzyl)-4,5-dimethyl-1H-pyrazol-3-yl)benzonitrile. LC-MS(ESI) m/z (M+H)⁺: 745.27; calcd: 745.27; >95% purity.

Cpd. No. 138: 2-chloro-4-(1-(4-(4-((1-(2-(2,6-dioxopiperidin-3-yl)-1,3-dioxoisoindolin-5-yl)azetidin-3-yl)methyl)piperazine-1-carbonyl)benzyl)-4,5-dimethyl-1H-pyrazol-3-yl)benzonitrile. LC-MS(ESI) m/z (M+H)⁺: 759.30; calcd: 759.28; >95% purity.

Cpd. No. 139: 2-chloro-4-(1-(4-(4-(1-(2-(2,6-dioxopiperidin-3-yl)-3-oxoisoindolin-5-yl)azetidin-3-yl)piperazine-1-carbonyl)benzyl)-4,5-dimethyl-1H-pyrazol-3-yl)benzonitrile. LC-MS(ESI) m/z (M+H)⁺: 731.31; calcd: 731.29; >95% purity.

Cpd. No. 165: 2-chloro-4-(1-(4-(4-(7-(2-(2,6-dioxopiperidin-3-yl)-1-oxoisoindolin-5-yl)hept-6-ynoyl)piperazin-1-yl)benzyl)-4,5-dimethyl-1H-pyrazol-3-yl)benzonitrile. LC-MS(ESI) m/z (M+H)⁺: 756.32; calcd: 756.31; >95% purity.

Cpd. No. 166: 2-chloro-4-(1-(4-(((1r,4r)-4-(4-(4-(2-(2,6-dioxopiperidin-3-yl)-1,3-dioxoisoindolin-4-yl)piperazin-1-yl)piperidine-1-carbonyl)cyclohexyl)ethynyl)benzyl)-4,5-dimethyl-1H-pyrazol-3-yl)benzonitrile. LC-MS(ESI) m/z (M+H)⁺: 879.40; calcd: 879.38; >95% purity.

Cpd. No. 177: (1r,4r)-4-((4-((3-(3-chloro-4-cyanophenyl)-4,5-dimethyl-1H-pyrazol-1-yl)methyl)phenyl)ethynyl)-N-(2-(4-(2-(2,6-dioxopiperidin-3-yl)-1,3-dioxoisoindolin-4-yl)piperazin-1-yl)ethyl)cyclohexane-1-carboxamide. LC-MS(ESI) m/z (M+H)⁺: 839.37; calcd: 839.35; >95% purity.

Cpd. No. 179: 2-chloro-4-(1-(4-(4-(2-(4-(2-(2,6-dioxopiperidin-3-yl)-1,3-dioxoisoindolin-4-yl)piperazin-1-yl)ethyl)piperazin-1-yl)benzyl)-4,5-dimethyl-1H-pyrazol-3-yl)benzonitrile. LC-MS(ESI) m/z (M+H)⁺: 774.32; calcd: 774.33; >95% purity.

Cpd. No. 180: 2-chloro-4-(1-(4-(((1r,4r)-4-(4-(1-(2-(2,6-dioxopiperidin-3-yl)-1,3-dioxoisoindolin-4-yl)piperidin-4-yl)piperazine-1-carbonyl)cyclohexyl)ethynyl)benzyl)-4,5-dimethyl-1H-pyrazol-3-yl)benzonitrile. LC-MS(ESI) m/z (M+H)⁺: 879.39; calcd: 879.38; >95% purity.

Cpd. No. 181: 2-chloro-4-(1-(4-(((1r,4r)-4-(4-(2-(2,6-dioxopiperidin-3-yl)-1,3-dioxoisoindolin-4-yl)piperazine-1-carbonyl)cyclohexyl)ethynyl)benzyl)-4,5-dimethyl-1H-pyrazol-3-yl)benzonitrile. LC-MS(ESI) m/z (M+H)⁺: 796.28; calcd: 796.30; >95% purity.

Cpd. No. 182: 2-chloro-4-(1-(4-(((1r,4r)-4-(4-(4-(2-(2,6-dioxopiperidin-3-yl)-1,3-dioxoisoindolin-5-yl)piperazin-1-yl)piperidine-1-carbonyl)cyclohexyl)ethynyl)benzyl)-4,5-dimethyl-1H-pyrazol-3-yl)benzonitrile. LC-MS(ESI) m/z (M+H)⁺: 879.39; calcd: 879.38; >95% purity.

Cpd. No. 183: 2-chloro-4-(1-(4-(((1r,4r)-4-(4-(2-(2,6-dioxopiperidin-3-yl)-1,3-dioxoisoindolin-5-yl)piperazine-1-carbonyl)cyclohexyl)ethynyl)benzyl)-4,5-dimethyl-1H-pyrazol-3-yl)benzonitrile. LC-MS(ESI) m/z (M+H)⁺: 796.32; calcd: 796.30; >95% purity.

Cpd. No. 184: 2-chloro-4-(1-(4-((4-((3-((6-(2,6-dioxopiperidin-3-yl)-5-oxo-6,7-dihydro-5H-pyrrolo[3,4-b]pyridin-3-yl)ethynyl)azetidin-1-yl)methyl)bicyclo[2.2.2]octan-1-yl)ethynyl)benzyl)-4,5-dimethyl-1H-pyrazol-3-yl)benzonitrile. LC-MS(ESI) m/z (M+H)⁺: 790.34; calcd: 790.33; >95% purity.

Cpd. No. 185: 2-chloro-4-(1-(4-(((1r,4r)-4-(4-(1-(2-(2,6-dioxopiperidin-3-yl)-1,3-dioxoisoindolin-5-yl)piperidin-4-yl)piperazine-1-carbonyl)cyclohexyl)ethynyl)benzyl)-4,5-dimethyl-1H-pyrazol-3-yl)benzonitrile. LC-MS(ESI) m/z (M+H)⁺: 879.37; calcd: 879.38; >95% purity.

Cpd. No. 186: 2-chloro-4-(1-(4-((1′-(2-(2,6-dioxopiperidin-3-yl)-1,3-dioxoisoindolin-5-yl)-[1,4′-bipiperidin]-4-yl)ethynyl)benzyl)-4,5-dimethyl-1H-pyrazol-3-yl)benzonitrile. LC-MS(ESI) m/z (M+H)⁺: 768.33; calcd: 768.31; >95% purity.

Cpd. No. 189: 2-chloro-4-(1-(4-(4-(2-(1-(2-(2,6-dioxopiperidin-3-yl)-1,3-dioxoisoindolin-4-yl)piperidin-4-yl)ethyl)piperazin-1-yl)benzyl)-4,5-dimethyl-1H-pyrazol-3-yl)benzonitrile. LC-MS(ESI) m/z (M+H)⁺: 773.33; calcd: 773.34; >95% purity.

Cpd. No. 193: 2-chloro-4-(1-(4-(4-(2-(1-(2-(2,6-dioxopiperidin-3-yl)-1,3-dioxoisoindolin-5-yl)piperidin-4-yl)ethyl)piperazin-1-yl)benzyl)-4,5-dimethyl-1H-pyrazol-3-yl)benzonitrile. LC-MS(ESI) m/z (M+H)⁺: 773.33; calcd: 773.34; >95% purity.

Cpd. No. 36: 2-chloro-4-((1r,3r)-3-(5-((1′-(2-(2,6-dioxopiperidin-3-yl)-1,3-dioxoisoindolin-5-yl)-[1,4′-bipiperidin]-4-yl)ethynyl)-1-oxoisoindolin-2-yl)-2,2,4,4-tetramethylcyclobutoxy)benzonitrile. LC-MS(ESI) m/z (M+H)⁺: 841.33; calcd: 841.35; >95% purity.

Cpd. No. 37: 2-chloro-4-((1r,3r)-3-(5-(4-(1-(2-(2,6-dioxopiperidin-3-yl)-1,3-dioxoisoindolin-5-yl)piperidin-4-yl)piperazin-1-yl)-1-oxoisoindolin-2-yl)-2,2,4,4-tetramethylcyclobutoxy)benzonitrile. LC-MS(ESI) m/z (M+H)⁺: 818.33; calcd: 818.35; >95% purity.

Cpd. No. 39: 2-chloro-4-((1r,3r)-3-(5-(4-(1-(1-(2-(2,6-dioxopiperidin-3-yl)-1,3-dioxoisoindolin-5-yl)piperidin-4-yl)azetidin-3-yl)piperazin-1-yl)-1-oxoisoindolin-2-yl)-2,2,4,4-tetramethylcyclobutoxy)benzonitrile. LC-MS(ESI) m/z (M+H)⁺: 873.40; calcd: 873.39; >95% purity.

Cpd. No. 40: 2-chloro-4-((1r,3r)-3-(6-((1′-(2-(2,6-dioxopiperidin-3-yl)-1,3-dioxoisoindolin-5-yl)-[1,4′-bipiperidin]-4-yl)ethynyl)-1-oxoisoindolin-2-yl)-2,2,4,4-tetramethylcyclobutoxy)benzonitrile. LC-MS(ESI) m/z (M+H)⁺: 841.33; calcd: 841.35; >95% purity.

Cpd. No. 41: 2-chloro-4-((1r,3r)-3-(5-((1-(2-(2-(2,6-dioxopiperidin-3-yl)-1,3-dioxoisoindolin-5-yl)-2-azaspiro[3.3]heptan-6-yl)piperidin-4-yl)ethynyl)-1-oxoisoindolin-2-yl)-2,2,4,4-tetramethylcyclobutoxy)benzonitrile. LC-MS(ESI) m/z (M+H)⁺: 853.36; calcd: 853.35; >95% purity.

Cpd. No. 42: 2-chloro-4-((1r,3r)-3-(5-((4-(4-(2-(2,6-dioxopiperidin-3-yl)-1,3-dioxoisoindolin-5-yl)piperazin-1-yl)cyclohexyl)ethynyl)-1-oxoisoindolin-2-yl)-2,2,4,4-tetramethylcyclobutoxy)benzonitrile. LC-MS(ESI) m/z (M+H)⁺: 841.37; calcd: 841.35; >95% purity.

Cpd. No. 49: 2-chloro-4-((1r,3r)-3-(6-((1-(2-(2-(2,6-dioxopiperidin-3-yl)-1,3-dioxoisoindolin-5-yl)-2-azaspiro[3.3]heptan-6-yl)piperidin-4-yl)ethynyl)-1-oxoisoindolin-2-yl)-2,2,4,4-tetramethylcyclobutoxy)benzonitrile. LC-MS(ESI) m/z (M+H)⁺: 853.36; calcd: 853.35; >95% purity.

Cpd. No. 50: 2-chloro-4-((1r,3r)-3-(5-(4-(2-(2-(2,6-dioxopiperidin-3-yl)-1,3-dioxoisoindolin-5-yl)-2-azaspiro[3.3]heptan-6-yl)piperazin-1-yl)-1-oxoisoindolin-2-yl)-2,2,4,4-tetramethylcyclobutoxy)benzonitrile. LC-MS(ESI) m/z (M+H)⁺: 830.33; calcd: 830.35; >95% purity.

Cpd. No. 51: 2-chloro-4-(((1r,4r)-4-(6-((1′-(2-(2,6-dioxopiperidin-3-yl)-1,3-dioxoisoindolin-5-yl)-[1,4′-bipiperidin]-4-yl)ethynyl)-1-oxoisoindolin-2-yl)cyclohexyl)oxy)benzonitrile. LC-MS(ESI) m/z (M+H)⁺: 813.33; calcd: 813.32; >95% purity.

Cpd. No. 140: 2-chloro-4-((1r,3r)-3-(5-(4-(1-(2-(2,6-dioxopiperidin-3-yl)-1,3-dioxoisoindolin-5-yl)azetidin-3-yl)piperazine-1-carbonyl)-1-oxoisoindolin-2-yl)-2,2,4,4-tetramethylcyclobutoxy)benzonitrile. LC-MS(ESI) m/z (M+H)⁺: 818.33; calcd: 818.31; >95% purity.

Cpd. No. 141: 2-chloro-4-((1r,3r)-3-(5-(4-(1-(2-(2,6-dioxopiperidin-3-yl)-1,3-dioxoisoindolin-5-yl)piperidin-4-yl)piperazine-1-carbonyl)-1-oxoisoindolin-2-yl)-2,2,4,4-tetramethylcyclobutoxy)benzonitrile. LC-MS(ESI) m/z (M+H)⁺: 846.33; calcd: 846.34; >95% purity.

Cpd. No. 142: 2-chloro-4-((1r,3r)-3-(5-((1-(1-(2-(2,6-dioxopiperidin-3-yl)-1,3-dioxoisoindolin-5-yl)azetidin-3-yl)piperidin-4-yl)ethynyl)-1-oxoisoindolin-2-yl)-2,2,4,4-tetramethylcyclobutoxy)benzonitrile. LC-MS(ESI) m/z (M+H)⁺: 813.33; calcd: 813.32; >95% purity.

Cpd. No. 143: 2-chloro-4-((1r,3r)-3-(5-(4-((1-(2-(2,6-dioxopiperidin-3-yl)-1,3-dioxoisoindolin-5-yl)piperidin-4-yl)methyl)piperazine-1-carbonyl)-1-oxoisoindolin-2-yl)-2,2,4,4-tetramethylcyclobutoxy)benzonitrile. LC-MS(ESI) m/z (M+H)⁺: 860.38; calcd: 860.36; >95% purity.

Cpd. No. 144: 2-chloro-4-((1r,3r)-3-(5-(4-((1-(2-(2,6-dioxopiperidin-3-yl)-1,3-dioxoisoindolin-5-yl)azetidin-3-yl)methyl)piperazine-1-carbonyl)-1-oxoisoindolin-2-yl)-2,2,4,4-tetramethylcyclobutoxy)benzonitrile. LC-MS(ESI) m/z (M+H)⁺: 832.33; calcd: 832.32; >95% purity.

Cpd. No. 145: 2-chloro-4-((1r,3r)-3-(5-(4-(1-(2-(2,6-dioxopiperidin-3-yl)-1,3-dioxoisoindolin-5-yl)azetidin-3-yl)piperazin-1-yl)-1-oxoisoindolin-2-yl)-2,2,4,4-tetramethylcyclobutoxy)benzonitrile. LC-MS(ESI) m/z (M+H)⁺: 790.33; calcd: 790.31; >95% purity.

Cpd. No. 146: 2-chloro-4-((1r,3r)-3-(5-(4-((2-(2-(2,6-dioxopiperidin-3-yl)-1,3-dioxoisoindolin-5-yl)-2-azaspiro[3.3]heptan-6-yl)methyl)piperazin-1-yl)-1-oxoisoindolin-2-yl)-2,2,4,4-tetramethylcyclobutoxy)benzonitrile. LC-MS(ESI) m/z (M+H)⁺: 844.37; calcd: 844.36; >95% purity.

Cpd. No. 147: 2-chloro-4-((1r,3r)-3-(5-(4-(6-(2-(2,6-dioxopiperidin-3-yl)-1,3-dioxoisoindolin-5-yl)-6-azaspiro[3.4]octan-2-yl)piperazin-1-yl)-1-oxoisoindolin-2-yl)-2,2,4,4-tetramethylcyclobutoxy)benzonitrile. LC-MS(ESI) m/z (M+H)⁺: 844.38; calcd: 844.36; >95% purity.

Cpd. No. 148: 2-chloro-4-((1r,3r)-3-(5-(4-(1′-(2-(2,6-dioxopiperidin-3-yl)-1,3-dioxoisoindolin-5-yl)-[1,3′-biazetidin]-3-yl)piperazin-1-yl)-1-oxoisoindolin-2-yl)-2,2,4,4-tetramethylcyclobutoxy)benzonitrile. LC-MS(ESI) m/z (M+H)⁺: 845.37; calcd: 845.36; >95% purity.

Cpd. No. 149: 2-chloro-4-((1r,3r)-3-(5-(4-(7-(2-(2,6-dioxopiperidin-3-yl)-1,3-dioxoisoindolin-5-yl)-7-azaspiro[3.5]nonan-2-yl)piperazin-1-yl)-1-oxoisoindolin-2-yl)-2,2,4,4-tetramethylcyclobutoxy)benzonitrile. LC-MS(ESI) m/z (M+H)⁺: 858.37; calcd: 858.38; >95% purity.

Cpd. No. 150: 2-chloro-4-((1r,3r)-3-(5-((1-(1-(2-(2,6-dioxopiperidin-3-yl)-1,3-dioxoisoindolin-5-yl)piperidin-4-yl)azetidin-3-yl)ethynyl)-1-oxoisoindolin-2-yl)-2,2,4,4-tetramethylcyclobutoxy)benzonitrile. LC-MS(ESI) m/z (M+H)⁺: 813.33; calcd: 813.32; >95% purity.

Cpd. No. 151: 2-chloro-4-((1r,3r)-3-(5-(4-(1-(2-(2,6-dioxopiperidin-3-yl)-1,3-dioxoisoindolin-5-yl)-1-azaspiro[3.3]heptan-6-yl)piperazin-1-yl)-1-oxoisoindolin-2-yl)-2,2,4,4-tetramethylcyclobutoxy)benzonitrile. LC-MS(ESI) m/z (M+H)⁺: 830.33; calcd: 830.35; >95% purity.

Cpd. No. 152: 2-chloro-4-((1r,3r)-3-(5-(4-(2-(2-(2,6-dioxopiperidin-3-yl)-1,3-dioxoisoindolin-5-yl)-2-azaspiro[3.5]nonan-7-yl)piperazin-1-yl)-1-oxoisoindolin-2-yl)-2,2,4,4-tetramethylcyclobutoxy)benzonitrile. LC-MS(ESI) m/z (M+H)⁺: 858.40; calcd: 858.38; >95% purity.

Cpd. No. 153: 2-chloro-4-((1r,3r)-3-(5-(4-((1-(2-(2,6-dioxopiperidin-3-yl)-1,3-dioxoisoindolin-5-yl)-1-azaspiro[3.3]heptan-6-yl)methyl)piperazin-1-yl)-1-oxoisoindolin-2-yl)-2,2,4,4-tetramethylcyclobutoxy)benzonitrile. LC-MS(ESI) m/z (M+H)⁺: 844.38; calcd: 844.36; >95% purity.

Cpd. No. 154: 2-chloro-4-((1r,3r)-3-(5-((1′-(2-(2,6-dioxopiperidin-3-yl)-1,3-dioxoisoindolin-5-yl)-[1,3′-biazetidin]-3-yl)ethynyl)-1-oxoisoindolin-2-yl)-2,2,4,4-tetramethylcyclobutoxy)benzonitrile. LC-MS(ESI) m/z (M+H)⁺: 785.30; calcd: 785.29; >95% purity.

Cpd. No. 155: 2-chloro-4-((1r,3r)-3-(5-((1-(2-(2,6-dioxopiperidin-3-yl)-1,3-dioxoisoindolin-5-yl)piperidin-4-yl)ethynyl)-1-oxoisoindolin-2-yl)-2,2,4,4-tetramethylcyclobutoxy)benzonitrile. LC-MS(ESI) m/z (M+H)⁺: 758.29; calcd: 758.28; >95% purity.

Cpd. No. 156: 2-chloro-4-((1r,3r)-3-(5-((1-(2-(2,6-dioxopiperidin-3-yl)-1,3-dioxoisoindolin-5-yl)azetidin-3-yl)ethynyl)-1-oxoisoindolin-2-yl)-2,2,4,4-tetramethylcyclobutoxy)benzonitrile. LC-MS(ESI) m/z (M+H)⁺: 730.27; calcd: 730.25; >95% purity.

Cpd. No. 157: 2-chloro-4-((1r,3r)-3-(5-((2-(1-(2-(2,6-dioxopiperidin-3-yl)-1,3-dioxoisoindolin-5-yl)azetidin-3-yl)-2-azaspiro[3.3]heptan-6-yl)ethynyl)-1-oxoisoindolin-2-yl)-2,2,4,4-tetramethylcyclobutoxy)benzonitrile. LC-MS(ESI) m/z (M+H)⁺: 825.33; calcd: 825.32; >95% purity.

Cpd. No. 158: 2-chloro-4-((1r,3r)-3-(5-((6-(1-(2-(2,6-dioxopiperidin-3-yl)-1,3-dioxoisoindolin-5-yl)azetidin-3-yl)-6-azaspiro[3.4]octan-2-yl)ethynyl)-1-oxoisoindolin-2-yl)-2,2,4,4-tetramethylcyclobutoxy)benzonitrile. LC-MS(ESI) m/z (M+H)⁺: 839.35; calcd: 839.33; >95% purity.

Cpd. No. 159: 2-chloro-4-((1r,3r)-3-(5-((2-(2-(2,6-dioxopiperidin-3-yl)-1,3-dioxoisoindolin-5-yl)-2-azaspiro[3.3]heptan-6-yl)ethynyl)-1-oxoisoindolin-2-yl)-2,2,4,4-tetramethylcyclobutoxy)benzonitrile. LC-MS(ESI) m/z (M+H)⁺: 770.29; calcd: 770.28; >95% purity.

Cpd. No. 160: 2-chloro-4-((1r,3r)-3-(5-((6-(2-(2,6-dioxopiperidin-3-yl)-1,3-dioxoisoindolin-5-yl)-6-azaspiro[3.4]octan-2-yl)ethynyl)-1-oxoisoindolin-2-yl)-2,2,4,4-tetramethylcyclobutoxy)benzonitrile. LC-MS(ESI) m/z (M+H)⁺: 784.31; calcd: 784.29; >95% purity.

Cpd. No. 161: 2-chloro-4-((1r,3r)-3-(5-((1-(2-(2-(2,6-dioxopiperidin-3-yl)-1,3-dioxoisoindolin-5-yl)-2-azaspiro[3.3]heptan-6-yl)azetidin-3-yl)ethynyl)-1-oxoisoindolin-2-yl)-2,2,4,4-tetramethylcyclobutoxy)benzonitrile. LC-MS(ESI) m/z (M+H)⁺: 825.33; calcd: 825.32; >95% purity.

Cpd. No. 167: 4-((1r,3r)-3-(5-((1′-(2-(2,6-dioxopiperidin-3-yl)-1,3-dioxoisoindolin-5-yl)-[1,3′-biazetidin]-3-yl)ethynyl)-1-oxoisoindolin-2-yl)-2,2,4,4-tetramethylcyclobutoxy)-2-(trifluoromethyl)benzonitrile. LC-MS(ESI) m/z (M+H)⁺: 819.33; calcd: 819.31; >95% purity.

Cpd. No. 168: 5-((1r,3r)-3-(5-((1′-(2-(2,6-dioxopiperidin-3-yl)-1,3-dioxoisoindolin-5-yl)-[1,3′-biazetidin]-3-yl)ethynyl)-1-oxoisoindolin-2-yl)-2,2,4,4-tetramethylcyclobutoxy)-3-(trifluoromethyl)picolinonitrile.

Cpd. No. 169: 2-chloro-4-((1r,3r)-3-(5-((1′-(2-(2,6-dioxopiperidin-3-yl)-1,3-dioxoisoindolin-5-yl)-[1,3′-biazetidin]-3-yl)ethynyl)-4-fluoro-1-oxoisoindolin-2-yl)-2,2,4,4-tetramethylcyclobutoxy)benzonitrile.

Cpd. No. 170: 2-chloro-4-((1r,3r)-3-(5-((1′-(2-(2,6-dioxopiperidin-3-yl)-1,3-dioxoisoindolin-5-yl)-[1,3′-biazetidin]-3-yl)ethynyl)-6-fluoro-1-oxoisoindolin-2-yl)-2,2,4,4-tetramethylcyclobutoxy)benzonitrile.

Cpd. No. 171: 2-chloro-4-((1r,3r)-3-(5-((1′-(2-(2,6-dioxopiperidin-3-yl)-1,3-dioxoisoindolin-5-yl)-[1,3′-biazetidin]-3-yl)ethynyl)-7-fluoro-1-oxoisoindolin-2-yl)-2,2,4,4-tetramethylcyclobutoxy)benzonitrile.

Cpd. No. 37: 2-chloro-4-(((1r,4r)-4-(5-((1′-(2-(2,6-dioxopiperidin-3-yl)-1,3-dioxoisoindolin-5-yl)-[1,4′-bipiperidin]-4-yl)ethynyl)-1-oxoisoindolin-2-yl)cyclohexyl)oxy)benzonitrile. LC-MS(ESI) m/z (M+H)⁺: 813.33; calcd: 813.32; >95% purity.

Cpd. No. 52: 2-chloro-4-(((1r,4r)-4-(6-((1-(2-(2-(2,6-dioxopiperidin-3-yl)-1,3-dioxoisoindolin-5-yl)-2-azaspiro[3.3]heptan-6-yl)piperidin-4-yl)ethynyl)-1-oxoisoindolin-2-yl)cyclohexyl)oxy)benzonitrile. LC-MS(ESI) m/z (M+H)⁺: 825.33; calcd: 825.32; >95% purity.

Cpd. No. 162: 2-chloro-4-(((1r,4r)-4-(5-((1-(1-(2-(2,6-dioxopiperidin-3-yl)-1,3-dioxoisoindolin-5-yl)azetidin-3-yl)piperidin-4-yl)ethynyl)-1-oxoisoindolin-2-yl)cyclohexyl)oxy)benzonitrile. LC-MS(ESI) m/z (M+H)⁺: 785.31; calcd: 785.29; >95% purity.

Cpd. No. 163: 2-chloro-4-(((1r,4r)-4-(5-((1-(1-(2-(2,6-dioxopiperidin-3-yl)-1,3-dioxoisoindolin-5-yl)piperidin-4-yl)azetidin-3-yl)ethynyl)-1-oxoisoindolin-2-yl)cyclohexyl)oxy)benzonitrile. LC-MS(ESI) m/z (M+H)⁺: 785.31; calcd: 785.29; >95% purity.

Cpd. No. 164: 2-chloro-4-(1-(4-((1′-(2-(2,6-dioxopiperidin-3-yl)-1,3-dioxoisoindolin-5-yl)-[1,4′-bipiperidin]-4-yl)ethynyl)benzyl)-4,5-dimethyl-1H-pyrazol-3-yl)benzonitrile. LC-MS(ESI) m/z (M+H): 757.28; calcd: 757.26; >95% purity.

The representative Compounds of the Disclosure of Table 6 were also prepared using the methods described in EXAMPLES 1-21

TABLE 6

| Cpd. | |
|---|---|
| No. | IUPAC Name, NMR Data, UPLC-MS Data, HPLC Data |
| 336 | 2-chloro-4-((1-(4-(4-(4-(2-(2,6-dioxopiperidin-3-yl)-1,3- |
| | dioxoisoindolin-5-yl)piperazin-1-yl)piperidin-1- |
| | yl)benzoyl)piperidin-4-yl)oxy)benzonitrile. ¹H NMR |
| | (acetonitrile-d₃): δ 9.11 (s, 1H), 8.96 (s, 1H), 7.72 (m, 2H), 7.39 |
| | (m, 2H), 7.23 (m, 2H), 7.03 (m, 3H), 5.00 (m, 1H), 4.77 (m, 1H), |
| | 4.01 (m, 2H), 3.86 (m, 2H), 3.69 (m, 4H), 3.44 (m, 3H), 3.35 (m, |
| | 2H), 3.14 (m, 4H), 2.79 (m, 5H), 2.21 (m, 1H), 2.12 (m, 1H), 2.02 |
| | (m, 2H), 1.88 (m, 1H), 1.74 (m, 2H). UPLC-MS: 3.8 min, MS |
| | [M + H]: found 764.00, calculated: 764.29. |
| | Prepn HPLC 42% ACN in water. |
| 337 | 2-chloro-4-((1-(4-(4-(4-(2-(2,6-dioxopiperidin-3-yl)-1,3- |
| | dioxoisoindolin-5-yl)piperazin-1-yl)piperidin-1- |
| | yl)benzoyl)azepan-4-yl)oxy)benzonitrile. ¹H NMR (acetonitrile- |
| | d₃): δ 8.91 (s, 1H), 7.96 (m, 1H), 7.71 (m, 2H), |
| | 7.59 (m, 1H), 7.40 (m, 2H), 7.20 (m, 2H), 6.97 (m, 2H), |
| | 4.99 (m, 2H), 4.70 (m, 1H), 4.49 (m, 1H), 4.08 (m, 1H), |
| | 3.94 (m, 2H), 3.58 (m, 7H), 3.29 (m, 2H), 3.12 (m, 3H), |
| | 2.77 (m, 5H), 1.58 (m, 3H), 1.09 (m, 3H), 0.91 |
| | (m, 3H). UPLC-MS: 4.1 min, MS [M + H]: found 778.21, |
| | calculated: 778.30. Prepn HPLC 41% ACN in water. |
| 338 | 2-chloro-4-(1-(4-(4-(1-(2-(2,6-dioxopiperidin-3-yl)-1,3- |
| | dioxoisoindolin-5-yl)piperidin-4-yl)piperazine-1- |
| | carbonyl)benzyl)-1H-pyrazol-3-yl)benzonitrile. ¹H NMR |
| | (acetonitrile-d₃): δ 8.89 (s, 1H), 8.05 (d, J = 1.6 Hz, 1H), 7.92 (dd, |
| | J = 8.2 Hz, 1.6 Hz, 1H), 7.82 (d, J = 8.2 Hz, 1H), |
| | 7.75 (d, J = 2.4 Hz,1H), 7.70 (d, J = 8.4 Hz, 1H), 7.46 |
| | (d, J = 8.4 Hz, 2H), 7.39 (s, 1H), 7.37 (s, 1H), 7.35 |
| | (d, J = 2.4 Hz, 1H), 7.22 (dd, J = 8.6 Hz, 2.4 Hz, |
| | 1H), 6.80 (d, J = 2.4 Hz, 1H), 5.44 (s, 2H), 4.96 (m, 1H), 4.16 (m, |
| | 2H), 3.45 (m, 3H), 3.01 (m, 3H), 2.72 (m, 4H), 2.17 (m, 5H), 1.82 |
| | (m, 3H), 1.37 (m, 1H). UPLC-MS: 3.7 min, MS [M + H]: found |
| | 745.22, calculated: 745.26. Prepn HPLC 42% ACN in water. |

### Example 23

### Biological Assays

### A. Western Blotting Methods

The appropriate cell line, e.g., prostate cancer LNCaP, Vcap, or MDA-MB-453, cell line, was treated with Compounds of the Disclosure. The treated cells were lysed with RIPA buffer. The AR level in the cell lysates was examined by western blotting and a specific AR antibody (ab 194196, Abeam, Cambridge, Mass. 02139) with concentration of 1:20,000. GAPDH was used as a loading control.

B. Band Quantification and DC₅₀ and DC₉₀ Value Calculation

Bands were quantified with ImageJ software. The relative numbers of each band obtained from normalization with its corresponding GAPDH level were compared with Prism 8 software. The DC₅₀ values were produced from Prism 8, and the DC₉₀ values were calculated with an equation=Bottom+(Top−Bottom)/(1+10\hat{ }((Log EC50−X)*HillSlope) based on DC₅₀ and Hillslope values.

The amount of AR protein degradation in LNCap, VCap, and MDA-MB-453 cells caused representative Compounds of the Disclosure at the concentrations indicated is presented in Table 4.

TABLE 4

| Cpd. | LNCap | | VCap | | MDA-MB-453 | |
|---|---|---|---|---|---|---|
| No. | 10 nM | 100 nM | 10 nM | 100 nM | 10 nM | 100 nM |
| 58 | | | D | A | | |
| 59 | | | D | B | | |
| 60 | | | C | A | | |
| 61 | | | B | B | | |
| 62 | | | D | C | | |
| 63 | | | | | D | D |
| 64 | | | | | D | D |
| 65 | | | | | D | D |
| 66 | | | | | D | C |
| 67 | | | | | D | C |
| 68 | | | | | D | C |
| 69 | | | | | A | A |
| 70 | | | | | A | A |
| 71 | | | | | D | C |
| 72 | | | | | D | C |
| 73 | | | | | D | D |
| 74 | | | | | D | D |
| 75 | | | | | C | B |
| 76 | | | | | C | B |
| 77 | | | | | C | B |
| 78 | | | | | C | B |
| 79 | | | | | C | A |
| 80 | | | | | C | A |
| 81 | | | | | D | B |
| 82 | | | | | D | D |
| 83 | | | | | D | C |
| 84 | | | | | A | A |
| 85 | | | | | D | D |
| 86 | | | | | D | C |
| 87 | | | | | D | C |
| 88 | | | | | A | A |
| 89 | | | | | B | A |
| 90 | | | | | C | B |
| 91 | | | | | A | A |
| 92 | | | | | C | A |
| 93 | | | | | C | B |
| 94 | | | | | A | A |
| 95 | | | | | B | B |
| 97 | | | | | C | B |
| 98 | | | | | B | A |
| 99 | | | | | C | C |
| 100 | | | | | D | B |
| 101 | | | | | D | A |
| 102 | | | | | D | A |
| 103 | | | | | D | B |
| 104 | | | | | D | B |
| 105 | | | | | D | |
| 106 | | | | | B | A |
| 107 | | | | | B | A |
| 108 | | | | | D | A |
| 109 | | | | | D | B |
| 110 | | | | | D | C |
| 111 | | | | | D | B |
| 112 | | | | | D | C |
| 113 | | | B | B | | |
| 114 | | | | | D | B |
| 115 | | | | | D | A |
| 116 | | | | | C | A |
| 117 | | | | | C | B |
| 118 | | | | | D | C |
| 119 | | | | | C | A |
| 120 | | | | | D | C |
| 121 | | | | | B | A |
| 122 | | | | | D | C |
| 123 | | | A | A | | |
| 124 | | | C | C | | |
| 125 | | | D | D | | |
| 126 | | | D | D | | |
| 127 | | | D | C | | |
| 128 | | | D | C | | |
| 197 | B | B | | | | |
| 198 | B | B | | | | |
| 199 | C | C | | | | |
| 200 | C | C | | | | |
| 201 | C | C | | | | |
| 202 | C | C | | | | |
| 203 | D | D | | | | |
| 204 | C | C | | | | |
| 205 | C | C | | | | |
| 206 | D | D | | | | |
| 207 | D | D | | | | |
| 208 | C | C | | | | |
| 209 | C | C | | | | |
| 210 | B | A | | | | |
| 211 | A | A | B | A | A | A |
| 212 | B | A | | | | |
| 213 | B | B | | | | |
| 214 | B | A | B | A | B | A |
| 215 | D | D | | | | |
| 216 | D | D | | | | |
| 217 | D | D | | | | |
| 218 | D | D | | | | |
| 219 | C | B | | | | |
| 220 | D | D | | | | |
| 221 | C | B | | | | |
| 222 | D | D | | | | |
| 223 | D | D | | | | |
| 224 | C | B | | | | |
| 225 | C | B | | | | |
| 226 | D | D | | | D | D |
| 227 | D | C | | | C | C |
| 228 | D | B | | | B | A |
| 229 | D | C | | | B | A |
| 230 | D | C | | | B | A |
| 231 | D | C | | | B | A |
| 232 | D | D | | | | |
| 233 | D | C | | | B | A |
| 234 | D | B | | | C | B |
| 235 | D | C | | | C | A |
| 236 | C | A | | | B | A |
| 237 | B | A | | | A | A |
| 238 | D | C | | | D | D |
| 239 | | | | | C | A |
| 240 | | | | | A | A |
| 241 | | | | | D | D |
| 242 | | | | | D | D |
| 243 | | | | | D | D |
| 244 | | | | | B | A |
| 245 | | | | | C | B |
| 246 | | | | | D | D |
| 247 | | | | | D | C |
| 248 | | | | | D | D |
| 249 | | | | | D | C |
| 250 | | | | | D | D |
| 251 | | | | | D | D |
| 252 | | | | | D | D |
| 253 | | | | | D | C |
| 254 | | | | | B | B |
| 255 | | | | | A | A |
| 256 | | | | | D | C |
| 257 | | | | | B | A |
| 258 | | | | | D | C |
| 259 | | | | | A | A |
| 260 | | | | | D | D |
| 261 | | | | | C | B |
| 262 | | | | | D | C |
| 263 | | | | | D | D |
| 264 | | | | | D | D |
| 265 | | | | | D | D |
| 278 | | | | | A | A |
| 279 | | | | | C | B |
| 280 | | | | | D | C |
| 281 | | | | | D | C |
| 282 | | | | | D | D |
| 283 | | | | | D | D |
| 284 | | | | | D | D |
| 285 | | | | | D | D |
| 286 | | | | | D | D |
| 287 | | | | | D | D |
| 288 | | | | | D | D |
| 289 | | | | | D | D |
| 290 | | | | | D | C |
| 291 | | | | | D | D |

TABLE 5

| | |
|---|---|
| | % AR protein degradation in VCaP^{a} |
| Cpd. | Cells (μM) |

| No. | 0.001 | 0.01 | 0.1 | 1 |
|---|---|---|---|---|
| — | 0 | 0 | 0 | 0 |
| 336 | 3 | −5 | −5 | 4 |
| 337 | 18 | 12 | 9 | 29 |
| 338 | −1 | −12 | 8 | 93 |

### VI. References

(1) Hamdy et al., “Outcomes after Monitoring, Surgery, or Radiotherapy for Localized Prostate Cancer,” N Engl J Med, 2016, 375, 1415-1424.
(2) Litwin, M. S.; Tan, H. J. The Diagnosis and Treatment of Prostate Cancer. JAMA, 2017, 317, 2532-2542.
(3) Karantanos et al., “Prostate cancer progression after androgen deprivation therapy: mechanisms of castrate resistance and novel therapeutic approaches,” Oncogene. 2013, 32, 5501-511.
(4) Harris et al., “Androgen deprivation therapy: progress in understanding mechanisms of resistance and optimizing androgen depletion,” Nat Clin Pract Urol, 2009, 6, 76-85.
(5) Narayanan et al., “Destroying the androgen receptor (AR)-potential strategy to treat advanced prostate cancer,” Oncoscience. 2017, 4, 175-177.
(6) Crowder et al., “Nuclear Androgen Receptor Regulates Testes Organization and Oocyte Maturation in Zebrafish,” Endocrinology. 2018, 159, 980-993.
(7) Sundén et al., “Synthesis and Biological Evaluation of Second-Generation Tropanol-Based Androgen Receptor Modulators,” J. Med. Chem. 2015, 58, 1569-1574.
(8) Oksala et al., “A Novel Nonsteroidal Compound for the Treatment of Castration-Resistant Prostate Cancer by blocking the Androgen Receptor and Inhibiting CYP17A1,” J Steroid Biochem Mol Biol. 2018, doi: 10.1016/j.jsbmb.2018.02.004.
(9) Watson et al., “Emerging mechanisms of resistance to androgen receptor inhibitors in prostate cancer,” Nat Rev Cancer. 2015, 15, 701-711.
(10) Guo et al., “Discovery of Aryloxy Tetramethylcyclobutanes as Novel Androgen Receptor Antagonists,” J. Med. Chem. 2011, 54, 7693-7704.
(11) Moilanen et al., “Discovery of ODM-201, a new generation androgen receptor inhibitor targeting resistance mechanisms to androgen signaling-directed prostate cancer therapies,” Sci Rep. 2015, 5, 12007.
(12) Guerrini et al., “A New Avenue toward Androgen Receptor Pan-antagonists: C2 Sterically Hindered Substitution of Hydroxy-propanamides,” J. Med. Chem. 2014, 57, 7263-7279.
(13) Jung et al., “Structure-activity relationship for thiohydantoin androgen receptor antagonists for castration-resistant prostate cancer (CRPC),” J. Med. Chem. 2010, 53, 2779-2796.
(14) Yamamoto et al., “Design, synthesis, and biological evaluation of 4-arylmethyl-1-phenylpyrazole and 4-aryloxy-1-phenylpyrazole derivatives as novel androgen receptor antagonists,” Bioorg Med Chem. 2012, 20, 2338-2352.
(15) Balbas et al., “Overcoming mutation-based resistance to antiandrogens with rational drug design,” Elife. 2013, 2, e00499.
(16) Lottrup et al., “Identification of a novel androgen receptor mutation in a family with multiple components compatible with the testicular dysgenesis syndrome,” J Clin Endocrinol Metab. 2013, 98, 2223-2229.
(17) Zhu et al., “BMI1 regulates androgen receptor in prostate cancer independently of the polycomb repressive complex 1,” Nat Commun. 2018, 9, 500.
(18) Munuganti et al., “Identification of a potent antiandrogen that targets the BF3 site of the androgen receptor and inhibits enzalutamide-resistant prostate cancer,” Chem Biol. 2014, 21, 1476-485.
(19) Raina et al., “PROTAC-induced BET protein degradation as a therapy for castration-resistant prostate cancer,” Proc Natl Acad Sci USA. 2016, 113, 7124-7129.
(20) Zhou et al., “Discovery of a Small-Molecule Degrader of Bromodomain and Extra-Terminal (BET) Proteins with Picomolar Cellular Potencies and Capable of Achieving Tumor Regression,” J. Med. Chem. 2018, 61, 462-481.
(21) Gadd et al., “Structural basis of PROTAC cooperative recognition for selective protein degradation,” Nat Chem. Biol. 2017, 13, 514-521.
(22) Toure et al., “Small-molecule PROTACS: new approaches to protein degradation,” Angew. Chem. Int. Edn. 2016, 55, 1966-1973.
(23) Qin et al., “Discovery of QCA570 as an Exceptionally Potent and Efficacious Proteolysis Targeting Chimera (PROTAC) Degrader of the Bromodomain and Extra-Terminal (BET) Proteins Capable of Inducing Complete and Durable Tumor Regression,” J. Med. Chem. 2018, 61, 6685-6704.
(24) Hatcher et al., “Development of Highly Potent and Selective Steroidal Inhibitors and Degraders of CDK8, “ACS Med. Chem. Lett. 2018, 9, 540-545.
(25) Gollavilli et al., “EWS/ETS-Driven Ewing Sarcoma Requires BET Bromodomain Proteins,” Cancer Res. 2018, 78, 4760-4773.
(26) Bondeson et al., “Targeted Protein Degradation by Small Molecules. Annu Rev Pharmacol Toxicol,” 2017, 57, 107-123.
(27) Salami et al., “Androgen receptor degradation by the proteolysis-targeting chimera ARCC-4 outperforms enzalutamide in cellular models of prostate cancer drug resistance,” Commun Biol. 2018, 1, 100.
(28) Pal et al., “Identification of mechanisms of resistance to treatment with abiraterone acetate or enzalutamide in patients with castration-resistant prostate cancer (CRPC),” Cancer. 2018, 124, 1216-1224.
(29) Wang et al., “Blocking the Feedback Loop between Neuroendocrine Differentiation and Macrophages Improves the Therapeutic Effects of Enzalutamide (MDV3100) on Prostate Cancer,” Clin Cancer Res. 2018, 24, 708-723.
(30) Gustafson et al., “Small-Molecule-Mediated Degradation of the Androgen Receptor through Hydrophobic Tagging,” Angew. Chem. Int. Ed. 2015, 54, 9659-9662.
(31) Shibata et al., “Development of Protein Degradation Inducers of Androgen Receptor by Conjugation of Androgen Receptor Ligands and Inhibitor of Apoptosis Protein Ligands,” J. Med. Chem. 2018, 61, 543-575.
(32) Crew et al., US 20170327469 A1
(33) Pereira de Jésus-Tran et al., “Comparison of crystal structures of human androgen receptor ligand-binding domain complexed with various agonists reveals molecular determinants responsible for binding affinity,” Protein Sci. 2006, 15, 987-999.
(34) Galdeano et al., “Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities, “J. Med. Chem. 2014, 57, 8657-8663.
(35) Soares et al., “Group-Based Optimization of Potent and Cell-Active Inhibitors of the von Hippel-Lindau (VHL) E3 Ubiquitin Ligase: Structure-Activity Relationships Leading to the Chemical Probe (2S,4R)-1-((S)-2-(1-Cyanocyclopropanecarboxamido)-3,3-dimethylbutanoyl)-4-hydroxy-N-(4-(4-methylthiazol-5-yl)benzyl)pyrrolidine-2-carboxamide (VH298),” J. Med. Chem. 2018, 61, 599-618.
(36) Buckley et al., “Targeting the von Hippel-Lindau E3 ubiquitin ligase using small molecules to disrupt the VHL/HIF-1α interaction, “J. Am. Chem. Soc. 2012, 134, 4465-4468.
(37) Frost et al., “Potent and selective chemical probe of hypoxic signalling downstream of HIF-alpha hydroxylation via VHL inhibition,” Nat Commun, 2016, 7, 13312-13312.
(38) Berlin et al., WO2016149668A1
(39) Ishoey et al., “Translation Termination Factor GSPT1 Is a Phenotypically Relevant Off-Target of Heterobifunctional Phthalimide Degraders,” ACS Chem. Biol. 2018, 13, 553-560.
(40) Powell et al., “Chemically Induced Degradation of Anaplastic Lymphoma Kinase (ALK),” J. Med. Chem. 2018, 61, 4249-4255.
(41) Liu et al., “Melatonin Inhibits Androgen Receptor Splice Variant-7 (AR-V7)-Induced Nuclear Factor-Kappa B (NF-κB) Activation and NF-κB Activator-Induced AR-V7 Expression in Prostate Cancer Cells: Potential Implications for the Use of Melatonin in Castration-Resistant Prostate Cancer (CRPC) Therapy,” Int J Mol Sci. 2017, 18, E¹¹³⁰.
(42) Sun et al., “Design, synthesis, and characterization of a potent, nonpeptide, cell-permeable, bivalent Smac mimetic that concurrently targets both the BIR2 and BIR3 domains in XIAP,” J. Am. Chem. Soc. 2007, 129, 15279-15294.
(43) Lu et al., “SM-164: a novel, bivalent Smac mimetic that induces apoptosis and tumor regression by concurrent removal of the blockade of cIAP-1/2 and XIAP,” Cancer Res. 2008, 68, 9384-9393.
(44) Bai et al., “Targeted Degradation of BET Proteins in Triple-Negative Breast Cancer,” Cancer Res. 2017, 77, 2476-2487.
(45) Stols et al., “A new vector for high-throughput, ligation-independent cloning encoding a tobacco etch virus protease cleavage site, “Protein Expr Purif. 2002, 25, 8-15.
(46) Benoit, et al., “Seamless Insert-Plasmid Assembly at High Efficiency and Low Cost,” PLoS One. 2016, 11, e0153158.

It is to be understood that the foregoing embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I:
A-L-B¹ I,
or a pharmaceutically acceptable salt or solvate thereof, wherein:
A is selected from the group consisting of: 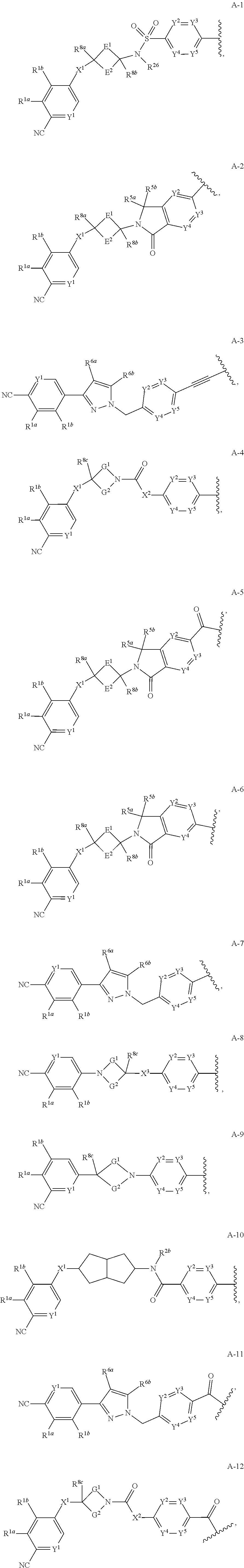 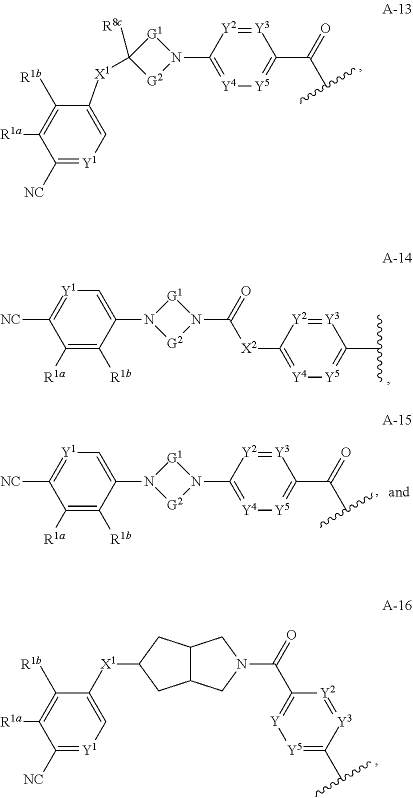
Y¹ is selected from the group consisting of —C(R^{1c})═ and —N═;
R^{1a}, R^{1b}, and R^{1c} are independently selected from the group consisting of hydrogen, halo, C₁-C₃ alkyl, and C₁-C₃ haloalkyl;
X¹ is selected from the group consisting of —O— and —N(R^{2a})—;
R^{2a} and R^{2b} are independently selected from the group consisting of hydrogen, C₁-C₄ alkyl, and C₃-C₆ cycloalkyl;
E¹ is —(CR^{3a}R^{3b})ₐ—;
E² is —(CR^{3c}R^{3d})_{b}—;
a and b are independently 1, 2, or 3;
each R^{3a}, R^{3b}, R^{3c}, and R^{3d} is independently selected from the group consisting of hydrogen and C₁-C₃ alkyl;
Y² is selected from the group consisting of —C(R^{4a})═ and —N═;
Y³ is selected from the group consisting of —C(R^{4b})═ and —N═;
Y⁴ is selected from the group consisting of —C(R^{4c})═ and —N═;
Y⁵ is selected from the group consisting of —C(R^{4d})═ and —N═;
R^{4a}, R^{4b}, R^{4c}, and R^{4d} are independently selected from the group consisting of hydrogen, halo, C₁-C₃ alkyl, C₁-C₃ haloalkyl, and C₁-C₃ alkoxy;
R^{8a} and R^{8b} are independently selected from the group consisting of hydrogen and C₁-C₃ alkyl; or R^{8a} and R^{8b} taken together form a C₁-C₃ alkylenyl;
R^{5a} and R^{5b} are independently selected from the group consisting of hydrogen and C₁-C₃ alkyl;
R^{6a} and R^{6b} are independently selected from the group consisting of hydrogen and C₁-C₃ alkyl; or R^{6a} and R^{6b} taken together with the carbon atoms to which they are attached from a C₅-C₇ cycloalkyl;
G¹ is —(CR^{7a}R^{7b})_{f}—;
G² is —(CR^{7c}R^{7d})_{g}—;
each R^{7a}, R^{7b}, R^{7c}, and R^{7d} is independently selected from the group consisting of hydrogen and C₁-C₃ alkyl; or one of R^{7a} and one of R^{7c} taken together with the carbon atoms to which they are attached form a C₁-C₃ alkylenyl or C₁-C₃ heteroalkylenyl; or one of R^{7a} and one of R^{7b} taken together with the carbon atom to which they are attached form a C₃-C₆ cycloalkyl;
f and g are independently 1, 2, or 3;
X² is selected from the group consisting of —O— and —N(R^{2c})—; or X² is absent, i.e., X² is a bond;
R^{2c} is selected from the group consisting of hydrogen, C₁-C₄ alkyl, and C₃-C₆ cycloalkyl
R^{8c} is selected from the group consisting of hydrogen and C₁-C₃ alkyl;
X³ is selected from the group consisting of —O— and —N(R^{2b})—;
L is -J¹-J²-J³-J⁴-J⁵-,
wherein J¹ is attached to A;
J¹ is selected from the group consisting of alkylenyl, cycloalkylenyl and heterocyclenyl; or
J¹ is absent;
J² is selected from the group consisting of —C(═O)—, —C(═O)NH—, —(CH₂)ₒ—, —CH═CH—, and —C≡C—;
o is 0, 1, 2, or 3;
J³ is selected from the group consisting of alkylenyl, heteroalkylenyl, cycloalkylenyl, heterocyclenyl, phenylenyl, and heteroarylenyl; or
J³ is absent;
J⁴ is selected from the group consisting of alkylenyl, cycloalkylenyl, and heterocyclenyl; or
J⁴ is absent;
J⁵ is selected from the group consisting of —C≡C—, —(CH₂)ₚ—, —O—, —N(R¹⁰)—, and —C(═O)—;
p is 0, 1, 2, or 3;
R¹⁰ is selected from the group consisting of hydrogen and C₁-C₃ alkyl;
B¹ is selected from the group consisting of: 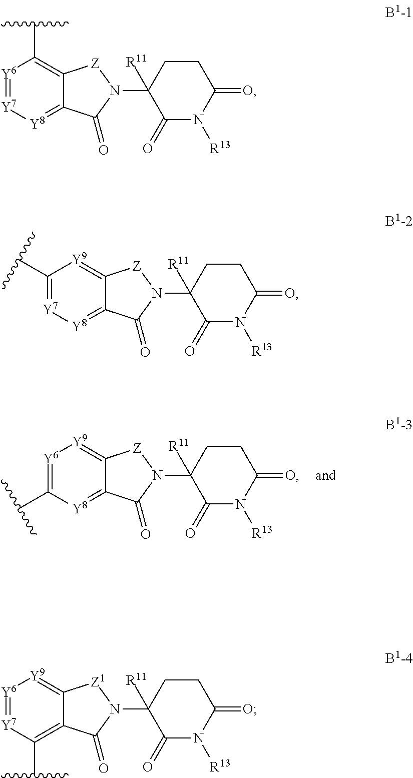
Y⁶ is selected from the group consisting of —C(R^{10a})═ and —N═;
Y⁷ is selected from the group consisting of —C(R^{10b})═ and —N═;
Y⁸ is selected from the group consisting of —C(R^{10c})═ and —N═;
Y⁹ is selected from the group consisting of —C(R^{10d})═ and —N═;
R^{10a}, R^{11b}, R^{10b}, and R^{10d} are independently selected from the group consisting of hydrogen, halo, C₁-C₃ alkyl, C₁-C₃ haloalkyl, and C₁-C₃ alkoxy;
R¹¹ is selected from the group consisting of hydrogen, deuterium, fluoro, and C₁-C₃alkyl;
Z is selected from the group consisting of —CR^{12a}R^{12b}— and —C(═O)—;
Z¹ is —CR^{12a}R^{12b}—;
R^{12a} and R^{12b} are independently selected from the group consisting of hydrogen and C₁-C₃ alkyl; or R^{12a} and R^{12b} taken together with the carbon to which they are attached from a C₃-C₆ cycloalkyl;
R¹³ is selected from the group consisting of hydrogen and C₁-C₃ alkyl.

2. The compound of claim 1, or a pharmaceutically acceptable salt or solvate thereof, wherein A is selected from the group consisting of: 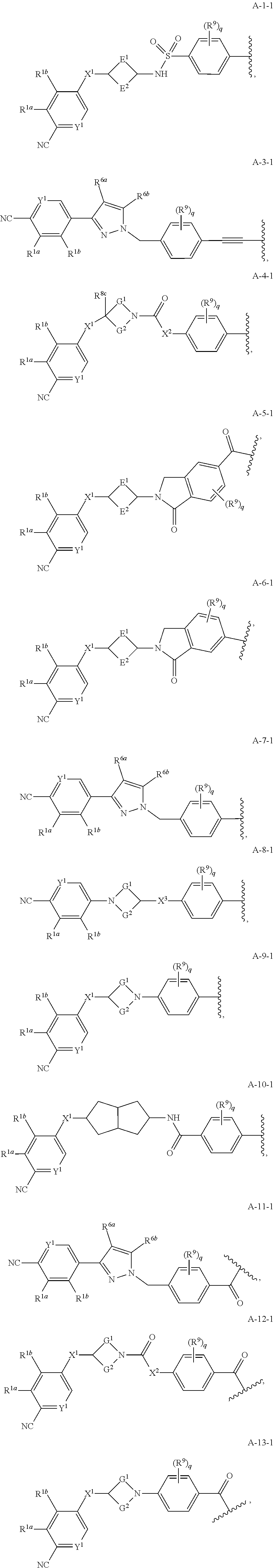 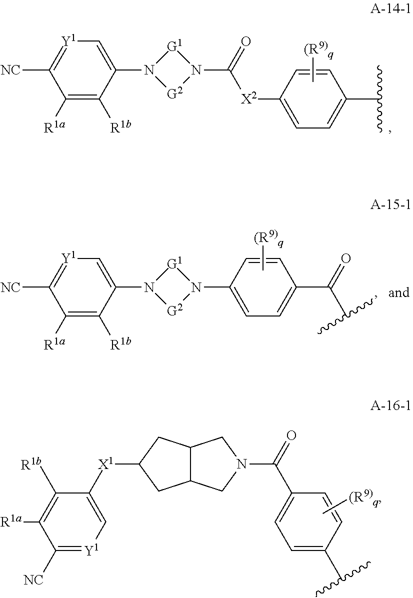
each R⁹ is independently selected from the group consisting of halo, C₁-C₃ alkyl, C₁-C₃ haloalkyl, and C₁-C₃ alkoxy; and
q is 0, 1, or 2.

3. The compound of claims 1 or 2, or a pharmaceutically acceptable salt or solvate thereof, wherein E¹ and E² are independently selected from the group consisting of —CH₂—, —C(CH₃)H—, —C(CH₃)₂—, —CH₂CH₂—, and —C(CH₃)(H)CH₂—.

4. The compound of any one of claims 1 or 2, or a pharmaceutically acceptable salt or solvate thereof, wherein G¹ and G² are independently selected from the group consisting of —CH₂—, —C(CH₃)H—, —C(CH₃)₂—, —CH₂CH₂—, —C(CH₃)(H)CH₂—, —CH₂CH₂CH₂—, and —C(CH₃)(H)CH₂CH₂—.

5. The compound of any one of claims 1-4, or a pharmaceutically acceptable salt or solvate thereof, wherein X¹ is —O—.

6. The compound of any one of claims 1-4, or a pharmaceutically acceptable salt or solvate thereof, wherein X¹ is —N(H)—.

7. The compound of any one of claims 1-4, or a pharmaceutically acceptable salt or solvate thereof, wherein X² is a bond.

8. The compound of any one of claims 1-4, or a pharmaceutically acceptable salt or solvate thereof, wherein X² is —O—.

9. The compound of any one of claims 1-4, or a pharmaceutically acceptable salt or solvate thereof, wherein X³ is —O—.

10. The compound of any one of claims 1-4, or a pharmaceutically acceptable salt or solvate thereof, wherein X³ is —N(H)—.

11. The compound of any one of claims 1-10, or a pharmaceutically acceptable salt or solvate thereof, wherein Y¹ is —CH═.

12. The compound of any one of claims 1-11, or a pharmaceutically acceptable salt or solvate thereof, wherein R^{1b} is hydrogen.

13. The compound of any one of claims 1-12, or a pharmaceutically acceptable salt or solvate thereof, wherein R^{1a} is chloro.

14. The compound of claim 1, or a pharmaceutically acceptable salt or solvate thereof, wherein A is selected from the group consisting of:

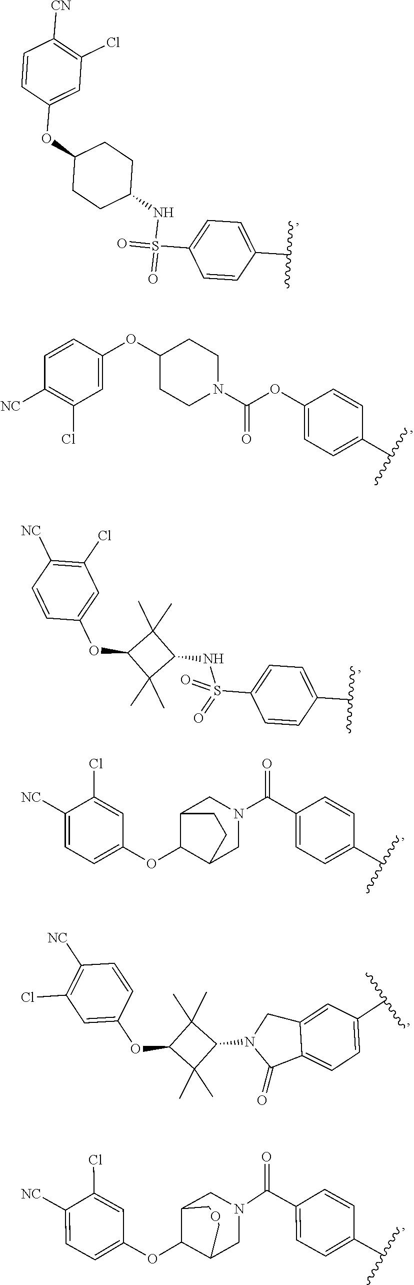

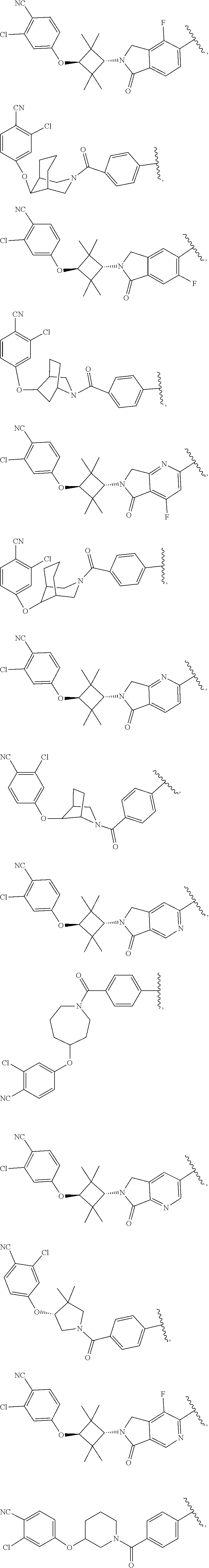

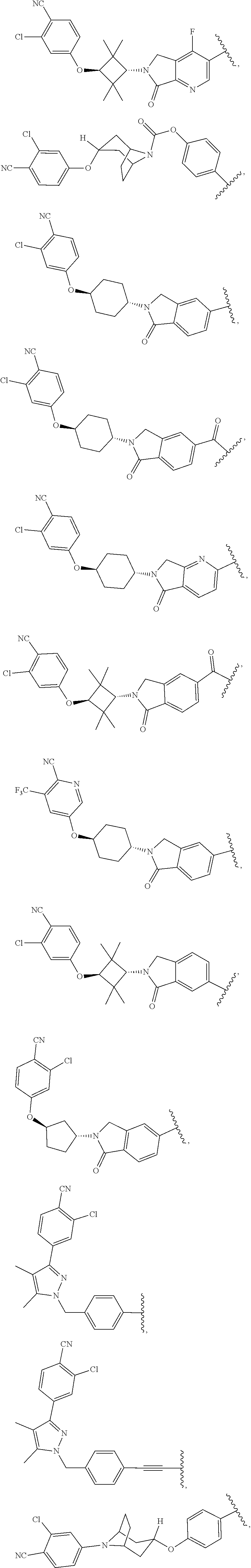

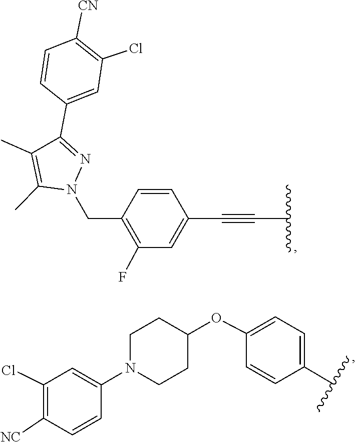 [SMILES: *C#Cc1ccc(Cn2nc(-c3ccc(C#N)c(Cl)c3)c(C)c2C)c(F)c1 ; *c1ccc(OC2CCN(c3ccc(C#N)c(Cl)c3)CC2)cc1]

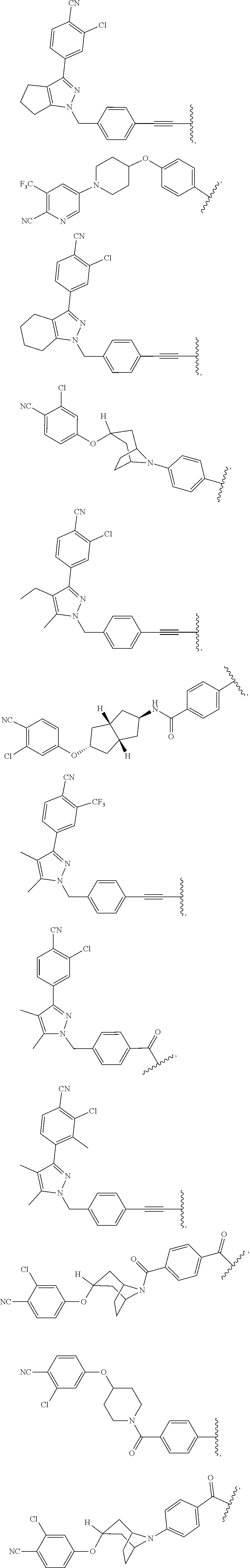

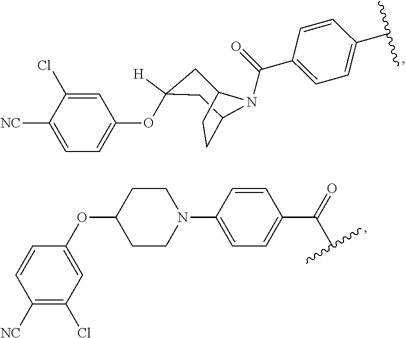 [SMILES: *c1ccc(C(=O)N2C3CCC2CC(Oc2ccc(C#N)c(Cl)c2)C3)cc1 ; *C(=O)c1ccc(N2CCC(Oc3ccc(C#N)c(Cl)c3)CC2)cc1]

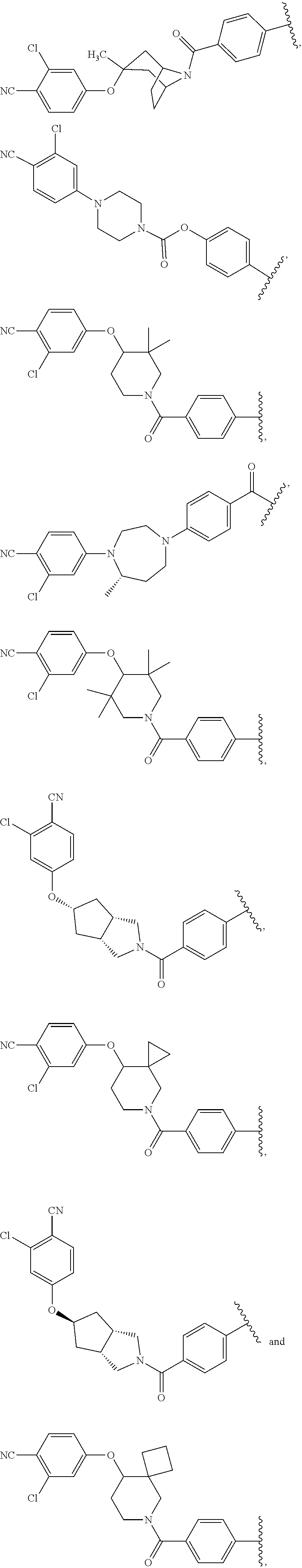

15. The compound of any one of claims 1-14, or a pharmaceutically acceptable salt or solvate thereof, wherein J¹ is heterocyclenyl.

16. The compound of claim 15, or a pharmaceutically acceptable salt or solvate thereof, wherein:
J¹ is selected from the group consisting of: 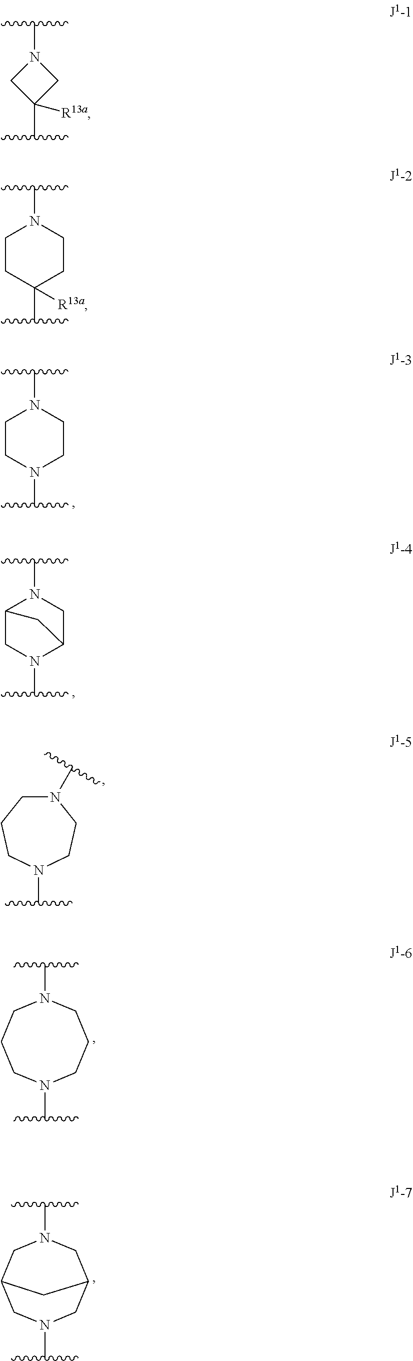 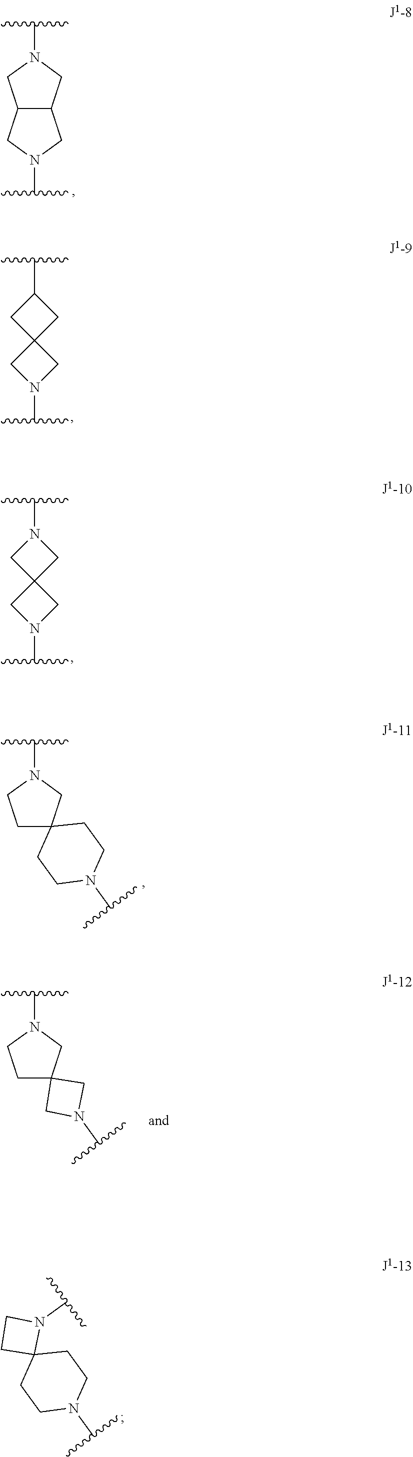 [SMILES: *N1CC2CN(*)CC2C1 ; *C1CC2(C1)CN(*)C2 ; *N1CC2(C1)CN(*)C2 ; *N1CCC2(CC1)CCN(*)C2 ; *N1CCC2(C1)CN(*)C2 ; *N1CCC2(CC1)CCN2*]
and
R^{13a} is selected from the group consisting of hydrogen, halo, hydroxy, cyano, C₁-C₄ alkyl, C₃-C₆ cycloalkyl, and C₁-C₄ alkoxy.

17. The compound of any one of claims 1-14, or a pharmaceutically acceptable salt or solvate thereof, wherein J¹ is cycloalkylenyl.

18. The compound of any one of claims 1-14, or a pharmaceutically acceptable salt or solvate thereof, wherein J¹ is absent.

19. The compound of any one of claims 1-18, or a pharmaceutically acceptable salt or solvate thereof, wherein J² is selected from the group consisting of —C(═O)—, —C(═O)NH—, —(CH₂)ₒ— and —C≡C—; and o is 0, 1, or 2.

20. The compound of claim 19, or a pharmaceutically acceptable salt or solvate thereof, wherein J² is —(CH₂)ₒ—; and o is 0.

21. The compound of claim 19, or a pharmaceutically acceptable salt or solvate thereof, wherein J² is —(CH₂)ₒ—; and o is 1.

22. The compound of claim 19, or a pharmaceutically acceptable salt or solvate thereof, wherein J² is —C≡C—.

23. The compound of any one of claims 1-22, or a pharmaceutically acceptable salt or solvate thereof, wherein J³ is selected from the group consisting of cycloalkylenyl and heterocyclenyl.

24. The compound of claim 23, or a pharmaceutically acceptable salt or solvate thereof, wherein:
J³ is selected from the group consisting of: 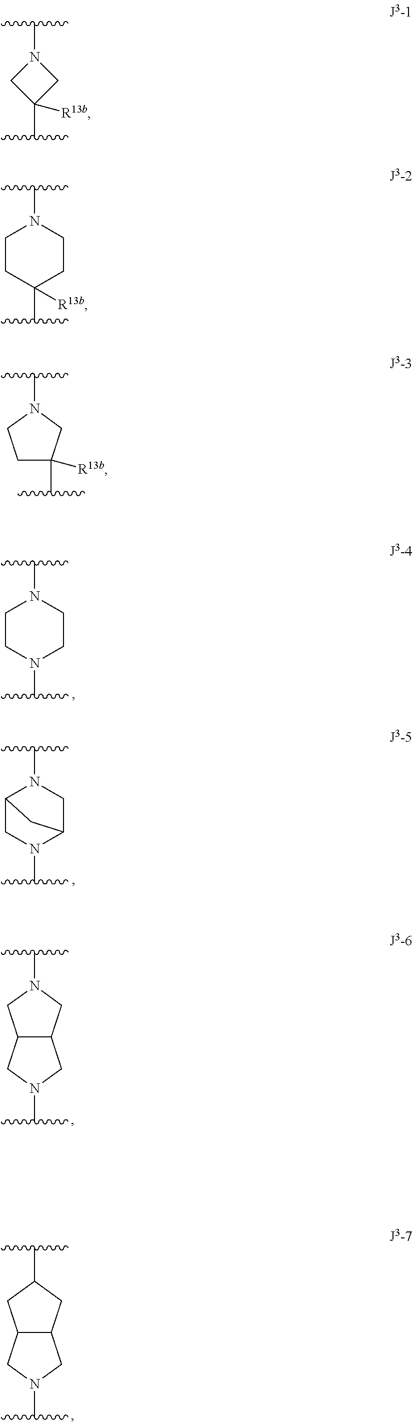 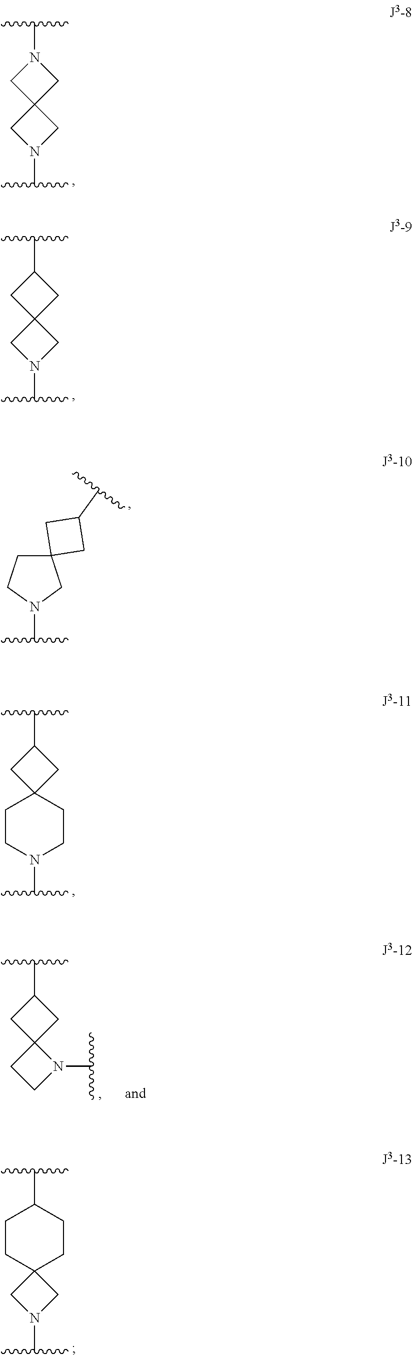 [SMILES: *N1CC2(C1)CN(*)C2 ; *C1CC2(C1)CN(*)C2 ; *C1CC2(CCN(*)C2)C1 ; *C1CC2(CCN(*)CC2)C1 ; *C1CC2(CCN2*)C1 ; *C1CCC2(CC1)CN(*)C2]
and
R^{13b} is selected from the group consisting of hydrogen, halo, hydroxy, cyano, C₁-C₄ alkyl, C₃-C₆ cycloalkyl, and C₁-C₄ alkoxy.

25. The compound of any one of claims 1-22, or a pharmaceutically acceptable salt or solvate thereof, wherein J³ is absent.

26. The compound of any one of claims 1-25, or a pharmaceutically acceptable salt or solvate thereof, wherein J⁴ is selected from the group consisting of alkylenyl, cycloalkylenyl, and heterocyclenyl.

27. The compound of any one of claims 1-25, or a pharmaceutically acceptable salt or solvate thereof, wherein J⁴ is absent.

28. The compound of any one of claims 1-27, or a pharmaceutically acceptable salt or solvate thereof, wherein J⁵ is selected from the group consisting of —C≡C—, —(CH₂)ₚ—, —N(H)—, and —C(═O)—; and p is 0, 1, or 2.

29. The compound of any one of claims 1-28, or a pharmaceutically acceptable salt or solvate thereof, wherein B¹ is B¹-1.

30. The compound of any one of claims 1-28, or a pharmaceutically acceptable salt or solvate thereof, wherein B¹ is B¹-2.

31. The compound of any one of claims 1-28, or a pharmaceutically acceptable salt or solvate thereof, wherein B¹ is B¹-3.

32. The compound of any one of claims 1-28, or a pharmaceutically acceptable salt or solvate thereof, wherein B¹ is selected from the group consisting of:

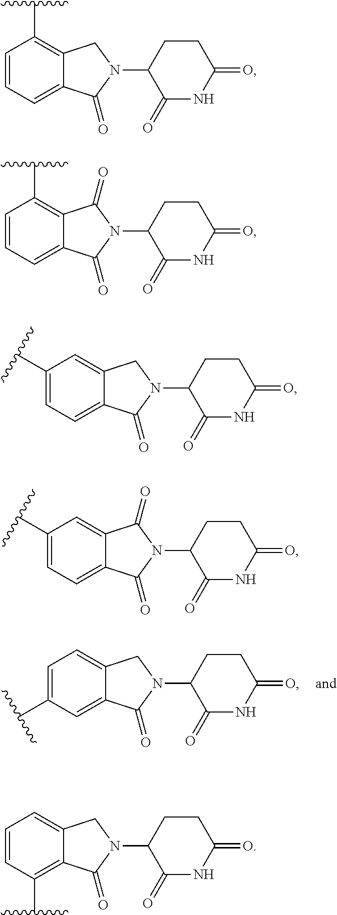

33. The compound of claim 32, or a pharmaceutically acceptable salt or solvate thereof, wherein B¹ is:

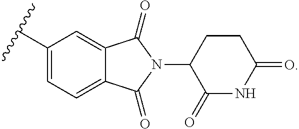 [SMILES: *c1ccc2c(c1)C(=O)N(C1CCC(=O)NC1=O)C2=O]

34. The compound of claim 1, or a pharmaceutically acceptable salt or solvate thereof, selected from any one of more of the compounds of Table 1.

35. A pharmaceutical composition comprising the compound of any one of claims 1-34, or a pharmaceutically acceptable salt or solvate thereof, and a pharmaceutically acceptable excipient.

36. A method of treating cancer in a subject in need thereof, the method comprising administering to the subject a therapeutically effective amount of the compound of any one of claims 1-34, or a pharmaceutically acceptable salt or solvate thereof.

37. The method of claim 36, wherein the cancer is breast cancer or prostate cancer.

38. The pharmaceutical composition of claim 35 for use in treating cancer.

39. The pharmaceutical composition of claim 38, wherein the cancer is breast cancer or prostate cancer.

40. A compound of any one of claims 1-34, or a pharmaceutically acceptable salt or solvate thereof, for use in treating of cancer.

41. The compound for use of claim 40, wherein the cancer is breast cancer or prostate cancer.

42. Use of a compound of any one of claims 1-34, or a pharmaceutically acceptable salt or solvate thereof, for the manufacture of a medicament for treatment of cancer.

43. The use of claim 40, wherein the cancer is breast cancer or prostate cancer.

44. A method of reducing androgen receptor protein within a cell of a patient in need thereof, the method comprising administering to the subject a compound of any one of claims 1-34, or a pharmaceutically acceptable salt or solvate thereof.

45. A kit comprising the compound of any one of claims 1-34, or a pharmaceutically acceptable salt or solvate thereof, and instructions for administering the compound, or a pharmaceutically acceptable salt or solvate thereof, to a subject having cancer.
